(19) United States
(12) Patent Application Publication
(10) Pub. No.: US 2023/0313187 A1
(43) Pub. Date: Oct. 5, 2023

(54) MODIFICATIONS OF MAMMALIAN CELLS USING ARTIFICIAL MICRO-RNA TO ALTER THEIR PROPERTIES AND THE COMPOSITIONS OF THEIR PRODUCTS

(71) Applicant: DNA TWOPOINTO INC., NEWARK, CA (US)

(72) Inventors: Jeremy MINSHULL, Los Altos, CA (US); Maggie LEE, San Jose, CA (US); Varsha SITARAMAN, San Mateo, CA (US); Oren BESKE, Aromas, CA (US); Ferenc BOLDOG, Newark, CA (US)

(73) Assignee: DNA TWOPOINTO INC., NEWARK, CA (US)

(21) Appl. No.: 18/044,057

(22) PCT Filed: Sep. 3, 2021
(86) PCT No.: PCT/US2021/049074

§ 371 Date: Mar. 3, 2023

Related U.S. Application Data
(60) Provisional application No. 63/074,803, filed on Sep. 4, 2020.
(60) Provisional application No. 63/111,139, filed on Nov. 9, 2020.

(51) Int. Cl. C12N 15/113; C12N 15/10

(52) U.S. Cl. CPC C12N 15/113; C12N 15/102; C12N 2310/141; C12N 2310/122

(57) ABSTRACT

The present invention provides methods and compositions for stable genetic modification of cultured mammalian cells. The genetic modifications can be used to produce cultured mammalian cells for therapeutic or diagnostic purposes.

### CROSS REFERENCE TO RELATED APPLICATIONS

The present application claims the benefit of U.S. 63/074,803 filed Sep. 4, 2020, and U.S. 63/111,139 filed Nov. 9, 2020, incorporated by reference in their entirety for all purposes.

### REFERENCE TO A SEQUENCE LISTING

The application includes an electronic sequence listing in a file named 565103WO_ST25.TXT, created Sep. 3, 2021, and containing 911,970 bytes, which is hereby incorporated by reference in its entirety for all purposes.

### BACKGROUND OF THE INVENTION

Introduction of heterologous nucleic acids into mammalian cells can be used to modify their properties, and the properties of molecules that they produce. Genetically modifiable properties of cultured mammalian cells include the glycosylation of proteins secreted by the cultured mammalian cell, proteolytic processing of proteins produced by the cultured mammalian call, intracellular trafficking of proteins produced by the cell, growth properties of the cell including which nutrients must be provided to the cell exogenously, and viability and susceptibility of the cells to apoptosis under various stresses including expression of high levels of heterologous proteins.

Stable genetic modifications of mammalian cells can be made by integrating a heterologous polynucleotide into the genome of the cultured mammalian cell. Heterologous DNA may be introduced into cells in different ways: by transfecting with naked plasmid DNA, by packaging the DNA into viral particles used to infect the cultured mammalian cells, or by transfecting cells with a transposon and its corresponding transposase.

Non-viral vector systems, including plasmid DNA, often suffer from inefficient cellular delivery, cellular toxicity and limited duration of transgene expression due to the lack of genomic insertion and resulting degradation and/or dilution of the vector in transfected cell populations. Transgenes delivered by non-viral approaches often form long, repeated arrays (concatemers) that are targets for transcriptional silencing by heterochromatin formation.

Viral packaging generally imposes limits on the size of the DNA that can be inserted. There are also safety concerns regarding viral integration sites, and the costs and complexities of viral manufacture.

The expression levels of genes encoded on a polynucleotide integrated into the genome of a cell depend on the configuration of sequence elements within the polynucleotide. The efficiency of integration and thus the number of copies of the polynucleotide that are integrated into each genome, and the genomic loci where integration occurs also influence the expression levels of genes encoded on the polynucleotide. The efficiency with which a polynucleotide may be integrated into the genome of a target cell can often be increased by placing the polynucleotide into a transposon. Transposons comprise two ends that are recognized by a transposase. The transposase acts on the transposon to excise it from one DNA molecule and integrate it into another. The DNA between the two transposon ends is transposed by the transposase along with the transposon ends. Heterologous DNA flanked by a pair of transposon ends, such that it is recognized and transposed by a transposase is referred to herein as a synthetic transposon. Introduction of a synthetic transposon and a corresponding transposase into the nucleus of a eukaryotic cell may result in transposition of the transposon into the genome of the cell. Transposon/transposase gene delivery platforms have the potential to overcome the limitations of naked DNA and viral delivery. The piggyBac-like transposons are attractive because of their unlimited gene cargo capacity, but Mariner transposons such as Sleeping Beauty, or hAT transposons such as TcBuster also provide efficient methods for integrating heterologous DNA into mammalian cell genomes.

The properties of mammalian cells can be favorably modified by inhibiting genes endogenous to the mammalian cells. RNA interference methods may be used to inhibit endogenous mammalian cell genes in order to favorably modify the properties of the mammalian cells. RNA interference is a promising technology for inhibiting endogenous genes of mammalian cells. The techniques currently being used suffer from limitations that prevent reliable long-term inhibition of gene expression. One widely used technique is to treat immune cells with siRNA, either by transfection of the siRNA or by treatment with chemically modified siRNA. This is useful as an experimental technique to determine phenotypic effects of gene knock-down or gene knock-out. RNA is labile, however, so any effects of siRNA administered as RNA are transient. A second technique is to transfect in genes encoding shRNAs which are operably linked to a promoter transcribed by RNA polymerase III. This technique is frequently limited by the variable efficacy of individual shRNA molecules, as well as the highly variable rate of random integration. The variable rate of random integration can be solved using lentiviral vectors, but the variability of shRNA efficacy is still highly problematic (Anastasov et. al., 2009. J. Hematop 2, 9-19. “Efficient shRNA delivery into B and T lymphoma cells using lentiviral vector-mediated transfer”).

MicroRNAs (miRNAs) are naturally occurring RNAs that are transcribed from their genes by RNA polymerase II. MicroRNAs comprise intramolecular double-stranded RNA hairpins, which are processed by cellular enzymes to produce a “guide strand” that is complementary to one or more mRNA targets. The guide strand is physically associated with the RISC complex, and acts through the RISC complex to inhibit expression of the target mRNA. Artificial miRNAs (amiRNAs) can be designed by using a natural scaffold and adapting it to produce guide strands that inhibit targets other than the natural target. Artificial miRNAs can also be transcribed by RNA polymerase III (Snyder et. al., 2009. Nucl. Acids. Res 37 e127 doi:10.1093/nar/gkp657. “RNA polymerase III can drive polycistronic expression of functional interfering RNAs designed to resemble microRNAs”). The use of miRNA scaffolds can improve the processing of interfering RNAs, but variability in effectiveness remains a challenge. There is thus a need in the art for a robust RNA interference method for the inhibition of genes endogenous to mammalian cells in order to modify the properties of mammalian cells, or of the proteins or other compounds that mammalian cells produce.

In some cases, it is advantageous to be able to modulate the expression of a heterologous polynucleotide within a mammalian cell. This can often be done directly by operably linking an inducible promoter to the heterologous polynucleotide to be expressed. There are circumstances, however, when expression of the heterologous polynucleotide should be inducible in one cell but constitutive in another. An example is when it is desired to produce a virus encoding a toxic gene intended for delivery to a target cell. Although the gene should be expressed constitutively in the target cell, expression in the cell line that is used to package the virus risks killing the packaging cell.

### SUMMARY OF THE INVENTION

Disclosed herein are methods and compositions for introducing into mammalian cells polynucleotides comprising artificial microRNAs to inhibit expression of heterologous polynucleotides.

Methods for modifying the genomes of mammalian cells to inhibit expression of endogenous genes are described. Mammalian cells may include mammalian cells cultured for the production of expressed proteins. They may also include immune cells including lymphocytes such as T-cells and B-cells and natural killer cells (NK cells), T-helper cells, antigen-presenting cells, dendritic cells, neutrophils and macrophages.

RNA interference methods may be used to inhibit expression of endogenous mammalian cell genes in order to favorably modify the properties of the mammalian cells. Here we describe methods for improving the efficiency of RNA interference: (i) the gene expressing the interfering RNA (for example the shRNA or amiRNA gene) may be incorporated into a transposon, wherein one or more copies of the transposon are integrated into transcriptionally active regions of the mammalian cell genome, and (ii) the interfering RNA comprises two or more different guide strands that are complementary to two or more different sequences within the same mRNA target. Providing two or more guide strands complementary to different sequences within the same mRNA target, either in a lentiviral vector or a transposon vector substantially improves the reliability of RNA interference.

Methods for designing polynucleotides for the inhibition of genes expressed in mammalian cells are described. A preferred polynucleotide for the inhibition of a target gene (the “inhibitory polynucleotide”) comprises two or more different hairpin sequences that can be expressed in the target mammalian cell to produce two or more different RNA guide strand sequences, each of which is complementary to a different region of the target mRNA. The first (guide) sequence comprises between 19 and 22 contiguous bases that are complementary to the target mRNA and the second (guide) sequence comprises between 19 and 22 contiguous bases that are complementary to the target mRNA. The first and second guide strand sequences are different from each other but complementary to the same target mRNA. Optionally the polynucleotide comprises a third hairpin sequence expressible in the target mammalian cell to produce an RNA guide strand sequence comprising between 19 and 22 contiguous bases that are complementary to the target mRNA and the first, second and third guide strand sequences are different from each other. Each hairpin sequence in the inhibitory polynucleotide comprises a guide strand sequence and a complementary passenger strand sequence. Each guide strand sequence is separated from its corresponding passenger strand sequence by a sequence that, in the expressed RNA, forms an unpaired loop of between 5 and 35 bases. Each passenger strand sequence comprises at least 19 bases that are at least 78% identical to the reverse complement of its corresponding guide strand sequence (i.e. within those 19 bases it comprises no more than 4 mismatches, including mutations, single base deletions or single base insertions, relative to the identical reverse complement of the corresponding guide strand sequence). The differences between the guide and passenger strand sequences are selected to favor processing of the transcribed hairpins by the mammalian RNA interference pathway and loading of the guide strand(s) into the RISC complex, to reduce expression of the target mRNA. Hairpin sequences of the invention (that is the combination of guide, loop and passenger strand sequences) in the polynucleotide are preferably sequences that are not naturally expressed sequences in mammalian cells, or from viruses that may infect mammalian cells. Hairpin sequences of the invention are preferably expressed from one or more artificial micro-RNAs.

The inhibitory polynucleotide comprises two or more hairpin sequences that are each operably linked to a heterologous promoter active in the target mammalian cell. Each hairpin sequence may be operably linked to the same promoter, or they may be linked to separate promoters. Preferably the promoter is transcribed by RNA polymerase II or RNA polymerase III, more preferably the promoter is transcribed by RNA polymerase II. In some embodiments, the promoter is an inducible promoter.

In some embodiments the inhibitory polynucleotide comprises (a) a segment encoding a multi-hairpin amiRNA sequence, wherein the segment comprises (i) a first guide strand sequence comprising a contiguous 19-22 nucleotide sequence that is perfectly complementary to a first target site of a natural mammalian cellular mRNA and a first passenger strand sequence comprising a contiguous 19-22 nucleotide sequence that is at least 78% complementary to the first guide strand sequence, wherein the first guide strand and first passenger strand sequence are separated by between 5 and 35 nucleotides; (ii) a second guide strand sequence comprising a contiguous 19-22 nucleotide sequence that is perfectly complementary to a second target site different than the first target site of the same natural mammalian cellular mRNA as the first guide strand sequence and a second passenger strand sequence comprising a contiguous 19-22 nucleotide sequence that is at least 78% complementary to the second guide strand sequence, wherein the second guide strand and second passenger strand sequence are separated by between 5 and 35 nucleotides, and wherein the first and second guide strand sequence are different from each other; and (b) a eukaryotic promoter that is active in a mammalian cell and is transcribed by RNA polymerase II or RNA polymerase III and that is operably linked to the segment encoding the amiRNA sequence, wherein the amiRNA sequence can be expressed and fold into multiple hairpins. The first passenger strand sequence may be the same length as the first guide strand sequence, or it may be shorter by 1-3 nucleotides. The first and second target sites in the mammalian cellular mRNA may have some overlap or not overlap. Optionally, the multi-hairpin amiRNA sequence reduces expression of the natural cellular mRNA to a greater extent that a control polynucleotide expressing tandem copies of the amiRNA hairpin comprising the first guide strand sequence or a control polynucleotide expressing tandem copies of the ami hairpin comprising the second guide strand sequence. Optionally, the segment encoding the multi-hairpin amiRNA sequence further comprises a third guide strand sequence comprising a contiguous 19 nucleotide sequence that is perfectly complementary to the same natural mammalian cellular mRNA as the first and second guide strand sequences and a third passenger strand sequence comprising a contiguous 19 nucleotide sequence that is at least 78% complementary to the third guide strand sequence, wherein the third guide strand and third passenger strand sequence are separated by between 5 and 35 nucleotides, and wherein the first, second and third guide strand sequences are different from each other. Optionally, the polynucleotide further comprises an open reading frame operably linked to the promoter, wherein the multi-hairpin amiRNA sequence is expressed from the promoter in a 3′ UTR following the open reading frame. Optionally, the open reading frame encodes a selectable marker, such as a fluorescent protein. Optionally, the selectable marker provides a growth advantage to the cell either by allowing the cell to synthesize a metabolically useful substance, or to survive in the presence of a harmful substance such as an antibiotic, enzyme inhibitor or cellular poison. Optionally, the selectable marker is selected from a dihydrofolate reductase, a glutamine synthetase, an aminoglycoside 3′-phosphotransferase, a puromycin acetyltransferase, a blasticidin acetyltransferase, a blasticidin deaminase, a hygromycin B phosphotransferase or a zeocin-binding protein. Optionally, the promoter is an EF1a promoter, a promoter from the immediate early genes 1, 2 or 3 of cytomegalovirus, a promoter for eukaryotic elongation factor 2, a glyceraldehyde 3-phosphate dehydrogenase promoter, an actin promoter, a phosphoglycerokinase promoter, a ubiquitin promoter, a herpes simplex virus thymidine kinase promoter or a simian virus 40 promoter. Optionally, the promoter is at least 95% or 100% identical to a nucleotide sequence selected from SEQ ID NOs: 310-399 and 404-40. Optionally, each passenger strand sequence is not complementary to its corresponding guide strand sequence at the position corresponding to the first base of the guide strand sequence. Optionally, each passenger strand sequence is not complementary to its corresponding guide strand sequence at the position corresponding to the twelfth base of the guide strand sequence. Optionally, each 5-35 nucleotide unstructured loop sequence between a guide strand sequence and its corresponding passenger strand sequence comprises a sequence selected from SEQ ID NOs: 241-250. Optionally, each guide strand-passenger strand hairpin further comprises additional sequences immediately to the 5′ and 3′ of the hairpin, wherein the additional sequence are SEQ ID NO: 255 to the 5′ and SEQ ID NO: 256 to the 3′, or SEQ ID NO: 257 to the 5′ and SEQ ID NO: 258 to the 3′, or SEQ ID NO: 259 to the 5′ and SEQ ID NO: 260 to the 3′, or SEQ ID NO: 261 to the 5′ and SEQ ID NO: 262 to the 3′, or SEQ ID NO: 263 to the 5′ and SEQ ID NO: 264 to the 3′, or SEQ ID NO: 265 to the 5′ and SEQ ID NO: 266 to the 3′, or SEQ ID NO: 267 to the 5′ and SEQ ID NO: 268 to the 3′, or SEQ ID NO: 269 to the 5′ and SEQ ID NO: 270 to the 3′.

Advantageous inhibitory polynucleotides are stably maintained in the mammalian cell, so that the target gene is permanently repressed. Preferably the inhibitory polynucleotide is integrated into the genome of the mammalian cell. To facilitate stable integration of the inhibitory polynucleotide into the genome of the mammalian cell it is advantageous to incorporate the hairpin sequences and regulatory elements required for their expression into a transposon such as a piggyBac-like transposon, or a Mariner transposons such as a Sleeping Beauty transposon, or an hAT transposon such as a TcBuster transposon, or into a viral vector such as a lentiviral vector. An advantageous inhibitory polynucleotide comprises two or more different hairpin sequences expressible in a mammalian cell, each hairpin sequence comprising a different sequence of at least 19 or 20 or 21 or 22 contiguous bases that are complementary to the target mRNA, wherein each hairpin is operably linked to a promoter that is active in a mammalian cell, and wherein the hairpins and their operably linked promoters are flanked by the inverted terminal repeats of a piggyBac-like transposon, or the inverted terminal repeats of a Mariner transposon such as a Sleeping Beauty transposon, or the inverted terminal repeats of an hAT transposon such as a TcBuster transposon such that the hairpins and their operably linked promoters are transposable by a corresponding transposase. Exemplary combinations of transposon ends are sequences selected from SEQ ID NOs: 421 and 422, or from SEQ ID NOs: 427 and 428, or from SEQ ID NOs: 431 and 432, or from SEQ ID NOs: 433 and 434, or from SEQ ID NOs: 439 and 440, or from SEQ ID NOs: 443 and 444, or from SEQ ID NOs: 564 and 447, or from SEQ ID NOs: 452 and 453, or from SEQ ID NOs: 456 and 457, or from SEQ ID NOs: 460 and 461, or from SEQ ID NOs: 528 and 529.

Alternatively, the hairpins and their operably linked promoters are flanked by the inverted terminal repeats of a lentivirus so that they can be packaged into a viral particle.

A method of the invention comprises introducing into a mammalian cell an inhibitory polynucleotide comprising two or more different hairpin sequences expressible in the mammalian cell to produce two or more different guide RNA sequences, each of which is complementary to a different region of the same target mRNA. Preferably the two or more different hairpin sequences are operably linked to the same promoter. For an inhibitory polynucleotide wherein the hairpin sequences are carried on a transposon vector, the method may further comprise introducing into the mammalian cell a corresponding transposase, either as protein or as a nucleic acid encoding the transposase. For an inhibitory polynucleotide wherein the hairpin sequences are carried on a viral vector, the method may further comprise packaging the polynucleotide into viral particles and contacting the mammalian cell with the viral particles.

A modified mammalian cell whose genome comprises an inhibitory polynucleotide comprising two or more different hairpin sequences expressible in the mammalian cell to produce two or more different guide RNA sequences each of which is complementary to a different region of the target mRNA are an aspect of the invention. A modified mammalian cell comprising an inhibitory polynucleotide that has been integrated through the action of a piggyBac-like transposase comprises at least two hairpins, each hairpin comprising a different sequence of at least 19 or 20 or 21 or 22 contiguous bases that are complementary to the a different region of the same target mRNA, and each hairpin is operably linked to a promoter that is active in a mammalian immune cell, wherein the hairpins and the promoter are flanked by the inverted terminal repeats of a piggyBac-like transposon. A modified mammalian cell, including a modified human immune cell comprising an inhibitory polynucleotide that has been integrated through the action of a Sleeping Beauty transposase comprises at least two hairpins, each hairpin comprising a different sequence of at least 19 or 20 or 21 or 22 contiguous bases that are complementary to the a different region of the same target mRNA, and each hairpin is operably linked to a promoter that is active in a mammalian immune cell, wherein the hairpins and the promoter are flanked by the inverted terminal repeats of a Sleeping Beauty transposon. A modified mammalian cell, including a modified human immune cell comprising an inhibitory polynucleotide that has been integrated through the action of a TcBuster transposase comprises at least two hairpins, each hairpin comprising a different sequence of at least 19 or 20 or 21 or 22 contiguous bases that are complementary to a different region of the same target mRNA, and each hairpin is operably linked to a promoter that is active in a mammalian immune cell, wherein the hairpins and the promoter are flanked by the inverted terminal repeats of a TcBuster transposon. A modified mammalian cell, including a modified human immune cell comprising an inhibitory polynucleotide that has been integrated through the action of a lentiviral system comprises at least two hairpins, each hairpin comprising a different sequence of at least 19 or 20 or 21 or 22 contiguous bases that are complementary to a different region of the same target mRNA, and each hairpin is operably linked to a promoter that is active in a mammalian immune cell, wherein the hairpins and the promoter are flanked by the inverted terminal repeats of a lentivirus. Preferably the immune cell whose genome comprises an inhibitory polynucleotide has improved proliferation, survival or functional properties relative to an immune cell whose genome does not comprise such an inhibitory polynucleotide.

Sequences of polynucleotides for effecting genomic modifications of mammalian cells are provided.

DHFR: The invention provides a polynucleotide comprising a) a segment encoding a multi-hairpin amiRNA sequence, wherein the segment comprises (i) a first guide strand sequence comprising a contiguous 19 nucleotide sequence that is perfectly complementary to a first target site in a natural mammalian cellular mRNA of SEQ ID NO: 11 and a first passenger strand sequence comprising a contiguous 19 nucleotide sequence that is at least 78% complementary to the first guide strand sequence, wherein the first guide strand and first passenger strand sequence are separated by between 5 and 35 nucleotides; (ii) a second guide strand sequence comprising a contiguous 19 nucleotide sequence that is perfectly complementary to a second target site different than the first target site in the same natural mammalian cellular mRNA as the first guide strand sequence and a second passenger strand sequence comprising a contiguous 19 nucleotide sequence that is at least 78% complementary to the second guide strand sequence, wherein the second guide strand and second passenger strand sequences are separated by between 5 and 35 nucleotides, and wherein the first and second guide strand sequences are different from each other; and b) a eukaryotic promoter that is active in a mammalian cell and is transcribed by RNA polymerase II or RNA polymerase III operably linked to the segment encoding the amiRNA sequence, wherein the amiRNA sequence can be expressed and fold into multiple hairpins; wherein the first and second guide strand sequences are selected from SEQ ID NOs: 82-84 and 607-616.

Optionally, the first guide strand sequence is a 19-22 nucleotide sequence perfectly complementary to the natural mammalian cellular mRNA and the first passenger strand sequence has the same length as the first guide sequence. Optionally, the first guide strand sequence is a 19-22 nucleotide sequence perfectly complementary to the natural mammalian cellular mRNA and the first passenger strand sequence is shorter than the first guide sequence. Optionally, the first and second target sites do not overlap. Optionally, the segment encoding the multi-hairpin amiRNA sequence further comprises a third guide strand sequence comprising a contiguous 19 nucleotide sequence that is perfectly complementary to the same natural mammalian cellular mRNA as the first and second guide strand sequences and a third passenger strand sequence comprising a contiguous 19 nucleotide sequence that is at least 78% complementary to the third guide strand sequence, wherein the third guide strand and third passenger strand sequences are separated by between 5 and 35 nucleotides, and wherein the first, second and third guide strand sequences are different from each other.

Optionally, the polynucleotides further comprises two transposon ends flanking the segment and the promoter, wherein the segment and the promoter are transposable by a corresponding transposase. Optionally, each transposon end comprises a sequence selected from SEQ ID NOs: 421 and 422, or from SEQ ID NOs: 427 and 428, or from SEQ ID NOs: 431 and 432, or from SEQ ID NOs: 433 and 434, or from SEQ ID NOs: 439 and 440, or from SEQ ID NOs: 443 and 444, or from SEQ ID NOs: 447 and 564, or from SEQ ID NOs: 452 and 453, or from SEQ ID NOs: 460 and 461, or from SEQ ID NOs: 528 and 529. Optionally, the polynucleotide comprises a sequence selected from SEQ ID NO: 210 and 627.

The invention further provides a mammalian cell comprising a polynucleotide as described above integrated into its genome. Optionally, the multi-hairpin amiRNA sequence is expressed and inhibits expression of the natural cellular mRNA, and whereby the growth of the cell in the presence of 50 nM methotrexate cell is inhibited relative to the growth of an otherwise identical cell whose genome does not comprise the multi-hairpin amiRNA.

The invention further provides a mammalian cell comprising a polynucleotide as described above integrated into its genome, wherein the multi-hairpin amiRNA sequence is expressed and inhibits expression of the natural cellular mRNA and a second polynucleotide comprising a gene encoding dihydrofolate reductase expressible in the mammalian cell, wherein expression of the gene compensates for the inhibition of the expression of the natural cellular mRNA, whereby the cell grows without the exogenous provision of hypoxanthine and thymidine and in the presence of at least 10 nM methotrexate. Optionally, the second polynucleotide further comprises a second gene expressible in the mammalian cell.

The invention further provides a method of selecting for integration of a nucleic acid encoding a target protein into the genome of a cell comprising: culturing a population of mammalian cells as described above in the presence of hypoxanthine and thymidine required by the cell to grow due to inhibition of expression of the natural cellular mRNA by the multi-hairpin amiRNA sequence; and transfecting the population of cells with a second polynucleotide comprising a gene encoding a dihydrofolate reductase expressible in the mammalian cells and a second gene encoding the target protein, wherein expression of the dihydrofolate reductase compensates for the inhibition of the expression of the natural cellular mRNA thereby restoring capacity to grow without hypoxanthine and thymidine and in the presence of at least 10 nM methotrexate; culturing the transfected cells with a reduced concentration or absence of the hypoxanthine and thymidine, and optionally the presence of between 10 nM and 2 uM methotrexate, wherein transfected cells surviving culturing have integrated the second polynucleotide into their genomes and can thereby express the target protein.

Synthetic amiRNA UTR: The invention further provides a polynucleotide comprising (a) an open reading frame operably linked to a first promoter that is active in a eukaryotic cell, (b) a polyadenylation signal sequence that is active in a eukaryotic cell, and (c) a sequence selected from SEQ ID NOs: 558-561, located between the open reading frame and the polyadenylation signal sequence, wherein the open reading frame does not encode Cricetulus griseus alpha-(1,6)-fucosyl transferase or Cricetulus griseus glutamine synthetase. The invention further provides a method of inhibiting expression of an open reading frame in a eukaryotic cell, comprising introducing into the eukaryotic cell (i) the polynucleotide described above and (ii) a polynucleotide encoding a multi-hairpin amiRNA comprising a sequence selected from SEQ ID NOs: 193, 194, 195 and 209 or the multi-hairpin amiRNA, wherein the multi-hairpin amiRNA inhibits expression of the open reading frame. Optionally, the polynucleotide encoding the multi-hairpin amiRNA is operably linked to a second promoter that is active in the cell. Optionally, the second promoter is inducible or constitutive. Optionally, the eukaryotic cell is a mammalian cell, a human cell, or a rodent cell. The invention further provides a cell comprising (i) the polynucleotide described above and (ii) a polynucleotide encoding a multi-hairpin amiRNA comprising a sequence selected from SEQ ID NOs: 193, 194, 195 and 209 or the multi-hairpin amiRNA.

Sialidase amiRNA: The invention further provides a polynucleotide comprising a segment encoding a) a multi-hairpin amiRNA sequence, wherein the segment comprises (i) a first guide strand sequence comprising a contiguous 19 nucleotide sequence that is perfectly complementary to a first target site of a natural mammalian cellular mRNA and a first passenger strand sequence comprising a contiguous 19 nucleotide sequence that is at least 78% complementary to the first guide strand sequence, wherein the first guide strand and first passenger strand sequence are separated by between 5 and 35 nucleotides; (ii) a second guide strand sequence comprising a contiguous 19 nucleotide sequence that is perfectly complementary to a second target site different than the first target site of the same natural mammalian cellular mRNA as the first guide strand sequence and a second passenger strand sequence comprising a contiguous 19 nucleotide sequence that is at least 78% complementary to the second guide strand sequence, wherein the second guide strand and second passenger strand sequence are separated by between 5 and 35 nucleotides, and wherein the first and second guide strand sequence are different from each other; and b) a eukaryotic promoter that is active in a mammalian cell and is transcribed by RNA polymerase II or RNA polymerase III, operably linked to the segment encoding the amiRNA sequence, wherein the amiRNA sequence can be expressed and fold into multiple hairpins, wherein the natural mammalian cellular mRNA encodes an enzyme that reduces protein sialylation.

Optionally, the natural mammalian cellular mRNA encodes a sialidase. Optionally, the natural mammalian cellular mRNA comprises a sequence that is at least 98% identical to a sequence selected from SEQ ID NOs: 13-18 or from SEQ ID NOs: 570-571. Optionally, the first and second guide strand sequences are selected from SEQ ID NOs: 85-89 or 565. Optionally, the first and second guide strand sequences are selected from SEQ ID NOs: 90-94. Optionally, the polynucleotide comprises a sequence selected from SEQ ID NOs: 212-225 or 567-569 comprising or encoding the multi-hairpin amiRNA sequence.

The invention further provides a method for increasing sialylation in a mammalian cell, comprising introducing into the mammalian cell the polynucleotide as described above flanked by transposon ends; and b) a corresponding transposase, wherein the transposase integrates the polynucleotide into the genome of the mammalian cell, whereby the mammalian cell produces a secreted protein with an increased level of sialylation relative to a control cell whose genome lacks the polynucleotide. Optionally, the corresponding transposase is introduced as a polynucleotide encoding the transposase. Optionally, the transposase is an mRNA. Optionally, the polynucleotide encoding the transposase is DNA, and comprises an open reading frame encoding the transposase operably linked to a promoter active in the mammalian cell. Optionally, the transposase is provided as transposase protein. Optionally, the genome of the mammalian cell further comprises a heterologous polynucleotide encoding the secreted protein, and the secreted protein is not naturally produced by the cell. Optionally, the method further comprises introducing into the cell the heterologous polynucleotide encoding the secreted protein, wherein the secreted protein is not naturally produced by the cell. The respective polynucleotides can be introduced in either order or at the same time, in which case they can be carried by the same DNA molecule. Optionally, the method further comprises purifying the secreted protein. Optionally, the method further comprise identifying the cell with the polynucleotide integrated into its genome. Optionally, the mammalian cell is a human cell or a CHO cell.

The invention further provides a mammalian cell produced by any of the above methods. The invention further provides a mammalian cell comprising polynucleotide as described above, wherein the polynucleotide is expressed to produce the multi-hairpin amiRNA sequence, which inhibits expression of the enzyme that reduces protein sialylation. Optionally, the cell further comprises a heterologous polynucleotide encoding a secreted protein not naturally produced by the cell, wherein sialylation of the secreted protein is increased compared with expression in a control cell lacking the polynucleotide expressed to produce the amiRNA sequence.

LPL amiRNA: The invention further provides a polynucleotide comprising a) a segment encoding a multi-hairpin amiRNA sequence, wherein the segment comprises (i) a first guide strand sequence comprising a contiguous 19 nucleotide sequence that is perfectly complementary to a first target site in a natural mammalian cellular mRNA of SEQ ID NO: 22 and a first passenger strand sequence comprising a contiguous 19 nucleotide sequence that is at least 78% complementary to the first guide strand sequence, wherein the first guide strand and first passenger strand sequence are separated by between 5 and 35 nucleotides; (ii) a second guide strand sequence comprising a contiguous 19 nucleotide sequence that is perfectly complementary to a second target site different than the first target site in the same natural mammalian cellular mRNA as the first guide strand sequence and a second passenger strand sequence comprising a contiguous 19 nucleotide sequence that is at least 78% complementary to the second guide strand sequence, wherein the second guide strand and second passenger strand sequences are separated by between 5 and 35 nucleotides, and wherein the first and second guide strand sequences are different from each other; and b) a eukaryotic promoter that is active in a mammalian cell and is transcribed by RNA polymerase II or RNA polymerase III operably linked to the segment encoding the amiRNA sequence, wherein the amiRNA sequence can be expressed and fold into multiple hairpins;

wherein the natural mammalian cellular mRNA encodes a fatty acid hydrolase. Optionally, the natural mammalian cellular mRNA comprises a sequence that is at least 98% identical to SEQ ID NO: 572 or 590-592. Optionally, the first and second guide strand sequences are selected from SEQ ID NOs: 573-578. Optionally, the polynucleotide comprises a sequence selected from SEQ ID NOs: 585-589.

The invention further provides a method for reducing lipoprotein lipase in a mammalian cell, comprising introducing into a mammalian cell a polynucleotide as described above; and a corresponding transposase, wherein the transposase integrates the polynucleotide into the genome of the cell, wherein expression of lipoprotein lipase is reduced. Optionally, a level of the lipoprotein contaminating a secreted protein produced by the cell is reduced. Optionally, transposase is introduced as a polynucleotide encoding the transposase. Optionally, the transposase is introduced as an mRNA encoding the transposase. Optionally, the polynucleotide encoding the transposase is DNA, and comprises an open reading frame encoding the transposase that is operably linked to a promoter that is active in the mammalian cell. Optionally, the transposase is provided as a transposase protein.

Optionally, the genome of the mammalian cell further comprises a gene encoding the secreted protein, and the secreted protein is not naturally produced by the cell. Optionally, the method further comprises introducing into the cell the gene encoding the secreted protein. The respective polynucleotides can be introduced in either order or at the same time, in which case they can be carried on the same DNA molecule. Optionally, the method further comprises purifying the secreted protein. Optionally, the method further comprises identifying a cell whose genome comprises the polynucleotide. Optionally, the cell is a CHO cell. The invention further provides a mammalian cell produced by any of the above methods.

IFN amiRNA: The invention provides a polynucleotide encoding a multi-hairpin amiRNA as described above, wherein the natural mammalian cellular mRNA encodes a subunit of an interferon receptor. Optionally, the natural mammalian cellular mRNA comprises a sequence that is at least 98% identical to a sequence selected from SEQ ID NOs: 19-22. Optionally, the first and second guide strand sequences are selected from SEQ ID NOs: 95-101. Optionally, the first and second guide strand sequences are selected from SEQ ID NOs: 102-107. Optionally, the polynucleotide comprises a sequence selected from SEQ ID NOs: 226-240. Optionally, the polynucleotide further comprises an open reading frame encoding an interferon polypeptide, operably linked to a promoter active in a mammalian cell.

The invention further provides a method for reducing expression of an interferon receptor in a mammalian cell, comprising introducing into the mammalian cell a polynucleotide described above flanked by transposon ends; and a corresponding transposase, wherein the transposase integrates the polynucleotide into the genome of the cell, and the polynucleotide expresses an amiRNA that reduces expression of the interferon receptor. Optionally, the corresponding transposase is introduced as a polynucleotide encoding the transposase. Optionally, the polynucleotide encoding the transposase is an mRNA. Optionally, the polynucleotide encoding the transposase is DNA, and comprises an open reading frame encoding the transposase that is operably linked to a promoter that is active in the mammalian cell. Optionally, the transposase is provided as transposase protein. Optionally, the genome of the mammalian cell further comprises a heterologous polynucleotide encoding an interferon polypeptide, expressible in the cell. Optionally, the method further comprises introducing into the cell the heterologous polynucleotide encoding the interferon polypeptide. The respective polynucleotides can be introduced in either order or at the same time, in which case they can be carried by the same DNA molecule. Optionally, the method further comprises purifying the interferon. Optionally, the method further comprises identifying the cell whose genome comprises the polynucleotide that expresses an amiRNA that reduces expression of an interferon receptor. Optionally, the mammalian cell is a human cell or a CHO cell. The invention further provides a mammalian cell produced by any of the above methods. The invention further provides a mammalian cell comprising a polynucleotide as described above, wherein the polynucleotide is expressed to produce the multi-hairpin amiRNA sequence, which inhibits expression of the subunit of the interferon receptor. Optionally, the mammalian cell further comprises a heterologous polynucleotide encoding an interferon, which is expressed with reduced toxicity to the cell compared with a control cell lacking the polynucleotide expressed to produce the amiRNA sequence.

Modification of gene to include target sites for amiRNA: The invention further provides a method of inhibiting expression of a gene in a mammalian cell, comprising modifying the mammalian cell so it expresses an mRNA encoded by the gene fused to a segment including first and second target sites different from each other; introducing in the mammalian cell a polynucleotide comprising a) a segment encoding a multi-hairpin amiRNA sequence, wherein the segment comprises i) a first guide strand sequence comprising a contiguous 19 nucleotide sequence that is perfectly complementary to the first target site and a first passenger strand sequence comprising a contiguous 19 nucleotide sequence that is at least 78% complementary to the first guide strand sequence, wherein the first guide strand and first passenger strand sequence are separated by between 5 and 35 nucleotides; (ii) a second guide strand sequence comprising a contiguous 19 nucleotide sequence that is perfectly complementary to the second target site and a second passenger strand sequence comprising a contiguous 19 nucleotide sequence that is at least 78% complementary to the second guide strand sequence, wherein the second guide strand and second passenger strand sequence are separated by between 5 and 35 nucleotides, and wherein the first and second guide strand sequence are different from each other; and b) a eukaryotic promoter that is active in a mammalian cell and is transcribed by RNA polymerase II or RNA polymerase III, operably linked to the segment encoding the amiRNA sequence, wherein the amiRNA sequence can be expressed and fold into multiple hairpins, wherein the multi-hairpin amiRNA sequence binds to the first and second target sites via the first and second guide strand sequences inhibiting expression of the gene. Optionally, the segment including the first and second target sites is fused within the 3′ UTR of the mRNA.

### BRIEF DESCRIPTION OF THE DRAWINGS

FIGS. 1A-B: Schematic representation of guide and passenger strand sequence organization. Nucleotides are shown for the coding strand of a single miRNA hairpin. The guide strand sequence is represented as 22 contiguous nucleotides N₁ to N₂₂. The sequence of the guide strand is preferably a perfect reverse complement of the target mRNA whose expression is to be reduced. The passenger strand sequence is represented as 22 contiguous nucleotides N′₁ to N′₂₂. The passenger strand sequence is preferably an imperfect reverse complement of the guide strand sequence. Corresponding bases in the guide strand sequence and passenger strand sequence are indicated by horizontal lines. For bases joined by a solid line, the base in the passenger strand is preferably the complementary base to the base in the guide strand. It is preferable if, for one or more of the bases joined by a dotted line, the base in the passenger strand is preferably not the complementary base to the base in the guide strand. If the base in the guide strand is an A or a T, the base in the passenger strand sequence is preferably a C. If the base in the guide strand sequence is a C or a G, the base in the passenger strand sequence is preferably an A. Most preferably the passenger strand sequence base at position N′₁ is not complementary to the guide strand sequence base N₁. Most preferably the passenger strand sequence base at position N′₁₂ is not complementary to the guide strand sequence base N₁₂. Mismatches may also be obtained if one or more base in the passenger strand are deleted. The guide strand sequence and the passenger strand sequence are joined by a 5-35 nucleotide unstructured loop sequence, represented as L₁-L_{Z}. The guide strand sequence may be to the 5′ of the loop sequence as shown in FIG. 1A, or to the 3′ of the loop sequence, as shown in FIG. 1B.

FIGS. 2A-B: Schematic representation of part of a multi-hairpin amiRNA gene. The processing of hairpin sequences comprising guide strand sequences, unstructured loops and passenger strand sequences to produce guide strand sequences loaded into the RISC complex for inhibition of target gene expression is improved if the amiRNA gene comprises additional features. These include additional stem structures to the 5′ and 3′ of the hairpin sequences. Element A is a sequence that is complementary to element E, and which stabilizes hairpin 1, although the complementarity between elements A and E does not need to be perfect to perform this function. Similarly, element G is a sequence that is complementary to element K, and which stabilizes hairpin 2, although the complementarity between elements A and E does not need to be perfect to perform this function. Optionally hairpins are separated by an unstructured spacer element F. Two or more hairpins are operably linked to the same promoter, and the first hairpin is separated from the promoter by a spacer sequence. Hairpin 1 is shown in a configuration with guide followed by loop followed by passenger, Hairpin 2 is shown in this same configuration in FIG. 2A, but in a passenger-loop-guide configuration in FIG. 2B. Any other combinations of configurations are acceptable. Additional hairpins may be placed following the second hairpin. Optionally the final hairpin in a multi-hairpin amiRNA gene is followed by a polyadenylation signal sequence.

FIGS. 3A-G: Mass spectra of antibodies comprising glycans produced by stably transfected CHO lines expressing multi-hairpin amiRNA genes targeting FUT8. Protein was purified from antibody-producing cells as described in Section 6.1.1.1 and analyzed by mass spectroscopy. Arrows indicate the predicted molecular weights of (i) 50,424 Da, the heavy chain modified by G₀: the conserved heptasaccharide core composed of 2 N-acetylglucosamine, 3 mannose and 2 other N-acetylglucosamine residues that are β-1,2 linked to α-6 mannose and α-3 mannose, forming two arms; (ii) 50,571 Da, the heavy chain modified by G_{0F}: the conserved heptasaccharide core plus a fucose residue; (iii) 50,586 Da, the heavy chain modified by G₁: the conserved heptasaccharide core plus a galactose residue and (iv) 50,733 Da, the heavy chain modified by G₁: the conserved heptasaccharide core plus a galactose residue plus a fucose residue. In all cases the heavy chain has also lost its C-terminal lysine residue. FIG. 3A: no amiRNA transposon; FIGS. 3B-G: multi-hairpin amiRNA transposons configured as shown in Table 1.

FIGS. 4A-D: Mass spectra of antibodies comprising glycans produced by stably transfected CHO lines expressing multi-amiRNA sequences linked to different promoters. Protein was purified from antibody-producing cells as described in Section 6.1.1.2 and analyzed by mass spectroscopy. Arrows indicate the predicted molecular weights of (i) 50,424 Da, the heavy chain modified by G₀: the conserved heptasaccharide core composed of 2 N-acetylglucosamine, 3 mannose and 2 other N-acetylglucosamine residues that are β-1,2 linked to α-6 mannose and α-3 mannose, forming two arms; (ii) 50,570 Da, the heavy chain modified by G_{0F}: the conserved heptasaccharide core plus a fucose residue; (iii) 23,443 Da, the light chain. In all cases the heavy chain has also lost its C-terminal lysine residue. FIG. 4A: no amiRNA transposon; FIG. 4B: multi-hairpin amiRNA with nucleotide sequence SEQ ID NO: 194 operably linked to an EEF2 promoter; FIG. 4C: multi-hairpin amiRNA with nucleotide sequence SEQ ID NO: 194 operably linked to a PGK promoter; FIG. 4D: multi-hairpin amiRNA with nucleotide sequence SEQ ID NO: 194 operably linked to a Ubb promoter.

FIGS. 5A-B: Mass spectra of antibodies comprising glycans produced by CHO lines expressing multi-amiRNA genes and subsequently transiently transfected with antibody genes. Protein was purified from antibody-producing cells as described in Section 6.1.1.3 and analyzed by mass spectroscopy. Arrows indicate the predicted molecular weights of (i) 50,521 Da, the heavy chain modified by G₀: the conserved heptasaccharide core composed of 2 N-acetylglucosamine, 3 mannose and 2 other N-acetylglucosamine residues that are β-1,2 linked to α-6 mannose and α-3 mannose, forming two arms; (ii) 50,668 Da, the heavy chain modified by G_{0F}: the conserved heptasaccharide core plus a fucose residue; (iii) 23,444 Da, the light chain. In all cases the heavy chain has also lost its C-terminal lysine residue. FIG. 5A: no amiRNA transposon; FIG. 5B: multi-hairpin amiRNA with nucleotide sequence SEQ ID NO: 194 operably linked to an EF1 promoter.

FIGS. 6A-B: Capillary isoelectric focusing of protein produced by CHO lines with and without sialidase-inhibiting multi-amiRNA genes. A fusion between the extracellular domain of a growth factor receptor and a human IgG1 Fc was expressed from FIG. 6A a CHO cell line and FIG. 6B the same CHO cell line transfected with a transposon comprising an amiRNA gene expressing guide RNAs complementary to the mRNA for CHO neu2 sialidase. In grey on both traces is a highly sialylated reference standard. Experimental details are given in Section 6.3.1.

### DESCRIPTION

### 5.1 Definitions

Use of the singular forms “a,” “an,” and “the” include plural references unless the context clearly dictates otherwise. Thus, for example, reference to “a polynucleotide” includes a plurality of polynucleotides, reference to “a substrate” includes a plurality of such substrates, reference to “a variant” includes a plurality of variants, and the like.

Terms such as “connected,” “attached,” “linked,” and “conjugated” are used interchangeably herein and encompass direct as well as indirect connection, attachment, linkage or conjugation unless the context clearly dictates otherwise. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values which are about the same quantity or amount as the recited value are also within the scope of the invention. Where a combination is disclosed, each sub combination of the elements of that combination is also specifically disclosed and is within the scope of the invention. Conversely, where different elements or groups of elements are individually disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Unless defined otherwise herein, all technical and scientific terms used herein have the same meaning as commonly understood by one of ordinary skill in the art to which this invention belongs. Singleton, et. al., Dictionary of Microbiology and Molecular Biology, 2nd Ed., John Wiley and Sons, New York (1994), and Hale & Marham, The Harper Collins Dictionary of Biology, Harper Perennial, N Y, 1991, provide one of skill with a general dictionary of many of the terms used in this invention. Although any methods and materials similar or equivalent to those described herein can be used in the practice or testing of the present invention, the preferred methods and materials are described. Unless otherwise indicated, nucleic acids are written left to right in 5′ to 3′ orientation; amino acid sequences are written left to right in amino to carboxy orientation, respectively. The terms defined immediately below are more fully defined by reference to the specification as a whole.

An “artificial micro-RNA” or “amiRNA” is a sequence comprising a natural microRNA scaffold in which the guide and/or passenger strand sequences have been modified such that the guide strand is directed to an mRNA target other than the natural target. Other parts of the natural micro-RNA scaffold may also be modified, for example to improve processing by enzymes in the RNA interference pathway. An amiRNA sequence that comprises two or more guide and passenger strands operably linked to the same promoter is referred to as a “multi-hairpin amiRNA gene”. RNA sequences including shRNA and amiRNA sequences, may be provided herein as the sequence of the RNA or of the DNA that encodes them. If the DNA sequence is provided, it is intended that the sequence of the RNA will be the same with the exception that thymine (T) is replaced with uracil (U), and vice versa.

The term “codon usage” or “codon bias” refers to the relative frequencies with which different synonymous codons are used to encode an amino acid within an open reading frame. A nucleic acid sequence having codon preferences for a particular target cell has a balance of synonymous codon choices that result in efficient translation in that cell type. This balance is often not calculable from observed genomic codon frequencies, but must be empirically determined, for example as described in U.S. Pat. Nos. 7,561,972 and 7,561,973 and 8,401,798 and in Welch et. al. (2009) “Design Parameters to Control Synthetic Gene Expression in Escherichia coli”. PLoS ONE 4(9): e7002. https://doi.org/10.1371/journal.pone.0007002. A nucleic acid originally isolated from one cell type to be introduced into a target cell of another type can undergo selection of codon preferences for the target site cell such that at least 1 and sometimes, 5, 20, 15, 20, 50, 100 or more choices among synonymous codons differ between the nucleic acid introduced into the target cell from the original nucleic acid.

Two polynucleotides are “complementary” if the bases of one hydrogen bond to the bases of the other. For perfect complementarity, adenine (A) in the first polynucleotide must correspond with thymine (T) (or uracil for RNA) in the second (and vice versa), and cytosine (C) in the first polynucleotide must correspond with guanine (G) in the second (and vice versa). The two polynucleotides must also be antiparallel. If two polynucleotides are complementary, one may be described as the “reverse complement” of the other to indicate that their bases are complementary when one is in the 5′ to 3′ direction and the other is in the 3′ to 5′ direction. As used herein, when one polynucleotide sequence is described as complementary to another, it is intended to indicate that the sequences are antiparallel and able to base-pair with one another.

The “configuration” of a polynucleotide means the functional sequence elements within the polynucleotide, and the order and direction of those elements.

The terms “corresponding transposon” and “corresponding transposase” are used to indicate an activity relationship between a transposase and a transposon. A transposase transposes its corresponding transposon. Many transposases may correspond with a single transposon, and many transposons may correspond with a single transposase.

The term “counter-selectable marker” means a polynucleotide sequence that confers a selective disadvantage on a host cell. Examples of counter-selectable markers include sacB, rpsL, tetAR, pheS, thyA, gata-1, ccdB, kid and barnase (Bernard, 1995, Journal/Gene, 162: 159-160; Bernard et. al., 1994. Journal/Gene, 148: 71-74; Gabant et. al., 1997, Journal/Biotechniques, 23: 938-941; Gababt et. al., 1998, Journal/Gene, 207: 87-92; Gababt et. al., 2000, Journal/Biotechniques, 28: 784-788; Galvao and de Lorenzo, 2005, Journal/Appl Environ Microbiol, 71: 883-892; Hartzog et. al., 2005, Journal/Yeat, 22:789-798; Knipfer et. al., 1997, Journal/Plasmid, 37: 129-140; Reyrat et. al., 1998, Journal/Infect Immun, 66: 4011-4017; Soderholm et. al., 2001, Journal/Biotechniques, 31: 306-310, 312; Tamura et. al., 2005, Journal/Appl Environ Microbiol, 71: 587-590; Yazynin et. al., 1999, Journal/FEBS Lett, 452: 351-354). Counter-selectable markers often confer their selective disadvantage in specific contexts. For example, they may confer sensitivity to compounds that can be added to the environment of the host cell, or they may kill a host with one genotype but not kill a host with a different genotype. Conditions which do not confer a selective disadvantage on a cell carrying a counter-selectable marker are described as “permissive”. Conditions which do confer a selective disadvantage on a cell carrying a counter-selectable marker are described as “restrictive”.

The term “coupling element” or “translational coupling element” means a DNA sequence that allows the expression of a first polypeptide to be linked to the expression of a second polypeptide. Internal ribosome entry site elements (IRES elements) and cis-acting hydrolase elements (CHYSEL elements) are examples of coupling elements.

The terms “DNA sequence”, “RNA sequence” or “polynucleotide sequence” mean a contiguous nucleic acid sequence. The sequence can be an oligonucleotide of 2 to 20 nucleotides in length to a full-length genomic sequence of thousands or hundreds of thousands of base pairs.

The term “expression construct” means any polynucleotide designed to transcribe an RNA. For example, a construct that contains at least one promoter which is or may be operably linked to a downstream gene, coding region, or polynucleotide sequence (for example, a cDNA or genomic DNA fragment that encodes a polypeptide or protein, or an RNA effector molecule, for example, an antisense RNA, triplex-forming RNA, ribozyme, an artificially selected high affinity RNA ligand (aptamer), a double-stranded RNA, for example, an RNA molecule comprising a stem-loop or hairpin dsRNA, or a bi-finger or multi-finger dsRNA or a microRNA, or any RNA). An “expression vector” is a polynucleotide comprising a promoter which can be operably linked to a second polynucleotide. Transfection or transformation of the expression construct into a recipient cell allows the cell to express an RNA effector molecule, polypeptide, or protein encoded by the expression construct. An expression construct may be a genetically engineered plasmid, virus, recombinant virus, or an artificial chromosome derived from, for example, a bacteriophage, adenovirus, adeno-associated virus, retrovirus, lentivirus, poxvirus, or herpesvirus. Such expression vectors can include sequences from bacteria, viruses or phages. Such vectors include chromosomal, episomal and virus-derived vectors, for example, vectors derived from bacterial plasmids, bacteriophages, yeast episomes, yeast chromosomal elements, and viruses, vectors derived from combinations thereof, such as those derived from plasmid and bacteriophage genetic elements, cosmids and phagemids. An expression construct can be replicated in a living cell, or it can be made synthetically. For purposes of this application, the terms “expression construct”, “expression vector”, “vector”, and “plasmid” are used interchangeably to demonstrate the application of the invention in a general, illustrative sense, and are not intended to limit the invention to a particular type of expression construct.

The term “expression polypeptide” means a polypeptide encoded by a gene on an expression construct.

The term “expression system” means any in vivo or in vitro biological system that is used to produce one or more gene product encoded by a polynucleotide.

A “gene” refers to a transcriptional unit including a promoter and sequence to be expressed from it as an RNA or protein. The sequence to be expressed can be genomic or cDNA or one or more non-coding RNAs including siRNAs or microRNAs among other possibilities. Other elements, such as introns, and other regulatory sequences may or may not be present.

Any of the inhibitory and other polynucleotides described herein can be incorporated into a gene transfer system. A “gene transfer system” comprises a vector or gene transfer vector, or a polynucleotide comprising a gene to be transferred which is cloned into a vector (a “gene transfer polynucleotide” or “gene transfer construct”). A gene transfer system may also comprise other features to facilitate the process of gene transfer. For example, a gene transfer system may comprise a vector and a lipid or viral packaging mix for enabling a first polynucleotide to enter a cell, or it may comprise a polynucleotide that includes a transposon and a second polynucleotide sequence encoding a corresponding transposase to enhance productive genomic integration of the transposon. For example, an inhibitory or other polynucleotide of the invention can be flanked by transposon inverted terminal repeats and transposon target integration sites to facilitate integration of the polynucleotide into the genome of a cell. The transposases and transposons of a gene transfer system may be on the same nucleic acid molecule or on different nucleic acid molecules. The transposase of a gene transfer system may be provided as a polynucleotide or as a polypeptide.

The “guide” strand of an inhibitory double stranded RNA such as an shRNA or a miRNA is the strand that binds to the RNA-induced silencing complex (RISC) and participates in gene silencing. The guide strand sequence is the reverse complement of a target mRNA sequence, whose expression it inhibits.

The term “hairpin” is used to describe a polynucleotide sequence in which two regions of the same strand are reverse complements of each other in nucleotide sequence, resulting in intramolecular base pairing to form a double-stranded region and an unpaired loop. The term is used herein to describe the DNA sequence that encodes such a structure, although normally DNA is double-stranded through intermolecular base-pairing. The term is also used to refer to the RNA sequence that adopts the hairpin structure. DNA hairpins of the present invention are intended for expression as RNA. An RNA hairpin of the present invention is intended as a substrate for the RNA interference pathway enzymes to be processed into a guide strand loaded onto the RISC complex. The “guide strand” of a hairpin is the sequence that, after transcription and processing, is loaded into the RISC complex. The guide strand is complementary to the target mRNA.

Two elements are “heterologous” to one another if not naturally associated. For example, a nucleic acid sequence encoding a protein linked to a heterologous promoter means a promoter other than that which naturally drives expression of the protein. A heterologous nucleic acid flanked by transposon ends or ITRs means a heterologous nucleic acid not naturally flanked by those transposon ends or ITRs, such as a nucleic acid encoding a polypeptide other than a transposase, including an antibody heavy or light chain. A nucleic acid is heterologous to a cell if not naturally found in the cell or if naturally found in the cell but in a different location (e.g., episomal or different genomic location) than the location described.

A “hyperactive” transposase is a transposase that is more active than the naturally occurring transposase from which it is derived. “Hyperactive” transposases are thus not naturally occurring sequences.

The term “host” means any prokaryotic or eukaryotic organism that can be a recipient of a nucleic acid. A “host,” as the term is used herein, includes prokaryotic or eukaryotic organisms that can be genetically engineered. For examples of such hosts, see Maniatis et. al., Molecular Cloning. A Laboratory Manual, Cold Spring Harbor Laboratory, Cold Spring Harbor, N.Y. (1982). As used herein, the terms “host,” “host cell,” “host system” and “expression host” can be used interchangeably.

An “IRES” or “internal ribosome entry site” means a specialized sequence that directly promotes ribosome binding, independent of a cap structure.

An ‘isolated’ polypeptide or polynucleotide means a polypeptide or polynucleotide that has been either removed from its natural environment, produced using recombinant techniques, or chemically or enzymatically synthesized. Polypeptides or polynucleotides of this invention may be purified, that is, essentially free from any other polypeptide or polynucleotide and associated cellular products or other impurities.

The terms “nucleoside” and “nucleotide” include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, for example, where one or more of the hydroxyl groups are replaced with halogen, aliphatic groups, or is functionalized as ethers, amines, or the like. The term “nucleotidic unit” is intended to encompass nucleosides and nucleotides.

An “Open Reading Frame” or “ORF” means a portion of a polynucleotide that, when translated into amino acids, contains no stop codons. The genetic code reads DNA sequences in groups of three base pairs, which means that a double-stranded DNA molecule can read in any of six possible reading frames-three in the forward direction and three in the reverse. An ORF typically also includes an initiation codon at which translation may start.

The term “operably linked” refers to functional linkage between two sequences such that one sequence modifies the behavior of the other. For example, a first polynucleotide comprising a nucleic acid expression control sequence (such as a promoter, IRES sequence, enhancer or array of transcription factor binding sites) and a second polynucleotide are operably linked if the first polynucleotide affects transcription and/or translation of the second polynucleotide. Similarly, a first amino acid sequence comprising a secretion signal or a subcellular localization signal and a second amino acid sequence are operably linked if the first amino acid sequence causes the second amino acid sequence to be secreted or localized to a subcellular location.

The term “orthogonal” refers to a lack of interaction between two systems. A first transposon and its corresponding first transposase and a second transposon and its corresponding second transposase are orthogonal if the first transposase does not excise or transpose the second transposon and the second transposase does not excise or transpose the first transposon.

The term “overhang” or “DNA overhang” means the single-stranded portion at the end of a double-stranded DNA molecule. Complementary overhangs are those which will base-pair with each other.

The “passenger” strand of an inhibitory double stranded RNA such as an shRNA or a miRNA is the strand that is degraded after transport to the cytoplasm and does not participate directly in gene silencing.

A “piggyBac-like transposase” means a transposase with at least 20% amino acid sequence identity as identified using the TBLASTP algorithm to the piggyBac transposase from Trichoplusia ni (SEQ ID NO: 463), and as more fully described in Sakar, A. et. al., 2003. Mol. Gen. Genomics 270: 173-180. “Molecular evolutionary analysis of the widespread piggyBac transposon family and related ‘domesticated’ species”, and further characterized by a DDE-like DDD motif, with aspartate residues at positions corresponding to D268, D346, and D447 of Trichoplusia ni piggyBac transposase on maximal alignment. PiggyBac-like transposases are also characterized by their ability to excise their transposons precisely with a high frequency. A “piggyBac-like transposon” means a transposon having transposon ends which are the same or at least 80% and preferably at least 90, 95, 96, 97, 98, 99% or 100% identical to the nucleotide sequences of the transposon ends of a naturally occurring transposon that encodes a piggyBac-like transposase. A piggyBac-like transposon includes an inverted terminal repeat (ITR) sequence of approximately 12-16 bases at each end. These repeats may be identical at the two ends, or the repeats at the two ends may differ at 1 or 2 or 3 or 4 positions in the two ITRs. The transposon is flanked on each side by a 4 base sequence corresponding to the integration target sequence which is duplicated on transposon integration (the Target Site Duplication or Target Sequence Duplication or TSD). PiggyBac-like transposons and transposases occur naturally in a wide range of organisms including Argyrogramma agnate (GU477713), Anopheles gambiae (XP_312615; XP_320414; XP_310729), Aphis gossypii (GU329918), Acyrthosiphon pisum (XP_001948139), Agrotis ipsilon (GU477714), Bombyx mori (BAD11135), Ciona intestinalis (XP_002123602), Chilo suppressalis (JX294476), Drosophila melanogaster (AAL39784), Daphnia pulicaria (AAM76342), Helicoverpa armigera (ABS18391), Homo sapiens (NP 689808), Heliothis virescens (ABD76335), Macdunnoughia crassisigna (EU287451), Macaca fascicularis (AB179012), Mus musculus (NP_741958), Pectinophora gossypiella (GU270322), Rattus norvegicus (XP_220453), Tribolium castaneum (XP_001814566) and Trichoplusia ni (AAA87375) and Xenopus tropicalis (BAF82026), although transposition activity has been described for almost none of these.

The terms “polynucleotide,” “oligonucleotide,” “nucleic acid” and “nucleic acid molecule” are used interchangeably to refer to a polymeric form of nucleotides of any length, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing non-nucleotidic backbones, for example, polyamide (for example, peptide nucleic acids (“PNAs”)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms are used interchangeably herein. These terms refer only to the primary structure of the molecule. Thus, these terms include, for example, 3′-deoxy-2′, 5′-DNA, oligodeoxyribonucleotide N3′ P5′ phosphoramidates, 2′-O-alkyl-substituted RNA, double- and single-stranded DNA, as well as double- and single-stranded RNA, and hybrids thereof including for example hybrids between DNA and RNA or between PNAs and DNA or RNA, and also include known types of modifications, for example, labels, alkylation, “caps,” substitution of one or more of the nucleotides with an analog, internucleotide modifications such as, for example, those with uncharged linkages (for example, methyl phosphonates, phosphotriesters, phosphoramidates, carbamates, or the like) with negatively charged linkages (for example, phosphorothioates, phosphorodithioates, or the like), and with positively charged linkages (for example, aminoalkylphosphoramidates, aminoalkylphosphotriesters), those containing pendant moieties, such as, for example, proteins (including enzymes (for example, nucleases), toxins, antibodies, signal peptides, poly-L-lysine, or the like), those with intercalators (for example, acridine, psoralen, or the like), those containing chelates (of, for example, metals, radioactive metals, boron, oxidative metals, or the like), those containing alkylators, those with modified linkages (for example, alpha anomeric nucleic acids, or the like), as well as unmodified forms of the polynucleotide or oligonucleotide.

A “promoter” means a nucleic acid sequence sufficient to direct transcription of an operably linked nucleic acid molecule. A promoter can be used with or without other transcription control elements (for example, enhancers) that are sufficient to render promoter-dependent gene expression controllable in a cell type-specific, tissue-specific, or temporal-specific manner, or that are inducible by external signals or agents; such elements, may be within the 3′ region of a gene or within an intron. Desirably, a promoter is operably linked to a nucleic acid sequence, for example, a cDNA or a gene sequence, or an effector RNA coding sequence, in such a way as to enable expression of the nucleic acid sequence, or a promoter is provided in an expression cassette into which a selected nucleic acid sequence to be transcribed can be conveniently inserted. A regulatory element such as promoter active in a mammalian cell means a regulatory element configurable to result in a level of expression of at least 1 transcript and optionally at least ten transcripts per cell in a mammalian cell into which the regulatory element has been introduced. A promoter or other regulatory element active in a eukaryotic cell or other cell is correspondingly described with respect to the relevant cell.

“RNA interference” is a biological process in which RNA molecules inhibit gene expression or translation, by neutralizing targeted mRNA molecules. Historically, RNAi was known by other names, including co-suppression, post-transcriptional gene silencing (PTGS), and quelling. Micro RNAs, including artificial micro RNAs, inhibit gene expression through RNA interference.

The term “selectable marker” means a polynucleotide segment or expression product thereof that allows one to select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions. Examples of selectable markers include but are not limited to: (1) DNA segments that encode products which provide resistance against otherwise toxic compounds (e.g., antibiotics); (2) DNA segments that encode products which are otherwise lacking in the recipient cell (e.g., tRNA genes, auxotrophic markers); (3) DNA segments that encode products which suppress the activity of a gene product; (4) DNA segments that encode products which can be readily identified (e.g., phenotypic markers such as beta-galactosidase, green fluorescent protein (GFP), and cell surface proteins); (5) DNA segments that bind products which are otherwise detrimental to cell survival and/or function; (6) DNA segments that otherwise inhibit the activity of any of the DNA segments described in Nos. 1-5 above (e.g., antisense oligonucleotides); (7) DNA segments that bind products that modify a substrate (e.g. restriction endonucleases); (8) DNA segments that can be used to isolate a desired molecule (e.g. specific protein binding sites); (9) DNA segments that encode a specific nucleotide sequence which can be otherwise non-functional (e.g., for PCR amplification of subpopulations of molecules); and/or (10) DNA segments, which when absent, directly or indirectly confer sensitivity to particular compounds.

Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

A “target nucleic acid” is a nucleic acid into which a transposon is to be inserted. Such a target can be part of a chromosome, episome or vector.

An “integration target sequence” or “target sequence” or “target site” for a transposase is a site or sequence in a target DNA molecule into which a transposon can be inserted by a transposase. The piggyBac transposase from Trichoplusia ni inserts its transposon predominantly into the target sequence 5′-TTAA-3′. Other useable target sequences for piggyBac transposons are 5′-CTAA-3′, 5′-TTAG-3′, 5′-ATAA-3′, 5′-TCAA-3′, 5′-AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-GTAA-3′, 5′-AGGT-3′, 5′-ATCA-3′, 5′-CTCC-3′, 5′-TAAA-3′, 5′-TCTC-3′, 5′-TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-CTGA-3′, 5′-GTCC-3′, 5′-TAAG-3′, 5′-TCTA-3′, 5′-TGAG-3′, 5′-TGTT-3′, 5′-TTCA-3′, 5′-TTCT-3′ and 5′-TTTT-3′ (Li et al., 2013. Proc. Natl. Acad. Sci vol. 110, no. 6, E478-487) and 5′-TTAT. PiggyBac-like transposases transpose their transposons using a cut-and-paste mechanism, which results in duplication of their 4 base pair target sequence on insertion into a DNA molecule. The target sequence is thus found on each side of an integrated piggyBac-like transposon.

The term “translation” refers to the process by which a polypeptide is synthesized by a ribosome ‘reading’ the sequence of a polynucleotide.

A ‘transposase’ is a polypeptide that catalyzes the excision of a corresponding transposon from a donor polynucleotide, for example a vector, and (providing the transposase is not integration-deficient) the subsequent integration of the transposon into a target nucleic acid.

The term “transposition” is used herein to mean the action of a transposase in excising a transposon from one polynucleotide and then integrating it, either into a different site in the same polynucleotide, or into a second polynucleotide.

The term “transposon” means a polynucleotide that can be excised from a first polynucleotide, for instance, a vector, and be integrated into a second position in the same polynucleotide, or into a second polynucleotide, for instance, the genomic or extrachromosomal DNA of a cell, by the action of a corresponding trans-acting transposase. A transposon comprises a first transposon end and a second transposon end, which are polynucleotide sequences recognized by and transposed by a transposase. A transposon usually further comprises a first polynucleotide sequence between the two transposon ends, such that the first polynucleotide sequence is transposed along with the two transposon ends by the action of the transposase. This first polynucleotide in natural transposons frequently comprises an open reading frame encoding a corresponding transposase that recognizes and transposes the transposon. Transposons of the present invention are “synthetic transposons” comprising a heterologous polynucleotide sequence which is transposable by virtue of its juxtaposition between two transposon ends. Synthetic transposons may or may not further comprise flanking polynucleotide sequence(s) outside the transposon ends, such as a sequence encoding a transposase, a vector sequence or sequence encoding a selectable marker.

The term “transposon end” means the cis-acting nucleotide sequences that are sufficient for recognition by and transposition by a corresponding transposase. Transposon ends of piggyBac-like transposons comprise perfect or imperfect repeats such that the respective repeats in the two transposon ends are reverse complements of each other. These are referred to as inverted terminal repeats (ITR) or terminal inverted repeats (TIR). A transposon end may or may not include additional sequence proximal to the ITR that promotes or augments transposition.

The term “vector” or “DNA vector” or “gene transfer vector” refers to a polynucleotide that is used to perform a “carrying” function for another polynucleotide. For example, vectors are often used to allow a polynucleotide to be propagated within a living cell, or to allow a polynucleotide to be packaged for delivery into a cell, or to allow a polynucleotide to be integrated into the genomic DNA of a cell. A vector may further comprise additional functional elements, for example it may comprise a transposon.

Any disclosure associating a polynucleotide with a SEQ ID NO. irrespective of transitional term used for the association, should be understood as providing disclosure of any of polynucleotide comprising the SEQ ID NO., consisting of the SEQ ID NO. or consisting essentially of the SEQ ID NO.

Unless the context requires otherwise reference to an amiRNA should be understood as alternatively disclosing a DNA encoding the amiRNA and vice versa.

### 5.2 Genetic Elements Useful for Expression in Cultured Mammalian Cells

### 5.2.1 Gene Transfer Systems

Gene transfer systems comprise a polynucleotide to be transferred to a host cell. The gene transfer system may comprise any of the polynucleotides describes herein. Some gene transfer systems are in the form of transposons described herein together with their corresponding transposases. Although transposons are preferred gene transfer systems because of their large cargo sizes and because multiple different open reading frames with all of their associated regulatory elements can be incorporated without compromising packaging and delivery of the gene transfer system, a gene transfer system for delivery of an inhibitory gene transfer polynucleotide may comprise one or more polynucleotides that have other features that facilitate efficient gene transfer without the need for a transposase or transposon, for example a viral system such as a lentiviral system, an adenoviral system or an adeno-associated viral system.

The components of the gene transfer system may be transfected into one or more cells by techniques such as particle bombardment, electroporation, microinjection, combining the components with lipid-containing vesicles, such as cationic lipid vesicles, DNA condensing reagents (example, calcium phosphate, polylysine or polyethyleneimine), and inserting the components (that is the nucleic acids thereof into a viral vector and contacting the viral vector with the cell. Where a viral vector is used, the viral vector can include any of a variety of viral vectors known in the art including viral vectors selected from the group consisting of a retroviral vector, an adenovirus vector or an adeno-associated viral vector. A retroviral vector may be a lentiviral vector comprising two LTRs each of which is at least 90% identical to a nucleotide sequence selected from SEQ ID NOs: 531-532. An adeno-associated viral vector may comprise two ITRs each of which is at least 90% identical to a nucleotide sequence selected from SEQ ID NOs: 533-539. The gene transfer system may be formulated in a suitable manner as known in the art, or as a pharmaceutical composition or kit.

The consistency of expression of a gene from a heterologous polynucleotide in a cultured mammalian cell can be improved if the heterologous polynucleotide is integrated into the genome of the host cell. Integration of a polynucleotide into the genome of a host cell also generally makes it stably heritable, by subjecting it to the same mechanisms that ensure the replication and division of genomic DNA. Such stable heritability is desirable for achieving good and consistent expression over long growth periods. For stable modification of cultured mammalian cells, including the consistent expression of inhibitory RNAs such as miRNAs and amiRNAs, the stability of the modification and consistency of expression levels are important, particularly for therapeutic applications.

### 5.2.2 Transposon Elements

Heterologous polynucleotides may be more efficiently integrated into a target genome if they are part of a transposon, for example so that they may be integrated by a transposase. A particular benefit of a transposon is that the entire polynucleotide between the transposon ITRs is integrated. This is in contrast with random integration, where a polynucleotide introduced into a eukaryotic cell is often fragmented at random in the cell, and only parts of the polynucleotide become incorporated into the target genome, usually at a low frequency. There are several different classes of transposon. piggyBac-like transposons include the piggyBac transposon from the looper moth Trichoplusia ni, Xenopus piggyBac-like transposons, Bombyx piggyBac-like transposons, Heliothis piggyBac-like transposons, Helicoverpa piggyBac-like transposons, Agrotis piggyBac-like transposons, Amyelois piggyBac-like transposons, piggyBat piggyBac-like transposons and Oryzias piggyBac-like transposons. hAT transposons include TcBuster. Mariner transposons include Sleeping Beauty. Each of these transposons can be integrated into the genome of a mammalian cell by a corresponding transposase. Heterologous polynucleotides incorporated into transposons may be integrated into cultured mammalian cells, as well as hepatocytes, neural cells, muscle cells, blood cells, embryonic stem cells, somatic stem cells, hematopoietic cells, embryos, zygotes and sperm cells (some of which are open to be manipulated in an in vitro setting). Preferred cells can also be pluripotent cells (cells whose descendants can differentiate into several restricted cell types, such as hematopoietic stem cells or other stem cells) or totipotent cells (i.e., a cell whose descendants can become any cell type in an organism, e.g., embryonic stem cells).

Preferred gene transfer systems, including inhibitory polynucleotides comprising sequences for the expression of inhibitory RNAs, comprise a transposon in combination with a corresponding transposase protein that transposases the transposon, or a nucleic acid that encodes the corresponding transposase protein and is expressible in the target cell.

When there are multiple components of a gene transfer system, for example one or more polynucleotides comprising transposon ends flanking genes for expression in the target cell, and a transposase (which may be provided either as a protein or encoded by a nucleic acid), these components can be transfected into a cell at the same time, or sequentially. For example, a transposase protein or its encoding nucleic acid may be transfected into a cell prior to, simultaneously with or subsequently to transfection of a corresponding transposon. Additionally, administration of either component of the gene transfer system may occur repeatedly, for example, by administering at least two doses of this component.

Transposase proteins may be encoded by polynucleotides including RNA or DNA.

Preferable RNA molecules include those with appropriate substitutions to reduce toxicity effects on the cell, for example substitution of uridine with pseudouridine, and substitution of cytosine with 5-methyl cytosine. mRNA encoding the transposase may be prepared such that it has a 5′-cap structure to improve expression in a target cell. Exemplary cap structures are a cap analog (G(5)ppp(5′)G), an anti-reverse cap analog (3′-O-Me-m⁷G(5′)ppp(5′)G, a clean cap (m7G(5)ppp(5′)(2′OMeA)pG), an mCap (m7G(5′)ppp(5′)G). mRNA encoding the transposase may be prepared such that some bases are partially or fully substituted, for example uridine may be substituted with pseudo-uridine, cytosine may be substituted with 5-methyl-cytosine. Any combinations of these caps and substitutions may be made. Similarly, the nucleic acid encoding the transposase protein, or the transposon of this invention can be transfected into the cell as a linear fragment or as a circularized fragment, either as a plasmid or as recombinant viral DNA. If the transposase is introduced as a DNA sequence encoding the transposase, then the open reading frame encoding the transposase is preferably operably linked to a promoter that is active in the target mammalian cell.

An advantageous piggyBac-like transposon for modifying the genome of a mammalian cell is a Xenopus transposon which comprises an ITR with the with nucleotide sequence of SEQ ID NO: 421, a heterologous polynucleotide to be transposed and a second ITR with nucleotide sequence of SEQ ID NO: 422. The transposon may further be flanked by a copy of the tetranucleotide 5′-TTAA-3′ on each side, immediately adjacent to the ITRs and distal to the heterologous polynucleotide. The transposon may further comprise a nucleotide sequence immediately adjacent to the ITR and proximal to the heterologous polynucleotide that is at least 95% identical to SEQ ID NO: 417 or 418 on one side of the heterologous polynucleotide, preferably the left side, and a nucleotide sequence immediately adjacent to the ITR and proximal to the heterologous polynucleotide that is at least 95% identical to SEQ ID NO: 419 or 420 on the other side of the heterologous polynucleotide, preferably the right side. This transposon may be transposed by a corresponding Xenopus transposase comprising a polypeptide sequence at least 90% identical to the polypeptide sequence of SEQ ID NO: 465 or 466, for example any of SEQ ID NOs: 465-497. Preferably the transposase is a hyperactive variant of a naturally occurring transposase. Preferably the hyperactive variant transposase comprises one or more of the following amino acid changes, relative to the polypeptide sequence of SEQ ID NO: 465: Y6L, Y6H, Y6V, Y6I, Y6C, Y6G, Y6A, Y6S, Y6F, Y6R, Y6P, Y6D, Y6N, S7G, S7V, S7D, E9W, E9D, E9E, M16E, M16N, M16D, M16S, M16Q, M16T, M16A, M16L, M16H, M16F, M16I, S18C, S18Y, S18M, S18L, S18Q, S18G, S18P, S18A, S18W, S18H, S18K, S18I, S18V, S19C, S19V, S19L, S19F, S19K, S19E, S19D, S19G, S19N, S19A, S19M, S19P, S19Y, S19R, S19T, S19Q, S20G, S20M, S20L, S20V, S20H, S20W, S20A, S20C, S20Q, S20D, S20F, S20N, S20R, E21N, E21W, E21G, E21Q, E21L, E21D, E21A, E21P, E21T, E21S, E21Y, E21V, E21F, E21M, E22C, E22H, E22R, E22L, E22K, E22S, E22G, E22M, E22V, E22Q, E22A, E22Y, E22W, E22D, E22T, F23Q, F23A, F23D, F23W, F23K, F23T, F23V, F23M, F23N, F23P, F23H, F23E, F23C, F23R, F23Y, S24L, S24W, S24H, S24V, S24P, S24I, S24F, S24K, S24Y, S24D, S24C, S24N, S24G, S24A, S26F, S26H, S26V, S26Q, S26Y, S26W, S28K, S28Y, S28C, S28M, S28L, S28H, S28T, S28Q, V31L, V31T, V31I, V31Q, V31K, A34L, A34E, L67A, L67T, L67M, L67V, L67C, L67H, L67E, L67Y, G73H, G73N, G73K, G73F, G73V, G73D, G73S, G73W, G73L, A76L, A76R, A76E, A76I, A76V, D77N, D77Q, D77Y, D77L, D77T, P88A, P88E, P88N, P88H, P88D, P88L, N91D, N91R, N91A, N91L, N91H, N91V, Y141I, Y141M, Y141Q, Y141S, Y141E, Y141W, Y141V, Y141F, Y141A, Y141C, Y141K, Y141L, Y141H, Y141R, N145C, N145M, N145A, N145Q, N145I, N145F, N145G, N145D, N145E, N145V, N145H, N145W, N145Y, N145L, N145R, N145S, P146V, P146T, P146W, P146C, P146Q, P146L, P146Y, P146K, P146N, P146F, P146E, P148M, P148R, P148V, P148F, P148T, P148C, P148Q, P148H, Y150W, Y150A, Y150F, Y150H, Y150S, Y150V, Y150C, Y150M, Y150N, Y150D, Y150E, Y150Q, Y150K, H157Y, H157F, H157T, H157S, H157W, A162L, A162V, A162C, A162K, A162T, A162G, A162M, A162S, A162I, A162Y, A162Q, A179T, A179K, A179S, A179V, A179R, L182V, L182I, L182Q, L182T, L182W, L182R, L182S, T189C, T189N, T189L, T189K, T189Q, T189V, T189A, T189W, T189Y, T189G, T189F, T189S, T189H, L192V, L192C, L192H, L192M, L192I, S193P, S193T, S193R, S193K, S193G, S193D, S193N, S193F, S193H, S193Q, S193Y, V196L, V196S, V196W, V196A, V196F, V196M, V196I, S198G, S198R, S198A, S198K, T200C, T200I, T200M, T200L, T200N, T200W, T200V, T200Q, T200Y, T200H, T200R, S202A, S202P, L210H, L210A, F212Y, F212N, F212M, F212C, F212A, N218V, N218R, N218T, N218C, N218G, N218I, N218P, N218D, N218E, A248S, A248L, A248H, A248C, A248N, A248I, A248Q, A248Y, A248M, A248D, L263V, L263A, L263M, L263R, L263D, Q270V, Q270K, Q270A, Q270C, Q270P, Q270L, Q270I, Q270E, Q270G, Q270Y, Q270N, Q270T, Q270W, Q270H, S294R, S294N, S294G, S294T, S294C, T297C, T297P, T297V, T297M, T297L, T297D, E304D, E304H, E304S, E304Q, E304C, S308R, S308G, L310R, L310I, L310V, L333M, L333W, L333F, Q336Y, Q336N, Q336M, Q336A, Q336T, Q336L, Q336I, Q336G, Q336F, Q336E, Q336V, Q336C, Q336H, A354V, A354W, A354D, A354C, A354R, A354E, A354K, A354H, A354G, C357Q, C357H, C357W, C357N, C357I, C357V, C357M, C357R, C357F, C357D, L358A, L358F, L358E, L358R, L358Q, L358V, L358H, L358C, L358M, L358Y, L358K, L358N, L358I, D359N, D359A, D359L, D359H, D359R, D359S, D359Q, D359E, D359M, L377V, L377I, V423N, V423P, V423T, V423F, V423H, V423C, V423S, V423G, V423A, V423R, V423L, P426L, P426K, P426Y, P426F, P426T, P426W, P426V, P426C, P426S, P426Q, P426H, P426N, K428R, K428Q, K428N, K428T, K428F, S434A, S434T, S438Q, S438A, S438M, T447S, T447A, T447C, T447Q, T447N, T447G, L450M, L450V, L450A, L450I, L450E, A462M, A462T, A462Y, A462F, A462K, A462R, A462Q, A462H, A462E, A462N, A462C, V467T, V467C, V467A, V467K, I469V, I469N, I472V, I472L, I472W, I472M, I472F, L476I, L476V, L476N, L476F, L476M, L476C, L476Q, P488E, P488H, P488K, P488Q, P488F, P488M, P488L, P488N, P488D, Q498V, Q498L, Q498G, Q498H, Q498T, Q498C, Q498E, Q498M, L502I, L502M, L502V, L502G, L502F, E517M, E517V, E517A, E517K, E517L, E517G, E517S, E517I, P520W, P520R, P520M, P520F, P520Q, P520V, P520G, P520D, P520K, P520Y, P520E, P520L, P520T, S521A, S521H, S521C, S521V, S521W, S521T, S521K, S521F, S521G, N523W, N523A, N523G, N523S, N523P, N523M, N523Q, N523L, N523K, N523D, N523H, N523F, N523C, I533M, I533V, I533T, I533S, I533F, I533G, I533E, D534E, D534Q, D534L, D534R, D534V, D534C, D534M, D534N, D534A, D534G, D534F, D534T, D534H, D534K, D534S, F576L, F576K, F576V, F576D, F576W, F576M, F576C, F576R, F576Q, F576A, F576Y, F576N, F576G, F576I, F576E, K577L, K577G, K577D, K577R, K577H, K577Y, K577I, K577E, K577V, K577N, I582V, I582K, I582R, I582M, I582G, I582N, I582E, I582A, I582Q, Y583L, Y583C, Y583F, Y583D, Y583Q, L587F, L587D, L587R, L587I, L587P, L587N, L587E, L587S, L587Y, L587M, L587Q, L587G, L587W, L587K or L587T.

An advantageous piggyBac-like transposon for modifying the genome of a mammalian cell is a Bombyx transposon which comprises an ITR with the nucleotide sequence of SEQ ID NO: 427, a heterologous polynucleotide to be transposed and a second ITR with the nucleotide sequence of SEQ ID NO: 428. The transposon may further be flanked by a copy of the tetranucleotide 5′-TTAA-3′ on each side, immediately adjacent to the ITRs and distal to the heterologous polynucleotide. The transposon may further comprise a nucleotide sequence immediately adjacent to the ITR and proximal to the heterologous polynucleotide that is at least 95% identical to SEQ ID NO: 425 on one side of the heterologous polynucleotide, preferably the left side, and a sequence immediately adjacent to the ITR and proximal to the heterologous polynucleotide that is at least 95% identical to SEQ ID NO: 426 on the other side of the heterologous polynucleotide, preferably the right side. This transposon may be transposed by a corresponding Bombyx transposase comprising a polypeptide sequence at least 90% identical to SEQ ID NO: 498, for example any of SEQ ID NOs: 498-520. Preferably the transposase is a hyperactive variant of a naturally occurring transposase. Preferably the hyperactive variant transposase comprises one or more of the following amino acid changes, relative to the sequence of SEQ ID NO: 498: Q85E, Q85M, Q85K, Q85H, Q85N, Q85T, Q85F, Q85L, Q92E, Q92A, Q92P, Q92N, Q92I, Q92Y, Q92H, Q92F, Q92R, Q92D, Q92M, Q92W, Q92C, Q92G, Q92L, Q92V, Q92T, V93P, V93K, V93M, V93F, V93W, V93L, V93A, V93I, V93Q, P96A, P96T, P96M, P96R, P96G, P96V, P96E, P96Q, P96C, F97Q, F97K, F97H, F97T, F97C, F97W, F97V, F97E, F97P, F97D, F97A, F97R, F97G, F97N, F97Y, H165E, H165G, H165Q, H165T, H165M, H165V, H165L, H165C, H165N, H165D, H165K, H165W, H165A, E178S, E178H, E178Y, E178F, E178C, E178A, E178Q, E178G, E178V, E178D, E178L, E178P, E178W, C189D, C189Y, C189I, C189W, C189T, C189K, C189M, C189F, C189P, C189Q, C189V, A196G, L200I, L200F, L200C, L200M, L200Y, A201Q, A201L, A201M, L203V, L203D, L203G, L203E, L203C, L203T, L203M, L203A, L203Y, N207G, N207A, L211G, L211M, L211C, L211T, L211V, L211A, W215Y, T217V, T217A, T217I, T217P, T217C, T217Q, T217M, T217F, T217D, T217K, G219S, G219A, G219C, G219H, G219Q, Q235C, Q235N, Q235H, Q235G, Q235W, Q235Y, Q235A, Q235T, Q235E, Q235M, Q235F, Q238C, Q238M, Q238H, Q238V, Q238L, Q238T, Q238I, R242Q, K246I, K253V, M258V, F261L, S263K, C271S, N303C, N303R, N303G, N303A, N303D, N303S, N303H, N303E, N303R, N303K, N303L, N303Q, I312F, I312C, I312A, I312L, I312I, I312V, I312G, I312M, F321H, F321R, F321N, F321Y, F321W, F321D, F321G, F321E, F321M, F321K, F321A, F321Q, V323I, V323L, V323T, V323M, V323A, V324N, V324A, V324C, V324I, V324L, V324T, V324K, V324Y, V324H, V324F, V324S, V324Q, V324M, V324G, A330K, A330V, A330P, A330S, A330C, A330T, A330L, Q333P, Q333T, Q333M, Q333H, Q333S, P337W, P337E, P337H, P337I, P337A, P337M, P337N, P337D, P337K, P337Q, P337G, P337S, P337C, P337L, P337V, F368Y, L373C, L373V, L373I, L373S, L373T, V389I, V389M, V389T, V389L, V389A, R394H, R394K, R394T, R394P, R394M, R394A, Q395P, Q395F, Q395E, Q395C, Q395V, Q395A, Q395H, Q395S, Q395Y, S399N, S399E, S399K, S399H, S399D, S399Y, S399G, S399Q, S399R, S399T, S399A, S399V, S399M, R402Y, R402K, R402D, R402F, R402G, R402N, R402E, R402M, R402S, R402Q, R402I, R402C, R402L, R402V, I403W, I403A, I403V, I403F, I403L, I403Y, I403N, I403G, I403C, I403I, I403S, I403M, I403Q, I403K, T403E, D404I, D404S, D404E, D404N, D404H, D404C, D404M, D404G, D404A, D404Q, D404L, D404P, D404V, D404W, D404F, N408F, N408I, N408A, N408E, N408M, N408S, N408D, N408Y, N408H, N408C, N408Q, N408V, N408W, N408L, N408P, N408K, S409H, S409Y, S409N, S409I, S409D, S409F, S409T, S409C, S409Q, N441F, N441R, N441M, N441G, N441C, N441D, N441L, N441A, N441V, N441W, G448W, G448Y, G448H, G448C, G448I, G448V, G448N, G448Q, E449A, E449P, E449T, E449L, E449H, E449G, E449C, E449I, V469T, V469A, V469H, V469C, V469L, L472K, L472Q, L472M, C473G, C473Q, C473T, C473I, C473M, R484H, R484K, T507R, T507D, T507S, T507G, T507K, T507I, T507M, T507E, T507C, T507L, T507V, G523Q, G523T, G523A, G523M, G523S, G523C, G523I, G523L, I527M, I527V, Y528N, Y528W, Y528M, Y528Q, Y528K, Y528V, Y528I, Y528G, Y528D, Y528A, Y528E, Y528R, Y543C, Y543W, Y543I, Y543M, Y543Q, Y543A, Y543R, Y543H, E549K, E549C, E549I, E549Q, E549A, E549H, E549C, E549M, E549S, E549F, E549L, K550R, K550M, K550Q, S556G, S556V, S556I, P557W, P557T, P557S, P557A, P557Q, P557K, P557D, P557G, P557N, P557L, P557V, H559K, H559S, H559C, H559I, H559W, V560F, V560P, V560I, V560H, V560Y, V560K, N561P, N561Q, N561G, N561A, V562Y, V562I, V562S, V562M, V567I, V567H, V567N, S583M, E601V, E601F, E601Q, E601W, E605R, E605W, E605K, E605M, E605P, E605Y, E605C, E605H, E605A, E605Q, E605S, E605V, E605I, E605G, D607V, D607Y, D607C, D607N, D607W, D607T, D607A, D607H, D607Q, D607E, D607L, D607K, D607G, S609R, S609W, S609H, S609V, S609Q, S609G, S609T, S609K, S609N, S609Y, L610T, L610I, L610K, L610G, L610A, L610W, L610D, L610Q, L610S, L610F or L610N.

An advantageous piggyBac-like transposon for modifying the genome of a mammalian cell is a piggyBat transposon which comprises an ITR with the nucleotide sequence of SEQ ID NO: 433, a heterologous polynucleotide to be transposed and a second ITR with the nucleotide sequence of SEQ ID NO: 434. The transposon may further be flanked by a copy of the tetranucleotide 5′-TTAA-3′ on each side, immediately adjacent to the ITRs and distal to the heterologous polynucleotide. The transposon may further comprise a nucleotide sequence immediately adjacent to the ITR and proximal to the heterologous polynucleotide that is at least 95% identical to SEQ ID NO: 435 on one side of the heterologous polynucleotide, preferably the left side, and a nucleotide sequence immediately adjacent to the ITR and proximal to the heterologous polynucleotide that is at least 95% identical to SEQ ID NO: 436 on the other side of the heterologous polynucleotide, preferably the right side. This transposon may be transposed by a corresponding piggyBat transposase comprising a polypeptide sequence at least 90% identical to SEQ ID NO: 462. Preferably the transposase is a hyperactive variant of a naturally occurring transposase. Preferably the hyperactive variant transposase comprises one or more of the following amino acid changes, relative to the sequence of SEQ ID NO: 462: A14V, D475G, P491Q, A561 T, T546T, T300A, T294A, A520T, G239S, S5P, S8F, S54N, D9N, D9G, 1345 V, M481V, E11G, K130T, G9G, R427H, S8P, S36G, D1OG, S36G.

An advantageous piggyBac-like transposon for modifying the genome of a mammalian cell is a piggyBac transposon which comprises an ITR with the nucleotide sequence of SEQ ID NO: 431, a heterologous polynucleotide to be transposed and a second ITR with the nucleotide sequence of SEQ ID NO: 432. The transposon may further be flanked by a copy of the tetranucleotide 5′-TTAA-3′ on each side, immediately adjacent to the ITRs and distal to the heterologous polynucleotide. The transposon may further comprise a nucleotide sequence immediately adjacent to the ITR and proximal to the heterologous polynucleotide that is at least 95% identical to SEQ ID NO: 429 on one side of the heterologous polynucleotide, preferably the left side, and a nucleotide sequence immediately adjacent to the ITR and proximal to the heterologous polynucleotide that is at least 95% identical to SEQ ID NO: 430 on the other side of the heterologous polynucleotide preferably the right side. This transposon may be transposed by a corresponding piggyBac transposase comprising a polypeptide sequence at least 90% identical to SEQ ID NO: 463. Preferably the transposase is a hyperactive variant of a naturally occurring transposase. Preferably the hyperactive variant transposase comprises one or more of the following amino acid changes, relative to the sequence of SEQ ID NO: 463: G2C, Q40R, I30V, G165S, T43A, S61R, S103P, S103T, M194V, R281G, M282V, G316E, I426V, Q497L, N505D, Q573L, S509G, N570S, N538K, Q591P, Q591R, F594L, M194V, 130V, S103P, G165S, M282V, S509G, N538K, N571S, C41T, A1424G, C1472A, G1681A, T150C, A351G, A279G, T1638C, A898G, A880G, G1558A, A687G, G715A, T13C, C23T, G161A, G25A, T1050C, A1356G, A26G, A1033G, A1441G, A32G, A389C, A32G, A389C, A32G, T1572A, G456A, T1641C, Tl 155C, G1280A, T22C, A106G, A29G, C137T, A14V, D475G, P491Q, A561T, T546T, T300A, T294A, A520T, G239S, S5P, S8F, S54N, D9N, D9G, 1345 V, M481V, E11G, K130T, G9G, R427H, S8P, S36G, D10G, S36G, A51T, C153A, C277T, G201A, G202A, T236A, A103T, A104C, T140C, G138T, T118A, C74T, A179C, S3N, 130V, A46S, A46T, I82W, S103P, R119P, C125A, C125L, G165S, Y177K, Y177H, F180L, F180I, F180V, M185L, A187G, F200W, V207P, V209F, M226F, L235R, V240K, F241L, P243K, N258S, M282Q, L296W, L296Y, L296F, M298V, M298A, M298L, P311V, P3111, R315K, T319G, Y327R, Y328V, C340G, C340L, D421H, V436I, M456Y, L470F, S486K, M5031, M503L, V552K, A570T, Q591P, Q591R, R65A, R65E, R95A, R95E, R97A, R97E, R135A, R135E, R161A, R161E, R192A, R192E, R208A, R208E, K176A, K176E, K195A, K195E, S171E, M14V, D270N, 130V, G165S, M282L, M2821, M282V or M282A.

An advantageous piggyBac-like transposon for modifying the genome of a cultured mammalian cell is an Amyelois transposon comprising an ITR with the nucleotide sequence of SEQ ID NO: 439, a heterologous polynucleotide and a second ITR with the nucleotide sequence of SEQ ID NO: 440. The transposon may further be flanked by a copy of the tetranucleotide 5′-TTAA-3′ on each side, immediately adjacent to the ITRs and distal to the heterologous polynucleotide. The transposon may further comprise a nucleotide sequence that is at least 95% identical to SEQ ID NO: 437 on one side of the heterologous polynucleotide, and a nucleotide sequence that is at least 95% identical to SEQ ID NO: 438 on the other side of the heterologous polynucleotide. This transposon may be transposed by a corresponding Amyelois transposase comprising a polypeptide sequence at least 90% identical to SEQ ID NO: 521. Preferably the transposase is a hyperactive variant of a naturally occurring transposase. Preferably the hyperactive variant transposase comprises one or more of the following amino acid changes, relative to the sequence of SEQ ID NO: 521: P65E, P65D, R95S, R95T, V100I, V100L, V100M, L115D, L115E, E116P, H121Q, H121N, K139E, K139D, T159N, T159Q, V166F, V166Y, V166W, G179N, G179Q, W187F, W187Y, P198R, P198K, L203R, L203K, I209L, I209V, I209M, N211R, N211K, E238D, L273I, L273V, L273M, D304K, D304R, I323L, I323M, I323V, Q329G, Q329R, Q329K, T345L, T345I, T345V, T345M, K362R, T366R, T366K, T380S, L408M, L408I, L408V, E413S, E413T, S416E, S416D, I426M, I426L, I426V, S435G, L458M, L458I, L458V, A472S, A472T, V475I, V475L, V475M, N483K, N483R, I491M, I491V, I491L, A529P, K540R, S560K, S560R, T562K, T562R, S563K, S563R.

An advantageous piggyBac-like transposon for modifying the genome of a cultured mammalian cell is a Heliothis transposon comprising an ITR with the nucleotide sequence of SEQ ID NO: 443, a heterologous polynucleotide and a second ITR with the nucleotide sequence of SEQ ID NO: 444. The transposon may further be flanked by a copy of the tetranucleotide 5′-TTAA-3′ on each side, immediately adjacent to the ITRs and distal to the heterologous polynucleotide. The transposon may further comprise a nucleotide sequence that is at least 95% identical to SEQ ID NO: 441 on one side of the heterologous polynucleotide, and a nucleotide sequence that is at least 95% identical to SEQ ID NO: 442 on the other side of the heterologous polynucleotide. This transposon may be transposed by a corresponding Heliothis transposase comprising a polypeptide sequence at least 90% identical to SEQ ID NO: 522. Preferably the transposase is a hyperactive variant of a naturally occurring transposase. Preferably the hyperactive variant transposase comprises one or more of the following amino acid changes, relative to the sequence of SEQ ID NO: 522: S41V, S41I, S41L, L43S, L43T, V81E, V81D, D83S, D83T, V85L, V851, V85M, P125S, P125T, Q126S, Q126T, Q131R, Q131K, Q131T, Q131S, S136V, S136I, S136L, S136M, E140C, E140A, N151Q, K169E, K169D, N212S, I239L, I239V, I239M, H241N, H241Q, T268D, T268E, T297C, M300R, M300K, M305N, M305Q, L312I, C316A, C316M, L321V, L321M, N322T, N322S, P351G, H357R, H357K, H357D, H357E, K360Q, K360N, E379P, K397S, K397T, Y421F, Y421W, V450I, V450L, V450M, Y495F, Y495W, A447N, A447D, A449S, A449V, K476L, V492A, I500M, L585K and T595K.

An advantageous piggyBac-like transposon for modifying the genome of a cultured mammalian cell is an Oryzias transposon comprising an ITR with the nucleotide sequence of SEQ ID NO: 564, a heterologous polynucleotide and a second ITR with the nucleotide sequence of SEQ ID NO: 447. The transposon may further be flanked by a copy of the tetranucleotide 5′-TTAA-3′ on each side, immediately adjacent to the ITRs and distal to the heterologous polynucleotide. The transposon may further comprise a nucleotide sequence that is at least 95% identical to SEQ ID NO: 445 on one side of the heterologous polynucleotide, and a nucleotide sequence that is at least 95% identical to SEQ ID NO: 446 on the other side of the heterologous polynucleotide. This transposon may be transposed by a corresponding Oryzias transposase comprising a polypeptide sequence at least 90% identical to SEQ ID NO: 523. Preferably the transposase is a hyperactive variant of a naturally occurring transposase. Preferably the hyperactive variant transposase comprises one or more of the following amino acid changes, relative to the sequence of SEQ ID NO: 523: E22D, A124C, Q131D, Q131E, L138V, L138I, L138M, D160E, Y164F, Y164W, I167L, I167V, I167M, T202R, T202K, I206L, I206V, I206M, I210L, I210V, I210M, N214D, N214E, V253I, V253L, V253M, V258L, V258I, V258M, A284L, A284I, A284M, A284V, V386I, V386M, V386L, M400L, M400I, M400V, S408E, S408D, L409I, L409V, L409M, V458L, V458M, V458I, V467I, V467M, V467L, L468I, L468V, L468M, A514R, A514K, V515I, V515M, V515L, R548K, D549K, D549R, D550R, D550K, S551K and S551R

An advantageous piggyBac-like transposon for modifying the genome of a cultured mammalian cell is an Agrotis transposon comprising an ITR with the nucleotide sequence of SEQ ID NO: 452, a heterologous polynucleotide and a second ITR with the nucleotide sequence of SEQ ID NO: 453. The transposon may further be flanked by a copy of the tetranucleotide 5′-TTAA-3′ on each side, immediately adjacent to the ITRs and distal to the heterologous polynucleotide. The transposon may further comprise a nucleotide sequence that is at least 95% identical to SEQ ID NO: 450 on one side of the heterologous polynucleotide, and a nucleotide sequence that is at least 95% identical to SEQ ID NO: 451 on the other side of the heterologous polynucleotide. This transposon may be transposed by a corresponding Agrotis transposase comprising a polypeptide sequence at least 90% identical to SEQ ID NO: 524. Preferably the transposase is a hyperactive variant of a naturally occurring transposase.

An advantageous piggyBac-like transposon for modifying the genome of a cultured mammalian cell is a Helicoverpa transposon comprising an ITR with the nucleotide sequence of SEQ ID NO: 456, a heterologous polynucleotide and a second ITR with the nucleotide sequence of SEQ ID NO: 457. The transposon may further be flanked by a copy of the tetranucleotide 5′-TTAA-3′ on each side, immediately adjacent to the ITRs and distal to the heterologous polynucleotide. The transposon may further comprise a nucleotide sequence that is at least 95% identical to SEQ ID NO: 454 on one side of the heterologous polynucleotide, and a nucleotide sequence that is at least 95% identical to SEQ ID NO: 455 on the other side of the heterologous polynucleotide. This transposon may be transposed by a corresponding Helicoverpa transposase comprising a polypeptide sequence at least 90% identical to SEQ ID NO: 525. Preferably the transposase is a hyperactive variant of a naturally occurring transposase.

An advantageous Mariner transposon for modifying the genome of a mammalian cell is a Sleeping Beauty transposon, for example one that comprises an ITR with the nucleotide sequence of SEQ ID NO: 460, a heterologous polynucleotide and a second ITR with the nucleotide sequence of SEQ ID NO: 461. An advantageous Mariner transposon for modifying the genome of a mammalian cell comprises a first transposon end with at least 90% sequence identity to SEQ ID NO: 458, and a second transposon end with at least 90% sequence identity to SEQ ID NO: 459. This transposon may be transposed by a corresponding Sleeping Beauty transposase comprising a polypeptide sequence at least 90% identical to SEQ ID NO: 464, including hyperactive variants thereof.

An advantageous hAT transposon for modifying the genome of a mammalian cell is a TcBuster transposon, for example one that comprises an ITR with the nucleotide sequence of SEQ ID NO: 528, a heterologous polynucleotide and a second ITR with the nucleotide sequence of SEQ ID NO: 529. An advantageous hAT transposon for modifying the genome of a mammalian cell comprises a first transposon end with at least 90% sequence identity to SEQ ID NO: 526, and a second transposon end with at least 90% sequence identity to SEQ ID NO: 527. This transposon may be transposed by a corresponding TcBuster transposase comprising a polypeptide sequence at least 90% identical to SEQ ID NO: 530, including hyperactive variants thereof.

A transposase protein can be introduced into a cell as a protein or as a nucleic acid encoding the transposase, for example as a ribonucleic acid, including mRNA or any polynucleotide recognized by the translational machinery of a cell; as DNA, e.g. as extrachromosomal DNA including episomal DNA; as plasmid DNA, or as viral nucleic acid. Furthermore, the nucleic acid encoding the transposase protein can be transfected into a cell as a nucleic acid vector such as a plasmid, or as a gene expression vector, including a viral vector. The nucleic acid can be circular or linear. DNA encoding the transposase protein can be stably inserted into the genome of the cell or into a vector for constitutive or inducible expression. Where the transposase protein is transfected into the cell or inserted into the vector as DNA, the transposase encoding sequence is preferably operably linked to a heterologous promoter. There are a variety of promoters that could be used including constitutive promoters, tissue-specific promoters, inducible promoters, species-specific promoters, cell-type specific promoters and the like. All DNA or RNA sequences encoding transposase proteins are expressly contemplated. Alternatively, the transposase may be introduced into the cell directly as protein, for example using cell-penetrating peptides (e.g. as described in Ramsey and Flynn, 2015. Pharmacol. Ther. 154: 78-86 “Cell-penetrating peptides transport therapeutics into cells”); using small molecules including salt plus propanebetaine (e.g. as described in Astolfo et. al., 2015. Cell 161: 674-690); or electroporation (e.g. as described in Morgan and Day, 1995. Methods in Molecular Biology 48: 63-71 “The introduction of proteins into mammalian cells by electroporation”).

### 5.2.3 Promoter Elements

Systems for expression of polypeptides or amiRNAs in cultured mammalian cells comprise a polynucleotide to be transferred to a host cell. The polynucleotide comprises a promoter that is active in the cultured mammalian cell, operably linked to a heterologous sequence to be expressed. Advantageous gene transfer polynucleotides for the expression of amiRNAs in mammalian cells comprise a Pol II promoter such as an EF1a promoter from any mammalian or avian species including human, rat, mice, chicken and Chinese hamster, (for example a nucleotide sequence selected from SEQ ID NOS: 310-331); a promoter from the immediate early genes 1, 2 or 3 of cytomegalovirus (CMV) from either human, primate or rodent cells (for example a nucleotide sequence selected from SEQ ID NOS: 332-343); a promoter for eukaryotic elongation factor 2 (EEF2) from any mammalian or avian species including human, rat, mice, chicken and Chinese hamster, (for example a nucleotide sequence selected from SEQ ID NOS: 344-354); a Glyceraldehyde 3-phosphate dehydrogenase (GAPDH) promoter from any mammalian or yeast species (for example a nucleotide sequence selected from SEQ ID NOS: 365-381), an actin promoter from any mammalian or avian species including human, rat, mice, chicken and Chinese hamster (for example a nucleotide sequence selected from SEQ ID NOS: 355-364); a PGK promoter from any mammalian or avian species including human, rat, mice, chicken and Chinese hamster (for example a nucleotide sequence selected from SEQ ID NOS: 382-391), or a ubiquitin promoter (for example nucleotide sequence SEQ ID NO: 392), or a viral promoter such as an HSV-TK promoter or an SV40 promoter (for example a nucleotide sequence selected from SEQ ID NOS: 393-399) operably linked to a multi-hairpin amiRNA sequence. Alternatively, a multi-hairpin amiRNA sequence may be operably linked to a Pol III promoter such as a U6 promoter (for example a nucleotide sequence selected from SEQ ID NOs: 404-408) or an H1 promoter (for example nucleotide sequence SEQ ID NO: 409).

### 5.2.4 Micro RNA Elements

Small inhibitory RNAs (siRNAs) have been used to reduce the activity of certain genes within mammalian culture cells through RNA interference. An siRNA can be expressed in a cell from a nucleic acid encoding a short hairpin RNA (shRNA) operably linked to a promoter naturally transcribed by RNA polymerase III (a “Pol III promoter”). Naturally occurring shRNAs may also be expressed from nucleic acids operably linked to a promoter naturally transcribed by RNA polymerase II (a “Pol II promoter”). The Pol II promoter is typically responsible for transcription of most protein-encoding genes. The products of natural Pol II-expressible shRNA genes are referred to as microRNAs (miRNAs).

Expression of targeted shRNAs within mammalian cells can be accomplished by engineering natural miRNAs, replacing the natural guide strand sequence with a sequence complementary to a target mRNA whose expression is to be reduced, thereby creating an artificial miRNA (amiRNA) as described for the miR-30 micro RNA (Zeng et. al., 2002. Both Natural and Designed Micro RNAs Technique Can Inhibit the Expression of Cognate mRNAs When Expressed in Human Cells. Molecular Cell: 9, 1327-1333).

The reduction in gene expression in mammalian cells that can be achieved through RNA interference using amiRNA is variable. Success is often limited because of the limited efficacy of any single inhibitory RNA. Strategies that have been described to improve the efficacy of RNA interference include the incorporation of mismatches in the intramolecular RNA duplex (Wu et. al., 2011. Improved siRNA/shRNA Functionality by Mismatched Duplex. PLoS ONE 6(12): e28580. doi:10.1371/journal.pone.0028580; Myburgh et. al., 2014. Optimization of Critical Hairpin Features Allows miRNA-based Gene Knockdown Upon Single-copy Transduction. Molecular Therapy-Nucleic Acids 3, e207; doi:10.1038/mtna.2014.58), insertion of spacer regions within the amiRNA genes, between the Pol II promoter and the sequences of the amiRNA hairpins (Rousset et. al., 2019. Optimizing Synthetic miRNA Minigene Architecture for Efficient miRNA Hairpin Concatenation and Multi-target Gene Knockdown. Molecular Therapy-Nucleic Acids 14, 351-363.), and the concatenation of amiRNA hairpins within an amiRNA gene (Sun et al., 2006. Multi-miRNA hairpin method that improves gene knockdown efficiency and provides linked multi-gene knockdown. BioTechniques 41:59-63 doi 10.2144/000112203).

Although amiRNA genes comprising multiple copies of the same hairpin have been shown to be more effective than amiRNA genes with only a single copy of the hairpin, even with three identical hairpins in a single lentiviral vector, it is difficult to reduce expression of the target gene to less than 10% of normal levels (Sun et al., 2006 ibid, Rousset et. al., 2019 ibid). The other application for genes comprising multiple amiRNA hairpins has been for simultaneous inhibition of multiple genes (Hu et. al., 2009. Construction of an Artificial MicroRNA Expression Vector for Simultaneous Inhibition of Multiple Genes in Mammalian Cells. Int. J. Mol. Sci. 10, 2158-2168; Choi et al, 2015. Mol. Ther. 23, 310-320. “Multiplexing Seven miRNA-Based shRNAs to Suppress HIV Replication”).

Instead of targeting one sequence in a target mRNA with multiple identical inhibitory RNAs derived from multiple identical hairpins, we have designed amiRNA genes comprising multiple different hairpins, each for the expression of a different inhibitory RNA guide strand complementary to different regions within the same target mRNA. Because the guide strand sequences derived from each hairpin target different areas of the gene, they are essentially independent. Furthermore, the processing of hairpins to produce RISC-associated guide strands is improved if multiple hairpins are contained within the same RNA transcript. In addition, the use of multiple independent guide strands reduces the risk of unwanted off-target effects since it is not necessary to express any individual guide strand at extremely high levels. It is thus advantageous to use a polynucleotide comprising two or three or four or five or more hairpins which will be expressed within a mammalian cell to produce two or three or four or five or more different inhibitory RNA guide strands, each of which is complementary to a different sequence within the same target mRNA. When more than one hairpin for the expression of inhibitory RNA guide strands are operably linked to the same promoter, we refer to them as a multi-hairpin amiRNA gene.

Instead of designing a new multi-hairpin amiRNA for inhibition of each new target gene, an alternative strategy is to use an existing well characterized multi-hairpin amiRNA with guide RNAs complementary to two or more different target sites within an existing polynucleotide. A cell containing a gene whose expression is to be inhibited is modified so the gene expresses its original mRNA fused to a segment including the target sites of the amiRNA. Optionally, the fusion occurs with a UTR of an mRNA to be inhibited, or corresponding portion of a polynucleotide encoding the mRNA. That is, the fusion can occur within a 3′ UTR between the coding sequence and polyadenylation sequence or within a 5′ UTR.

Preferably, when integrated into the genome of a mammalian cell, the multi-hairpin amiRNA gene reduces the expression of the target gene to a level lower than the level of expression of the target gene in a mammalian cell whose genome comprises an amiRNA gene comprising a hairpin for expression of a single inhibitory RNA guide strand. Preferably, when integrated into the genomes of a population of mammalian cells, the multi-hairpin amiRNA gene reduces the average expression of the target gene within the population to a level lower than the level of expression of the target gene in a population of mammalian cells whose genomes comprises an amiRNA gene comprising a hairpin for expression of a single inhibitory RNA guide strand. Preferably, when integrated into the genomes of a population of mammalian cells, the multi-hairpin amiRNA gene reduces the expression of the target gene to less than 50% of the natural level in a greater fraction of the population than the fraction of the population in which expression is reduced to less than 50% in a population of mammalian cells whose genomes comprises an amiRNA gene comprising a hairpin for expression of a single inhibitory RNA guide strand. Preferably, when integrated into the genomes of a population of mammalian cells, the multi-hairpin amiRNA gene reduces the expression of the target gene to less than 40% of the natural level in a greater fraction of the population than the fraction of the population in which expression is reduced to less than 40% in a population of mammalian cells whose genomes comprises an amiRNA gene comprising a hairpin for expression of a single inhibitory RNA guide strand. Preferably, when integrated into the genomes of a population of mammalian cells, the multi-hairpin amiRNA gene reduces the expression of the target gene to less than 30% of the natural level in a greater fraction of the population than the fraction of the population in which expression is reduced to less than 30% in a population of mammalian cells whose genomes comprises an amiRNA gene comprising a hairpin for expression of a single inhibitory RNA guide strand. Preferably, when integrated into the genomes of a population of mammalian cells, the multi-hairpin amiRNA gene reduces the expression of the target gene to less than 20% of the natural level in a greater fraction of the population than the fraction of the population in which expression is reduced to less than 20% in a population of mammalian cells whose genomes comprises an amiRNA gene comprising a hairpin for expression of a single inhibitory RNA guide strand. Preferably, when integrated into the genomes of a population of mammalian cells, the multi-hairpin amiRNA gene reduces the expression of the target gene to less than 10% of the natural level in a greater fraction of the population than the fraction of the population in which expression is reduced to less than 10% in a population of mammalian cells whose genomes comprises an amiRNA gene comprising a hairpin for expression of a single inhibitory RNA guide strand. Preferably, when integrated into the genomes of a population of mammalian cells, the multi-hairpin amiRNA gene reduces the expression of the target gene to less than 5% of the natural level in a greater fraction of the population than the fraction of the population in which expression is reduced to less than 5% in a population of cultured mammalian cells whose genomes comprises an amiRNA gene comprising a hairpin for expression of a single inhibitory RNA guide strand. Preferably, the hairpin for expression of the single inhibitor RNA guide strand for comparison with a multihairpin is either of the individual hairpins in the multihairpin. Some multi-hairpins achieve a more than additive level of inhibition compared with their component hairpins expressed under the same conditions. For example, if each individual hairpin of a two-hairpin multi-hairpin results in 10% inhibition, then a 21% or higher level of inhibition by the multi-hairpin under the same conditions is more than additive. Some multi-hairpins of the invention including multiple hairpins to different segments of a target mRNA achieve greater inhibition of a target gene than control multi-hairpins including the same number of hairpins but as tandem copies of the same hairpin, when the same hairpin is any of the component hairpins of a multi-hairpin of the invention. For purposes of such a comparison, a hairpin of the invention and control hairpin are the same except for their respective hairpin compositions and are tested for inhibition in the same circumstances (e.g., expressed from the same promoter and in the same cell type). Preferably a multi-hairpin of the invention achieves greater inhibition of a target gene than control tandem hairpins formed of any one of the component hairpins of the multi-hairpin of the invention.

Preferably, when integrated into the genome of a mammalian cell, the multi-hairpin amiRNA gene reduces the expression of the target gene to less than 50% or 40% or 30% or 20% or 10% or 5% or 2% or 1% of the natural expression level of the target gene. Such reduction of expression may be detected directly as a reduction in mRNA levels or of protein levels, but it may also be detected as a corresponding decrease in the function or activity for which the target gene is responsible. For example, if the product of the target gene is an intracellular protein, preferably, when integrated into the genome of a mammalian cell, the multi-hairpin amiRNA gene reduces the activity of the product of the target gene within the cell to less than 50% or 40% or 30% or 20% or 10% or 5% or 2% or 1% of the natural activity of the product of the target gene within the cell. If the product of the target gene is an extracellular protein, preferably, when integrated into the genome of a mammalian cell, the multi-hairpin amiRNA gene reduces the activity of the product of the target gene secreted from the cell to less than 50% or 40% or 30% or 20% or 10% or 5% or 2% or 1% of the natural activity of the product of the target gene secreted from the cell. If the product of the target gene is a transmembrane protein such as a receptor protein with a signaling function, preferably, when integrated into the genome of a mammalian cell, the multi-hairpin amiRNA gene reduces signal transduction by the product of the target gene to less than 50% or 40% or 30% or 20% or 10% or 5% or 2% or 1% of the natural signal transduction by the product of the target gene. If normal expression of the target gene results in modification of a product made by the mammalian cell, when the multi-hairpin amiRNA gene is integrated into the genome of a mammalian cell, expression of the target gene is preferably reduced such that less than 50% or 40% or 30% or 20% or 10% or 5% or 2% or 1% of the product made by the mammalian cell is modified by the action of the target gene product. If normal expression of the target gene results in modification of a product made by the mammalian cell, when the multi-hairpin amiRNA gene is integrated into the genome of a mammalian cell, expression of the target gene is preferably reduced such that the extent of product modification resulting from the expression of the target gene is reduced to less than 50% or 40% or 30% or 20% or 10% or 5% or 2% or 1% of the extent to which the product would be modified in the absence of the multi-hairpin amiRNA gene. Product modifications include the proteolytic cleavage, or glycosylation or other post-translational modification of a protein produced by the mammalian cell.

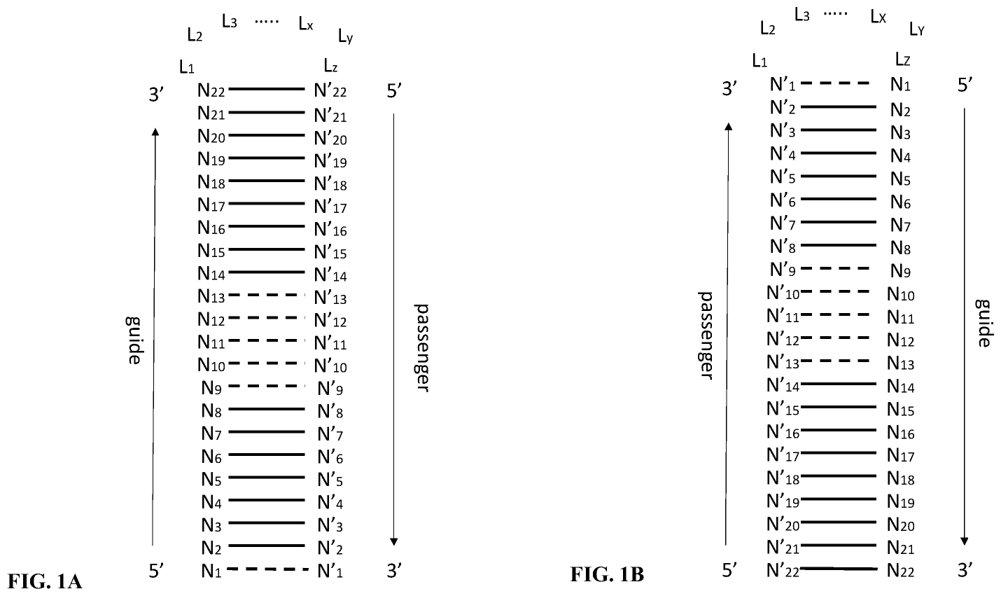

The guide strand sequence of an amiRNA comprises 19 or 20 or 21 or 22 bases that are complementary to the mRNA of the target gene. The guide strand sequence may be complementary to any part of the mRNA, preferably it is complementary to the 3′ UTR of the mRNA or the 5′ UTR of the mRNA or the coding region of the mRNA. Preferably the 5′ base of the guide strand sequence is a thymine (T). The passenger strand sequence of an amiRNA is complementary to the guide strand sequence. It is often advantageous for appropriate processing of an amiRNA if the passenger strand sequence is not perfectly complementary to the guide strand sequence. Processing is often improved if the passenger strand sequence is mismatched at the base complementary to the 5′ base of the guide strand sequence. A general schematic of an exemplary amiRNA hairpin is shown in FIGS. 1A-B. Preferably the passenger strand sequence comprises a mismatch in complementarity with the guide strand sequence at the base corresponding to the 5′ base of the guide strand sequence (base N₁ in FIG. 1). If the 5′ base of the guide strand sequence is an adenine (A) or thymine (T), the passenger strand sequence preferably comprises a cytosine (C) in the corresponding complementary position (base N′₁ in FIG. 1). If the 5′ base of the guide strand sequence is a cytosine (C) or guanine (G), the passenger strand sequence preferably comprises an adenine (A) in the corresponding complementary position. One, two or three additional mismatches may be incorporated into the passenger strand sequence as mismatched bases, insertions or deletions. Most favorable mismatches are made in the passenger strand sequence that create mismatches at one or more of the corresponding positions complementary to positions 9, 10, 11, 12 or 13 in the guide strand sequence (bases N9, N10, N11, N12 and N13 in FIGS. 1A-B). Most preferably, the passenger strand sequence comprises a mismatch at the base corresponding to position 12 in the guide strand sequence (base N′12 in FIGS. 1A-B). The guide and the passenger strand sequences of an amiRNA are typically separated by an unstructured loop of between 5 and 35 nucleotides (bases L₁-L_{Z} in FIGS. 1A-B.). Preferably the loop comprises a sequence derived from a naturally occurring miRNA, for example a nucleotide sequence selected from SEQ ID NO: 241-250.

A preferred polynucleotide for the inhibition of a target gene (“the inhibitory polynucleotide”) comprises a multi-hairpin amiRNA gene comprising at least two different amiRNA hairpin sequences whose guide strand sequences are different and are each complementary to a different sequence in the same target mRNA. An mRNA can be subdivided into a 5′ UTR, coding region and 3′ UTR. The target sites for hairpin inhibitors can be in the same or different of these regions. For example, there can be target sites for two hairpins both in the 3′ UTR or one site in the 3′ UTR and another in the coding region. Spacing between target sites can vary from over 5000 nucleotides to overlapping. A preferred spacing is between 5 and 2,000 nucleotides. Spacing is measured as the number of nucleotides between proximate 3′ and 5′ ends of target sites.

The multi-hairpin amiRNA gene comprises a first (guide strand) sequence of at least 19 or 20 or 21 or 22 contiguous bases that are complementary to the target mRNA and a first (passenger strand) sequence of at least 19 or 20 or 21 or 22 bases that are at least 78% identical to the reverse complement of the first guide strand sequence (i.e. within 19 bases it comprises no more than 4 mismatches, including mutations, single base deletions or single base insertions, relative to the identical reverse complement of the first guide strand sequence). The first guide strand sequence and the first passenger strand sequence are separated by between 5 and 35 bases. The first guide strand sequence, the first passenger strand sequence and the sequence separating them are collectively the first hairpin. The multi-hairpin amiRNA gene further comprises a second (guide strand) sequence of at least 19 or 20 or 21 or 22 contiguous bases that are complementary to the target mRNA and a second (passenger strand) sequence of at least 19 or 20 or 21 or 22 bases that are at least 78% identical to the reverse complement of the second guide strand sequence (i.e. within 19 bases it comprises no more than 4 mismatches, including mutations, single base deletions or single base insertions, relative to the identical reverse complement of the second guide strand sequence). The second guide strand sequence and the second passenger strand sequence are separated by between 5 and 35 bases. The second guide strand sequence, the second passenger strand sequence and the sequence separating them are collectively the second hairpin. The first and second guide strand sequences are different from each other but complementary to the same target mRNA.

The multi-hairpin amiRNA gene may further comprise a third guide strand sequence of at least 19 or 20 or 21 or 22 bases that is complementary to the target mRNA and a third passenger strand sequence of at least 19 or 20 or 21 or 22 bases that is at least 78% identical to the reverse complement of the third guide strand sequence (i.e. within 19 bases it comprises no more than 4 mismatches, including mutations, single base deletions or single base insertions, relative to the identical reverse complement of the third guide strand sequence). The third guide strand sequence and the third passenger strand sequence are separated by between 5 and 35 bases. The third guide strand sequence, the third passenger strand sequence and the sequence separating them are collectively the third hairpin. The first and second and third guide strand sequences are each complementary to a different region of the same target mRNA.

The multi-hairpin amiRNA gene further comprises a promoter that is active in mammalian cells, preferably transcribable by RNA polymerase II or RNA polymerase III. Each hairpin is operably linked to the promoter. Preferably the promoter is heterologous to the hairpins. It the promoter is transcribed by RNA polymerase II, it is advantageous for the inhibitory polynucleotide further comprises a spacer polynucleotide that is operably linked to the promoter: the amiRNA hairpins may be placed to the 3′ UTR of the spacer polynucleotide, or they may be placed into an intron that is transcribed by the Pol II promoter. The spacer polynucleotide may comprise an open reading frame encoding an expressible polypeptide, or it may comprise a sequence that does not encode an expressible polypeptide. Preferably the spacer polynucleotide comprises between 50 and 3,000 nucleotides, more preferably the spacer is between 100 and 1,500 nucleotides. Optionally the spacer comprises an open reading frame to be expressed in the mammalian cell, such as a chimeric antigen receptor or a selectable marker. Example spacer polynucleotide sequences are given as SEQ ID NO: 279-284.

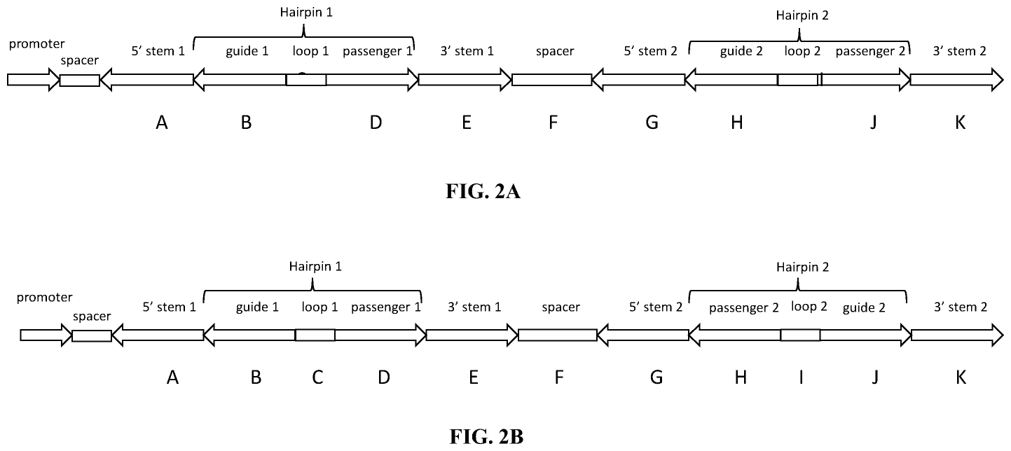

Each hairpin may comprise nucleotide sequences in addition to the guide and passenger strand sequences to enhance the stem-loop structure of the transcribed RNA, in order to increase the chance of processing and loading the guide strand into the RISC complex. A schematic of an exemplary multi-hairpin amiRNA gene is shown in FIGS. 2A-B. Short sequences (between 5 and 20 bases) may be added to the 5′ and 3′ of the guide-loop-passenger hairpin in order to stabilize it and improve processing of the RNA into the RISC complex. These are shown in FIGS. 2A-B as elements A and E stabilizing hairpin 1 and elements G and K stabilizing hairpin 2. For example, short nucleotide sequence SEQ ID NO: 255 may be added to the 5′ side of the guide-loop-passenger hairpin sequence and short nucleotide sequence SEQ ID NO: 256 may be added to the 3′ side of the guide-loop-passenger hairpin sequence to enhance RNA hairpin formation. Alternative exemplary pairs of stem-stabilizing nucleotide sequences that can be added to the 5′ and 3′ of the guide-loop-passenger strand sequence respectively to enhance RNA hairpin formation are SEQ ID NOs: 257 and 258, or SEQ ID NOs: 259 and 260, or SEQ ID NOs: 261 and 262, or SEQ ID NOs: 263 and 264, or SEQ ID NOs: 265 and 266, or SEQ ID NOs: 267 and 268, or SEQ ID NOs: 269 and 270, or a 5′ additional stem with sequence 5′-GTAGCAC-3′ and a 3′ additional stem with sequence 5′-TACTGC-3′. These stem sequences are derived from the nucleotide sequences flanking the guide-loop-passenger hairpin portion of the miRNA sequence in naturally occurring miRNAs. The corresponding sequences from other miRNAs may also be used. Although most of the exemplary sequences given herein have the guide strand sequence preceding the passenger strand sequence, the order may be 5′-guide-loop-passenger-3′ or it may be 5′-passenger-loop-guide-3′, as shown in FIGS. 1A-B. The RNA sequence that is loaded into the RISC complex is not determined by the order in which they occur. It is intended that “guide-loop-passenger” be read as meaning a sequence comprising these three elements in either configuration 5′-guide-loop-passenger-3′ or 5′-passenger-loop-guide-3′.

It is advantageous to provide some separation between hairpins in a polynucleotide comprising multiple hairpins, to improve the processing of the RNA (see for example element F in FIGS. 2A-B). The sequence separating the hairpins should be relatively unstructured. Exemplary unstructured sequences that may be incorporated between hairpins in an inhibitory polynucleotide include nucleotide sequences SEQ ID NOs: 271-278.

It is advantageous to provide some unstructured sequence to the 5′ of the first hairpin in an inhibitory polynucleotide. Exemplary unstructured sequences that may be incorporated to the 5′ of the first hairpin an inhibitory polynucleotide include nucleotide sequences SEQ ID NOs: 251-252. It is advantageous to provide some unstructured sequence to the 3′ of the last hairpin in an inhibitory polynucleotide. Exemplary unstructured sequences that may be incorporated to the 3′ of the last hairpin an inhibitory polynucleotide include nucleotide sequences SEQ ID NOs:253-254.

Although some sequence elements of artificial miRNAs are derived from naturally occurring miRNAs, the combination of guide, loop and passenger strand sequences in each artificial miRNA of the invention, or the combination of guide, loop and passenger strand sequences together with the 5′ and 3′ hairpin-stabilizing sequences in each artificial miRNA of the invention, are not naturally occurring miRNA sequences.

An exemplary general structure for a multi-hairpin amiRNA gene is shown in FIGS. 2A-B. It comprises (i) a promoter, operably linked to (ii) a spacer sequence preferably of between 50 and 3,000 nucleotides; (iii) an unstructured sequence, optionally from the 5′ region of a naturally occurring miRNA; (iv) a first hairpin comprising (a) a first 5′ stem sequence (FIGS. 2A-B, element A) which may optionally be derived from the 5′ stem (but preferably not the guide or passenger strand sequence) of a naturally occurring miRNA; (b) a first guide (or passenger) strand sequence (FIGS. 2A-B, element B); (c) a first loop sequence (FIGS. 2A-B, element C); (d) a first passenger (if the sequence in (b) was a guide strand sequence) or guide (if the sequence in (b) was a passenger strand sequence) strand sequence (FIGS. 2A-B, element D); (e) a first 3′ stem sequence (FIGS. 2A-B, element E) which may optionally be derived from the 3′ stem (but preferably not the guide or passenger strand sequence) of a naturally occurring miRNA, and wherein the first 5′ stem sequence and the first 3′ stem sequence increase the stability of the hairpin formed by the first guide strand sequence and the first passenger strand sequence; (v) optionally an unstructured sequence to separate the first hairpin from the second hairpin (FIGS. 2A-B, element F); (vi) a second hairpin comprising (f) a second 5′ stem sequence (FIGS. 2A-B, element G) which may optionally be derived from the 5′ stem (but preferably not the guide or passenger strand sequence) of a naturally occurring miRNA; (g) a second guide (or passenger) strand sequence (FIGS. 2A-B, element H); (h) a second loop sequence (FIGS. 2A-B, element I); (j) a second passenger (if the sequence in (g) was a guide strand sequence) or guide (if the sequence in (g) was a passenger strand sequence) strand sequence (FIGS. 2A-B, element J); (k) a second 3′ stem sequence (FIGS. 2A-B, element K) which may optionally be derived from the 3′ stem (but preferably not the guide or passenger strand sequence) of a naturally occurring miRNA, and wherein the second 5′ stem sequence and the second 3′ stem sequence increase the stability of the hairpin formed by the second guide strand sequence and the second passenger strand sequence; and wherein the first guide strand sequence and the second guide strand sequence are complementary to the same target mRNA expressed from an endogenous mammalian cell gene, and the first and second guide strand sequences are different from each other.

The inhibitory polynucleotide may be incorporated into cultured mammalian cells either on a transient vector, on a viral vector such as an adenovirus associated viral vector (an AAV vector), on a lentiviral vector or on a vector that integrates into the cell's genome through a process of random integration. The number of copies of an inhibitory polynucleotide comprising a multi-hairpin amiRNA gene that are integrated into the genome of a cultured mammalian cell may be increased by incorporating it into a transposon and then using a corresponding transposase to insert multiple copies of the transposon into the mammalian cell genome. An advantageous inhibitory polynucleotide comprises two transposon ends, as described in Section 5.2.2.

An inhibitory polynucleotide comprising a multi-hairpin amiRNA gene flanked by transposon ends may be stably integrated into the genome of a eukaryotic cell by introducing into the eukaryotic cell the transposon and a corresponding transposase (as described in Section 5.2.2), either as a transposase protein or as a polynucleotide encoding the transposase. Optionally the inhibitory polynucleotide may further comprise a selectable marker, which may be used to identify cells whose genome comprises the inhibitory polynucleotide and the multi-hairpin amiRNA gene. These cells may also be tested phenotypically to determine the degree by which expression of the target mRNA has been reduced. In some cases, inhibition of the target mRNA may result in a selectable phenotype.

Although it is preferable to incorporate two or more amiRNA hairpins to express guides complementary to the same target mRNA into a single polynucleotide, one can alternatively express two or more amiRNA guides complementary to different target sites of the same target mRNA within the same by using two separate inhibitory polynucleotides, providing that both polynucleotides become integrated into the genome of the cultured mammalian cell. Preferably the inhibitory polynucleotides comprise transposon ends or lentiviral repeats. A cultured mammalian cell whose genome comprises a first and second amiRNA hairpin, wherein the first and second guide strand sequences are complementary to first and second target sites of the same mRNA, and wherein the first and second guide strand sequences are different from each other is also an aspect of the invention. Preferably the expression of a target gene encoding the mRNA is reduced to a level lower than the level of expression of the target gene in a cultured mammalian control cell whose genome comprises only the first or the second amiRNA hairpin.

A cell whose genome comprises an inhibitory polynucleotide comprising a multi-hairpin amiRNA may have permanently reduced or eliminated activity of the gene encoded by the target mRNA. Such a cell is then useful and valuable for producing molecules that would otherwise be modified as a result of the direct or indirect action of the target mRNA. Such produced molecules may include proteins, sugars, metabolites, and other cellular products. Mammalian cell phenotypes that may be modified by inhibitory polynucleotides include the glycosylation of proteins, the intracellular trafficking of proteins, the proteolytic cleavage of proteins, the requirement for particular nutrients to be provided in order for the cell to grow, and the ability of the cell to survive under various conditions. Immune cell phenotypes that may be modified by inhibitory polynucleotides include the proliferation, survival, longevity, anergy and exhaustion of the immune cell.

### 5.2.5 Insulator Elements

When a heterologous polynucleotide is integrated into the genome of a mammalian cell, it is often desirable to prevent genetic elements within the heterologous polynucleotide from influencing expression of endogenous immune cell genes. Similarly, it is often desirable to prevent genes within the heterologous polynucleotide from being influenced by elements in the immune cell genome, for example from being silenced by incorporation into heterochromatin. Insulator elements are known to have enhancer-blocking activity (helping to prevent the genes in the heterologous polynucleotide from influencing the expression of endogenous immune cell genes) and barrier activity (helping to prevent genes within the heterologous polynucleotide from being silenced by incorporation into heterochromatin). Enhancer-blocking activity can result from binding of transcriptional repressor CTCF protein. Barrier activity can result from binding of vertebrate barrier proteins such as USF1 and VEZF1. Useful insulator sequences comprise binding sites for CTCF, USF1 or VEZF1. An advantageous gene transfer system comprises a polynucleotide comprising an insulator sequence comprising a binding site for CTCF, USF1 or VEZF1. More preferably a gene transfer system comprises a polynucleotide comprising two insulator sequences, each comprising a binding site for CTCF, USF1 or VEZF1, wherein the two insulator sequences flank any promoters or enhancers within the heterologous polynucleotide. Advantageous examples of insulator nucleotide sequences are SEQ ID NOs: 410-416.

If a heterologous polynucleotide comprising a promoter or enhancer is integrated into the genome of a mammalian cell without insulator sequences, there is a risk that either the promoter or enhancer elements within the heterologous polynucleotide will influence expression of endogenous immune cell genes (for example oncogenes), or that promoter or enhancer elements within the heterologous polynucleotide will be silenced by incorporation into heterochromatin. When a heterologous polynucleotide is integrated into a target genome following random fragmentation, some genetic elements are often lost, and others may be rearranged. There is thus a significant risk that, if the heterologous polynucleotide comprises insulator elements flanking enhancer and promoter elements, the insulator elements may be rearranged or lost, and the enhancer and promoter elements may be able to influence and be influenced by the genomic environment into which they integrate. It is therefore advantageous to use a transposon gene transfer system, wherein the entire sequence between the two transposon ITRs is integrated, without rearrangement, into the mammalian cell genome. Advantageous gene transfer systems for integration into mammalian cell genomes thus comprise a transposon in which elements are arranged in the following order: left transposon end; a first insulator sequence; sequences for expression within the immune cell; a second insulator sequence; right transposon end. The sequences for expression within the mammalian cell may include any number of regulatory sequences operably linked to any number of open reading frames.

### 5.2.6 Selection of Target Cells Comprising Polynucleotides

A target cell whose genome comprises a stably integrated polynucleotide may be identified, if the polynucleotide comprises a gene encoding a selectable marker, by exposing the target cells to conditions that favor cells expressing the selectable marker (“selection conditions”). It may therefore be advantageous for a polynucleotide to comprise a gene encoding a selectable marker.

One class of selectable markers that may be advantageously incorporated into a polynucleotide are those that provide a growth advantage to the cell by allowing a cell to survive in the presence of a harmful substance such as an antibiotic, enzyme inhibitor or cellular poison such as neomycin (resistance conferred by an aminoglycoside 3′-phosphotransferase e.g. a polypeptide with sequence selected from SEQ ID NOs: 294-297), puromycin (resistance conferred by puromycin acetyltransferase e.g. a polypeptide with sequence selected from SEQ ID NOs: 300-302), blasticidin (resistance conferred by a blasticidin acetyltransferase and a blasticidin deaminase e.g. a polypeptide with sequence SEQ ID NO: 303), hygromycin B (resistance conferred by hygromycin B phosphotransferase e.g. a polypeptide with sequence selected from SEQ ID NO: 298-299 and zeocin (resistance conferred by a binding protein encoded by the ble gene, for example a polypeptide with sequence SEQ ID NO: 291).

Another class of selectable markers that may be advantageously incorporated into a polynucleotide are those that provide a growth advantage to the cell by allowing the cell to synthesize a metabolically useful substance. One example of such a selectable marker is glutamine synthetase (GS, for example a polypeptide with sequence selected from SEQ ID NOs: 304-308) which allows selection via glutamine metabolism. Glutamine synthase is the enzyme responsible for the biosynthesis of glutamine from glutamate and ammonia, it is a crucial component of the only pathway for glutamine formation in a mammalian cell. In the absence of glutamine in the growth medium, the GS enzyme is essential for the survival of mammalian cells in culture. Some cell lines, for example mouse myeloma cells do not express enough GS enzyme to survive without added glutamine. In these cells a transfected GS gene can function as a selectable marker by permitting growth in a glutamine-free medium. In other cell lines, for example Chinese hamster ovary (CHO) cells express enough GS enzyme to survive without exogenously added glutamine. These cell lines can be manipulated by genome editing techniques including CRISPR/Cas9 to reduce or eliminate the activity of the GS enzyme. In all these cases, GS inhibitors such as methionine sulphoximine (MSX) can be used to inhibit a cell's endogenous GS activity. Selection protocols include introducing a polynucleotide comprising sequences encoding a first polypeptide and a glutamine synthase selectable marker, and then treating the cell with inhibitors of glutamine synthase such as methionine sulphoximine. The higher the levels of methionine sulphoximine that are used, the higher the level of glutamine synthase expression is required to allow the cell to synthesize enough glutamine to survive. Some of these cells will also show an increased expression of the first polypeptide.

Preferably the GS gene is operably linked to a weak promoter or other sequence elements that attenuate expression as described herein, such that high levels of expression can only occur if many copies of the polynucleotide are present, or if they are integrated in a position in the genome where high levels of expression occur. In such cases it may be unnecessary to use the inhibitor methionine sulphoximine: simply synthesizing enough glutamine for cell survival may provide a sufficiently stringent selection if expression of the glutamine synthetase is attenuated.

Another example of a selectable marker gene that may be advantageously incorporated into a polynucleotide to provide a growth advantage to the cell by allowing the cell to synthesize a metabolically useful substance is a gene encoding dihydrofolate reductase (DHFR, for example a polypeptide with sequence selected from SEQ ID NO: 292-293) which is required for catalyzing the reduction of 5,6-dihydrofolate (DHF) to 5,6,7,8-tetrahydrofolate (THF). Some cell lines do not express enough DHFR to survive without added hypoxanthine and thymidine (HT). In these cells a transfected DHFR gene can function as a selectable marker by permitting growth in a hypoxanthine and thymidine-free medium. DHFR-deficient cell lines, for example Chinese hamster ovary (CHO) cells can be produced by genome editing techniques including CRISPR/Cas9 to reduce or eliminate the activity of the endogenous DHRF enzyme. DHFR confers resistance to methotrexate (MTX). DHFR can be inhibited by higher levels of methotrexate. Selection protocols include introducing a construct comprising sequences encoding a first polypeptide and a DHFR selectable marker into a cell with or without an endogenous DHFR gene, and then treating the cell with inhibitors of DHFR such as methotrexate. The higher the levels of methotrexate that are used, the higher the level of DHFR expression is required to allow the cell to synthesize enough DHFR to survive. Some of these cells will also show an increased expression of the first polypeptide. Preferably the DHFR gene is operably linked to a weak promoter or other sequence elements that attenuate expression as described above, such that high levels of expression can only occur if many copies of the polynucleotide are present, or if they are integrated in a position in the genome where high levels of expression occur.

Another class of selectable markers include those that may be visually detected and then selected, but which do not provide any inherent growth advantage to the cell. Examples include fluorescent or chromogenic proteins (such as genes encoding GFP, RFP etc.) which can be selected for example using flow cytometry. Other selectable markers which do not provide any inherent growth advantage to the cell include genes encoding transmembrane proteins that can bind to a second molecule (protein or small molecule) that can be fluorescently labelled so that the presence of the transmembrane protein can be selected using flow cytometry. Other selectable markers which do not provide any inherent growth advantage to the cell include genes encoding luciferases.

High levels of expression may be obtained from genes encoded on polynucleotides that are integrated at regions of the genome that are highly transcriptionally active, or that are integrated into the genome in multiple copies, or that are present extrachromosomally in multiple copies. It is often advantageous to operably link the gene encoding the selectable marker to expression control elements that result in low levels of expression of the selectable polypeptide from the polynucleotide and/or to use conditions that provide more stringent selection. Under these conditions, for the expression cell to produce sufficient levels of the selectable polypeptide encoded on the polynucleotide to survive the selection conditions, the polynucleotide must either be present in a favorable location in the cell's genome for high levels of expression, or a sufficiently high number of copies of the polynucleotide must be present, such that these factors compensate for the low levels of expression achievable because of the expression control elements.

Genomic integration of transposons in which a selectable marker is operably linked to regulatory elements that only weakly express the marker usually requires that the transposon be inserted into the target genome by a transposase. By operably linking the selectable marker to elements that result in weak expression, cells are selected which either incorporate multiple copies of the transposon, or in which the transposon is integrated at a favorable genomic location for high expression. Using a gene transfer system that comprises a transposon and a corresponding transposase increases the likelihood that cells will be produced with multiple copies of the transposon, or in which the transposon is integrated at a favorable genomic location for high expression. Gene transfer systems comprising a transposon and a corresponding transposase are thus particularly advantageous when the transposon comprises a selectable marker operably linked to weak promoters.

A gene to be expressed from the polynucleotide may be included on the same polynucleotide as the selectable marker, with the two genes operably linked to different promoters. In this case low expression levels of the selectable marker may be achieved by using a weakly active constitutive promoter such as the phosphoglycerokinase (PGK) promoter (such as a promoter with nucleotide sequence selected from SEQ ID NOS: 382-391), the Herpes Simplex Virus thymidine kinase (HSV-TK) promoter (e.g. nucleotide sequence SEQ ID NO: 394), the MC1 promoter (for example nucleotide sequence SEQ ID NO: 395), the ubiquitin promoter (for example nucleotide sequence SEQ ID NO: 392). Other weakly active promoters maybe deliberately constructed, for example a promoter attenuated by truncation, such as a truncated promoter from simian virus 40 (SV40) (for example a nucleotide sequence selected from SEQ ID NO: 396-397), or a truncated HSV-TK promoter (for example nucleotide sequence SEQ ID NO: 393 or 399).

Expression of the selectable marker may also be reduced by destabilizing the mRNA, for example by incorporating amiRNA hairpins into the 3′UTR of the selectable marker. Insertion of multiple miRNA hairpins into the 3′ UTR of a GFP gene may reduce expression of the GFP, even though the miRNA is not targeting the GFP (Sun et al., 2006. Multi-miRNA hairpin method that improves gene knockdown efficiency and provides linked multi-gene knockdown. BioTechniques 41:59-63 doi 10.2144/000112203). This is likely because miRNA processing removes the stabilizing 3′UTR structures such as the polyA tail of the gene.

Expression levels of a selectable marker may be advantageously reduced by the insertion of miRNA hairpin sequences into the 3′ UTR of the gene encoding the selectable marker. For a selectable marker that encodes a protein that provides an essential nutrient to the cell, for example a gene encoding glutamine synthetase or a gene encoding dihydrofolate reductase, expression of the selectable marker must exceed a threshold level in order to provide enough of the essential nutrient to the cell, and thus for the cell to survive restrictive conditions (for example withdrawal of the essential nutrient from the media). Similarly if the selectable marker encodes a protein that provides a resistance mechanism to an inhibitory molecule, for example a protein that confers resistance to an antibiotic such as puromycin, neomycin/G418, blasticidin, hygromycin or zeocin, expression of the selectable marker must exceed a threshold level in order to enable the cell to survive restrictive conditions (for example the presence of a certain level of antibiotic in the media). If expression of the selectable marker is attenuated to below this threshold level, for example by the placement of miRNA hairpins into the 3′UTR of the selectable marker open reading frame, cells will only survive if they are able to increase expression of the selectable marker to above the threshold level. One way that this can be achieved is if a cell contains a higher number of copies of the selectable marker, so that the sum of expression of all copies allows the cell to exceed the needed threshold level of expression of the selectable marker. Typically, a higher number of copies of the selectable marker will be accompanied by a higher number of copies of all other genes on the polynucleotide comprising the selectable marker. A higher number of copies of these genes will result in higher levels of expression of these genes. Thus attenuation of a selectable marker by incorporation of miRNA hairpins into the 3′UTR following the selectable marker ORF will increase the expression of other genes encoded on the same polynucleotide.

An example in which inclusion of amiRNA hairpins in the 3′UTR of a gene encoding a metabolic enzyme increases the yield of another gene encoded on the same polynucleotide is shown in Sections 6.1.2.1 and 6.1.2.2. Inclusion of 2 or 3 amiRNA hairpins results in substantially higher expression levels of the other genes encoded on the polynucleotide than does inclusion of a single amiRNA hairpin. This is because inclusion of more than one hairpin Two and three hairpins are also more effective at inhibiting their target gene. An advantageous polynucleotide comprises a gene encoding a selectable marker operably linked to a Pol II promoter, and further comprises a first and second amiRNA hairpin in the 3′UTR of the selectable marker.

Preferably the first amiRNA hairpin comprises a first guide strand sequence of at least 19 or 20 or 21 or 22 contiguous bases complementary to an mRNA target, and the second amiRNA hairpin comprises a second guide strand sequence of at least 19 or 20 or 21 or 22 contiguous bases complementary to a different sequence within the same mRNA target as the first guide strand sequence. Preferably the first guide strand sequence is different from the second guide strand sequence. Optionally the polynucleotide comprises a third amiRNA hairpin in the 3′UTR of the selectable marker wherein the third amiRNA hairpin comprises a third guide strand sequence of at least 19 or 20 or 21 or 22 contiguous bases complementary to a different sequence within the same mRNA target, and wherein the third guide strand sequence is different from the first and second guide strand sequences. Preferably the selectable marker provides a growth advantage to the cell, for example by allowing the cell to synthesize a metabolically useful substance, or to survive in the presence of a harmful substance such as an antibiotic, enzyme inhibitor or cellular poison. Preferably the selectable marker is other than a fluorescent protein or chromogenic protein or a protein that catalyzes a fluorogenic or chromogenic reaction and that does not directly benefit the cell.

An advantageous selectable marker gene comprises an open reading frame encoding a polypeptide with sequence at least 90% identical to a sequence selected from SEQ ID NOs: 291-308, operably linked to a weak promoter, for example a nucleotide sequence selected from SEQ ID NOs: 382-399. Optionally there is an attenuating 5′UTR between the promoter and the glutamine synthetase open reading frame, for example a nucleotide sequence selected from SEQ ID NOs: 400-403. The 3′ UTR of the selectable marker gene comprises a multi-hairpin amiRNA sequence between the open reading frame and the polyadenylation sequence. Preferably the selectable marker gene is part of a transposon, transposable by a corresponding transposase.

The use of transposons and transposases in conjunction with weakly expressed selectable markers has several advantages over non-transposon constructs. One is that linkage between expression of the selectable marker and other genes on the transposon is very high, because a transposase will integrate the entire sequence that lies between the two transposon ends into the genome. In contrast when heterologous DNA is introduced into the nucleus of a eukaryotic cell, for example a mammalian cell, it is gradually broken into random fragments which may either be integrated into the cell's genome or degraded. Thus, if a polynucleotide comprising sequences to be expressed and a selectable marker is introduced into a population of cells, some cells will integrate the sequences encoding the selectable marker but not those encoding the other sequences to be expressed, and vice versa. In these circumstances, selection of cells expressing high levels of selectable marker is thus only somewhat correlated with cells that also express high levels of the other genes to be expressed. In contrast, because the transposase integrates all of the sequences between the transposon ends, cells expressing high levels of selectable marker are highly likely to also express high levels of the other genes to be expressed.

A second advantage of transposons and transposases is that they are much more efficient at integrating DNA sequences into the genome. A much higher fraction of the cell population is therefore likely to integrate one or more copies of a polynucleotide into their genomes, so there will be a correspondingly higher likelihood of good stable expression of both the selectable marker and the first polypeptide.

A third advantage of piggyBac-like transposons and transposases is that piggyBac-like transposases are biased toward inserting their corresponding transposons into transcriptionally active chromatin. Each cell is therefore likely to integrate the polynucleotide into a region of the genome from which genes are well expressed, so there will be a correspondingly higher likelihood of good stable expression of both the selectable marker and the first polypeptide.

### 5.3 Micro RNA for Inhibiting Fucosylation of Secreted Proteins

Fucosylation of antibodies inhibits antibody-dependent cell-mediated cytotoxicity (ADCC). Attempts have therefore been made to use RNA interference to reduce core fucosylation in cultured mammalian cells, including by targeting fucosyl transferase 8 (FUT8) the enzyme that catalyzes the transfer of α1,6-linked fucose to the first N-acetylglucosamine in N-linked glycans. Mori et. al. identified two siRNAs directed against FUT8 that resulted in 60% of a secreted antibody being afucosylated, compared with 10% afucosylated in the absence of siRNA (Mori et. al., 2004. Engineering Chinese hamster ovary cells to maximize effector function of produced antibodies using FUT8 siRNA. Biotechnol Bioeng. 88:901-8.). Beuger et. al. identified a FUT8-targeting shRNA that could produce as much as 88% afucosylated antibody (Beuger et. al., 2009. Short-hairpin-RNA-mediated silencing of fucosyltransferase 8 in Chinese-hamster ovary cells for the production of antibodies with enhanced antibody immune effector function. Biotechnol Appl Biochem. 53:31-7). U.S. Pat. Nos. 6,946,292, 7,737,325, 7,749,753, 7,846,725 and 8,003,781 describe strategies of inhibiting one or more genes in the fucosylation pathway including GDP-Mannose 4,6-dehydratase (GMD), alpha-(1,6)-fucosyl transferase (FUT8) and GDP-fucose transporter 1 (GFT) using RNA interference. Imai-Nishiya et. al. designed a pair of siRNA molecules targeting FUT8 and GDP-mannose 4,6-dehydratase (GMD) which was able to completely abolish fucosylation providing no fucose were present in the media (Imai-Nishiya et. al., 2007. Double knockdown of α1,6-fucosyltransferase (FUT8) and GDP-mannose 4,6-dehydratase (GMD) in antibody-producing cells: a new strategy for generating fully non-fucosylated therapeutic antibodies with enhanced ADCC. BMC Biotechnology 2007, 7:84). However, the presence of fucose compromises the synergistic effect of knocking down these two genes. Natural microRNAs that target FUT8, including miR-122 and miR-34a, have also been identified (Bernardi C. et. al., 2013. Effects of MicroRNAs on Fucosyltransferase 8 (FUT8) Expression in Hepatocarcinoma Cells. PLoS ONE 8(10): e76540. https.//doi.org/10.1371/journal.pone.0076540), though the effects of these microRNAs were modest.

In many of the RNA interference examples given above, cells with high levels of afucosylation were selected by treating the cells with a fucose-specific lectin such as Lens culinaris agglutinin that kills cells with fucosylated surface molecules. This is because the effectiveness of any individual siRNA sequence is less than 100%, and when genes expressing the siRNAs are introduced into cells, variation in expression levels leads to significant cell-to-cell variability. To overcome these limitations, we designed multi-hairpin amiRNA genes comprising one, two or more guide strand sequences complementary to different sequences within the same mRNA target (the mRNA for FUT8). We also used a piggyBac-like transposon vector to ensure that the amiRNA genes were integrated into transcriptionally active regions of the genome.

Examples described in Section 6.1.1 (including Sections 6.1.1.1, 6.1.1.2 and 6.1.1.3) show that integration into the CHO genome of a transposon comprising multi-hairpin amiRNAs with guide strand sequences complementary to the 3′ UTR of CHO FUT8 resulted in a complete lack of fucose (detected by highly sensitive mass spectroscopy) on antibodies produced by the cells. In contrast to previously reported methods, no subsequent lectin-based selection to kill cells that were still producing fucosylated proteins was necessary. Cells were selected only for incorporation of the transposon comprising the multi-hairpin amiRNA gene into the genome. By combining the effectiveness of multiple guide strand sequences targeting multiple different sequences within the same target mRNA, with highly efficient transposase-catalyzed transposon integration into the mammalian genome, the result was elimination of detectable target enzyme expression within the entire population of cells without further selection steps. Each multi-hairpin amiRNA sequence used in these examples comprised a first guide strand sequence comprising a contiguous 19 or 20 or 21 or 22 nucleotide sequence complementary to SEQ ID NO: 1 and a first passenger strand sequence comprising a contiguous 19 or 20 or 21 or 22 nucleotide sequence that is at least 78% identical to the reverse complement of the first guide strand sequence. Each multi-hairpin amiRNA sequence further comprised a second guide strand sequence comprising a contiguous 19 or 20 or 21 or 22 nucleotide sequence complementary to SEQ ID NO: 1 and a second passenger strand sequence comprising a contiguous 19 or 20 or 21 or 22 nucleotide sequence that is at least 78% identical to the reverse complement of the second guide strand sequence, and wherein the first and second guide strand sequences are different from each other. Each multi-hairpin amiRNA sequence further comprised a third guide strand sequence comprising a contiguous 19 or 20 or 21 or 22 nucleotide sequence complementary to SEQ ID NO: 1 and a third passenger strand sequence comprising a contiguous 19 or 20 or 21 or 22 nucleotide sequence that is at least 78% identical to the reverse complement of the third guide strand sequence, and wherein the first, second and third guide strand sequences are all different from each other. Each guide strand sequence was separated from its respective passenger strand sequence by between 5 and 35 bases. For multi-hairpin amiRNA genes with nucleotide sequences SEQ ID NOs 193 and 194, each guide strand sequence was separated from its respective passenger strand sequence by a nucleotide sequence comprising SEQ ID NO: 241. For multi-hairpin amiRNA gene with nucleotide sequence SEQ ID NO 195, each guide strand sequence was separated from its respective passenger strand sequence by a nucleotide sequence comprising SEQ ID NO: 242.

An advantageous polynucleotide for inhibition of fucosylation in Cricetulus griseus cells comprises or encodes a FUT8-inhibiting multi-hairpin amiRNA sequence. FUT8 is an alpha-(1,6)-fucosyltransferase. The FUT8-inhibiting multi-hairpin amiRNA sequence comprises a first guide strand sequence comprising a contiguous 19 or 20 or 21 or 22 nucleotide sequence complementary to SEQ ID NO: 1 and a first passenger strand sequence comprising a contiguous 19 or 20 or 21 or 22 nucleotide sequence that is at least 78% identical to the reverse complement of the first guide strand sequence. The FUT8-inhibiting multi-hairpin amiRNA sequence further comprises a second guide strand sequence comprising a contiguous 19 or 20 or 21 or 22 nucleotide sequence complementary to SEQ ID NO: 1 and a second passenger strand sequence comprising a contiguous 19 or 20 or 21 or 22 nucleotide sequence that is at least 78% identical to the reverse complement of the second guide strand sequence, and wherein the first and second guide strand sequences are different from each other. The FUT8-inhibiting multi-hairpin amiRNA sequence may optionally comprise a third guide strand sequence comprising a contiguous 19 or 20 or 21 or 22 nucleotide sequence complementary to SEQ ID NO: 1 and a third passenger strand sequence comprising a contiguous 19 or 20 or 21 or 22 nucleotide sequence that is at least 78% identical to the reverse complement of the third guide strand sequence, and wherein the first, second and third guide strand sequences are all different from each other. Each guide strand sequence is separated from its respective passenger strand sequence by between 5 and 35 bases. Exemplary sequences for separating a guide strand sequence from its passenger strand sequence are sequences that comprise a nucleotide sequence selected from SEQ ID NO: 241-250. Exemplary guide strand nucleotide sequences for inhibiting Cricetulus griseus FUT8 and their respective passenger strand nucleotide sequences are SEQ ID NOs: 23 and 108, SEQ ID NOs: 24 and 109, SEQ ID NOs: 25 and 110, SEQ ID NOs: 26 and 111, SEQ ID NOs: 27 and 112, and SEQ ID NOs: 28 and 113.

An advantageous polynucleotide for inhibition of fucosylation in Cricetulus griseus cells comprises or encodes a GDP-mannose-4, 6-dehydratase (GMD)-inhibiting multi-hairpin amiRNA sequence. A GMD-inhibiting multi-hairpin amiRNA sequence comprises a first guide strand sequence comprising a contiguous 19 or 20 or 21 or 22 nucleotide sequence complementary to SEQ ID NO: 3 and a first passenger strand sequence comprising a contiguous 19 or 20 or 21 or 22 nucleotide sequence that is at least 78% identical to the reverse complement of the first guide strand sequence. The GMD-inhibiting multi-hairpin amiRNA sequence further comprises a second guide strand sequence comprising a contiguous 19 or 20 or 21 or 22 nucleotide sequence complementary to SEQ ID NO: 3 and a second passenger strand sequence comprising a contiguous 19 or 20 or 21 or 22 nucleotide sequence that is at least 78% identical to the reverse complement of the second guide strand sequence, and wherein the first and second guide strand sequences are different from each other. The GMD-inhibiting multi-hairpin amiRNA sequence may optionally comprise a third guide strand sequence comprising a contiguous 19 or 20 or 21 or 22 nucleotide sequence complementary to SEQ ID NO: 3 and a third passenger strand sequence comprising a contiguous 19 or 20 or 21 or 22 nucleotide sequence that is at least 78% identical to the reverse complement of the third guide strand sequence, and wherein the first, second and third guide strand sequences are all different from each other. Each guide strand sequence is separated from its respective passenger strand sequence by between 5 and 35 bases. Exemplary sequences for separating a guide strand sequence from its passenger strand sequence are sequences that comprise a nucleotide sequence selected from SEQ ID NO: 241-250. Exemplary guide strand nucleotide sequences for inhibiting Cricetulus griseus GMD and their respective passenger strand nucleotide sequences are SEQ ID NOs: 35 and 120, SEQ ID NOs: 36 and 121, SEQ ID NOs: 37 and 122, SEQ ID NOs: 38 and 123, SEQ ID NOs: 39 and 124, and SEQ ID NOs: 40 and 125.

An advantageous polynucleotide for inhibition of fucosylation in Cricetulus griseus cells comprises or encodes a GDP-fucose transporter (GFT)-inhibiting multi-hairpin amiRNA sequence. The GFT-inhibiting multi-hairpin amiRNA nucleotide sequence comprises a first guide strand sequence comprising a contiguous 19 or 20 or 21 or 22 nucleotide sequence complementary to SEQ ID NO: 5 and a first passenger strand sequence comprising a contiguous 19 or 20 or 21 or 22 nucleotide sequence that is at least 78% identical to the reverse complement of the first guide strand sequence. The GFT-inhibiting multi-hairpin amiRNA sequence further comprises a second guide strand sequence comprising a contiguous 19 or 20 or 21 or 22 nucleotide sequence complementary to SEQ ID NO: 5 and a second passenger strand sequence comprising a contiguous 19 or 20 or 21 or 22 nucleotide sequence that is at least 78% identical to the reverse complement of the second guide strand sequence, and wherein the first and second guide strand sequences are different from each other. The GFT-inhibiting multi-hairpin amiRNA sequence may optionally comprise a third guide strand sequence comprising a contiguous 19 or 20 or 21 or 22 nucleotide sequence complementary to SEQ ID NO: 5 and a third passenger strand sequence comprising a contiguous 19 or 20 or 21 or 22 nucleotide sequence that is at least 78% identical to the reverse complement of the third guide strand sequence, and wherein the first, second and third guide strand sequences are all different from each other. Each guide strand sequence is separated from its respective passenger strand sequence by between 5 and 35 bases. Exemplary sequences for separating a guide strand sequence from its passenger strand sequence are sequences that comprise a nucleotide sequence selected from SEQ ID NO: 241-250. Exemplary guide strand nucleotide sequences for inhibiting Cricetulus griseus GFT and their respective passenger strand nucleotide sequences are SEQ ID NOs: 41 and 126, SEQ ID NOs: 42 and 127, SEQ ID NOs: 43 and 128, SEQ ID NOs: 44 and 129, SEQ ID NOs: 45 and 130, and SEQ ID NOs: 46 and 131.

An advantageous inhibitory polynucleotide for inhibition of fucosylation in Cricetulus griseus cells comprises or encodes a first guide strand sequence comprising a contiguous 19 or 20 or 21 or 22 nucleotide sequence that is perfectly complementary to a natural mammalian cellular mRNA and a first passenger strand sequence comprising a contiguous 19 or 20 or 21 or 22 nucleotide sequence that is at least 78% complementary to the first guide strand sequence, wherein the first guide strand and first passenger strand sequence are separated by between 5 and 35 nucleotides and a second guide strand sequence comprising a contiguous 19 or 20 or 21 or 22 nucleotide sequence that is perfectly complementary to the same natural mammalian cellular mRNA as the first guide strand sequence and a second passenger strand sequence comprising a contiguous 19 or 20 or 21 or 22 nucleotide sequence that is at least 78% complementary to the second guide strand sequence, wherein the second guide strand and second passenger strand sequence are separated by between 5 and 35 nucleotides, and wherein the first and second guide strand sequence are different from each other, and wherein the natural mammalian cellular mRNA comprises a sequence that is at least 98% identical or at least 99% identical to, or perfectly identical to a nucleotide sequence selected from SEQ ID NOs: 1-6. Exemplary multi-hairpin amiRNAs for inhibition of fucosylation in Cricetulus griseus cells include nucleotide sequences SEQ ID NOs: 193-201.

An advantageous polynucleotide for inhibition of fucosylation in human cells comprises or encodes a FUT8-inhibiting multi-hairpin amiRNA sequence. The FUT8-inhibiting multi-hairpin amiRNA sequence comprises a first guide strand sequence comprising a contiguous 19 or 20 or 21 or 22 nucleotide sequence complementary to SEQ ID NO: 7 and a first passenger strand sequence comprising a contiguous 19 or 20 or 21 or 22 nucleotide sequence that is at least 78% identical to the reverse complement of the first guide strand sequence. The FUT8-inhibiting multi-hairpin amiRNA sequence further comprises a second guide strand sequence comprising a contiguous 19 or 20 or 21 or 22 nucleotide sequence complementary to SEQ ID NO: 7 and a second passenger strand sequence comprising a contiguous 19 or 20 or 21 or 22 nucleotide sequence that is at least 78% identical to the reverse complement of the second guide strand sequence, and wherein the first and second guide strand sequences are different from each other. The FUT8-inhibiting multi-hairpin amiRNA sequence may optionally comprise a third guide strand sequence comprising a contiguous 19 or 20 or 21 or 22 nucleotide sequence complementary to SEQ ID NO: 7 and a third passenger strand sequence comprising a contiguous 19 or 20 or 21 or 22 nucleotide sequence that is at least 78% identical to the reverse complement of the third guide strand sequence, and wherein the first, second and third guide strand sequences are all different from each other. Each guide strand sequence is separated from its respective passenger strand sequence by between 5 and 35 bases. Exemplary sequences for separating a guide strand sequence from its passenger strand sequence are sequences that comprise a nucleotide sequence selected from SEQ ID NO: 241-250. Exemplary guide strand nucleotide sequences for inhibiting human FUT8 and their respective passenger strand nucleotide sequences are SEQ ID NOs: 29 and 114, SEQ ID NOs: 30 and 115, SEQ ID NOs: 31 and 116, SEQ ID NOs: 32 and 117, SEQ ID NOs: 33 and 118, and SEQ ID NOs: 34 and 119.

An advantageous polynucleotide for inhibition of fucosylation in human cells comprises or encodes a GDP-mannose-4, 6-dehydratase (GMD)-inhibiting multi-hairpin amiRNA sequence. The GMD-inhibiting multi-hairpin amiRNA sequence comprises a first guide strand sequence comprising a contiguous 19 or 20 or 21 or 22 nucleotide sequence complementary to SEQ ID NO: 8 and a first passenger strand sequence comprising a contiguous 19 or 20 or 21 or 22 nucleotide sequence that is at least 78% identical to the reverse complement of the first guide strand sequence. The GMD-inhibiting multi-hairpin amiRNA sequence further comprises a second guide strand sequence comprising a contiguous 19 or 20 or 21 or 22 nucleotide sequence complementary to SEQ ID NO: 8 and a second passenger strand sequence comprising a contiguous 19 or 20 or 21 or 22 nucleotide sequence that is at least 78% identical to the reverse complement of the second guide strand sequence, and wherein the first and second guide strand sequences are different from each other. The GMD-inhibiting multi-hairpin amiRNA sequence may optionally comprise a third guide strand sequence comprising a contiguous 19 or 20 or 21 or 22 nucleotide sequence complementary to SEQ ID NO: 8 and a third passenger strand sequence comprising a contiguous 19 or 20 or 21 or 22 nucleotide sequence that is at least 78% identical to the reverse complement of the third guide strand sequence, and wherein the first, second and third guide strand sequences are all different from each other. Each guide strand sequence is separated from its respective passenger strand sequence by between 5 and 35 bases. Exemplary sequences for separating a guide strand sequence from its passenger strand sequence are sequences that comprise a nucleotide sequence selected from SEQ ID NO: 241-250. Exemplary guide strand nucleotide sequences for inhibiting human GMD and their respective passenger strand nucleotide sequences are SEQ ID NOs: 47 and 132, SEQ ID NOs: 48 and 133, and SEQ ID NOs: 49 and 134.

An advantageous polynucleotide for inhibition of fucosylation in human cells comprises or encodes a GDP-fucose transporter (GFT)-inhibiting multi-hairpin amiRNA sequence. The GFT-inhibiting multi-hairpin amiRNA sequence comprises a first guide strand sequence comprising a contiguous 19 or 20 or 21 or 22 nucleotide sequence complementary to SEQ ID NO: 9 and a first passenger strand sequence comprising a contiguous 19 or 20 or 21 or 22 nucleotide sequence that is at least 78% identical to the reverse complement of the first guide strand sequence. The GFT-inhibiting multi-hairpin amiRNA sequence further comprises a second guide strand sequence comprising a contiguous 19 or 20 or 21 or 22 nucleotide sequence complementary to SEQ ID NO: 9 and a second passenger strand sequence comprising a contiguous 19 or 20 or 21 or 22 nucleotide sequence that is at least 78% identical to the reverse complement of the second guide strand sequence, and wherein the first and second guide strand sequences are different from each other. The GFT-inhibiting multi-hairpin amiRNA sequence may optionally comprise a third guide strand sequence comprising a contiguous 19 or 20 or 21 or 22 nucleotide sequence complementary to SEQ ID NO: 9 and a third passenger strand sequence comprising a contiguous 19 or 20 or 21 or 22 nucleotide sequence that is at least 78% identical to the reverse complement of the third guide strand sequence, and wherein the first, second and third guide strand sequences are all different from each other. Each guide strand sequence is separated from its respective passenger strand sequence by between 5 and 35 bases. Exemplary sequences for separating a guide strand sequence from its passenger strand sequence are sequences that comprise a nucleotide sequence selected from SEQ ID NO: 241-250. Exemplary guide strand nucleotide sequences for inhibiting human GFT and their respective passenger strand nucleotide sequences are SEQ ID NOs: 50 and 135, SEQ ID NOs: 51 and 136, and SEQ ID NOs: 52 and 137.

An advantageous inhibitory polynucleotide for inhibition of fucosylation in human cells comprises or encodes a first guide strand sequence comprising a contiguous 19 or 20 or 21 or 22 nucleotide sequence that is perfectly complementary to a natural mammalian cellular mRNA and a first passenger strand sequence comprising a contiguous 19 or 20 or 21 or 22 nucleotide sequence that is at least 78% complementary to the first guide strand sequence, wherein the first guide strand and first passenger strand sequence are separated by between 5 and 35 nucleotides and a second guide strand sequence comprising a contiguous 19 or 20 or 21 or 22 nucleotide sequence that is perfectly complementary to the same natural mammalian cellular mRNA as the first guide strand sequence and a second passenger strand sequence comprising a contiguous 19 or 20 or 21 or 22 nucleotide sequence that is at least 78% complementary to the second guide strand sequence, wherein the second guide strand and second passenger strand sequence are separated by between 5 and 35 nucleotides, and wherein the first and second guide strand sequence are different from each other, and wherein the natural mammalian cellular mRNA comprises a sequence that is at least 98% identical or at least 99% identical to, or perfectly identical to a nucleotide sequence selected from SEQ ID NOs: 7-9. Exemplary multi-hairpin amiRNAs for inhibition of fucosylation in human cells include SEQ ID NOs: 202-204.

A method for producing secreted proteins with reduced fucose levels from mammalian cells comprises (i) introducing into a mammalian cell an inhibitory polynucleotide for inhibition of fucosylation in mammalian cells, wherein the inhibitory polynucleotide comprises or encodes a multi-hairpin amiRNA for expression of two or more interfering RNA guide sequences complementary to the same natural mammalian mRNA, and wherein the natural mammalian mRNA encodes an enzyme involved in the fucosylation of proteins (for example GDP-Mannose 4,6-dehydratase (GMD), alpha-(1,6)-fucosyl transferase (FUT8) and GDP-fucose transporter 1 (GFT)), and (ii) introducing into the same mammalian cell a gene encoding a protein to be secreted, the gene expressible in the mammalian cell. The two sequences may be introduced in any order: for example, the inhibitory polynucleotide may be introduced first and the gene encoding a protein to be secreted may be introduced second, the gene encoding a protein to be secreted may be introduced first and the inhibitory polynucleotide may be introduced second, or the two sequences may be introduced to the mammalian cell at the same time. In some instances, the protein to be secreted is an antibody or an Fc fusion.

### 5.4 Glutamine Synthetase

Disruption of a natural mammalian gene that normally provides to the cell a protein that is essential for growth, division or survival, such as a gene that encodes an essential metabolic enzyme, can provide an opportunity to develop a metabolic selection system. Some exemplary metabolic selection systems are described in Section 5.2.6. Typically, this is accomplished by permanent irreversible disruption of the gene encoding the essential metabolic enzyme, which can be accomplished using a targeted disruption method such as zinc finger nucleases, TALE effector nucleases, CRISPR Cas9-directed nucleases and AAV-directed nucleases, or a random method such as irradiation or other random mutagenesis of the cells with subsequent identification of cells in which the gene encoding the essential metabolic enzyme is disrupted. Cells in which expression of the essential metabolic gene has been disrupted can survive, grow and divide in the absence of this otherwise essential gene if an enzyme, growth factor, nutrient or other molecule is provided exogenously to compensate for the lack of the product of the missing essential metabolic enzyme. Cells in which expression of the essential metabolic gene has been disrupted can then be used as hosts for subsequent introduction of expression polynucleotides which comprise a selectable marker whose function is to complement or compensate for the lack of function of the essential metabolic gene, and one or more other gene to be expressed in the cell. These cells are then subjected to conditions where the enzyme, growth factor, nutrient or other molecule that was provided to allow the cell to grow, are removed. Only cells that have taken up the expression polynucleotide comprising the gene encoding the complementing selectable marker will survive. Previously described examples include CRISPR disruption of the glutamine synthetase gene in human culture cells (Yu et al, 2018. Biotechnol Bioeng. 115: 1367-1372. “Glutamine synthetase gene knockout-human embryonic kidney 293E cells for stable production of monoclonal antibodies.”), zinc finger disruption of glutamine synthetase in CHO cells (Fan et al 2012. Biotechnol Bioeng. 109: 1007-15. “Improving the efficiency of CHO cell line generation using glutamine synthetase gene knockout cells.”), zinc finger disruption of the DHFR gene in mammalian cells (Santiago et al 2008. Proc Natl Acad Sci USA. 105: 5809-5814. “Targeted gene knockout in mammalian cells by using engineered zinc-finger nucleases”), and deletion of DHFR in CHO cells by irradiation (Urlaub et al, 1983. Cell. 33: 405-12. “Deletion of the diploid dihydrofolate reductase locus from cultured mammalian cells.”).

Permanent disruption of the gene sequence has been the method previously used to inhibit expression of essential metabolic enzymes because, in order to provide an appropriate selective pressure, expression of the essential metabolic enzyme must be reduced to below a level that would allow cells to grow. There must also be no “leakiness”: if some cells are able to resume expressing the essential metabolic enzyme, then they will grow in the absence of the expression polynucleotide comprising the complementing selectable marker, which will create a background of cells not expressing the genes to be expressed that are encoded on the expression polynucleotide. RNA interference has not generally been sufficiently effective at inhibiting the expression of essential metabolic genes, nor sufficiently stable as to ensure the continued inhibition of expression of the essential metabolic gene. However, the benefit of an RNA interference approach is that it can be extremely fast to implement, and it can inhibit all copies of a gene in a diploid or polyploid cell simultaneously, without having to independently determine that each genomic copy has been mutationally inactivated. Furthermore, as shown in Examples in Section 6.2, a method comprising introduction of a multi-hairpin amiRNA gene for inhibition of an essential metabolic gene into the genome of a pool of cells, and selection of cells whose genomes comprise the multi-hairpin amiRNA gene, can result in a pool of cells in which expression of the essential metabolic enzyme is inhibited to a level that prevents growth of the cell in more than 70% or 80% or 90% or 91% or 92% or 93% or 94% or 95% or 96% or 97% or 98% or 99% of the cells in the pool. This is in contrast with directed cleavage methods such as zinc finger nucleases, TAL effector nucleases (TALENs), CRISPR/Cas9 nucleases or AAV. Such methods are considered effective if they can mutate and inactivate a target gene in between 1% and 10% of the cells into which they are transfected. The multi-hairpin amiRNA approach is thus at least 10-fold more efficient than these nuclease-based gene disruption techniques.

A multi-hairpin amiRNA gene can be integrated into the genome of a mammalian cell to inhibit a natural mammalian gene that normally provides to the cell a protein that is essential for growth (including survival and division). The multi-hairpin amiRNA may be placed into the 3′UTR of a second gene to be expressed within the cell. Preferably the gene encodes an essential metabolic enzyme, such that the cell cannot grow in the absence of this otherwise essential gene unless an enzyme, growth factor, nutrient or other molecule is provided exogenously (we refer to this as an exogenously provided complementing factor). Cells will often have intracellular reserves of various nutrients, so a cell is considered not to grow if the cell can divide only 1, 2, 3 or 4 times after the removal of the exogenously provided complementing factor. A population of cells in which expression of the essential metabolic enzyme has been successfully inhibited will thus increase its viable cell density by no more than 2-fold, 4-fold, 8-fold or 16-fold following removal of the exogenously provided complementing factor. Preferably expression of the essential metabolic enzyme is inhibited such that less than 50% or 40% or 30% or 20% or 10% or 5% or 2% or 1% of the natural enzyme activity remains in the cell. Examples of such proteins include an essential metabolic enzyme involved in the synthesis of an amino acid, an essential metabolic enzyme involved in the synthesis of an amino acid precursor, an essential metabolic enzyme involved in the synthesis of a nucleotide, an essential metabolic enzyme involved in the synthesis of a nucleotide precursor, an essential metabolic enzyme involved in the synthesis of a fatty acid and an essential metabolic enzyme involved in the synthesis of a vitamin. If the multi-hairpin amiRNA gene is stably integrated into the mammalian cell genome, and stably expressed, the essential metabolic enzyme is stably inhibited. A second gene that complements or compensates for the inhibited essential metabolic enzyme may then be used as a selectable marker in the mammalian cell. The second gene may encode an alternative version of the inhibited essential metabolic enzyme that is resistant to inhibition by the multi-hairpin amiRNA, for example by containing differences in its mRNA sequence at the positions of complementarity between the mRNA for the essential metabolic enzyme and the guide strand sequences encoded by the multi-hairpin amiRNA gene. The second gene may alternatively encode one or more enzymes that provide an alternative metabolic pathway to provide the missing essential nutrient. A second polynucleotide comprising the second complementing gene may then be introduced into the mammalian cell, and selection pressure can be applied by withdrawal, at once or by tapered reduction of the exogenously provided enzyme, growth factor, nutrient or other molecule. The only cells that survive such selection are those that have taken up the second polynucleotide and expressed the second gene. The second polynucleotide may comprise other genes that will also be expressed. Preferably the second polynucleotide is a transposon or a viral vector. One advantage of this strategy is that nutrient withdrawal is often a very gentle selection compared with the addition of a drug. Drugs that are commonly used as selectable markers often have pleiotropic effects which may have undesired effects on the mammalian cell. For example, the use of methionine sulfoxamine to inhibit the glutamine synthetase gene reduces the cellular growth rate and increases production of toxic metabolic wastes lactate and ammonia in CHO cells (Noh et al (2018). Comprehensive characterization of glutamine synthetase-mediated selection for the establishment of recombinant CHO cells producing monoclonal antibodies. Scientific Reports, 8, [5361]. https://doi.org/10.1038/s41598-018-23720-9)

A method for stably introducing into a mammalian cell a polynucleotide for expression comprises (a) introducing into the mammalian cell an inhibitory polynucleotide comprising a gene, expressible in the mammalian cell, which encodes an interfering RNA with guide strand sequence(s) complementary to the mRNA for an essential metabolic enzyme; (b) growing the cell in the presence of an enzyme, growth factor, nutrient or other molecule that is provided exogenously to enable the cell to survive, grow and divide while expression of the essential metabolic enzyme is inhibited; (c) introducing into the cell a second polynucleotide comprising (i) a gene encoding a selectable marker expressible in the mammalian cell, wherein the selectable marker functionally complements the lack of the essential metabolic enzyme and removes the requirement for the exogenous provision of the enzyme, growth factor, nutrient or other molecule that enabled the cell to survive, grow and divide while expression of the essential metabolic enzyme was inhibited, and (ii) a second gene expressible in the mammalian cell; and (d) growing the cell in the absence of the enzyme, growth factor, nutrient or other molecule that was provided exogenously in (b) to enable the cell to survive, grow and divide while expression of the essential metabolic enzyme is inhibited, thereby making the cell's survival, growth and division dependent upon the expression of the selectable marker from the second polynucleotide. Preferably the first and second polynucleotides are integrated into the mammalian cell genome. The method optionally also comprises (e) growing the cell under conditions where the second gene in the second polynucleotide is expressed. Optionally the second gene encodes a protein product, and the method further comprises (f) collecting or purifying the protein product encoded by the second gene.

One class of selectable markers that may be advantageously incorporated into a polynucleotide are those that provide a growth advantage to the cell by allowing the cell to synthesize a metabolically useful substance. One example of such a selectable marker is glutamine synthetase (GS, for example a polypeptide sequence selected from SEQ ID NOs: 304-308) which allows selection via glutamine metabolism. Glutamine synthase is the enzyme responsible for the biosynthesis of glutamine from glutamate and ammonia, it is a crucial component of the only pathway for glutamine formation in a mammalian cell. In the absence of glutamine in the growth medium, the glutamine synthetase enzyme is essential for the survival of mammalian cells in culture. Some cell lines, for example mouse myeloma cells do not express enough glutamine synthetase enzyme to survive without added glutamine.

In some cell lines, for example HEK cells and Chinese hamster ovary (CHO) cells, there is enough glutamine synthetase enzyme expressed to enable the cell to survive without exogenously added glutamine. These cells can be manipulated by genome editing techniques including CRISPR/Cas9 to reduce or eliminate the activity of the endogenous glutamine synthetase enzyme. However even with CRISPR this is a laborious process that may introduce off-target mutations in other genes. An alternative method is to stably integrate into the cell genome a polynucleotide comprising a multi-hairpin amiRNA that targets the endogenous glutamine synthetase gene. An exogenously provided glutamine synthetase gene may then be used as a selectable marker, provided the exogenously provided gene does not comprise the sequences targeted by the guide strand sequence. This may be accomplished by altering the codon used to encode the glutamine synthetase if the guide targets sequences within the open reading frame. It may be accomplished by altering the 5′ UTR if the guide targets sequences within the 5′ UTR. It may be accomplished by altering the polyadenylation signal of the 3′ UTR if the guide targets sequences within the polyadenylation signal sequence/3′ UTR.

### 5.4.1 Micro RNA to Reduce Endogenous Glutamine Synthetase

An advantageous polynucleotide for inhibition of glutamine synthetase in Cricetulus griseus cells through RNA interference comprises or encodes a glutamine synthetase-inhibiting multi-hairpin amiRNA sequence. The glutamine synthetase-inhibiting multi-hairpin amiRNA sequence comprises a first guide strand sequence comprising a contiguous 19 or 20 or 21 or 22 nucleotide sequence complementary to SEQ ID NO: 10 and a first passenger strand sequence comprising a contiguous 19 or 20 or 21 or 22 nucleotide sequence that is at least 78% identical to the reverse complement of the first guide strand sequence. The glutamine synthetase-inhibiting multi-hairpin amiRNA sequence further comprises a second guide strand sequence comprising a contiguous 19 or 20 or 21 or 22 nucleotide sequence complementary to SEQ ID NO: 10 and a second passenger strand sequence comprising a contiguous 19 or 20 or 21 or 22 nucleotide sequence that is at least 78% identical to the reverse complement of the second guide strand sequence, and wherein the first and second guide strand sequences are different from each other. The glutamine synthetase-inhibiting multi-hairpin amiRNA sequence may optionally comprise a third guide strand sequence comprising a contiguous 19 or 20 or 21 or 22 nucleotide sequence complementary to SEQ ID NO: 10 and a third passenger strand sequence comprising a contiguous 19 or 20 or 21 or 22 nucleotide sequence that is at least 78% identical to the reverse complement of the third guide strand sequence, and wherein the first, second and third guide strand sequences are all different from each other. Each guide strand sequence is separated from its respective passenger strand sequence by between 5 and 35 bases. Exemplary sequences for separating a guide strand sequence from its passenger strand sequence are sequences that comprise a nucleotide sequence selected from SEQ ID NO: 241-250. Exemplary guide strand nucleotide sequences for inhibiting Cricetulus griseus glutamine synthetase and their respective passenger strand nucleotide sequences are SEQ ID NOs: 53 and 138, SEQ ID NOs: 54 and 139, SEQ ID NOs: 55 and 140, SEQ ID NOs: 56 and 141, SEQ ID NOs: 57 and 142, SEQ ID NOs: 58 and 143, SEQ ID NOs: 59 and 144, SEQ ID NOs: 60 and 145, SEQ ID NOs: 61 and 146, SEQ ID NOs: 62 and 147, SEQ ID NOs: 63 and 148, SEQ ID NOs: 64 and 149, SEQ ID NOs: 65 and 150, SEQ ID NOs: 66 and 151, SEQ ID NOs: 67 and 152 and SEQ ID NOs: 68 and 153.

Multi-hairpin amiRNA with nucleotide sequence SEQ ID NO: 209 comprises guide strand sequences complementary to three different sequences within the CHO glutamine synthetase mRNA target (nucleotide sequence SEQ ID NO: 10). Multi-hairpin amiRNA nucleotide sequence SEQ ID NO: 209 comprises a first guide strand sequence with nucleotide sequence SEQ ID NO: 53 and a first passenger strand sequence with nucleotide sequence SEQ ID NO: 138; nucleotide sequence SEQ ID NO: 209 further comprises a second guide strand sequence with nucleotide sequence SEQ ID NO: 54 and a second passenger strand sequence with nucleotide sequence SEQ ID NO: 139; nucleotide sequence SEQ ID NO: 209 further comprises a third guide strand sequence with nucleotide sequence SEQ ID NO: 55 and a third passenger strand sequence with nucleotide sequence SEQ ID NO: 140. Guide strand nucleotide sequences SEQ ID NO: 53, SEQ ID NO: 54, and SEQ ID NO: 55 are all different from each other. Each guide strand sequence is separated from its respective passenger strand sequence by a nucleotide sequence comprising SEQ ID NO: 241.

Incorporation of the multi-hairpin amiRNA into a transposon vector enhances the likelihood that the amiRNA genes will be integrated into transcriptionally active regions of the genome. As described in Section 6.2, integration of the multi-hairpin amiRNA with nucleotide sequence SEQ ID NO: 209, operably linked to a promoter active in a mammalian cell, into the genome of a Cricetulus griseus cell reduces expression of glutamine synthetase such that the cell becomes completely dependent upon exogenously supplied glutamine for its survival.

A similar strategy can be used to create glutamine synthetase-deficient human cell lines, such as HEK cell lines. An advantageous polynucleotide for inhibition of glutamine synthetase in human cells through RNA interference comprises or encodes a glutamine synthetase-inhibiting multi-hairpin amiRNA sequence. The glutamine synthetase-inhibiting multi-hairpin amiRNA sequence comprises a first guide strand sequence comprising a contiguous 19 or 20 or 21 or 22 nucleotide sequence complementary to the mRNA for human glutamine synthetase (e.g. nucleotide sequence SEQ ID NO: 12) and a first passenger strand sequence comprising a contiguous 19 or 20 or 21 or 22 nucleotide sequence that is at least 78% identical to the reverse complement of the first guide strand sequence. The glutamine synthetase-inhibiting multi-hairpin amiRNA sequence further comprises a second guide strand sequence comprising a contiguous 19 or 20 or 21 or 22 nucleotide sequence complementary to the mRNA for human glutamine synthetase (e.g. nucleotide sequence SEQ ID NO: 12) and a second passenger strand sequence comprising a contiguous 19 or 20 or 21 or 22 nucleotide sequence that is at least 78% identical to the reverse complement of the second guide strand sequence, and wherein the first and second guide strand sequences are different from each other. The glutamine synthetase-inhibiting multi-hairpin amiRNA sequence may optionally comprise a third guide strand sequence comprising a contiguous 19 or 20 or 21 or 22 nucleotide sequence complementary to the mRNA for human glutamine synthetase (e.g. nucleotide sequence SEQ ID NO: 12) and a third passenger strand sequence comprising a contiguous 19 or 20 or 21 or 22 nucleotide sequence that is at least 78% identical to the reverse complement of the third guide strand sequence, and wherein the first, second and third guide strand sequences are all different from each other. Each guide strand sequence is separated from its respective passenger strand sequence by between 5 and 35 bases. Exemplary sequences for separating a guide strand sequence from its passenger strand sequence are sequences that comprise a nucleotide sequence selected from SEQ ID NO: 241-250. Exemplary guide strand nucleotide sequences for inhibiting human glutamine synthetase and their respective passenger strand nucleotide sequences are SEQ ID NOs: 69 and 154, SEQ ID NOs: 70 and 155, SEQ ID NOs: 71 and 156, SEQ ID NOs: 72 and 157, SEQ ID NOs: 73 and 158, SEQ ID NOs: 74 and 159, SEQ ID NOs: 75 and 160, SEQ ID NOs: 76 and 161, SEQ ID NOs: 77 and 162, SEQ ID NOs: 78 and 163, SEQ ID NOs: 79 and 164, SEQ ID NOs: 80 and 165, SEQ ID NOs: 81 and 166, SEQ ID NOs: 628 and 631, SEQ ID NOs: 629 and 632, SEQ ID NOs: 630 and 633.

Multi-hairpin amiRNA with nucleotide sequence SEQ ID NO: 634 comprises guide strand sequences complementary to four different sequences within the human glutamine synthetase mRNA target (nucleotide sequence SEQ ID NO: 11). Multi-hairpin amiRNA nucleotide sequence SEQ ID NO: 634 comprises a first guide strand sequence with nucleotide sequence SEQ ID NO: 628 and a first passenger strand sequence with nucleotide sequence SEQ ID NO: 631; nucleotide sequence SEQ ID NO: 634 further comprises a second guide strand sequence with nucleotide sequence SEQ ID NO: 69 and a second passenger strand sequence with nucleotide sequence SEQ ID NO: 154; nucleotide sequence SEQ ID NO: 634 further comprises a third guide strand sequence with nucleotide sequence SEQ ID NO: 629 and a third passenger strand sequence with nucleotide sequence SEQ ID NO: 632; nucleotide sequence SEQ ID NO: 634 further comprises a fourth guide strand sequence with nucleotide sequence SEQ ID NO: 630 and a third passenger strand sequence with nucleotide sequence SEQ ID NO: 633. Guide strand nucleotide sequences SEQ ID NO: 69, SEQ ID NO: 628, SEQ ID NO: 629 and SEQ ID NO: 630 are all different from each other. Each guide strand sequence is separated from its respective passenger strand sequence by a nucleotide sequence comprising SEQ ID NO: 241. Incorporation of the multi-hairpin amiRNA into a transposon vector enhances the likelihood that the amiRNA genes will be integrated into transcriptionally active regions of the genome.

In one embodiment, a multi-hairpin amiRNA gene to express amiRNAs that inhibit glutamine synthetase are introduced into a cell lacking a functional dihydrofolate reductase gene. The resulting cell requires media supplemented with glutamine, hypoxanthine and thymidine (HT) in order to grow.

### 5.4.2 Complementation of Glutamine Synthetase Auxotrophy

In cells lacking a functional glutamine synthetase gene, including cells in which endogenous glutamine synthetase expression is reduced by RNA interference (for example by integration of multi-hairpin amiRNA gene comprising nucleotide sequence SEQ ID NO: 209 operably linked to a promoter active in a mammalian cell, into the genome of the cell) an exogenously added glutamine synthetase gene can function as a selectable marker by permitting growth in a glutamine-free medium (i.e., rescuing the cell from glutamine auxotrophy). Preferably a polynucleotide comprising the exogenous glutamine synthetase gene is introduced into the cell. Preferably the exogenous glutamine synthetase gene comprises sequence features that prevent its expression from being inhibited by any RNA interference that has been used to make the host cell auxotrophic for glutamine. If RNA interference molecules, including amiRNA guide strands, are complementary to the coding portion of the endogenous glutamine synthetase, an exogenous gene encoding glutamine synthetase can avoid inhibition if the coding sequence is changed, for example by silent mutations in the targeted region. If RNA interference molecules, including amiRNA guide strands, are complementary to the 3′ UTR or 5′ UTR portions of the endogenous glutamine synthetase, an exogenous gene encoding glutamine synthetase can avoid inhibition by replacing the natural UTR sequences with alternative sequences, for example synthetic sequences or UTR sequences taken or adapted from other natural genes.

Selection protocols include introducing a polynucleotide comprising sequences encoding a glutamine synthase selectable marker, and then growing the cell in media that does not contain enough glutamine for the cells to survive in the absence of an exogenous gene encoding glutamine synthetase.

Preferably the exogenous gene encoding glutamine synthetase gene is operably linked to a weak promoter or other sequence elements that attenuate expression, such that high levels of expression can only occur if many copies of the polynucleotide are present, or if they are integrated in a position in the genome where high levels of expression occur. In such cases it may be unnecessary to use a glutamine synthetase inhibitor such as methionine sulphoximine: simply synthesizing enough glutamine for cell survival may provide a sufficiently stringent selection if expression of the glutamine synthetase is attenuated. Exemplary glutamine synthetase polypeptide sequences are given as SEQ ID NOs: 304-308.

### 5.5 Synthetic Amirna Target Sequences

As described in Sections 5.3 and 6.1, multi-hairpin amiRNA sequences are described that effectively inhibit expression of Cricetulus griseus alpha-(1,6)-fucosyl transferase (FUT8). DNA encoding the amiRNA sequences, for example nucleotide sequences SEQ ID NOs: 193-195, can be operably linked to a promoter active in a mammalian cell. The guide RNAs in these multihairpin amiRNAs are complementary to the 3′ UTR of the FUT8 mRNA (with nucleotide sequence SEQ ID NO: 2). Expression of a polypeptide from a polynucleotide that comprises a promoter operably linked to an open reading frame encoding the polypeptide and a polyadenylation signal sequence is inhibitable by these multi-hairpin amiRNAs if the polynucleotide further comprises nucleotide sequence SEQ ID NO: 2, such that SEQ ID NO:2 is transcribed and incorporated into the primary transcript. Preferably SEQ ID NO: 2 is located to the 3′ of the open reading frame encoding the polypeptide, and to the 5′ of the polyadenylation signal sequence. Preferably the open reading frame is operably linked to a promoter active in a eukaryotic cell, more preferably the promoter is active in a mammalian cell. Preferably the open reading frame does not encode Cricetulus griseus alpha-(1,6)-fucosyl transferase or Cricetulus griseus glutamine synthetase. The polynucleotide comprising nucleotide sequence SEQ ID NO: 2 and the polynucleotide encoding multi-hairpin amiRNA comprising a nucleotide sequence selected from SEQ ID NOs: 193-195 may be introduced into the same eukaryotic cell. Preferably the polynucleotide encoding the multi-hairpin amiRNA is operably linked to a promoter that is active in the cell; the promoter may be inducible or constitutive. The eukaryotic cell is preferably a mammalian cell, more preferably a human cell or a rodent cell. The cell is an aspect of the invention.

Multi-hairpin amiRNA with nucleotide sequence SEQ ID NO: 193 comprises guide strand sequences complementary to a region of the 3′ UTR of Cricetulus griseus FUT8 with sequence SEQ ID NO: 560. Expression of a polypeptide from a polynucleotide that comprises a promoter operably linked to an open reading frame encoding the polypeptide and a polyadenylation signal sequence is inhibitable by a multi-hairpin amiRNA comprising sequence SEQ ID NO: 193, if the polynucleotide further comprises nucleotide sequence SEQ ID NO: 560, such that SEQ ID NO: 560 is transcribed and incorporated into the primary transcript. Preferably SEQ ID NO: 560 is located to the 3′ of the open reading frame encoding the polypeptide, and to the 5′ of the polyadenylation signal sequence. Preferably the open reading frame is operably linked to a promoter active in a eukaryotic cell, more preferably the promoter is active in a mammalian cell. Preferably the open reading frame does not encode Cricetulus griseus alpha-(1,6)-fucosyl transferase or Cricetulus griseus glutamine synthetase. The polynucleotide comprising nucleotide sequence SEQ ID NO: 560 and a polynucleotide encoding the multi-hairpin amiRNA comprising nucleotide sequence SEQ ID NO: 193 or the amiRNA may be introduced into the same eukaryotic cell. Preferably a polynucleotide encoding the multi-hairpin amiRNA is operably linked to a promoter that is active in the cell; the promoter may be inducible or constitutive. The eukaryotic cell is preferably a mammalian cell, a human cell or a rodent cell. The cell is an aspect of the invention.

Multi-hairpin amiRNA with nucleotide sequence SEQ ID NO: 194 comprises guide strand sequences complementary to a region of the 3′ UTR of Cricetulus griseus FUT8 with sequence SEQ ID NO: 560. Expression of a polypeptide from a polynucleotide that comprises a promoter operably linked to an open reading frame encoding the polypeptide and a polyadenylation signal sequence is inhibitable by a multi-hairpin amiRNA comprising SEQ ID NO: 194 if the polynucleotide further comprises nucleotide sequence SEQ ID NO: 560, such that SEQ ID NO: 560 is transcribed and incorporated into the primary transcript. Preferably SEQ ID NO: 560 is located to the 3′ of the open reading frame encoding the polypeptide, and to the 5′ of the polyadenylation signal sequence. Preferably the open reading frame is operably linked to a promoter active in a eukaryotic cell, more preferably the promoter is active in a mammalian cell. Preferably the open reading frame does not encode Cricetulus griseus alpha-(1,6)-fucosyl transferase or Cricetulus griseus glutamine synthetase. The polynucleotide comprising nucleotide sequence SEQ ID NO: 560 and a polynucleotide encoding the multi-hairpin amiRNA comprising nucleotide sequence SEQ ID NO: 194 or the amiRNA may be introduced into the same eukaryotic cell. Preferably a polynucleotide encoding the multi-hairpin amiRNA is operably linked to a promoter that is active in the cell; the promoter may be inducible or constitutive. The eukaryotic cell is preferably a mammalian cell, a human cell or a rodent cell. The cell is an aspect of the invention.

Multi-hairpin amiRNA with nucleotide sequence SEQ ID NO: 195 comprises guide strand sequences complementary to a region of the 3′ UTR of Cricetulus griseus FUT8 with sequence SEQ ID NO: 561. Expression of a polypeptide from a polynucleotide that comprises a promoter operably linked to an open reading frame encoding the polypeptide and a polyadenylation signal sequence is inhibitable by a multi-hairpin amiRNA comprising SEQ ID NO: 195 if the polynucleotide further comprises nucleotide sequence SEQ ID NO: 561, such that SEQ ID NO: 561 is transcribed and incorporated into the primary transcript. Preferably SEQ ID NO: 561 is located to the 3′ of the open reading frame encoding the polypeptide, and to the 5′ of the polyadenylation signal sequence. Preferably the open reading frame is operably linked to a promoter active in a eukaryotic cell, more preferably the promoter is active in a mammalian cell. Preferably the open reading frame does not encode Cricetulus griseus alpha-(1,6)-fucosyl transferase or Cricetulus griseus glutamine synthetase. The polynucleotide comprising nucleotide sequence SEQ ID NO: 561 and a polynucleotide encoding the multi-hairpin amiRNA comprising nucleotide sequence SEQ ID NO: 195 or the amiRNA may be introduced into the same eukaryotic cell. Preferably a polynucleotide encoding the multi-hairpin amiRNA is operably linked to a promoter that is active in the cell; the promoter may be inducible or constitutive. The eukaryotic cell is preferably a mammalian cell, a human cell or a rodent cell. The cell is an aspect of the invention.

As described in Sections 5.4 and 6.2, multi-hairpin amiRNA sequences are described that effectively inhibit expression of Cricetulus griseus glutamine synthetase, for example nucleotide sequence SEQ ID NO: 209. The guide RNAs in this multihairpin amiRNA are complementary to the 3′ UTR of the mRNA (with nucleotide sequence SEQ ID NO: 558). Expression of a polypeptide from a polynucleotide that comprises a promoter operably linked to an open reading frame encoding the polypeptide and a polyadenylation signal sequence is inhibitable by these multi-hairpin amiRNAs if the polynucleotide further comprises nucleotide sequence SEQ ID NO: 558, such that SEQ ID NO: 558 is transcribed and incorporated into the primary transcript. Preferably SEQ ID NO: 558 is located to the 3′ of the open reading frame encoding the polypeptide, and to the 5′ of the polyadenylation signal sequence. Preferably the open reading frame is operably linked to a promoter active in a eukaryotic cell, more preferably the promoter is active in a mammalian cell. Preferably the open reading frame does not encode Cricetulus griseus alpha-(1,6)-fucosyl transferase or Cricetulus griseus glutamine synthetase. The polynucleotide comprising nucleotide sequence SEQ ID NO: 558 and a polynucleotide encoding the multi-hairpin amiRNA comprising nucleotide sequence SEQ ID NO: 209 or the amiRNA may be introduced into the same eukaryotic cell. Preferably the multi-hairpin amiRNA is operably linked to a promoter that is active in the cell; the promoter may be inducible or constitutive. The eukaryotic cell is preferably a mammalian cell, a human cell or a rodent cell. The cell is an aspect of the invention.

Multi-hairpin amiRNA with nucleotide sequence SEQ ID NO: 209 comprises guide strand sequences complementary to a region of the 3′ UTR of Cricetulus griseus glutamine synthetase with sequence SEQ ID NO: 559. Expression of a polypeptide from a polynucleotide that comprises a promoter operably linked to an open reading frame encoding the polypeptide and a polyadenylation signal sequence is inhibitable by a multi-hairpin amiRNA comprising SEQ ID NO: 209 if the polynucleotide further comprises nucleotide sequence SEQ ID NO: 559, such that SEQ ID NO: 559 is transcribed and incorporated into the primary transcript. Preferably SEQ ID NO: 559 is located to the 3′ of the open reading frame encoding the polypeptide, and to the 5′ of the polyadenylation signal sequence. Preferably the open reading frame is operably linked to a promoter active in a eukaryotic cell, more preferably the promoter is active in a mammalian cell. Preferably the open reading frame does not encode Cricetulus griseus alpha-(1,6)-fucosyl transferase or Cricetulus griseus glutamine synthetase. The polynucleotide comprising nucleotide sequence SEQ ID NO: 559 and a polynucleotide encoding the multi-hairpin amiRNA comprising nucleotide sequence SEQ ID NO: 209 or the amiRNA may be introduced into the same eukaryotic cell. Preferably a polynucleotide encoding the multi-hairpin amiRNA is operably linked to a promoter that is active in the cell; the promoter may be inducible or constitutive. The eukaryotic cell is preferably a mammalian cell, a human cell or a rodent cell. The cell is an aspect of the invention.

Use of an inducible promoter from which to express the multi-hairpin amiRNA allows the controlled suppression of expression of the open reading frame to which the polynucleotide comprising the amiRNA target is linked. If a cell comprises a constitutive promoter operably linked to the multi-hairpin amiRNA, the cell will constitutively reduce expression of an open reading frame to which the polynucleotide comprising the amiRNA target is linked. This is useful when the open reading frame encodes a toxic protein and when the cell needs to be able to tolerate the open reading frame, for example if the open reading frame encodes a toxic protein produced by an oncolytic virus, and the cell is to be used to package the virus.

### 5.6 Micro RNA for Inhibiting Sialidases

Sialic acid plays an important role in regulating the serum half-life, stability, and solubility of the therapeutic glycoproteins by preventing the degradation of the terminal glycan structure. Sialic acid is removed from proteins produced by Chinese hamster ovary (CHO) cells by sialidases that are present in the plasma membrane of live cells (Neu3), and those that are released by lysed dead cells (Neu1 and Neu2). The sialic acid content of proteins secreted by CHO cells can be increased by inhibiting expression of endogenous sialidases. Sialylation of CHO-produced proteins has been improved by inhibiting the expression of Neu2 (Ngantung et. al., 2006. Biotechnol. Bioeng. 95, 106-119. “RNA interference of sialidase improves glycoprotein sialic acid content consistency.”) or Neu1, Neu2 or Neu3 (Zhang et. al., 2010. Biotechnol. Bioeng. 105, 1094-1105. “Enhancing glycoprotein sialylation by targeted gene silencing in mammalian cells.”) using RNA interference. In all cases, each siRNA or shRNA was tested independently to identify the most effective, but multiple sequences within the same sialidase mRNA were not targeted. Although an in vitro sialidase test suggested that Zhang et. al. had reduced detectable enzyme activity by −98%, the mRNA was only reduced to 30% of the original levels, and effects on sialic acid content of an interferon molecule produced by the cell were modest. An alternative approach used CRISPR to knock out the sialidase genes (Ha et. al., 2020. Metabolic Engineering 57, 182-192. “Knockout of sialidase and pro-apoptotic genes in Chinese hamster ovary cells enables the production of recombinant human erythropoietin in fedbatch cultures”). The benefit of an RNA interference approach is that it facilitates modification of cell lines that have already been developed to express a protein product: artificial micro RNAs can be easily added subsequently to inhibit sialidase expression.

An advantageous inhibitory polynucleotide for inhibition of sialidases in mammalian cells comprise (i) a first guide strand sequence comprising a contiguous 19 or 20 or 21 or 22 nucleotide sequence that is perfectly complementary to a natural mammalian cellular mRNA encoding a sialidase and (ii) a first passenger strand sequence comprising a contiguous 19 or 20 or 21 or 22 nucleotide sequence that is at least 78% complementary to the first guide strand sequence, wherein the first guide strand and first passenger strand sequence are separated by between 5 and 35 nucleotides and (iii) a second guide strand sequence comprising a contiguous 19 or 20 or 21 or 22 nucleotide sequence that is perfectly complementary to the same natural mammalian cellular mRNA as the first guide strand sequence and (iv) a second passenger strand sequence comprising a contiguous 19 or 20 or 21 or 22 nucleotide sequence that is at least 78% complementary to the second guide strand sequence, wherein the second guide strand and second passenger strand sequence are separated by between 5 and 35 nucleotides, and wherein the first and second guide strand sequences are different from each other and are operably linked to the same promoter that is active in a mammalian cell. Exemplary natural mammalian cellular mRNAs encoding sialidases comprise a nucleotide sequence that is at least 95%, 96%, 97%, 98% or 99% identical to, or 100% identical to a sequence selected from SEQ ID NOs: 13-18 and SEQ ID NOs: 570-571.

One CHO sialidase that removes sialic acid from proteins produced by CHO cells is Neu3. An advantageous polynucleotide for increasing the sialic acid content of proteins produced by CHO cells comprises or encodes a Neu3-inhibiting multi-hairpin amiRNA sequence. The Neu3-inhibiting multi-hairpin amiRNA sequence comprises a first guide strand sequence comprising a contiguous 19 or 20 or 21 or 22 nucleotide sequence complementary to CHO Neu3 mRNA (a sequence selected from SEQ ID NOs: 13 and 571) and a first passenger strand sequence comprising a contiguous 19 or 20 or 21 or 22 nucleotide sequence that is at least 78% identical to the reverse complement of the first guide strand sequence. The Neu3-inhibiting multi-hairpin amiRNA sequence further comprises a second guide strand sequence comprising a contiguous 19 or 20 or 21 or 22 nucleotide sequence complementary to CHO Neu3 mRNA and a second passenger strand sequence comprising a contiguous 19 or 20 or 21 or 22 nucleotide sequence that is at least 78% identical to the reverse complement of the second guide strand sequence, and wherein the first and second guide strand sequences are different from each other and operably linked to the same promoter. The Neu3-inhibiting multi-hairpin amiRNA sequence may optionally comprise a third guide strand sequence comprising a contiguous 19 or 20 or 21 or 22 nucleotide sequence complementary to CHO Neu3 mRNA and a third passenger strand sequence comprising a contiguous 19 or 20 or 21 or 22 nucleotide sequence that is at least 78% identical to the reverse complement of the third guide strand sequence, and wherein the first, second and third guide strand sequences are all different from each other and operably linked to the same promoter. Each guide strand sequence is separated from its respective passenger strand sequence by between 5 and 35 bases. Exemplary sequences for separating a guide strand sequence from its passenger strand sequence are sequences that comprise a nucleotide sequence selected from SEQ ID NO: 241-250. Exemplary guide strand sequences for inhibiting Cricetulus griseus Neu3 and their respective passenger strand sequences are SEQ ID NOs: 85 and 170, SEQ ID NOs: 86 and 171, SEQ ID NOs: 87 and 172, SEQ ID NOs: 88 and 173, SEQ ID NOs: 89 and 174 and SEQ ID NOs: 565 and 566. Exemplary multi-hairpin amiRNAs for inhibition of Neu3 sialidase in CHO cells include nucleotide sequences SEQ ID NOs: 212-216 and 567. A CHO cell comprising one of these multi-hairpin amiRNAs or a polynucleotide encoding them is an aspect of the invention.

One CHO sialidase that removes sialic acid from proteins produced by CHO cells is Neu2. An advantageous polynucleotide for increasing the sialic acid content of proteins produced by CHO cells comprises or encodes a Neu2-inhibiting multi-hairpin amiRNA sequence. The Neu2-inhibiting multi-hairpin amiRNA sequence comprises a first guide strand sequence comprising a contiguous 19 or 20 or 21 or 22 nucleotide sequence complementary to the CHO Neu2 mRNA (i.e. a sequence selected from SEQ ID NOs: 15 and 570) and a first passenger strand sequence comprising a contiguous 19 or 20 or 21 or 22 nucleotide sequence that is at least 78% identical to the reverse complement of the first guide strand sequence. The Neu2-inhibiting multi-hairpin amiRNA sequence further comprises a second guide strand sequence comprising a contiguous 19 or 20 or 21 or 22 nucleotide sequence complementary to the CHO Neu2 mRNA and a second passenger strand sequence comprising a contiguous 19 or 20 or 21 or 22 nucleotide sequence that is at least 78% identical to the reverse complement of the second guide strand sequence, and wherein the first and second guide strand sequences are different from each other and operably linked to the same promoter. The Neu2-inhibiting multi-hairpin amiRNA sequence may optionally comprise a third guide strand sequence comprising a contiguous 19 or 20 or 21 or 22 nucleotide sequence complementary to the CHO Neu2 mRNA and a third passenger strand sequence comprising a contiguous 19 or 20 or 21 or 22 nucleotide sequence that is at least 78% identical to the reverse complement of the third guide strand sequence, and wherein the first, second and third guide strand sequences are all different from each other and operably linked to the same promoter. Each guide strand sequence is separated from its respective passenger strand sequence by between 5 and 35 bases. Exemplary sequences for separating a guide strand sequence from its passenger strand sequence are sequences that comprise a sequence selected from SEQ ID NO: 241-250. Exemplary guide strand nucleotide sequences for inhibiting Cricetulus griseus Neu2 and their respective passenger strand nucleotide sequences are SEQ ID NOs: 90 and 175, SEQ ID NOs: 91 and 176, SEQ ID NOs: 92 and 177, SEQ ID NOs: 93 and 178, SEQ ID NOs: 94 and 179. Exemplary multi-hairpin amiRNAs for inhibition of Neu2 sialidase in CHO cells include nucleotide sequences SEQ ID NOs: 217-221, 568, 595 and 596. A CHO cell comprising one of these multi-hairpin amiRNAs or a polynucleotide encoding them is an aspect of the invention.

Expression of sialidases Neu2 and Neu3 in a mammalian cell can be simultaneously inhibited using an inhibitory polynucleotide comprising or encoding multi-amiRNA hairpins with guides complementary to each of Neu2 and Neu3 mRNAs. This inhibitory polynucleotide comprises (i) a first guide strand sequence comprising a contiguous 19 or 20 or 21 or 22 nucleotide sequence that is perfectly complementary to a natural mammalian cellular mRNA encoding a Neu2 sialidase and (ii) a first passenger strand sequence comprising a contiguous 19 or 20 or 21 or 22 nucleotide sequence that is at least 78% complementary to the first guide strand sequence, wherein the first guide strand and first passenger strand sequence are separated by between 5 and 35 nucleotides and (iii) a second guide strand sequence comprising a contiguous 19 or 20 or 21 or 22 nucleotide sequence that is perfectly complementary to the same natural mammalian cellular mRNA encoding a Neu2 sialidase as the first guide strand sequence and (iv) a second passenger strand sequence comprising a contiguous 19 or 20 or 21 or 22 nucleotide sequence that is at least 78% complementary to the second guide strand sequence, wherein the second guide strand and second passenger strand sequence are separated by between 5 and 35 nucleotides, and wherein the first and second guide strand sequences are different from each other and are operably linked to the same promoter that is active in a mammalian cell; and (v) a third guide strand sequence comprising a contiguous 19 or 20 or 21 or 22 nucleotide sequence that is perfectly complementary to a natural mammalian cellular mRNA encoding a Neu3 sialidase and (vi) a third passenger strand sequence comprising a contiguous 19 or 20 or 21 or 22 nucleotide sequence that is at least 78% complementary to the third guide strand sequence, wherein the third guide strand and third passenger strand sequence are separated by between 5 and 35 nucleotides and (vii) a fourth guide strand sequence comprising a contiguous 19 or 20 or 21 or 22 nucleotide sequence that is perfectly complementary to the same natural mammalian cellular mRNA encoding a Neu3 sialidase as the third guide strand sequence and (viii) a fourth passenger strand sequence comprising a contiguous 19 or 20 or 21 or 22 nucleotide sequence that is at least 78% complementary to the fourth guide strand sequence, wherein the fourth guide strand and fourth passenger strand sequence are separated by between 5 and 35 nucleotides, and wherein the third and fourth guide strand sequences are different from each other and are operably linked to the same promoter that is active in a mammalian cell. Optionally the first, second, third and fourth guide strand sequences are all operably linked to the same promoter active in a mammalian cell. Exemplary natural mammalian cellular mRNAs encoding Neu2 sialidases comprise a sequence that is at least 95%, 96%, 97%, 98% or 99% identical to, or 100% identical to a sequence selected from SEQ ID NOs: 15-17 and 570. Exemplary natural mammalian cellular mRNAs encoding Neu3 sialidases comprise a sequence that is at least 95%, 96%, 97%, 98% or 99% identical to, or 100% identical to a sequence selected from SEQ ID NOs: 13, 14, 18 and 571. Exemplary multi-hairpin amiRNAs and polynucleotides encoding them for inhibition of Neu2 and Neu3 sialidases in CHO cells include nucleotide sequences SEQ ID NOs: 222-225 and 569. A CHO cell comprising one of these multi-hairpin amiRNAs or a polynucleotide encoding them is an aspect of the invention.

A method for producing secreted proteins with increased sialic acid levels from mammalian cells comprises (i) introducing into a mammalian cell an inhibitory polynucleotide for inhibiting expression of a sialidase, wherein the inhibitory polynucleotide comprises or encodes a multi-hairpin amiRNA for expression of two or more interfering RNA guide sequences complementary to the same natural mammalian mRNA, and wherein the natural mammalian mRNA encodes a sialidase (for example but not limited to Neu2 or Neu3), and (ii) introducing into the same mammalian cell a gene encoding a protein to be secreted, the gene expressible in the mammalian cell. The two sequences may be introduced in any order: for example, the inhibitory polynucleotide may be introduced first and the gene encoding a protein to be secreted may be introduced second, the gene encoding a protein to be secreted may be introduced first and the inhibitory polynucleotide may be introduced second, or the two sequences may be introduced to the mammalian cell at the same time. The inhibitory polynucleotide and the gene encoding the protein to be secreted may be introduced to the mammalian cell on the same DNA molecule. In some instances, the protein to be secreted is a therapeutic protein. Preferably the secreted protein is not naturally produced by the cell. Preferably the cell is a CHO cell. The method may further comprise growing the cell under conditions where it produces the secreted protein. The method may further comprise purifying the secreted protein. Examples of therapeutic proteins that may benefit from sialylation include, but are not limited to, erythropoietin (EPO), clotting factors such as Factor VII, Factor IX, Factor X, Protein C, antithrombin III or thrombin, carbohydrate antigens and serum biomarkers, cytokines such as interferon α, interferon β, interferon γ, interferon ω, Granulocyte-colony Stimulating Factor (GCSF) or Granulocyte Macrophage Colony-Stimulating Factor (GM-CSF), receptors, antibodies or immunoglobulins such as IgG, IgG fragments, IgG fusions, and IgM, soluble IgE receptor α-chain, immuno-adhesion proteins and other Fc fusion proteins such as soluble TNF receptor-Fc fusion proteins; RAGE-Fc fusion protein, interleukins; urokinase; chymase; and urea trypsin inhibitor, IGF-binding protein; growth factors such as epidermal growth factor (EGF) or vascular endothelial growth factor (VEGF), annexin V fusion protein; angiostatin, myeloid progenitor inhibitory factor-1; osteoprotegerin, α-1-antitrypsin; α-fetoproteins, DNaseII, human plasminogen, Kringle 3 domain of human plasminogen; glucocerebrosidase; TNF binding protein 1; Follicle stimulating hormone, Thyroid-stimulating hormone, Chorionogonadotropin, Luteinizing Hormone, cytotoxic T lymphocyte associated antigen 4-Ig, transmembrane activator and calcium modulator and cyclophilin ligand; glucagon like protein 1, IL-15 or IL-2 receptor agonist. A therapeutic protein may comprise an antibody, a functional fragment or derivative thereof and more specifically any antibody, functional fragment or derivative thereof that functions to deplete target cells or molecules in a patient. Specific examples of such target cells include tumor cells, virus-infected cells, allogenic cells, pathological immunocompetent cells {e.g., B lymphocytes, T lymphocytes, antigen-presenting cells, etc.) involved in cancers, allergies, autoimmune diseases, allogenic reactions. Most preferred target cells within the context of this invention are immune cells, tumor cells and virus-infected cells. The therapeutic antibodies Mays, for instance, mediate B-lymphocyte depletion (anti-inflammatory antibodies such as anti-CD20 antibodies) or a cytotoxic effect or cell lysis (pro-inflammatory antibodies), particularly by antibody-dependent cell-mediated cytotoxicity (ADCC). Therapeutic antibodies according to the invention may be directed to circulatory mediators of inflammation, cell surface epitopes overexpressed by cancer cells, or viral epitopes. A therapeutic antibody may be selected from the group comprising rituximab, trastuzumab, cetuximab, motavizumab, palivizumab, alemtuzumab, but also comprising for instance, abciximab, adalimumab, alemtuzumab, basiliximab, belimumab, benralizumab, bevacizumab, brentuximab, canakinumab, catumaxomab, daratumumab, elotuzumab, epratuzumab, farletuzumab, galiximab, gemtuzumabozogamicin, golimumab, ibritumomabtiuxetan, ipilimumab, lumiliximab, necitumumab, nimotuzumab, ocrelizumab, ofatumumab, omalizumab, oregovomab, pertuzumab, raxibacumab, tocilizumab, tositumomab, ustekinumab, zalutumumab, and zanolimumab, preferably infliximab.

The invention also includes a cell comprising a polynucleotide, which comprises or encodes a multi-hairpin amiRNA. The amiRNA introduced directly into the cell or expressed from the polynucleotide in the cell can inhibit expression of a sialidase. The cell can also include a heterologous polynucleotide encoding a secreted protein. When the polynucleotide encoding the secreted protein is expressed, the amiRNA inhibits expression of the sialidase resulting in increased sialylation of the secreted proteins. The cell is preferably a mammalian cell line and can be one of a population of such cells, such as a cell line.

### 5.7 Micro RNA for Inhibiting the Interferon Receptor

Interferons are produced in response to viral attacks on cells, and their effect is to reduce cell growth and proliferation. Attempts to produce interferons in large quantities using mammalian cells are often stymied by the action of the interferons to slow growth. Interferons act on cells through an interferon receptor which has two subunits: IFNAR1 and IFNAR2. Inhibiting or reducing expression of the interferon receptor in a producer cell reduces the susceptibility of that cell to the inhibitory effects of interferons, thereby enhancing its ability to produce interferons. Reducing expression of either subunit disrupts the ability of interferons to signal and reduce cellular growth, thereby enhancing the ability of a cell to produce interferons for example beta interferon. Preferably the expression of a subunit of the interferon receptor is reduced or inhibited by RNA interference. The RNA interference may be mediated by an artificial micro-RNA gene.

A gene encoding an interferon polypeptide may be introduced into a cell that contains an inhibitory polynucleotide comprising a gene expressing one or more interfering RNA sequences to reduce expression of a subunit of an interferon receptor. A gene encoding an interferon polypeptide may be introduced into a cell as part of a DNA molecule that also contains an inhibitory polynucleotide comprising a gene expressing one or more interfering RNA sequences to reduce expression of a subunit of the interferon receptor. As described in Section 5.2.6, it is advantageous to incorporate the multi-amiRNA hairpins encoding the interfering RNAs into the 3′ UTR of the selectable marker.

An advantageous inhibitory polynucleotide for inhibition of the interferon receptor in mammalian cells comprises or encodes (i) a first guide strand sequence comprising a contiguous 19 or 20 or 21 or 22 nucleotide sequence that is perfectly complementary to a natural mammalian cellular mRNA encoding a subunit of the interferon receptor and (ii) a first passenger strand sequence comprising a contiguous 19 or 20 or 21 or 22 nucleotide sequence that is at least 78% complementary to the first guide strand sequence, wherein the first guide strand and first passenger strand sequence are separated by between 5 and 35 nucleotides and (iii) a second guide strand sequence comprising a contiguous 19 or 20 or 21 or 22 nucleotide sequence that is perfectly complementary to the same natural mammalian cellular mRNA as the first guide strand sequence and (iv) a second passenger strand sequence comprising a contiguous 19 or 20 or 21 or 22 nucleotide sequence that is at least 78% complementary to the second guide strand sequence, wherein the second guide strand and second passenger strand sequence are separated by between 5 and 35 nucleotides, and wherein the first and second guide strand sequences are different from each other and are operably linked to the same promoter that is active in a mammalian cell. Exemplary natural mammalian cellular mRNAs encoding interferon receptor subunits comprise a nucleotide sequence that is at least 95%, 96%, 97%, 98% or 99% identical to, or 100% identical to a sequence selected from SEQ ID NOs: 19-22.

The CHO IFNAR1 gene is encoded by an mRNA comprising a nucleotide sequence that is at least 95%, 96%, 97%, 98% or 99% identical to, or 100% identical to SEQ ID NO: 19. An advantageous polynucleotide for inhibiting expression of the interferon receptor in CHO cells comprises an IFNAR1-inhibiting multi-hairpin amiRNA sequence. The IFNAR1-inhibiting multi-hairpin amiRNA sequence comprises a first guide strand sequence comprising a contiguous 19 or 20 or 21 or 22 nucleotide sequence complementary to SEQ ID NO: 19 and a first passenger strand sequence comprising a contiguous 19 or 20 or 21 or 22 nucleotide sequence that is at least 78% identical to the reverse complement of the first guide strand sequence. The IFNAR1-inhibiting multi-hairpin amiRNA sequence further comprises a second guide strand sequence comprising a contiguous 19 or 20 or 21 or 22 nucleotide sequence complementary to SEQ ID NO: 19 and a second passenger strand sequence comprising a contiguous 19 or 20 or 21 or 22 nucleotide sequence that is at least 78% identical to the reverse complement of the second guide strand sequence, and wherein the first and second guide strand sequences are different from each other and operably linked to the same promoter. The IFNAR1-inhibiting multi-hairpin amiRNA sequence may optionally comprise a third guide strand sequence comprising a contiguous 19 or 20 or 21 or 22 nucleotide sequence complementary to SEQ ID NO: 19 and a third passenger strand sequence comprising a contiguous 19 or 20 or 21 or 22 nucleotide sequence that is at least 78% identical to the reverse complement of the third guide strand sequence, and wherein the first, second and third guide strand sequences are all different from each other and operably linked to the same promoter. Each guide strand sequence is separated from its respective passenger strand sequence by between 5 and 35 bases. Exemplary sequences for separating a guide strand sequence from its passenger strand sequence are sequences that comprise a nucleotide sequence selected from SEQ ID NO: 241-250. Exemplary guide strand sequences for inhibiting CHO IFNAR1 and their respective passenger strand sequences are SEQ ID NOs: 95 and 180, SEQ ID NOs: 96 and 181, SEQ ID NOs: 97 and 182, SEQ ID NOs: 98 and 183, SEQ ID NOs: 99 and 184, SEQ ID NOs: 100 and 185 and SEQ ID NOs: 101 and 186. Exemplary multi-hairpin amiRNAs for inhibition of IFNAR1 in CHO cells include SEQ ID NOs: 226-230.

The CHO IFNAR2 gene is encoded by an mRNA comprising a nucleotide sequence that is at least 95%, 96%, 97%, 98% or 99% identical to, or 100% identical to SEQ ID NO: 20. An advantageous polynucleotide for inhibiting expression of the interferon receptor in CHO cells comprises or encodes an IFNAR2-inhibiting multi-hairpin amiRNA sequence. The IFNAR2-inhibiting multi-hairpin amiRNA sequence comprises a first guide strand sequence comprising a contiguous 19 or 20 or 21 or 22 nucleotide sequence complementary to SEQ ID NO: 20 and a first passenger strand sequence comprising a contiguous 19 or 20 or 21 or 22 nucleotide sequence that is at least 78% identical to the reverse complement of the first guide strand sequence. The IFNAR2-inhibiting multi-hairpin amiRNA sequence further comprises a second guide strand sequence comprising a contiguous 19 or 20 or 21 or 22 nucleotide sequence complementary to SEQ ID NO: 20 and a second passenger strand sequence comprising a contiguous 19 or 20 or 21 or 22 nucleotide sequence that is at least 78% identical to the reverse complement of the second guide strand sequence, and wherein the first and second guide strand sequences are different from each other and operably linked to the same promoter. The IFNAR2-inhibiting multi-hairpin amiRNA sequence may optionally comprise a third guide strand sequence comprising a contiguous 19 or 20 or 21 or 22 nucleotide sequence complementary to SEQ ID NO: 20 and a third passenger strand sequence comprising a contiguous 19 or 20 or 21 or 22 nucleotide sequence that is at least 78% identical to the reverse complement of the third guide strand sequence, and wherein the first, second and third guide strand sequences are all different from each other and operably linked to the same promoter. Each guide strand sequence is separated from its respective passenger strand sequence by between 5 and 35 bases. Exemplary sequences for separating a guide strand sequence from its passenger strand sequence are sequences that comprise a sequence selected from SEQ ID NO: 241-250. Exemplary guide strand sequences for inhibiting IFNAR2 and their respective passenger strand sequences are SEQ ID NOs: 102 and 187, SEQ ID NOs: 103 and 188, SEQ ID NOs:104 and 189, SEQ ID NOs: 105 and 190, SEQ ID NOs: 106 and 191 and SEQ ID NOs: 107 and 192. Exemplary multi-hairpin amiRNAs for inhibition of IFNAR2 in CHO cells include SEQ ID NOs: 231-235.

Expression of both interferon receptor subunits in a mammalian cell can be simultaneously inhibited using an inhibitory polynucleotide comprising or encoding multi-amiRNA hairpins with guides complementary to each of IFNAR1 and IFNAR2 mRNAs. This inhibitory polynucleotide comprises or encodes (i) a first guide strand sequence comprising a contiguous 19 or 20 or 21 or 22 nucleotide sequence that is perfectly complementary to a natural mammalian cellular mRNA encoding IFNAR1 and (ii) a first passenger strand sequence comprising a contiguous 19 or 20 or 21 or 22 nucleotide sequence that is at least 78% complementary to the first guide strand sequence, wherein the first guide strand and first passenger strand sequence are separated by between 5 and 35 nucleotides and (iii) a second guide strand sequence comprising a contiguous 19 or 20 or 21 or 22 nucleotide sequence that is perfectly complementary to the same natural mammalian cellular mRNA encoding IFNAR1 as the first guide strand sequence and (iv) a second passenger strand sequence comprising a contiguous 19 or 20 or 21 or 22 nucleotide sequence that is at least 78% complementary to the second guide strand sequence, wherein the second guide strand and second passenger strand sequence are separated by between 5 and 35 nucleotides, and wherein the first and second guide strand sequences are different and the polynucleotides encoding them are operably linked to the same promoter that is active in a mammalian cell; and (v) a third guide strand sequence comprising a contiguous 19 or 20 or 21 or 22 nucleotide sequence that is perfectly complementary to a natural mammalian cellular mRNA encoding IFNAR2 and (vi) a third passenger strand sequence comprising a contiguous 19 or 20 or 21 or 22 nucleotide sequence that is at least 78% complementary to the third guide strand sequence, wherein the third guide strand and third passenger strand sequence are separated by between 5 and 35 nucleotides and (vii) a fourth guide strand sequence comprising a contiguous 19 or 20 or 21 or 22 nucleotide sequence that is perfectly complementary to the same natural mammalian cellular mRNA encoding IFNAR2 as the third guide strand sequence and (viii) a fourth passenger strand sequence comprising a contiguous 19 or 20 or 21 or 22 nucleotide sequence that is at least 78% complementary to the fourth guide strand sequence, wherein the fourth guide strand and fourth passenger strand sequence are separated by between 5 and 35 nucleotides, and wherein the third and fourth guide strand sequences are different and the polynucleotides encoding them are operably linked to the same promoter that is active in a mammalian cell. Optionally the polynucleotides encoding the first, second, third and fourth guide strand sequences are all operably linked to the same promoter active in a mammalian cell. Exemplary natural mammalian cellular mRNAs encoding IFNAR1 comprise a sequence that is at least 95%, 96%, 97%, 98% or 99% identical to, or 100% identical to a sequence selected from SEQ ID NOs: 19 and 21. Exemplary natural mammalian cellular mRNAs encoding IFNAR2 comprise a sequence that is at least 95%, 96%, 97%, 98% or 99% identical to, or 100% identical to a sequence selected from SEQ ID NOs: 20 and 22. Exemplary multi-hairpin amiRNAs for inhibition of IFNAR1 and IFNAR2 in CHO cells include SEQ ID NOs: 236-240.

A method for producing interferons from mammalian cells comprises (i) introducing into a mammalian cell an inhibitory polynucleotide for inhibiting expression of the interferon receptor, wherein the inhibitory polynucleotide comprises or encodes a multi-hairpin amiRNA for expression of two or more interfering RNA guide sequences complementary to the same natural mammalian mRNA, and wherein the natural mammalian mRNA encodes a subunit of the interferon receptor (for example IFNAR1 or IFNAR2), and (ii) introducing into the same mammalian cell a gene encoding an interferon, the gene expressible in the mammalian cell. The two sequences may be introduced in any order: for example, the inhibitory polynucleotide may be introduced first and the gene encoding the interferon may be introduced second, the gene encoding the interferon may be introduced first and the inhibitory polynucleotide may be introduced second, or the two sequences may be introduced to the mammalian cell at the same time. The gene expressing the interferon may be carried on the same DNA molecule as the multi-hairpin amiRNA, or they may be on separate DNA molecules. The method may further comprise growing the cell under conditions that result in expression of the interferon. The method may further comprise purifying the interferon.

The invention also includes a cell comprising a polynucleotide, which comprises or encodes a multi-hairpin amiRNA. The amiRNA introduced into the cell directly or expressed from the polynucleotide in the cell can inhibit expression of a subunit of the interferon receptor. The cell can also include a heterologous polynucleotide encoding an interferon. When the polynucleotide encoding the interferon is expressed, the interferon causes reduced toxicity to the cell compared with a control cell lacking the polynucleotide comprising or encoding the amiRNA sequence. The cell is preferably a mammalian cell line and can be one of a population of such cells, such as a cell line.

### 5.8 Micro RNA for Inhibiting Lipases

Lipoprotein lipase is a protein produced by CHO cells that is often difficult to purify away from biopharmaceuticals manufactured in CHO cells. Residual lipoprotein lipase can degrade polysorbate that is often used in final product formulations. Deletion of the lipoprotein lipase genes from CHO cells can be used to reduce lipoprotein lipase contaminants from proteins purified from CHO cell cultures (Chui et. al., 2017. Biotechnol Bioeng. 114: 1006-1015. “Knockout of a difficult-to-remove CHO host cell protein, lipoprotein lipase, for improved polysorbate stability in monoclonal antibody formulations”). Other fatty acid hydrolases implicated in polysorbate degradation include phospholipase B-like 2 (exemplary mRNA nucleotide sequence SEQ ID NO: 590), lysozomal acid lipase (exemplary mRNA nucleotide sequence SEQ ID NO: 591) and acid ceramidase (exemplary mRNA nucleotide sequence SEQ ID NO: 592). Reducing the expression of these proteins is also advantageous for reducing contaminating host cell protein in protein therapeutics and reducing degradation of polysorbate in final formulations. The benefit of an RNA interference approach is that it facilitates modification of cell lines that have already been developed to express a protein product: artificial micro RNAs or polynucleotides encoding them can be easily added subsequently to inhibit lipoprotein lipase expression.

An advantageous inhibitory polynucleotide for inhibition of fatty acid hydrolase expression in mammalian cells comprise or encodes (i) a first guide strand sequence comprising a contiguous 19 or 20 or 21 or 22 nucleotide sequence that is perfectly complementary to a natural mammalian cellular mRNA encoding a fatty acid hydrolase and (ii) a first passenger strand sequence comprising a contiguous 19 or 20 or 21 or 22 nucleotide sequence that is at least 78% complementary to the first guide strand sequence, wherein the first guide strand and first passenger strand sequence are separated by between 5 and 35 nucleotides and (iii) a second guide strand sequence comprising a contiguous 19 or 20 or 21 or 22 nucleotide sequence that is perfectly complementary to the same natural mammalian cellular mRNA as the first guide strand sequence and (iv) a second passenger strand sequence comprising a contiguous 19 or 20 or 21 or 22 nucleotide sequence that is at least 78% complementary to the second guide strand sequence, wherein the second guide strand and second passenger strand sequence are separated by between 5 and 35 nucleotides, and wherein the first and second guide strand sequences are different from each other and are operably linked to the same promoter that is active in a mammalian cell. Exemplary natural mammalian cellular mRNA encoding lipoprotein lipase comprise a nucleotide sequence that is at least 95%, 96%, 97%, 98% or 99% identical to, or 100% identical to a sequence selected from SEQ ID NO: 572 or 590-592.

An advantageous polynucleotide for reducing lipoprotein lipase contamination in proteins produced by CHO cells comprises or encodes a lipoprotein lipase-inhibiting multi-hairpin amiRNA sequence. The lipoprotein lipase-inhibiting multi-hairpin amiRNA sequence comprises a first guide strand sequence comprising a contiguous 19 or 20 or 21 or 22 nucleotide sequence complementary to SEQ ID NO: 572 and a first passenger strand sequence comprising a contiguous 19 or 20 or 21 or 22 nucleotide sequence that is at least 78% identical to the reverse complement of the first guide strand sequence. The lipoprotein lipase-inhibiting multi-hairpin amiRNA sequence further comprises a second guide strand sequence comprising a contiguous 19 or 20 or 21 or 22 nucleotide sequence complementary to SEQ ID NO: 572 and a second passenger strand sequence comprising a contiguous 19 or 20 or 21 or 22 nucleotide sequence that is at least 78% identical to the reverse complement of the second guide strand sequence, and wherein the first and second guide strand sequences are different from each other and operably linked to the same promoter. The lipoprotein lipase-inhibiting multi-hairpin amiRNA sequence may optionally comprise a third guide strand sequence comprising a contiguous 19 or 20 or 21 or 22 nucleotide sequence complementary to SEQ ID NO: 572 and a third passenger strand sequence comprising a contiguous 19 or 20 or 21 or 22 nucleotide sequence that is at least 78% identical to the reverse complement of the third guide strand sequence, and wherein the first, second and third guide strand sequences are all different from each other and operably linked to the same promoter. Each guide strand sequence is separated from its respective passenger strand sequence by between 5 and 35 bases. Exemplary sequences for separating a guide strand sequence from its passenger strand sequence are sequences that comprise a nucleotide sequence selected from SEQ ID NO: 241-250. Exemplary guide strand sequences for inhibiting Cricetulus griseus lipoprotein lipase and their respective passenger strand sequences are SEQ ID NOs: 573 and 579, SEQ ID NOs: 574 and 580, SEQ ID NOs: 575 and 581, SEQ ID NOs: 576 and 582, SEQ ID NOs: 577 and 583 and SEQ ID NOs:578 and 584. Exemplary multi-hairpin amiRNAs for inhibition of lipoprotein lipase in CHO cells include nucleotide sequences SEQ ID NOs: 585-589.

A method for producing secreted proteins with reduced lipoprotein lipase contaminants from mammalian cells comprises (i) introducing into a mammalian cell an inhibitory polynucleotide for inhibiting expression of lipoprotein lipase, wherein the inhibitory polynucleotide comprises or encodes a multi-hairpin amiRNA for expression of two or more interfering RNA guide sequences complementary to the same natural mammalian mRNA, and wherein the natural mammalian mRNA encodes a lipoprotein lipase, and (ii) introducing into the same mammalian cell a gene encoding a protein to be secreted, the gene expressible in the mammalian cell. The two sequences may be introduced in any order: for example the inhibitory polynucleotide may be introduced first and the gene encoding a protein to be secreted may be introduced second, the gene encoding a protein to be secreted may be introduced first and the inhibitory polynucleotide may be introduced second, or the two sequences may be introduced to the mammalian cell at the same time. In some instances, the protein to be secreted is a therapeutic protein. Preferably the secreted protein is not naturally produced by the cell. Preferably the cell is a CHO cell. The method may further comprise growing the cell under conditions where it produces the secreted protein. The method may further comprise purifying the secreted protein. Examples of therapeutic proteins that may benefit from reduced lipoprotein lipase levels include, but are not limited to, erythropoietin (EPO), clotting factors such as Factor VII, Factor IX, Factor X, Protein C, antithrombin III or thrombin, carbohydrate antigens and serum biomarkers, cytokines such as interferon α, interferon β, interferon γ, interferon ω, Granulocyte-colony Stimulating Factor (GCSF) or Granulocyte Macrophage Colony-Stimulating Factor (GM-CSF), receptors, antibodies or immunoglobulins such as IgG, IgG fragments, IgG fusions, and IgM, soluble IgE receptor α-chain, immuno-adhesion proteins and other Fc fusion proteins such as soluble TNF receptor-Fc fusion proteins; RAGE-Fc fusion protein, interleukins; urokinase, chymase; and urea trypsin inhibitor; IG-binding protein; growth factors such as epidermal growth factor (EGF) or vascular endothelial growth factor (VEGF); annexin V fusion protein; angiostatin, myeloid progenitor inhibitory factor-1; osteoprotegerin, α-1-antitrypsin; α-fetoproteins, DNaseII, human plasminogen, Kringle 3 domain of human plasminogen; glucocerebrosidase, TNF binding protein 1; Follicle stimulating hormone; Thyroid-stimulating hormone, Chorionogonadotropin, Luteinizing Hormone, cytotoxic T lymphocyte associated antigen 4-11 g; transmembrane activator and calcium modulator and cyclophilin ligand; glucagon like protein 1, IL-15 or IL-2 receptor agonist. A therapeutic protein may comprise an antibody, a functional fragment or derivative thereof and more specifically any antibody, functional fragment or derivative thereof that functions to deplete target cells or molecules in a patient. Specific examples of such target cells include tumor cells, virus-infected cells, allogenic cells, pathological immunocompetent cells {e.g., B lymphocytes, T lymphocytes, antigen-presenting cells, etc.) involved in cancers, allergies, autoimmune diseases, allogenic reactions. Most preferred target cells within the context of this invention are immune cells, tumor cells and virus-infected cells. The therapeutic antibodies may, for instance, mediate 13 lymphocyte depletion (anti-inflammatory antibodies such as anti-CD20 antibodies) or a cytotoxic effect or cell lysis (pro-inflammatory antibodies), particularly by antibody-dependent cell-mediated cytotoxicity (ADCC). Therapeutic antibodies according to the invention may be directed to circulatory mediators of inflammation, cell surface epitopes overexpressed by cancer cells, or viral epitopes. A therapeutic antibody may be selected from the group comprising rituximab, trastuzumab, cetuximab, motavizumab, palivizumab, alemtuzumab, but also comprising for instance, abciximab, adalimumab, alemtuzumab, basiliximab, belimumab, benralizumab, bevacizumab, brentuximab, canakinumab, catumaxomab, daratumumab, elotuzumab, epratuzumab, farletuzumab, galiximab, gemtuzumabozogamicin, golimumab, ibritumomabtiuxetan, ipilimumab, lumiliximab, necitumumab, nimotuzumab, ocrelizumab, ofatumumab, omalizumab, oregovomab, pertuzumab, raxibacumab, tocilizumab, tositumomab, ustekinumab, zalutumumab, and zanolimumab, preferably infliximab.

### 5.9 Dihydrofolate Reductase

Another example of a selectable marker gene that may be advantageously incorporated into a gene transfer polynucleotide to provide a growth advantage to the cell by allowing the cell to synthesize a metabolically useful substance is a gene encoding dihydrofolate reductase (DHFR, for example a polypeptide sequence selected from SEQ ID NO: 292-293). DHFR is required for catalyzing the reduction of 5,6-dihydrofolate (DHF) to 5,6,7,8-tetrahydrofolate (THF), which is a proton shuttle required for the de novo synthesis of purines, thymidylic acid, and certain amino acids. Some cell lines do not express enough DHFR to survive without added nucleoside precursors hypoxanthine and thymidine (HT). In these cells a transfected DHFR gene can function as a selectable marker by permitting growth in a hypoxanthine and thymidine-free medium. DHFR confers resistance to methotrexate (MTX). DHFR can be inhibited by higher levels of methotrexate. Selection protocols include introducing a construct comprising sequences encoding a DHFR selectable marker into a cell with or without an endogenous DHFR gene, and then treating the cell with inhibitors of DHFR such as methotrexate. The higher the levels of methotrexate that are used, the higher the level of DHFR expression is required to allow the cell to synthesize enough DHFR to survive. Preferably the DHFR gene is operably linked to a weak promoter or other sequence elements that attenuate expression as described above, such that high levels of expression can only occur if many copies of the gene transfer polynucleotide are present, or if they are integrated in a position in the genome where high levels of expression occur. In such cases it may be unnecessary to use a DHFR inhibitor such as methotrexate: simply synthesizing enough tetrahydrofolate for cell survival may provide a sufficiently stringent selection if expression of the DHFR is attenuated.

In some cell lines, for example HEK cells and Chinese hamster ovary (CHO) cells, there is enough DHFR enzyme expressed to enable the cell to survive without exogenously added HT. These cells can be manipulated by genome editing techniques including CRISPR/Cas9 to reduce or eliminate the activity of the DHFR enzyme. However even with CRISPR this is a laborious process that may introduce off-target mutations in other genes. An alternative method is to stably integrate into the cell genome a polynucleotide comprising a multi-hairpin amiRNA that targets the endogenous DHFR mRNA.

### 5.9.1 Micro RNA to Reduce Endogenous Dihydrofolate Reductase

An advantageous gene transfer polynucleotide for inhibition of dihydrofolate reductase in hamster cells comprises a dihydrofolate reductase-inhibiting multi-hairpin amiRNA sequence. The dihydrofolate reductase-inhibiting multi-hairpin amiRNA sequence comprises a first guide strand sequence comprising a contiguous 19 or 20 or 21 or 22 nucleotide sequence complementary to hamster DHFR mRNA (whose nucleotide sequence is given by SEQ ID NO: 22) and a first passenger strand sequence comprising a contiguous 19 or 20 or 21 or 22 nucleotide sequence that is at least 78% identical to the reverse complement of the first guide strand sequence. The dihydrofolate reductase-inhibiting multi-hairpin amiRNA sequence further comprises a second guide strand sequence comprising a contiguous 19 or 20 or 21 or 22 nucleotide sequence complementary to SEQ ID NO: 22 and a second passenger strand sequence comprising a contiguous 19 or 20 or 21 or 22 nucleotide sequence that is at least 78% identical to the reverse complement of the second guide strand sequence, and wherein the first and second guide strand sequences are different from each other. The dihydrofolate reductase-inhibiting multi-hairpin amiRNA sequence may optionally comprise a third guide strand sequence comprising a contiguous 19 or 20 or 21 or 22 nucleotide sequence complementary to SEQ ID NO: 22 and a third passenger strand sequence comprising a contiguous 19 or 20 or 21 or 22 nucleotide sequence that is at least 78% identical to the reverse complement of the third guide strand sequence, and wherein the first, second and third guide strand sequences are all different from each other. Each guide strand sequence is separated from its respective passenger strand sequence by between 5 and 35 bases. Exemplary sequences for separating a guide strand sequence from its passenger strand sequence are sequences that comprise a sequence selected from SEQ ID NOs: 241-250. Exemplary guide strand nucleotide sequences for inhibiting hamster dihydrofolate reductase and their respective passenger strand nucleotide sequences are SEQ ID NOs: 82 and 167, SEQ ID NOs: 83 and 168, SEQ ID NOs: 84 and 169, SEQ ID NOs: 607 and 617, SEQ ID NOs: 608 and 618, SEQ ID NOs: 609 and 619, SEQ ID NOs: 610 and 620, SEQ ID NOs: 611 and 621, SEQ ID NOs: 612 and 622, SEQ ID NOs: 613 and 623, SEQ ID NOs: 614 and 624, SEQ ID NOs: 615 and 625, and SEQ ID NOs: 616 and 626.

Multi-hairpin amiRNAs with nucleotide sequences SEQ ID NO: 210 and 627 each comprise guide strand sequences complementary to different sequences within the CHO dihydrofolate reductase mRNA target (nucleotide sequence SEQ ID NO: 22). These multi-hairpin amiRNA sequences each comprise a first guide strand sequence comprising a contiguous 19 or 20 or 21 or 22 nucleotide sequence complementary to SEQ ID NO: 22 and a first passenger strand sequence comprising a contiguous 19 or 20 or 21 or 22 nucleotide sequence that is at least 78% identical to the reverse complement of the first guide strand sequence. These multi-hairpin amiRNA sequences each further comprise a second guide strand sequence comprising a contiguous 19 or 20 or 21 or 22 nucleotide sequence complementary to SEQ ID NO: 22 and a second passenger strand sequence comprising a contiguous 19 or 20 or 21 or 22 nucleotide sequence that is at least 78% identical to the reverse complement of the second guide strand sequence, and wherein the first and second guide strand sequences are different from each other. These multi-hairpin amiRNA sequences each further comprise a third guide strand sequence comprising a contiguous 19 or 20 or 21 or 22 nucleotide sequence complementary to SEQ ID NO: 22 and a third passenger strand sequence comprising a contiguous 19 or 20 or 21 or 22 nucleotide sequence that is at least 78% identical to the reverse complement of the third guide strand sequence, and wherein the first, second and third guide strand sequences are all different from each other. Each guide strand sequence in each of these multi-hairpin amiRNA sequences is separated from its respective passenger strand sequence by between 5 and 35 bases. For multi-hairpin amiRNA with nucleotide sequences SEQ ID NO 210 and 627, each guide strand sequence is separated from its respective passenger strand sequence by a nucleotide sequence comprising SEQ ID NO: 241. Multi-hairpin amiRNA with nucleotide sequence SEQ ID NO: 210 comprises a first guide strand with nucleotide sequence SEQ ID NO: 82 and a first passenger strand with nucleotide sequence SEQ ID NO: 167; SEQ ID NO: 210 further comprises a second guide strand with nucleotide sequence SEQ ID NO: 83 and a second passenger strand with nucleotide sequence SEQ ID NO: 168; SEQ ID NO: 210 further comprises a third guide strand with nucleotide sequence SEQ ID NO: 84 and a third passenger strand with nucleotide sequence SEQ ID NO: 169. Guide strand sequences SEQ ID NO: 82, SEQ ID NO: 83, and SEQ ID NO: 84 are all complementary to the same natural cellular mRNA and are all different from each other. Multi-hairpin amiRNA with nucleotide sequence SEQ ID NO: 627 comprises a first guide strand sequence with nucleotide sequence SEQ ID NO: 612 and a first passenger strand sequence with nucleotide sequence SEQ ID NO: 622; SEQ ID NO: 627 further comprises a second guide strand with nucleotide sequence SEQ ID NO: 82 and a second passenger strand with nucleotide sequence SEQ ID NO: 167; SEQ ID NO: 627 further comprises a third guide strand with nucleotide sequence SEQ ID NO: 83 and a third passenger strand with nucleotide sequence SEQ ID NO: 168; SEQ ID NO: 627 further comprises a fourth guide strand with nucleotide sequence SEQ ID NO: 84 and a fourth passenger strand with nucleotide sequence SEQ ID NO: 169. Guide strand sequences SEQ ID NO: 612, SEQ ID NO: 82, SEQ ID NO: 83, and SEQ ID NO: 84 are all complementary to the same natural cellular mRNA and are all different from each other.

In one embodiment, a multi-hairpin amiRNA gene to express amiRNAs that inhibit dihydrofolate reductase are introduced into a cell lacking a functional glutamine synthetase gene. The resulting cell requires media supplemented with glutamine, hypoxanthine and thymidine (HT) in order to grow. In some embodiments dihydrofolate reductase expression is reduced but cells can still synthesize enough tetrahydrofolate to survive and thus do not require media supplemented with hypoxanthine and thymidine. However the amiRNA-mediated DHFR inhibition sensitizes the cells to methotrexate, which can then be used as a selective agent.

### 5.9.2 Complementation of THF Auxotrophy

In cells lacking a functional DHFR gene, including cells in which endogenous DHFR expression is reduced by RNA interference, an exogenously provided DHFR gene can function as a selectable marker by permitting THF synthesis and cell growth in hypoxanthine-thymidine (HT)-free medium. Preferably a gene transfer polynucleotide comprising the exogenous DHFR gene is introduced into the cell. Preferably the exogenous DHFR gene comprises sequence features that prevent its expression from being inhibited by any RNA interference that has been used to make the host cell auxotrophic for HT. If RNA interference molecules, including amiRNA guide strands, are directed against the coding portion of the endogenous DHFR, an exogenous gene encoding DHFR can avoid inhibition if the coding sequence is changed, for example by silent mutations in the targeted region. If RNA interference molecules, including amiRNA guide strands, are directed against the 3′ UTR or 5′ UTR portions of the endogenous DHFR, an exogenous gene encoding DHFR can avoid inhibition if the natural UTR sequences are replaced with alternative sequences, for example UTR sequences taken or adapted from other natural genes.

One selection method comprises introducing a gene transfer polynucleotide comprising sequences encoding a DHFR selectable marker, and then growing the cell in media that does not contain enough HT for the cells to survive in the absence of an exogenous gene encoding DHFR. Cells lacking a functional DHFR gene are also more susceptible to growth inhibition by methotrexate. Thus, an alternative selection method comprises introducing a gene transfer polynucleotide comprising sequences encoding a DHFR selectable marker, and then growing the cell in media that does not contain added HT, and where the media further comprises methotrexate, for example between 10 nM and 2 μM methotrexate.

Complementation of DHFR-deficient cells with an exogenously provided DHFR gene often includes an amplification step. That is, a gene transfer polynucleotide is introduced into DHFR-deficient mammalian cells, for example DHFR-deficient CHO cells. The gene transfer polynucleotide comprises a DHFR gene expressible in the mammalian cell, and a second gene also expressible in the mammalian cell. The cells are then grown in the absence of hypoxanthine and thymidine and over time is passaged into successively higher concentrations of methotrexate, for example the initial concentration of methotrexate is 10 nM, and after 5 days it is increased to 25 nM and after a further 5 days it is increased to 100 nM and so on. The result of this is to amplify the copy number of the DHFR gene provided on the gene transfer polynucleotide within the genome of the mammalian cell. This also amplifies the copy number of the second gene, and thus increases the expression level of the second gene. Gene amplification tends to result in concatemeric structures that are unstable. A preferable method for expressing a gene in a DHFR-deficient cell comprises the following steps: a gene transfer polynucleotide comprises a transposon comprising a DHFR gene expressible in the mammalian cell, and a second gene also expressible in the mammalian cell. The gene transfer polynucleotide is introduced into DHFR-deficient mammalian cells, for example DHFR-deficient CHO cells, together with a corresponding transposase that can integrate the transposon into the genome of the cell. After recovery from transfection (typically 24-72 hours) the cells are grown in selective media lacking hypoxanthine and thymidine and with the addition of the maximum desired concentrations of methotrexate, for example a concentration between 10 nM and 2 μM methotrexate. This selects for a high number of copies of the transposon, but each of these copies is independently integrated and thus will not experience concatemer-related instability. The cells are propagated in the selective media until the viability has exceeded a threshold value for example 80 or 90 of 95%, and then grown under appropriate conditions for expression of the expressible genes encoded on the transposon.

Preferably the exogenous open reading frame encoding DHFR is operably linked to a weak promoter or other sequence elements that attenuate expression, such that high levels of expression can only occur if many copies of the gene transfer polynucleotide are present, or if they are integrated in a position in the genome where high levels of expression occur. In such cases it may be unnecessary to use a DHFR inhibitor such as MTX: simply synthesizing enough HT for cell survival may provide a sufficiently stringent selection if expression of the DHFR is attenuated.

In embodiments where doubly auxotrophic cells cannot express enough glutamine synthetase or dihydrofolate reductase to survive unless they are grown in media supplemented with glutamine, hypoxanthine and thymidine (HT), glutamine synthetase and dihydrofolate reductase genes may be provided on separate polynucleotides. For example, a first gene transfer polynucleotide comprising a first gene expressible in the cell and a glutamine synthetase gene expressible in the cell, and a second gene transfer polynucleotide comprising a second gene expressible in the cell and a dihydrofolate reductase gene expressible in the cell, may be introduced into the doubly auxotrophic cell. Optionally the first gene transfer polynucleotide and the second gene transfer polynucleotide are both transposons such that the expressible gene and the selectable marker are both transposed by a corresponding transposase. The transposons may be transposable by the same transposase or by different transposases. The transposons may be introduced into the cell at the same time or at different times. Following introduction of the first gene transfer polynucleotide comprising a first gene expressible in the cell and a glutamine synthetase gene, glutamine is removed from the media, and optionally methionine sulphoximine is added to the media thereby selecting for cells in which the first gene transfer polynucleotide has integrated into the genome. Following introduction of the second gene transfer polynucleotide comprising a second gene expressible in the cell and a dihydrofolate reductase gene expressible in the cell, hypoxanthine and thymidine are removed from the media and optionally methotrexate is added thereby selecting for cells in which the second gene transfer polynucleotide has integrated into the genome. Optionally expression of glutamine synthetase in the doubly auxotrophic cell is inhibited by a multihairpin amiRNA. Optionally expression of dihydrofolate reductase in the doubly auxotrophic cell is inhibited by a multihairpin amiRNA.

### 5.10 Target Combinations

It may be advantageous to inhibit the expression of multiple genes endogenous to a cultured mammalian cell simultaneously. This may be done by combining guide strand sequences targeting different mRNAs with the appropriate loops and passenger strand sequences to form hairpins, preferably stabilized with hairpin-stabilizing sequences to the 5′ and 3′ of the guide-loop-passenger strand sequence as described in Section 5.2.4. Any number of genes may be targeted by an inhibitory polynucleotide, and multiple inhibitory polynucleotides may be integrated into the genome of a cultured mammalian cell.

### 5.11 Kits

The present invention also features kits comprising a transposase as a protein or encoded by a nucleic acid, and/or a transposon; or a gene transfer system as described herein comprising a transposase as a protein or encoded by a nucleic acid as described herein, in combination with a transposon; optionally together with a pharmaceutically acceptable carrier, adjuvant or vehicle, and optionally with instructions for use. Any of the components of the inventive kit may be administered and/or transfected into cells in a subsequent order or in parallel, e.g. a transposase protein or its encoding nucleic acid may be administered and/or transfected into a cell as defined above prior to, simultaneously with or subsequent to administration and/or transfection of a transposon. Alternatively, a transposon may be transfected into a cell as defined above prior to, simultaneously with or subsequent to transfection of a transposase protein or its encoding nucleic acid. If transfected in parallel, preferably both components are provided in a separated formulation and/or mixed with each other directly prior to administration to avoid transposition prior to transfection. Additionally, administration and/or transfection of at least one component of the kit may occur in a time staggered mode, e.g. by administering multiple doses of this component.

### 6. EXAMPLES

The following examples illustrate the methods, compositions and kits disclosed herein and should not be construed as limiting in any way. Various equivalents will be apparent from the following examples: such equivalents are also contemplated to be part of the invention disclosed herein.

### 6.1 Reducing Fucosylation of Secreted Proteins

### 6.1.1 Micro RNA Reduction of Antibody Fucosylation

### 6.1.1.1 Elimination of Fucosylation of a Stably Expressed Antibody

We used multi-hairpin amiRNA genes to suppress fucosylation of an antibody. The antibody had mature light chain sequence given by SEQ ID NO: 286 and mature heavy chain sequence given by SEQ ID NO: 285, the genes encoding the antibody were integrated into the genome of a CHO cell line on a transposon which further comprised a left end comprising a 5′-TTAA-3′ target sequence immediately followed by an ITR with SEQ ID NO: 423 (which is an embodiment of SEQ ID NO: 421) and additional sequence with SEQ ID NO: 417 and a right end comprising SEQ ID NO: 419 immediately followed by an ITR with SEQ ID NO: 424 (which is an embodiment of SEQ ID NO: 422) immediately followed by a 5′-TTAA-3′ target sequence. The transposon further comprised a gene encoding a glutamine synthetase selectable marker.

Three different multi-hairpin amiRNA genes targeting Cricetulus griseus alpha-(1,6)-fucosyl transferase (FUT8) mRNA, (which has nucleotide sequence SEQ ID NO: 1) were constructed. Two multi-hairpin amiRNAs, with nucleotide sequences SEQ ID NO: 193 and 194, each comprised three hairpins; the first hairpin comprised guide strand sequence SEQ ID NO: 23, immediately followed by loop sequence SEQ ID NO: 241 and passenger strand sequence SEQ ID NO: 108, the second hairpin comprised guide strand sequence SEQ ID NO: 24, immediately followed by loop sequence SEQ ID NO: 241 and passenger strand sequence SEQ ID NO: 109, the third hairpin comprised guide strand sequence SEQ ID NO: 25, immediately followed by loop sequence SEQ ID NO: 241 and passenger strand sequence SEQ ID NO: 110. Each of these three guide strand sequences was a 22 base sequence that was an exact reverse complement of a different region within the Cricetulus griseus alpha-(1,6)-fucosyl transferase (FUT8) mRNA. Each passenger strand sequence was complementary to its corresponding guide strand sequence, except that the bases in the passenger strand sequences corresponding to the 5′ base of the guide strand and the twelfth base of the guide strand were changed to be non-complementary. The first and twelfth bases of guide strand with SEQ ID NO:23 are G and C respectively, the corresponding bases in the corresponding passenger strand sequence SEQ ID NO: 108 are A and A respectively. The first and twelfth bases of guide strand with SEQ ID NO: 24 are T and A respectively, the corresponding bases in the corresponding passenger strand sequence SEQ ID NO: 109 are C and C respectively. The first and twelfth bases of guide strand with SEQ ID NO: 25 are T and G respectively, the corresponding bases in the corresponding passenger strand sequence SEQ ID NO: 110 are C and A respectively. Each hairpin in multi-hairpin amiRNA sequences SEQ ID NOs: 193 and 194 further comprised additional stem-stabilizing sequences, with stem sequence SEQ ID NO: 255 immediately preceding the guide strand sequence, and stem sequence SEQ ID NO: 256 immediately following the passenger strand sequence. Multi-hairpin amiRNA sequences SEQ ID NOs: 193 and 194 further comprised an unstructured sequence with SEQ ID NO: 251 to the 5′ of the first hairpin, and an unstructured sequence with SEQ ID NO: 253 to the 3′ of the third hairpin. Multi-hairpin amiRNA sequences SEQ ID NO: 194 further comprised an unstructured sequence with SEQ ID NO: 272 between the first and second hairpins, and an unstructured sequence with SEQ ID NO: 273 between the second and third hairpins. Each guide strand sequence is different, and each is complementary to the mRNA for Cricetulus griseus FUT8 (SEQ ID NO: 1).

The third multi-hairpin amiRNA with sequence given by SEQ ID NO: 195 also comprised three hairpins; the first hairpin comprised guide strand sequence SEQ ID NO: 26, immediately followed by loop sequence SEQ ID NO: 243 and passenger strand sequence SEQ ID NO: 111, the second hairpin comprised guide strand sequence SEQ ID NO: 27, immediately followed by loop sequence SEQ ID NO: 243 and passenger strand sequence SEQ ID NO: 112, the third hairpin comprised guide strand sequence SEQ ID NO: 28, immediately followed by loop sequence SEQ ID NO: 243 and passenger strand sequence SEQ ID NO: 113. Each of these three guide strand sequences was a 21 base sequence that was an exact reverse complement of a different region within the Cricetulus griseus alpha-(1,6)-fucosyl transferase (FUT8) mRNA. Each passenger strand sequence was complementary to its corresponding guide strand sequence, except that the bases in the passenger strand sequences corresponding to the twelfth and thirteenth bases of the guide strand were deleted. Each hairpin in multi-hairpin amiRNA sequences SEQ ID NO: 195 further comprised additional stem-stabilizing sequences, with stem sequence SEQ ID NO: 257 immediately preceding the guide strand sequence, and stem sequence SEQ ID NO: 258 immediately following the passenger strand sequence. Multi-hairpin amiRNA sequence SEQ ID NO: 195 further comprised an unstructured sequence with SEQ ID NO: 252 to the 5′ of the first hairpin, and an unstructured sequence with SEQ ID NO: 254 to the 3′ of the third hairpin. Each guide strand sequence is different, and each is complementary to the mRNA for Cricetulus griseus FUT8 (SEQ ID NO: 1).

Each of the three multi-hairpin amiRNA sequences was placed to the 3′ of an open reading frame encoding a red fluorescent protein (with nucleotide sequence SEQ ID NO: 279) and followed by a rabbit globin polyadenylation sequence. Each multi-hairpin amiRNA sequence was cloned into a transposon vector in which it was operably linked to a Pol II promoter (either the CMV promoter (with nucleotide sequence SEQ ID NO: 343) or the EF1 promoter (with nucleotide sequence SEQ ID NO: 314), as shown in Table 1). The transposon comprised a left end comprising a 5′-TTAA-3′ target sequence immediately adjacent to ITR with nucleotide sequence SEQ ID NO: 427, immediately followed by an additional nucleotide sequence SEQ ID NO: 425 and a right end comprising nucleotide sequence SEQ ID NO: 426 immediately followed by an ITR with nucleotide sequence SEQ ID NO: 428 immediately followed by a 5′-TTAA-3′ target sequence. It further comprised a gene encoding a puromycin selectable marker (with polypeptide sequence SEQ ID NO: 302). The transposons were configured so that the multi-hairpin amiRNA, the fluorescent protein gene, as well as all necessary operably linked control elements were transposable by a corresponding transposase.

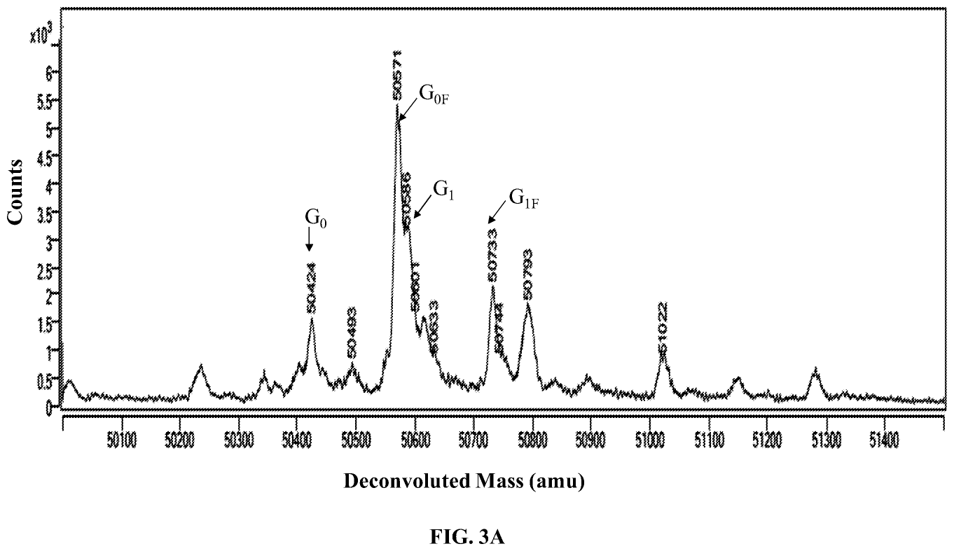

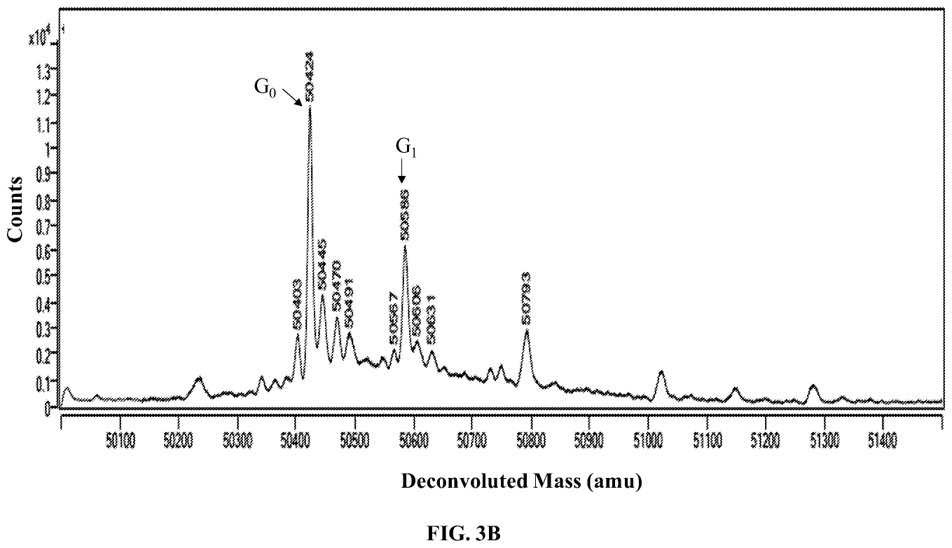

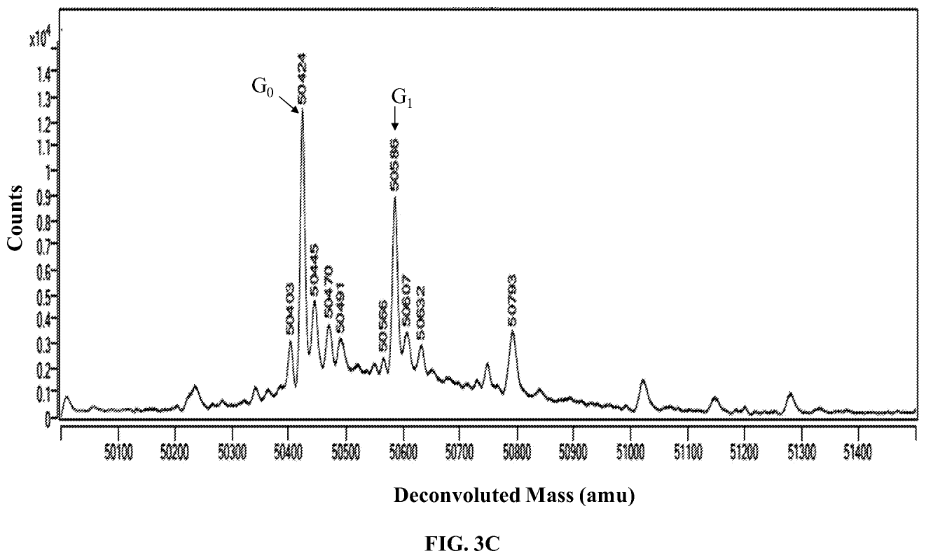

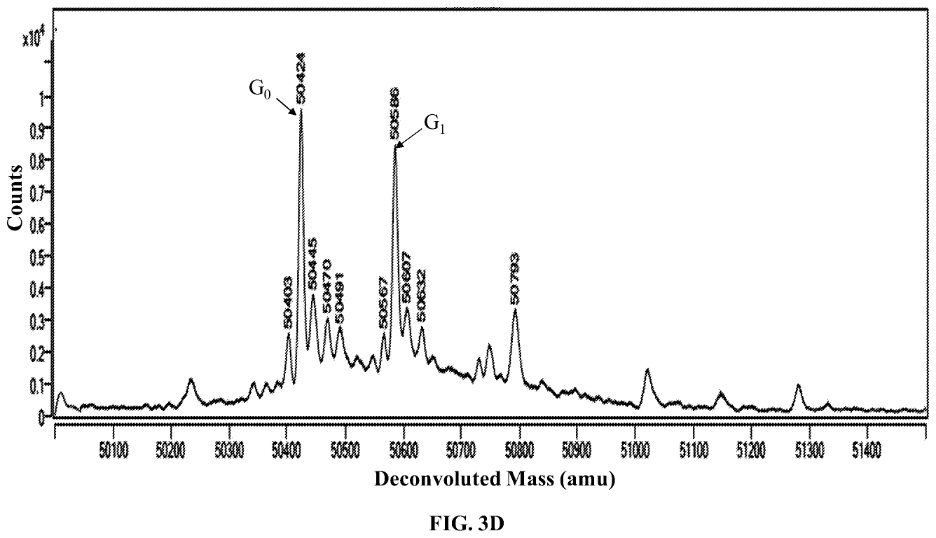

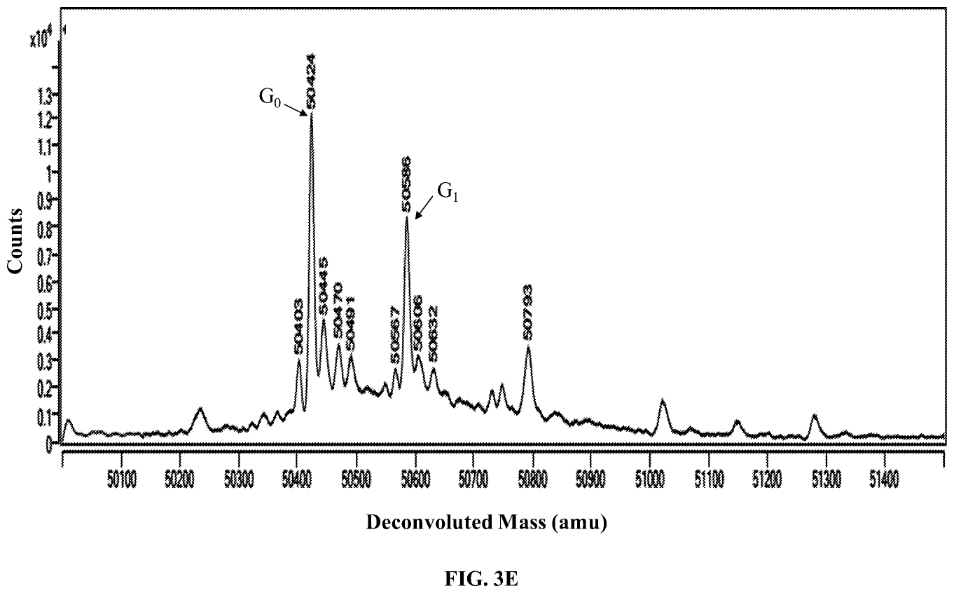

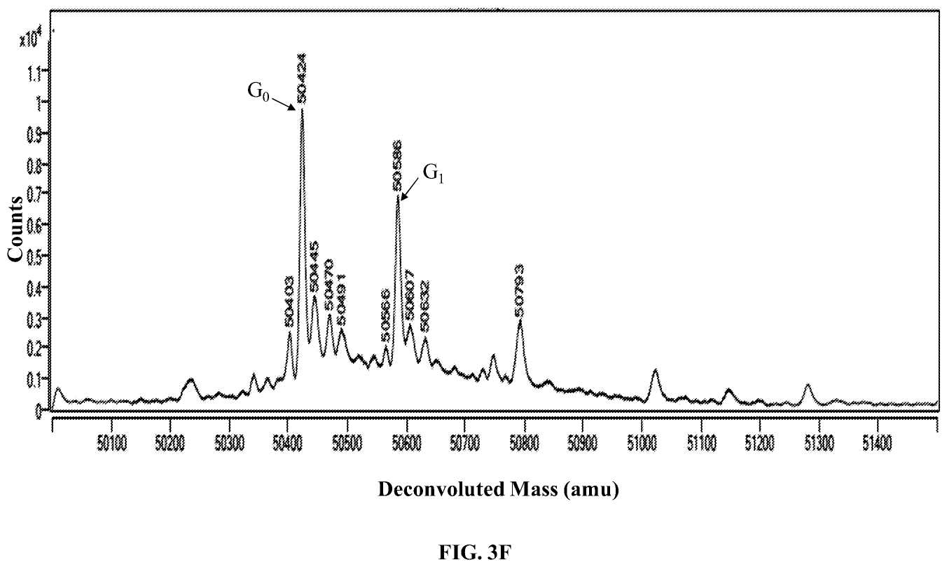

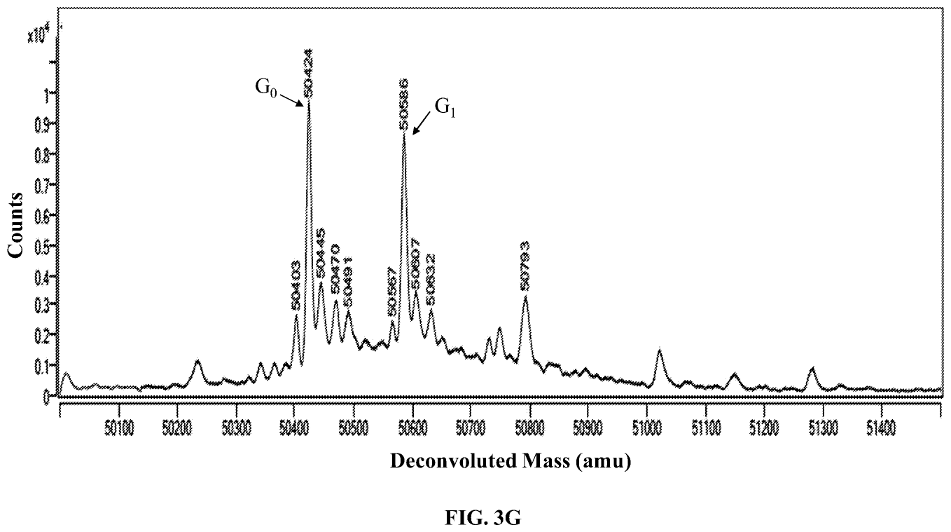

Transposons were co-transfected with mRNA encoding transposase with polypeptide sequence SEQ ID NO: 502 into a clonal CHO cell line expressing an antibody with mature light chain polypeptide sequence SEQ ID NO: 286 and mature heavy chain polypeptide sequence SEQ ID NO: 285. The pool of transfected cells were grown in the presence of 10 μg/ml puromycin until their viability reached 95%. They were then grown in a 14 day fed-batch using Sigma Advanced Fed Batch media. Protein was purified from the culture supernatant using protein A affinity chromatography, reduced with dithiothreitol, and analyzed on an Agilent QTOF mass spectrometer. The mass spectroscopy traces are shown in FIGS. 3A-G. Table 1 shows the varying transposon components used for each trace shown in FIGS. 3A-G.

Four mass spectroscopy peaks are identified by arrows in FIGS. 3A-G: (i) at 50,424 Da is the heavy chain modified by G₀: the conserved hepta-saccharide core composed of 2 N-acetylglucosamine, 3 mannose and 2 other N-acetylglucosamine residues that are β-1,2 linked to α-6 mannose and α-3 mannose, forming two arms; (ii) at 50,571 Da is the heavy chain modified by G_{0F}: the conserved heptasaccharide core plus a fucose residue; (iii) at 50,586 Da is the heavy chain modified by G₁: the conserved heptasaccharide core plus a galactose residue and (iv) at 50,733 Da is the heavy chain modified by G_{1F}: the conserved heptasaccharide core plus a galactose residue and a fucose residue. FIG. 3A shows that in the starting clonal CHO line, there is a small G₀ peak at 50,424 Da and a much larger G_{0F} peak at 50,571, showing that the majority of the antibody is fucosylated (approximately 80% using relative peak height or integration under the curves). Similarly, for the starting clonal CHO line there is a significant G1F peak at 50,733. FIGS. 3B-G all show a much larger G₀ peak at 50,424 Da, and no detectable G_{0F} peak at 50,571, nor any detectable G1F peak at 50,733. We conclude that all three multi-hairpin amiRNA configurations, with the hairpins operably linked to a PolII promoter active in mammalian cells (either a CMV promoter or an EF1 promoter), inhibited FUT8 expression sufficiently to completely suppress antibody fucosylation.

### 6.1.1.2 Multi-Hairpin amiRNAs Operably Linked to Different Pol II Promoters

We used multi-hairpin amiRNA genes to suppress fucosylation of an antibody with mature light chain polypeptide sequence SEQ ID NO: 286 and mature heavy chain polypeptide sequence given by SEQ ID NO: 285, where the antibody was stably expressed from the clonal CHO cell line as described in Section 6.1.1.1.

The multi-hairpin amiRNA with nucleotide sequence SEQ ID NO: 194 comprised three hairpins with guides complementary to the mRNA for Cricetulus griseus alpha-(1,6)-fucosyl transferase (FUT8), as described in Section 6.1.1.1. The multi-hairpin amiRNA sequence was placed to the 3′ of an open reading frame encoding a red fluorescent protein (with nucleotide sequence SEQ ID NO: 279) and followed by a rabbit globin polyadenylation sequence. The multi-hairpin amiRNA gene was cloned into three different Bombyx transposon vectors in each of which it was operably linked to a different Pol II promoter that was weaker than the strong EF1 and CMV promoters used in Section 6.1.1.1: a rat EEF2 promoter (with nucleotide sequence SEQ ID NO: 350), a PGK promoter (with nucleotide sequence SEQ ID NO: 386) and a Ubb promoter (with nucleotide sequence SEQ ID NO:392). The transposon comprised a left end comprising a 5′-TTAA-3′ target sequence immediately adjacent to an ITR with nucleotide sequence SEQ ID NO: 427 immediately followed by additional nucleotide sequence SEQ ID NO: 425 and a right end comprising nucleotide sequence SEQ ID NO: 426 immediately followed by an ITR with nucleotide sequence SEQ ID NO: 428 immediately followed by a 5′-TTAA-3′ target sequence. It further comprised an open reading frame encoding puromycin selectable marker with polypeptide sequence given by SEQ ID NO: 302. The transposons were configured so that the multi-hairpin amiRNA, the fluorescent protein gene and the selectable marker gene, as well as all necessary operably linked control elements were transposable by a corresponding transposase.

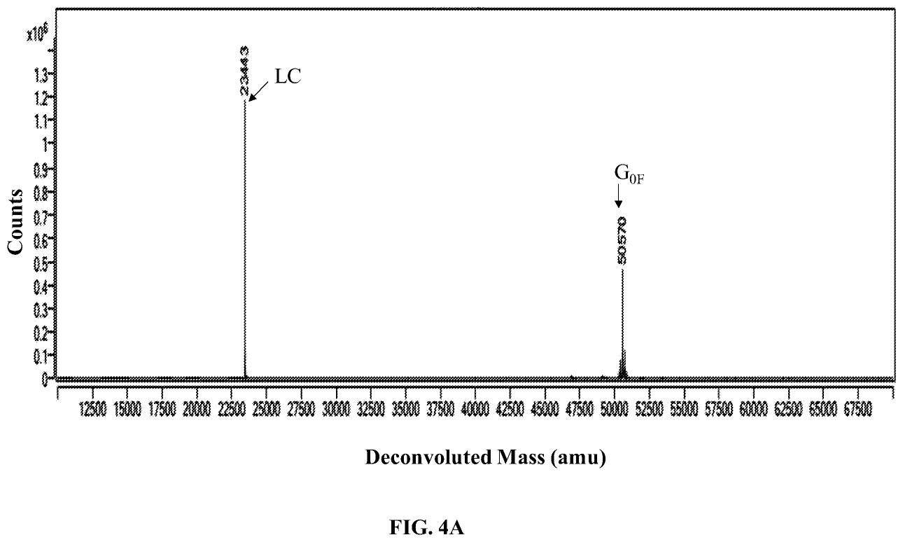

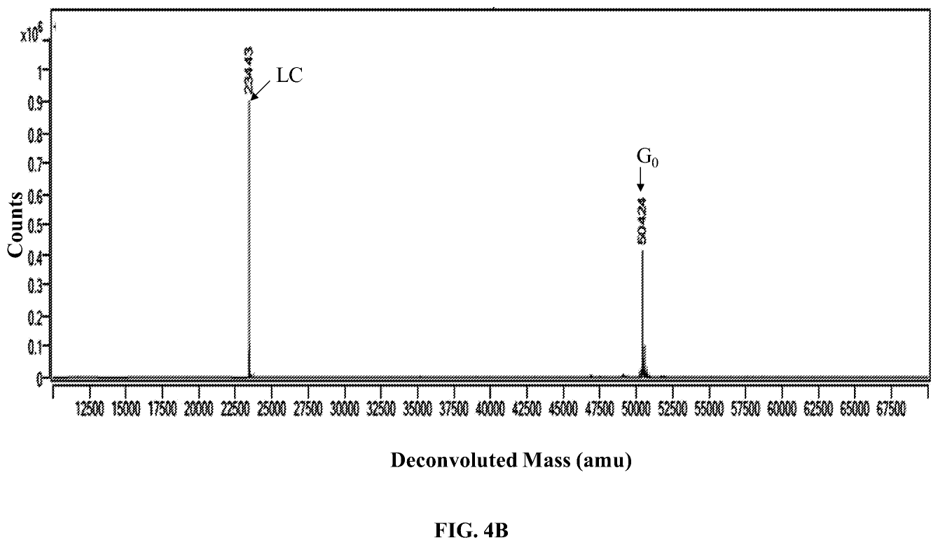

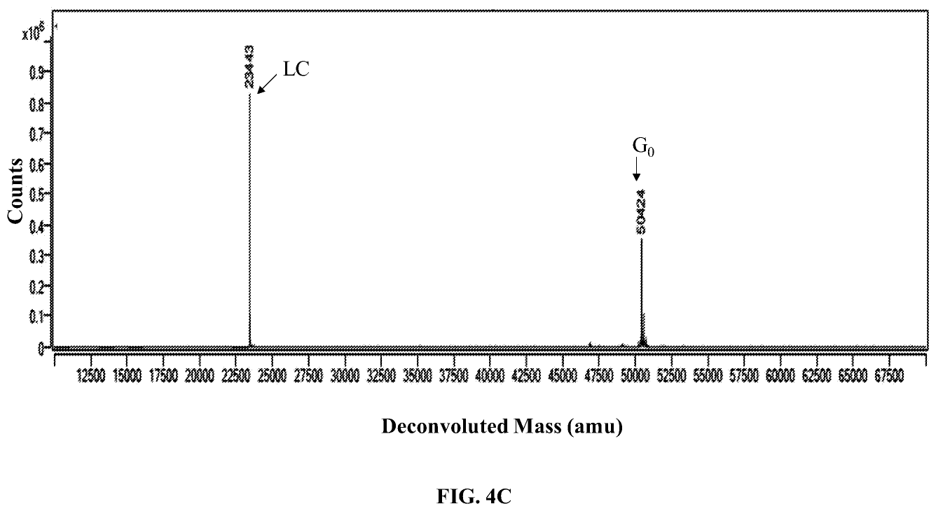

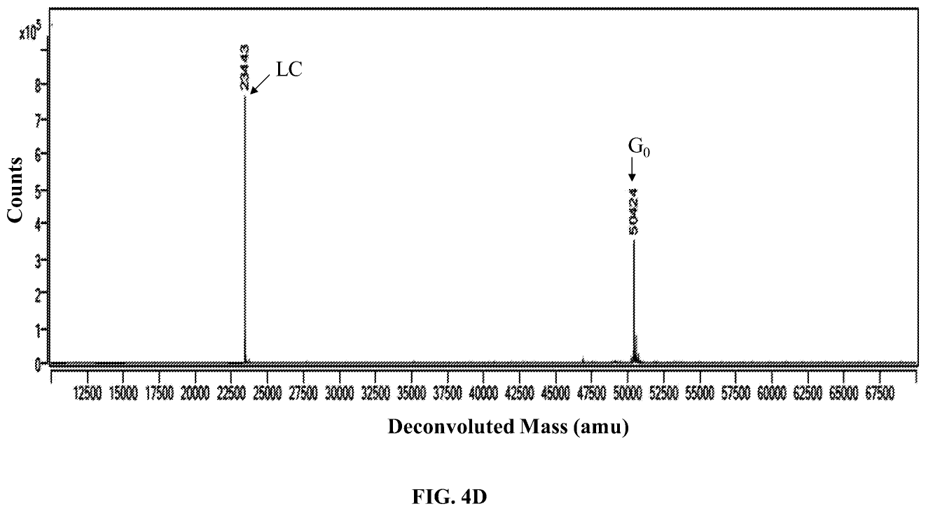

Transposons were co-transfected with mRNA encoding transposase with polypeptide sequence SEQ ID NO: 502 into a clonal CHO cell line expressing an antibody with mature light chain polypeptide sequence SEQ ID NO: 286 and mature heavy chain polypeptide sequence SEQ ID NO: 285. The pool of transfected cells were grown in the presence of 10 μg/ml puromycin until their viability reached 95%. They were then grown in a 14 day fed-batch using Sigma Advanced Fed Batch media. Protein was purified from the culture supernatant using protein A affinity chromatography, reduced with dithiothreitol, and analyzed on an Agilent QTOF mass spectrometer. The mass spectroscopy traces are shown in FIGS. 4A-D.

Three mass spectroscopy peaks are identified by arrows in FIGS. 4A-D: (i) at 50,424 Da is the heavy chain modified by G₀: the conserved heptasaccharide core composed of 2 N-acetylglucosamine, 3 mannose and 2 other N-acetylglucosamine residues that are β-1,2 linked to α-6 mannose and α-3 mannose, forming two arms; (ii) at 50,570 Da is the heavy chain modified by G_{0F}: the conserved heptasaccharide core plus a fucose residue; (iii) at 23,443 Da is the light chain. FIG. 4A shows that in the starting clonal CHO line, the heavy chain is present primarily as a single G_{0F} peak at 50,570, showing that the majority of the antibody is fucosylated (approximately 85% using relative peak height or integration under the curves). FIGS. 4B-D all show a single G₀ peak at 50,424 Da, and no detectable G_{0F} peak at 50,570. We conclude that all three of these Pol II promoters, an EEF2 promoter, a PGK promoter or a ubiquitin promoter are capable of driving enough amiRNA expression from a multi-hairpin amiRNA to inhibit FUT8 expression sufficiently to completely suppress antibody fucosylation.

### 6.1.1.3 Modification of a CHO Cell Line to Act as a Host for Transient Production of Afucosylated Antibodies

We used multi-hairpin amiRNA genes to suppress FUT 8 expression in a pool of CHO cells. The cells were subsequently used to express antibodies, which were tested for fucosylation.

The multi-hairpin amiRNA with nucleotide sequence SEQ ID NO: 194 comprised three hairpins with guides complementary to the mRNA for Cricetulus griseus alpha-(1,6)-fucosyl transferase (FUT8), as described in Section 6.1.1.1. The multi-hairpin amiRNA sequence was placed to the 3′ of an open reading frame encoding a red fluorescent protein (with nucleotide sequence SEQ ID NO: 279) and followed by a rabbit globin polyadenylation sequence. The multi-hairpin amiRNA gene was cloned into a Bombyx transposon vector in which it was operably linked to an EF1 promoter (with nucleotide sequence SEQ ID NO: 314). The transposon comprised a left end comprising a 5′-TTAA-3′ target sequence immediately adjacent to an ITR with nucleotide sequence SEQ ID NO: 427 immediately followed by additional nucleotide sequence SEQ ID NO: 425 and a right end comprising nucleotide sequence SEQ ID NO: 426 immediately followed by an ITR with nucleotide sequence SEQ ID NO: 428 immediately followed by a 5′-TTAA-3′ target sequence. It further comprised an open reading frame encoding puromycin selectable marker with polypeptide sequence SEQ ID NO: 302. The transposons were configured so that the multi-hairpin amiRNA, the fluorescent protein gene and the selectable marker gene, as well as all necessary operably linked control elements were transposable by a corresponding transposase.

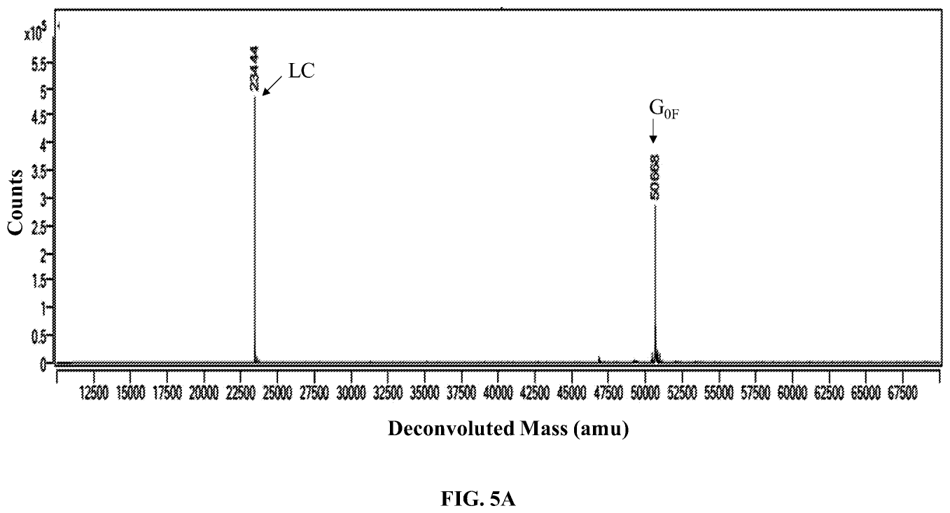

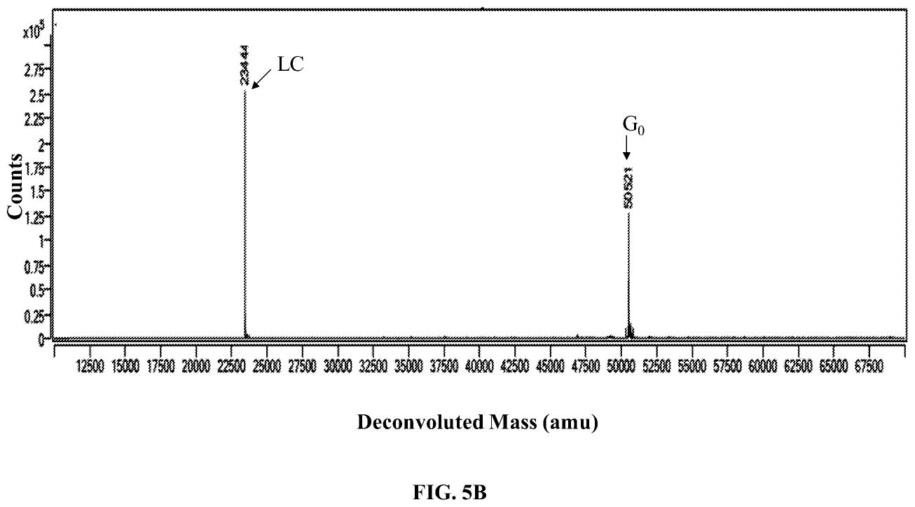

Transposons were co-transfected with mRNA encoding transposase with polypeptide sequence SEQ ID NO: 502 into a CHO cell line expressing no heterologous antibody sequences. The pool of transfected cells were grown in the presence of 10 μg/ml puromycin until their viability reached 95%. The pool of cells was then transfected with genes encoding an antibody with mature light chain polypeptide sequence SEQ ID NO: 286 and mature heavy chain polypeptide sequence SEQ ID NO: 287. The parental CHO line containing no amiRNA was also transfected with these antibody-encoding plasmids a control. Transfected cell pools were grown in a 7-day transient culture using ThermoFisher ExpiCHO media. Protein was purified from the culture supernatant using protein A affinity chromatography, reduced with dithiothreitol, and analyzed on an Agilent QTOF mass spectrometer. The mass spectroscopy traces are shown in FIGS. 5A-B.

Three mass spectroscopy peaks are identified by arrows in FIGS. 5A-B: (i) at 50,521 Da is the heavy chain modified by G₀: the conserved heptasaccharide core composed of 2 N-acetylglucosamine, 3 mannose and 2 other N-acetylglucosamine residues that are β-1,2 linked to α-6 mannose and α-3 mannose, forming two arms; (ii) at 50,668 Da is the heavy chain modified by G_{0F}: the conserved heptasaccharide core plus a fucose residue; (iii) at 23,444 Da is the light chain. FIG. 5A shows that in antibodies produced by the parental CHO line, the heavy chain is present primarily as a single G_{0F} peak at 50,668, with no detectable afucosylated heavy chain. FIG. 5B shows when the same antibody is produced from the pool of cells whose genomes comprise the multi-hairpin amiRNA gene, there is a single heavy chain G₀ peak at 50,521 Da, and no detectable G_{0F} peak at 50,668. We conclude that stable integration of a multi-hairpin amiRNA gene, comprising nucleotide sequence SEQ ID NO: 194 operably linked to a PolII promoter, into the CHO genome resulted in a pool of cells in which FUT8 expression was reduced to such a level that they produced only afucosylated antibodies.

### 6.1.1.4 Elimination of Fucosylation of a Stably Expressed Antibody Using a Multi-Hairpin amiRNA Gene Directed Against Multiple Different Genes

Fucosylation occurs within the Golgi apparatus. As an alternative to inhibiting fucosyl transferase, fucosylation of secreted antibodies could in principle be prevented by blocking cellular synthesis of fucose. GDP-mannose 4,6-dehydratase (GMD) is a key enzyme in fucose synthesis, and thus a potential target for RNA interference. However, there is also a fucose salvage pathway which could circumvent blockade at the GMD step. This can in turn be inhibited by preventing uptake of fucose into the Golgi by inhibiting the GDP-fucose transporter 1 (GFT).

A multi-hairpin amiRNA gene was designed to target both Cricetulus griseus GDP-Mannose 4,6-dehydratase (GMD), and GDP-fucose transporter 1 (GFT). The multi-hairpin amiRNA, with nucleotide sequence SEQ ID NO: 200, comprised four hairpins; the first hairpin comprised guide strand sequence SEQ ID NO: 35 (complementary to the mRNA for GMD, with nucleotide sequence SEQ ID NO: 3), immediately followed by loop sequence SEQ ID NO: 241 and passenger strand sequence SEQ ID NO: 120; the second hairpin comprised guide strand sequence SEQ ID NO: 41 (complementary to the mRNA for GFT, with nucleotide sequence SEQ ID NO: 5), immediately followed by loop sequence SEQ ID NO: 241 and passenger strand sequence SEQ ID NO: 126; the third hairpin comprised guide strand sequence SEQ ID NO:36 (complementary to the mRNA for GMD, with nucleotide sequence SEQ ID NO: 3), immediately followed by loop sequence SEQ ID NO: 241 and passenger strand sequence SEQ ID NO: 121 and the fourth hairpin comprised guide strand sequence SEQ ID NO: 42 (complementary to the mRNA for GFT, with nucleotide sequence SEQ ID NO: 5), immediately followed by loop sequence SEQ ID NO: 241 and passenger strand sequence SEQ ID NO: 127. Each passenger strand sequence was complementary to its corresponding guide strand sequence, except that the bases in the passenger strand sequences corresponding to the 5′ base of the guide strand and the twelfth base of the guide strand were changed to be non-complementary. The first and twelfth bases of guide strand with SEQ ID NO: 35 are T and G respectively, the corresponding bases in the corresponding passenger strand sequence SEQ ID NO: 120 are C and A respectively. The first and twelfth bases of guide strand with SEQ ID NO: 41 are T and C respectively, the corresponding bases in the corresponding passenger strand sequence SEQ ID NO: 126 are C and A respectively. The first and twelfth bases of guide strand with SEQ ID NO: 36 are T and T respectively, the corresponding bases in the corresponding passenger strand sequence SEQ ID NO: 121 are C and C respectively. The first and twelfth bases of guide strand with SEQ ID NO: 42 are T and G respectively, the corresponding bases in the corresponding passenger strand sequence SEQ ID NO: 127 are C and A respectively. Each hairpin in the multi-hairpin amiRNA with nucleotide sequence SEQ ID NO: 200 further comprised additional stem-stabilizing sequences, with stem sequence SEQ ID NO: 255 immediately preceding the guide strand sequence, and stem sequence SEQ ID NO: 256 immediately following the passenger strand sequence. Multi-hairpin amiRNA sequences SEQ ID NO: 200 further comprised an unstructured sequence with SEQ ID NO: 251 to the 5′ of the first hairpin, and an unstructured sequence with SEQ ID NO: 253 to the 3′ of the fourth hairpin. Multi-hairpin amiRNA sequences SEQ ID NO: 200 further comprised an unstructured sequence with SEQ ID NO: 272 between the first and second hairpins, and an unstructured sequence with SEQ ID NO: 273 between the second and third hairpins, and an unstructured sequence with SEQ ID NO: 274 between the third and fourth hairpins. Multi-hairpin amiRNA SEQ ID NO: 200 thus comprises two guide strand sequences complementary to Cricetulus griseus GMD mRNA, and two guide strand sequences complementary to Cricetulus griseus GFT mRNA, wherein each guide strand sequence is different.

Multi-hairpin amiRNA sequence with SEQ ID NO: 200 was placed to the 3′ of an open reading frame encoding a red fluorescent protein (with nucleotide sequence SEQ ID NO: 279) and followed by a rabbit globin polyadenylation sequence. The multi-hairpin amiRNA was then cloned into a transposon vector in which it was operably linked to a Pol II promoter (the human CMV promoter). The transposon comprised a left end comprising a 5′-TTAA-3′ target sequence immediately adjacent to ITR with nucleotide sequence SEQ ID NO: 427, immediately followed by an additional nucleotide sequence SEQ ID NO: 425 and a right end comprising nucleotide sequence SEQ ID NO: 426 immediately followed by an ITR with nucleotide sequence SEQ ID NO: 428 immediately followed by a 5′-TTAA-3′ target sequence. It further comprised a gene encoding a puromycin selectable marker (with polypeptide sequence SEQ ID NO: 302). The transposons were configured so that the multi-hairpin amiRNA, the fluorescent protein gene, as well as all necessary operably linked control elements were transposable by a corresponding transposase.

Transposons were co-transfected with mRNA encoding transposase with polypeptide sequence SEQ ID NO: 502 into a clonal CHO cell line expressing an antibody with mature light chain polypeptide sequence SEQ ID NO: 286 and mature heavy chain polypeptide sequence SEQ ID NO: 285. The pool of transfected cells were grown in the presence of 10 μg/ml puromycin until their viability reached 95%. They were then grown in a 14 day fed-batch using Sigma Advanced Fed Batch media. Protein was purified from the culture supernatant using protein A affinity chromatography, reduced with dithiothreitol, and analyzed on an Agilent QTOF mass spectrometer. Table 2 shows the percentage of the antibody heavy chain that was modified by G₀ (the conserved heptasaccharide core composed of 2 N-acetylglucosamine, 3 mannose and 2 other N-acetylglucosamine residues that are β-1,2 linked to α-6 mannose and α-3 mannose, forming two arms) or G₁ (the conserved heptasaccharide core plus a galactose residue), compared with the percentage of the antibody heavy chain that was modified by G_{0F} or G1F: G₀ and G₁ with the addition of a fucose residue.

As shown in Table 2, antibody expressed from the control cell line which had not been transfected with a multi-hairpin amiRNA had a fucosylation level of about 75%. In contrast, no fucose was detectable by mass spectroscopy in the pool of cells whose genomes comprised multi-hairpin amiRNA with SEQ ID NO: 200. We conclude that both of these multi-hairpin amiRNAs completely suppressed antibody fucosylation. We conclude that stable integration of a multi-hairpin amiRNA gene, comprising SEQ ID NO: 200 operably linked to a PolII promoter, into the CHO genome resulted in a pool of cells in which GMD and GFT expression were reduced to such a level that they produced only afucosylated antibodies.

### 6.1.1.5 Modification of a Human Cell Line to Act as a Host for Transient Production of Afucosylated Antibodies

Two different multi-hairpin amiRNA sequences were designed to target genes involved in the fucosylation pathway in human cells: alpha-(1,6)-fucosyl transferase (FUT8), GDP-Mannose 4,6-dehydratase (GMD), and GDP-fucose transporter 1 (GFT). One Multi-hairpin amiRNA, with nucleotide sequence SEQ ID NO: 202 comprised three hairpins; the first hairpin comprised guide strand sequence SEQ ID NO: 29, immediately followed by loop sequence SEQ ID NO: 241 and passenger strand sequence SEQ ID NO: 114, the second hairpin comprised guide strand sequence SEQ ID NO: 30, immediately followed by loop sequence SEQ ID NO: 241 and passenger strand sequence SEQ ID NO: 115, the third hairpin comprised guide strand sequence SEQ ID NO: 31, immediately followed by loop sequence SEQ ID NO: 241 and passenger strand sequence SEQ ID NO: 116. Each of these three guide strand sequences was a 22 base sequence that was an exact reverse complement of a different region within the Homo sapiens alpha-(1,6)-fucosyl transferase (FUT8) mRNA. Each passenger strand sequence was complementary to its corresponding guide strand sequence, except that the bases in the passenger strand sequences corresponding to the 5′ base of the guide strand and the twelfth base of the guide strand were changed to be non-complementary. The first and twelfth bases of guide strand with SEQ ID NO: 29 are T and T respectively, the corresponding bases in the corresponding passenger strand sequence SEQ ID NO: 114 are C and C respectively. The first and twelfth bases of guide strand with SEQ ID NO: 30 are T and T respectively, the corresponding bases in the corresponding passenger strand sequence SEQ ID NO: 115 are C and C respectively. The first and twelfth bases of guide strand with SEQ ID NO: 31 are T and A respectively, the corresponding bases in the corresponding passenger strand sequence SEQ ID NO: 116 are C and C respectively. Each hairpin in multi-hairpin amiRNA nucleotide sequence SEQ ID NO: 202 further comprised additional stem-stabilizing sequences, with stem sequence SEQ ID NO: 255 immediately preceding the guide strand sequence, and stem sequence SEQ ID NO: 256 immediately following the passenger strand sequence. Multi-hairpin amiRNA nucleotide sequence SEQ ID NO: 202 further comprised an unstructured sequence with SEQ ID NO: 251 to the 5′ of the first hairpin, and an unstructured sequence with SEQ ID NO: 253 to the 3′ of the third hairpin. Multi-hairpin amiRNA nucleotide sequence SEQ ID NO: 202 further comprised an unstructured sequence with SEQ ID NO: 272 between the first and second hairpins, and an unstructured sequence with SEQ ID NO: 273 between the second and third hairpins. Each guide strand sequence is different, and each is complementary to the mRNA for Homo sapiens FUT8 (SEQ ID NO: 7).

A second multi-hairpin amiRNA gene was designed to target both Homo sapiens GDP-Mannose 4,6-dehydratase (GMD) with mRNA sequence given by SEQ ID NO: 8, and GDP-fucose transporter 1 (GFT) with mRNA sequence given by SEQ ID NO: 9. The multi-hairpin amiRNA, with nucleotide sequence SEQ ID NO: 204, comprised four hairpins; the first hairpin comprised guide strand sequence SEQ ID NO: 47 (complementary to the mRNA for human GMD, with sequence SEQ ID NO: 8), immediately followed by loop sequence SEQ ID NO: 241 and passenger strand sequence SEQ ID NO: 132; the second hairpin comprised guide strand sequence SEQ ID NO: 52 (complementary to the mRNA for human GFT, with sequence given by SEQ ID NO: 9), immediately followed by loop sequence SEQ ID NO: 241 and passenger strand sequence SEQ ID NO: 137; the third hairpin comprised guide strand sequence SEQ ID NO: 50 (complementary to the mRNA for human GFT, with sequence SEQ ID NO: 9), immediately followed by loop sequence SEQ ID NO: 241 and passenger strand sequence SEQ ID NO: 135 and the fourth hairpin comprised guide strand sequence SEQ ID NO: 49 (complementary to the mRNA for human GMD, with sequence given by SEQ ID NO: 8), immediately followed by loop sequence SEQ ID NO: 241 and passenger strand sequence SEQ ID NO: 134. Each passenger strand sequence was complementary to its corresponding guide strand sequence, except that the bases in the passenger strand sequences corresponding to the 5′ base of the guide strand and the twelfth base of the guide strand were changed to be non-complementary. The first and twelfth bases of guide strand with SEQ ID NO: 47 are T and G respectively, the corresponding bases in the corresponding passenger strand sequence SEQ ID NO: 132 are C and A respectively. The first and twelfth bases of guide strand with SEQ ID NO: 52 are T and A respectively, the corresponding bases in the corresponding passenger strand sequence SEQ ID NO: 137 are C and C respectively. The first and twelfth bases of guide strand with SEQ ID NO: 50 are T and G respectively, the corresponding bases in the corresponding passenger strand sequence SEQ ID NO: 135 are C and A respectively. The first and twelfth bases of guide strand with SEQ ID NO: 49 are T and C respectively, the corresponding bases in the corresponding passenger strand sequence SEQ ID NO: 134 are C and A respectively. Each hairpin in multi-hairpin amiRNA sequence SEQ ID NO: 204 further comprised additional stem-stabilizing sequences, with stem sequence SEQ ID NO: 255 immediately preceding the guide strand sequence, and stem sequence SEQ ID NO: 256 immediately following the passenger strand sequence. Multi-hairpin amiRNA sequence SEQ ID NO: 204 further comprised an unstructured sequence with SEQ ID NO: 251 to the 5′ of the first hairpin, and an unstructured sequence with SEQ ID NO: 253 to the 3′ of the fourth hairpin. Multi-hairpin amiRNA sequences SEQ ID NO: 200 further comprised an unstructured sequence with SEQ ID NO: 272 between the first and second hairpins, and an unstructured sequence with SEQ ID NO: 273 between the second and third hairpins, and an unstructured sequence with SEQ ID NO: 274 between the third and fourth hairpins. Multi-hairpin amiRNA SEQ ID NO: 204 thus comprises two guide strand sequences complementary to Homo sapiens GMD mRNA, and two guide strand sequences complementary to Homo sapiens GFT mRNA, wherein each guide strand sequence is different.

The multi-hairpin amiRNA sequences were placed to the 3′ of an open reading frame encoding a red fluorescent protein (with nucleotide sequence SEQ ID NO: 279) and followed by a rabbit globin polyadenylation sequence. Each multi-hairpin amiRNA sequence was cloned into a transposon vector in which it was operably linked to a Pol II promoter (the CMV promoter with nucleotide sequence SEQ ID NO: 343). The transposon comprised a left end comprising a 5′-TTAA-3′ target sequence immediately adjacent to ITR with nucleotide sequence SEQ ID NO: 427, immediately followed by an additional nucleotide sequence SEQ ID NO: 425 and a right end comprising nucleotide sequence SEQ ID NO: 426 immediately followed by an ITR with nucleotide sequence SEQ ID NO: 428 immediately followed by a 5′-TTAA-3′ target sequence. It further comprised a gene encoding a puromycin selectable marker (with polypeptide sequence SEQ ID NO: 302). The transposons were configured so that the multi-hairpin amiRNA, the fluorescent protein gene, as well as all necessary operably linked control elements were transposable by a corresponding transposase.

Transposons were co-transfected with mRNA encoding transposase with polypeptide sequence SEQ ID NO: 502 into a human embryonic kidney (HEK) cell line expressing no heterologous antibody sequences. The pool of transfected cells were grown in the presence of 10 μg/ml puromycin until their viability reached 95%. Each pool of cells was then transfected in two independent reactions with genes encoding an antibody with mature light chain polypeptide sequence SEQ ID NO: 286 and mature heavy chain polypeptide sequence SEQ ID NO: 287. The antibody genes were operably linked to a human CMV promoter and a rabbit globin polyadenylation signal sequence. Transfected cell pools were grown in a 7-day transient culture using ThermoFisher Expi293 media. Protein was purified from the culture supernatant using protein A affinity chromatography, reduced with dithiothreitol, and analyzed on an Agilent QTOF mass spectrometer. Peaks were identified and quantified corresponding to (i) the heavy chain modified by G₀: the conserved heptasaccharide core composed of 2 N-acetylglucosamine, 3 mannose and 2 other N-acetylglucosamine residues that are β-1,2 linked to α-6 mannose and α-3 mannose, forming two arms, (ii) the heavy chain modified by G₀ plus fucose (G_{0F}), (iii) the heavy chain modified by G₀ plus an additional galactose residue (G₁), and (iv) the heavy chain modified by G₀ plus an additional galactose residue plus fucose (G1F). Table 3 shows the titer of antibody produced by the transfected HEK cell pools, and the fucosylation observed in each case.

In the absence of multi-hairpin amiRNAs, the antibody produced by HEK cells was between 93 and 100% fucosylated (Table 3 rows 1 and 2). Both replicates of cell pools whose genomes comprised the anti-GMD/GFT multi-hairpin amiRNA genes with nucleotide sequence SEQ ID NO: 204 (Table 3 rows 5 and 6) showed complete abolition of antibody fucosylation. Both replicates of cell pools whose genomes comprised the anti-FUT8 multi-hairpin amiRNA nucleotide sequence SEQ ID NO: 202 (Table 3 rows 3 and 4) showed approximately 90% reduction of antibody fucosylation. We conclude that stable integration of multi-hairpin amiRNA genes comprising nucleotide sequence SEQ ID NO: 202 or 204 into the HEK genome inhibit expression of genes in the fucosylation pathway such that the resulting pool of cells produce largely or entirely afucosylated antibodies.

One of the pools of HEK cells whose genomes comprised the anti-FUT8 multi-hairpin amiRNA with nucleotide sequence SEQ ID NO: 202 was subjected to single cell cloning. Four monoclonal cell lines were produced. Each of these cell lines was transfected in two independent reactions with genes encoding an antibody with mature light chain polypeptide sequence SEQ ID NO: 286 and mature heavy chain polypeptide sequence SEQ ID NO: 287. The antibody genes were operably linked to a human CMV promoter and a rabbit globin polyadenylation signal sequence. Transfected cells were grown in a 7 day transient culture using ThermoFisher Expi293 media. Protein was purified from the culture supernatant using protein A affinity chromatography, reduced with dithiothreitol, and analyzed on an Agilent QTOF mass spectrometer for the presence of fucosylated antibody, as described above. Table 4 shows the fucosylation level of the antibodies prepared from the clones.

Cells whose genomes did not comprise multi-hairpin amiRNA genes produced antibody that was between 90 and 94% fucosylated (Table 4 rows 1 and 2). The four different clones produced antibodies with significantly different levels of fucosylation, though the level was very similar between replicates made in the same clonal cell line. Clonal cell line 1 produced antibodies that were about 40% fucosylated, antibodies from clonal line 2 were about 20% fucosylated, clonal line 3 produced antibodies about 13% fucosylated, and clonal line 4 produced antibodies with between 6 and 10% fucosylation. Inhibition of fucosylation was stably maintained in at least one of the four clonal lines.

A transposon comprising a multi-hairpin amiRNA gene comprising multiple guide strand sequences, each complementary to a different sequence within the human FUT8 mRNA (with nucleotide sequence SEQ ID NO: 7), can be integrated into the genome of an HEK293 cell to reduce the fucosylation of antibodies produced by the HEK cell. Preferably less than 40% of an antibody produced by the cell line is fucosylated, more preferably less than 20% of an antibody produced by the cell line is fucosylated, more preferably less than 10% of an antibody produced by the cell line is fucosylated.

### 6.1.2 Dual Functional Micro RNAs: Gene Knockdown and Selectable Marker Attenuation

### 6.1.2.1 Fucosylation-Targeting microRNAs Incorporated into the 3′ UTR of the Selectable Marker Gene

As described in Section 5.2.6, it can be advantageous to incorporate multi-hairpin amiRNA sequences into the 3′UTR of a selectable marker gene, particularly when the selectable marker is part of a transposon. After transcription, processing of the amiRNA sequences destabilizes the selectable marker mRNA because it leads to removal of the stabilizing polyA sequences. This means that to supply enough of the selectable marker protein encoded by the selectable marker gene, expression from the transposon will need to be higher than from a transposon without the amiRNA sequences in the selectable marker 3′UTR. Including amiRNA sequences in the 3′UTR of the selectable marker thus either selects for cells whose genomes comprise more copies of the transposon, or for cells in which transposons are integrated in more transcriptionally active regions of the genome. Another advantage is that only a very small addition to transposon size (less than an additional 1,000 bp) can effect a phenotypic change by inhibiting the expression of one or more host genes. For example, this can be done simultaneously with introduction of a gene encoding a protein to be expressed. To demonstrate this, amiRNA sequences were placed into the 3′UTR of a gene for expression of glutamine synthetase in a mammalian cell.

One- two- or three-hairpin amiRNAs were incorporated into the 3′ UTR of a glutamine synthetase selectable marker on a transposon. The multi-hairpin amiRNA with nucleotide sequence SEQ ID NO: 194 comprised three hairpins as described in Section 6.1.1.1.

The multi-hairpin amiRNA with nucleotide sequence SEQ ID NO: 196 comprised two hairpins, the first hairpin comprised guide strand sequence SEQ ID NO: 23, immediately followed by loop sequence SEQ ID NO: 241 and passenger strand sequence SEQ ID NO: 108, the second hairpin comprised guide strand sequence SEQ ID NO: 24, immediately followed by loop sequence SEQ ID NO: 241 and passenger strand sequence SEQ ID NO: 109. Each of these two guide strand sequences was a 22 base sequence that was an exact reverse complement of a different region within the Cricetulus griseus alpha-(1,6)-fucosyl transferase (FUT8) mRNA. Mismatches between guide and passenger strand sequences are as described in Section 6.1.1.1. Each hairpin in multi-hairpin amiRNA nucleotide sequence SEQ ID NO: 196 further comprised additional stem-stabilizing sequences, with stem sequence SEQ ID NO: 255 immediately preceding the guide strand sequence, and stem sequence SEQ ID NO: 256 immediately following the passenger strand sequence. Multi-hairpin amiRNA with nucleotide sequence SEQ ID NO: 196 further comprised an unstructured sequence with SEQ ID NO: 251 to the 5′ of the first hairpin, and an unstructured sequence with SEQ ID NO: 253 to the 3′ of the third hairpin. Multi-hairpin amiRNA nucleotide sequence SEQ ID NO: 196 further comprised an unstructured sequence with SEQ ID NO: 272 between the first and second hairpins. Each guide strand sequence is different, and each is complementary to the mRNA for Cricetulus griseus FUT8 (SEQ ID NO: 1).

We also designed and synthesized a single hairpin amiRNA with sequence given by SEQ ID NO: 197 comprising one hairpin which comprised guide strand sequence SEQ ID NO: 23, immediately followed by loop sequence SEQ ID NO: 241 and passenger strand sequence SEQ ID NO: 108. Mismatches between guide and passenger strand sequences are as described in Section 6.1.1.1. The hairpin in amiRNA sequence SEQ ID NOs: 197 further comprised additional stem-stabilizing sequences, with stem sequence SEQ ID NO: 255 immediately preceding the guide strand sequence, and stem sequence SEQ ID NO: 256 immediately following the passenger strand sequence. The amiRNA nucleotide sequence SEQ ID NO: 197 further comprised an unstructured sequence with SEQ ID NO: 251 to the 5′ of the hairpin, and an unstructured sequence with SEQ ID NO: 253 to the 3′ of the hairpin.

These amiRNA sequences were placed to the 3′ of an open reading frame encoding a glutamine synthetase protein (with polypeptide sequence SEQ ID NO: 307) and followed by a human globin polyadenylation sequence. The amiRNA genes were cloned into a transposon vector in which they were operably linked to a Pol II promoter. The transposon further comprised genes encoding an antibody with mature light chain polypeptide sequence SEQ ID NO: 286 and mature heavy chain polypeptide sequence SEQ ID NO: 288. The transposon further comprised a left end comprising a 5′-TTAA-3′ target sequence immediately followed by an ITR with nucleotide sequence SEQ ID NO: 423 (which is an embodiment of SEQ ID NO: 421) and additional nucleotide sequence SEQ ID NO: 417 and a right end comprising nucleotide sequence SEQ ID NO: 419 immediately followed by an ITR with nucleotide sequence SEQ ID NO: 424 (which is an embodiment of SEQ ID NO: 422) immediately followed by a 5′-TTAA-3′ target sequence. The transposon was configured so that the multi-hairpin amiRNA, the glutamine synthetase gene and the genes for both antibody chains, as well as all necessary operably linked control elements were transposable by a corresponding transposase.

Transposons were co-transfected with mRNA encoding transposase with polypeptide sequence SEQ ID NO: 472 into a CHO cell line with no functional glutamine synthetase gene. The pool of transfected cells were grown in the absence of glutamine added to the media until their viability reached 95%. They were then grown in a 14 day fed-batch using Sigma Advanced Fed Batch media. Protein was purified from the culture supernatant using protein A affinity chromatography, reduced with dithiothreitol, and analyzed on an Agilent QTOF mass spectrometer. We integrated the area under the peaks at 50,456 Da (corresponding to the heavy chain modified by G₀: the conserved heptasaccharide core composed of 2 N-acetylglucosamine, 3 mannose and 2 other N-acetylglucosamine residues that are β-1,2 linked to α-6 mannose and α-3 mannose, forming two arms) and 50,602 (corresponding to the heavy chain modified by G_{0F}: the conserved heptasaccharide core plus a fucose residue) to calculate the relative proportion of fucosylated and afucosylated antibody. Results are shown in Table 5.

Table 5 shows that when the strong CMV or EEF2 promoters are operably linked to the glutamine synthetase gene and to the multi-hairpin amiRNAs in its 3′ UTR, the antibody is fully afucosylated (Table 5 rows 1 and 2). This is in contrast to the approximately 80-85% fucosylation seen when an equivalent transposon in which there were no amiRNA sequences in the 3′UTR of the glutamine synthetase gene (as described in Sections 6.1.1.1 and 6.1.1.2). Because these promoters are strong, they express high levels of glutamine synthetase, which means that cells do not require many copies of the integrated transposon in order to synthesize enough glutamine to survive. The antibody titer in the culture supernatant is therefore lower: lowest (163 mg/L) in the case of the strongest (CMV) promoter (Table 5 column E), and higher (443 mg/L) with the weaker EEF2 promoter. The CMV and the EEF2 promoter, operably linked to multi-hairpin amiRNA with nucleotide sequence SEQ ID NO: 194 (by incorporating the amiRNA hairpins after the open reading frame encoding the selectable marker, but before the polyA signal sequence) completely eliminated fucosylation of the antibody (Table 5, columns F and G).

When a weaker promoter is operably linked to the glutamine synthetase, and the 3′UTR comprises only a single amiRNA hairpin (amiRNA with nucleotide sequence SEQ ID NO: 197, Table 5 row 3), the antibody titer is 514 mg/L: about 3-fold higher than when the CMV promoter is used, but the antibody is still about 50% fucosylated, compared with the natural level of around 80-85% as described in Sections 6.1.1.1 and 6.1.1.2. Adding a second amiRNA hairpin to the 3′ UTR of the glutamine synthetase (amiRNA with nucleotide sequence SEQ ID NO: 196) has the twin effects of increasing antibody titer (to 770 mg/L) and reducing antibody fucosylation (to 10%), as shown in Table 5 row 4. These effects result from more processing of the selectable marker 3′ UTR, which produces more FUT8-targeting RNA in the RISC complex and also increases destabilization of the glutamine synthetase selectable marker mRNA. This trend continues when the PGK promoter is operably linked to a glutamine synthetase gene with a three-hairpin amiRNA in its 3′ UTR (nucleotide sequence SEQ ID NO: 194), as shown in Table 5 row 5. The antibody titer is further increased to 835 mg/L, and fucosylation of the antibody is completely prevented.

This example also demonstrates the benefit of using multi-hairpin amiRNA sequences, wherein two or more different guide strand sequences are complementary to two or more different sequences in the same target mRNA. Use of a single hairpin amiRNA with one guide strand sequence complementary to FUT8 mRNA reduced FUT8 expression which resulted in reduction of antibody fucosylation from approximately 80% to 50%. Use of a multi-hairpin with two different guide strand sequences complementary to different sequences within the FUT8 mRNA reduced FUT8 expression more and resulted in reduction of antibody fucosylation to 10%. Use of a multi-hairpin with three different guide strand sequences complementary to different sequences within the FUT8 mRNA reduced FUT8 expression even more and resulted in reduction of antibody fucosylation to below the limit of detection.

### 6.1.2.2 Fucosylation-Targeting microRNAs Incorporated into the 3′ UTR of the Selectable Marker Gene and Driven by Different Promoters

As described in Section 6.1.2.1, the multi-hairpin amiRNA with nucleotide sequence SEQ ID NO: 194 was capable of completely suppressing the fucosylation of the antibody. However, we also wished to increase the titer of the antibody. As described in Section 5.2.6, attenuation of expression of the glutamine synthetase selectable marker can improve expression of genes encoded on a transposon. Transcription of the multi-hairpin amiRNA sequences from the PGK promoter as described in Section 6.1.2.1 provided enough guide strand associated with the RISC complex to reduce fucosylation through FUT8 below detectable levels. We therefore wished to attenuate glutamine synthetase expression in a way that would not reduce transcription of the multi-hairpin amiRNA. To do this we tested incorporation of inhibitory 5′ UTRs before the glutamine synthetase gene. These should reduce expression of the glutamine synthetase without affecting transcription of the multi-hairpin amiRNA. We also tested expressing glutamine synthetase and multi-hairpin amiRNA with nucleotide sequence SEQ ID NO: 194 by operably linking it to the weaker HSV-TK promoter in the presence of inhibitory 5′ UTRs.

The three-hairpin amiRNA with nucleotide sequence SEQ ID NO: 194 was incorporated into the 3′ UTR of a glutamine synthetase selectable marker on a transposon. The amiRNA sequence was placed to the 3′ of an open reading frame encoding a glutamine synthetase protein with polypeptide sequence SEQ ID NO: 307 and was followed by a human globin polyadenylation sequence. The amiRNA gene was cloned into different transposon vectors in which it was operably linked to different Pol II promoters. Each transposon further comprised genes encoding an antibody with mature light chain polypeptide sequence SEQ ID NO: 286 and mature heavy chain polypeptide sequence SEQ ID NO: 288. The transposon further comprised a left end comprising a 5′-TTAA-3′ target sequence immediately followed by an ITR with nucleotide sequence SEQ ID NO: 423 (which is an embodiment of SEQ ID NO: 421) and additional nucleotide sequence SEQ ID NO: 417 and a right end comprising nucleotide sequence SEQ ID NO: 419 immediately followed by an ITR with nucleotide sequence SEQ ID NO: 424 (which is an embodiment of SEQ ID NO: 422) immediately followed by a 5′-TTAA-3′ target sequence. The transposon was configured so that the multi-hairpin amiRNA, the glutamine synthetase gene and the genes for both antibody chains, as well as all necessary operably linked control elements were transposable by a corresponding transposase.

Transposons were co-transfected with mRNA encoding transposase with polypeptide sequence SEQ ID NO: 472 into a CHO cell line with no functional glutamine synthetase gene. The pool of transfected cells were grown in the absence of glutamine added to the media until their viability reached 95%. They were then grown in a 14 day fed-batch using Sigma Advanced Fed Batch media. Protein was purified from the culture supernatant using protein A affinity chromatography, reduced with dithiothreitol, and analyzed on an Agilent QTOF mass spectrometer. We integrated the area under the peaks at 50,456 Da (corresponding to the heavy chain modified by G₀: the conserved heptasaccharide core composed of 2 N-acetylglucosamine, 3 mannose and 2 other N-acetylglucosamine residues that are β-1,2 linked to α-6 mannose and α-3 mannose, forming two arms) and 50,602 (corresponding to the heavy chain modified by G_{0F}: the conserved heptasaccharide core plus a fucose residue) to calculate the relative proportion of fucosylated and afucosylated antibody. Results are shown in Table 6.

Table 6 shows that when the inhibitory 5′ UTR sequences with SEQ ID NOs 402 or 403 are placed between the PGK promoter and the glutamine synthetase gene, the antibody titer is approximately 2 g/L (Table 6 rows 2 and 3). This is very similar to the titer seen with a more highly attenuated glutamine synthetase but no amiRNA hairpins in the 3′ UTR of the gene (Table 6 row 1), and more than twice the titer seen in the absence of this attenuating 5′ UTR element in Section 6.1.2.1 and Table 5 row 5. However, in the absence of the amiRNA, 82% of the antibody is fucosylated (Table 6 column G), consistent with the 80-85% fucosylation seen I Sections 6.1.1.1 and 6.1.1.2. When the transposons contained the amiRNA in the 3′UTR of the glutamine synthetase gene, the antibody is fully afucosylated (Table 6 column F). Use of the weaker HSV-TK promoter also resulted in fully afucosylated antibody (Table 6 rows 4 and 5), although the titer was not as high as with the PGK promoter.

The antibody open reading frames in transposons shown in rows 1-5 were operably liked to EF1 promoters. In rows 6-7 the antibody open reading frames were operably linked to CMV promoters. In row 6 the glutamine synthetase gene lacked multi-hairpin amiRNA sequences in the 3′ UTR. As with the EF1-driven antibody in row 1, the antibody was approximately 80% fucosylated, with a titer of 4.2 g/L. In row 7 the glutamine synthetase gene comprised multi-hairpin amiRNA sequence with nucleotide sequence SEQ ID NO: 194 in the 3′ UTR. As with the EF1-driven antibody in rows 2-5, antibody fucosylation was completely suppressed, while the titer exceeded 3 g/L.

We conclude that it is possible to incorporate multi-hairpin amiRNAs into the 3′ UTR of a selectable marker on a transposon, integrate the transposon into the genome of a cultured mammalian cell and obtain good titers of genes expressed from the transposon while simultaneously completely inhibiting genes endogenous to the cultured mammalian cell. Exemplary sequences of glutamine synthetase genes comprising multi-hairpin amiRNA sequences targeting CHO FUT8 mRNA are nucleic acid sequences SEQ ID NOs: 548-557.

### 6.2 Engineering of Glutamine Synthetase Knockdown with Micro RNAs

### 6.2.1 Glutamine Synthetase-Targeting microRNAs

As described in Section 5.4, multi-hairpin amiRNA with nucleotide sequence SEQ ID NO: 209 comprised 3 guide strand sequences complementary to 3 different sequences in the Chinese Hamster Cricetulus griseus glutamine synthetase mRNA. Multi-hairpin amiRNA, with nucleotide sequence SEQ ID NO: 209 comprised three hairpins; the first hairpin comprised guide strand sequence SEQ ID NO: 53, immediately followed by loop sequence SEQ ID NO: 241 and passenger strand sequence SEQ ID NO: 138, the second hairpin comprised guide strand sequence SEQ ID NO: 54, immediately followed by loop sequence SEQ ID NO: 241 and passenger strand sequence SEQ ID NO: 139, the third hairpin comprised guide strand sequence SEQ ID NO: 55, immediately followed by loop sequence SEQ ID NO: 241 and passenger strand sequence SEQ ID NO: 140. Each of these three guide strand sequences was a 22 base sequence that was an exact reverse complement of a different region within the Cricetulus griseus glutamine synthetase mRNA. Each passenger strand sequence was complementary to its corresponding guide strand sequence, except that the bases in the passenger strand sequences corresponding to the 5′ base of the guide strand and the twelfth base of the guide strand were changed to be non-complementary. The first and twelfth bases of guide strand with SEQ ID NO: 53 are T and T respectively, the corresponding bases in the corresponding passenger strand sequence SEQ ID NO: 138 are C and C respectively. The first and twelfth bases of guide strand with SEQ ID NO: 54 are T and A respectively, the corresponding bases in the corresponding passenger strand sequence SEQ ID NO: 139 are C and C respectively. The first and twelfth bases of guide strand with SEQ ID NO: 55 are T and G respectively, the corresponding bases in the corresponding passenger strand sequence SEQ ID NO: 140 are C and A respectively. Each hairpin in multi-hairpin amiRNA with nucleotide sequence SEQ ID NO: 209 further comprised additional stem-stabilizing sequences, with stem sequence SEQ ID NO: 255 immediately preceding the guide strand sequence, and stem sequence SEQ ID NO: 256 immediately following the passenger strand sequence. Multi-hairpin amiRNA sequences with nucleotide sequence SEQ ID NO: 209 further comprised an unstructured sequence with SEQ ID NO: 251 to the 5′ of the first hairpin, and an unstructured sequence with SEQ ID NO: 253 to the 3′ of the third hairpin. Multi-hairpin amiRNA sequence with nucleotide sequence SEQ ID NO: 209 further comprised an unstructured sequence with SEQ ID NO: 272 between the first and second hairpins, and an unstructured sequence with SEQ ID NO: 273 between the second and third hairpins. Each guide strand sequence is different, and each is complementary to the mRNA for Cricetulus griseus glutamine synthetase (SEQ ID NO: 10).

The multi-hairpin amiRNA was cloned into a piggyBac-like transposon to the 3′ of a spacer polynucleotide with nucleotide sequence SEQ ID NO: 280, and operably linked to a PGK promoter with nucleotide sequence SEQ ID NO: 386. The nucleotide sequence of the multi-hairpin amiRNA gene is given as SEQ ID NO: 540. The piggyBac-like transposon further comprised a selectable marker conferring resistance to G418/neomycin with amino acid sequence SEQ ID NO: 296. The piggyBac-like transposon further comprised a target sequence 5′-TTAA-3′ immediately followed by an ITR with nucleotide sequence SEQ ID NO: 448 (which is an embodiment of SEQ ID NO: 564), immediately followed by further transposon end sequences with nucleotide sequence SEQ ID NO: 445. The piggyBac-like transposon further comprised nucleotide sequence SEQ ID NO: 446, immediately followed by a second ITR with nucleotide sequence SEQ ID NO: 449 (which is an embodiment of SEQ ID NO: 447), immediately followed by the target sequence 5′-TTAA-3′. The transposon was configured so that the multi-hairpin amiRNA, the spacer polynucleotide and the gene encoding the selectable marker, as well as all necessary operably linked control elements, were transposable by a corresponding transposase. The full sequence of the transposon comprising the multi-hairpin amiRNA gene and selectable marker is given as SEQ ID NO: 544.

The transposon was co-transfected with mRNA encoding transposase with polypeptide sequence SEQ ID NO: 523 into a CHO cell line with intact glutamine synthetase genes. The pool of transfected cells were grown in the presence of 600 or 1,000 μg/ml G418 plus 5 mM glutamine until their viability reached 95%.

A control transposon comprised an open reading frame encoding RFP and a selectable marker gene conferring resistance to puromycin but lacked any multi-hairpin amiRNA sequences. The control transposon was introduced with mRNA encoding its corresponding transposase into the same CHO cell line with an intact glutamine synthetase gene. The pool of transfected cells were grown in the presence of 6 or 8 μg/ml puromycin plus 5 mM glutamine until their viability reached 95%.

After the transfected cell pools had recovered to >95% viability, we tested their ability to grow in the absence of glutamine. Cells were transferred to Sigma Advanced Fed Batch media lacking glutamine to an initial a density of 0.3×10⁶ live cells/ml. The viable cell density was measured at various times after the removal of glutamine. On the fourth day, cells were diluted back to a density of 0.3×10⁶ live cells/ml in media lacking glutamine, to ensure that growing cells had sufficient nutrients. Table 7 shows that the pool of cells transfected with the control transposon lacking a multi-hairpin amiRNA experienced an initial period of slow growth as they adapted to the glutamine-free media, but by day 4 the viable cell density had increased approximately 3-fold (Table 7 columns D and E, compare rows 3 and 5). After this, the viable cell density approximately tripled between dilution on day 4 and day 6 and doubled again between day 6 and day 8. In contrast, the pool of cells transfected with a transposon comprising the multi-hairpin amiRNA with nucleotide sequence SEQ ID NO: 209 and selected with 600 μg/ml G418 increased their viable cell density by less than 50% between day 1 and day 4 (Table 7 column C, compare rows 3 and 5), while the pool of cells transfected with a transposon comprising the multi-hairpin amiRNA with nucleotide sequence SEQ ID NO: 209 and selected with 1,000 μg/ml G418 failed to increase their viable cell density at all (Table 7 column B, compare rows 3 and 5). The viable cell density then began to fall for both pools transfected with a transposon comprising the multi-hairpin amiRNA with nucleotide sequence SEQ ID NO: 209 at day 6 (Table 7 columns B and C, compare rows 6, 7 and 8). By day 8 the viable cell density had fallen precipitously to less than 0.02×10⁶ live cells/ml. There was no difference between the growth of cells transfected with the control transposon or the transposon comprising the multi-hairpin amiRNA with nucleotide sequence SEQ ID NO: 209 in the presence of glutamine: all pools grew well. We conclude that a multi-hairpin amiRNA comprising guide strand sequences complementary to three different sequences within the CHO glutamine synthetase mRNA target (nucleotide sequence SEQ ID NO: 10) can be used to make a CHO cell dependent upon exogenously provided glutamine. The cells in this pool had been selected with neomycin/G418, which allowed growth of cells whose genomes comprised the transposon comprising the multi-hairpin amiRNA. By day 8 the viable cell density had fallen from 300,000 cells/ml to less than 20,000 cells/ml, indicating that less than 7% of the cells were still alive. By using the multi-hairpin amiRNA gene we were able to produce a pool of cells in which expression of the essential metabolic enzyme glutamine synthetase was inhibited to a level that prevents growth of the cell in greater than 93% of the cells in the pool.

The multi-hairpin amiRNA with nucleotide sequence SEQ ID NO: 209 was also cloned into three other piggyBac-like transposons, also to the 3′ of a spacer polynucleotide with nucleotide sequence SEQ ID NO: 280. In the first transposon the multi-hairpin amiRNA was operably linked to a PGK promoter with nucleotide sequence SEQ ID NO: 385. The nucleotide sequence of this multi-hairpin amiRNA gene is given as SEQ ID NO: 542. In the second transposon the multi-hairpin amiRNA was operably linked to an EF1 promoter with nucleotide sequence SEQ ID NO: 314. The nucleotide sequence of this multi-hairpin amiRNA gene is given as SEQ ID NO: 541. In the third transposon the multi-hairpin amiRNA was operably linked to an EEF2 promoter with nucleotide sequence SEQ ID NO: 350. The sequence of this multi-hairpin amiRNA gene is given as SEQ ID NO: 543. Each of these three piggyBac-like transposons further comprised a selectable marker conferring resistance to puromycin with amino acid sequence given by SEQ ID NO: 302. The piggyBac-like transposon further comprised a target sequence 5′-TTAA-3′ immediately followed by an ITR with the nucleotide sequence SEQ ID NO: 427, immediately followed by further transposon end s nucleotide sequence SEQ ID NO: 425. The piggyBac-like transposon further comprised a nucleotide sequence SEQ ID NO: 426, immediately followed by an ITR with the nucleotide sequence SEQ ID NO: 428, immediately followed by the target sequence 5′-TTAA-3′. The transposon was configured so that the multi-hairpin amiRNA, the spacer polynucleotide and the gene encoding the selectable marker, as well as all necessary operably linked control elements, were transposable by a corresponding transposase. The full sequence of the first, second and third transposons comprising the multi-hairpin amiRNA gene and selectable marker are given as nucleotide sequences SEQ ID NO: 546, 545 and 547 respectively. Each transposon was separately co-transfected with mRNA encoding transposase with polypeptide sequence SEQ ID NO: 502 into a CHO cell line with intact glutamine synthetase genes. The pool of transfected cells were grown in the presence of 10 μg/ml puromycin plus 5 mM glutamine until their viability reached 95%. After the cell pools had recovered to >95% viability, we tested their ability to grow in the absence of glutamine. Cells were transferred to Sigma Advanced Fed Batch media lacking glutamine to an initial a density of 0.3×10⁶ live cells/ml. The pool of cells derived from each transposon behaved essentially as shown in Table 7 for the pools selected with 600 or 1,000 ug/ml neomycin. We conclude that multi-hairpin amiRNA sequence with nucleotide sequence SEQ ID NO: 209 can be operably linked to a variety of different promoters, placed into a variety of different piggyBac-like transposons and integrated into the host genome by the corresponding transposase, in order to inhibit glutamine synthetase expression in CHO cells and make those cells dependent upon exogenously provided glutamine.

### 6.2.2 Clonal Cell Lines Comprising Genomically Integrated Multi-Hairpin amiRNA Directed Toward Glutamine Synthetase

Three monoclonal lines (#23, #38 and #129) were derived from the pool transfected with the transposon comprising multi-hairpin amiRNA with nucleotide sequence SEQ ID NO: 209 and selected with 1,000 μg/ml G418 described in Section 6.2.1. Growth of these clonal cell lines in the presence and absence of glutamine was compared with the growth of a cell line in which both genomic copies of the glutamine synthetase gene comprised inactivating mutations.

Cells were transferred to Sigma Advanced Fed Batch media lacking glutamine to an initial a density of 0.3×10⁶ live cells/ml. The viable cell density was measured at various times after the removal of glutamine. Table 8 shows that the clonal cell lines behaved similarly to the cell pool shown in Table 7. All three clonal lines showed a decrease in viable cell density beginning around day 6 (Table 8, columns B, C and D). The cell line in which both genomic copies of the glutamine synthetase gene comprised inactivating mutations showed a somewhat earlier decline in viable cell density, beginning around day 4 (Table 8, column E). In contrast, in the presence of glutamine, the viable cell density in all of the cell lines remained high until between day 7 and day 10. We observed some decrease in viable cell density at day 10. We believe that this is because in this experiment the cells were not diluted into fresh media at day 4. By day 4 in the presence of glutamine all cells had reached their maximum viable cell densities (Table 8 row 5), so by day 10 they were running out of nutrients. We conclude that all three monoclonal cell lines are dependent upon exogenously provided glutamine, and we expect that a glutamine synthetase gene can therefore be used as a selectable marker to select for integration of a second transposon into the genome of the cell.

### 6.2.3 Expression of an Antibody by Using Glutamine Synthetase Selection in a CHO Cell where Glutamine Synthetase has been Knocked Down Using a Multi-Hairpin amiRNA

Glutamine synthetase selection was used to integrate transposons for antibody expression into the monoclonal lines and the cell line in which both genomic copies of the glutamine synthetase gene comprised inactivating mutations described in Section 6.2.2.

One transposon (with nucleotide sequence SEQ ID NO: 290) comprised an open reading frame encoding a polypeptide comprising a mature light chain with polypeptide sequence SEQ ID NO: 286 operably linked to a murine EF1 promoter and a polyadenylation sequence, and an open reading frame encoding a polypeptide comprising a mature heavy chain with polypeptide sequence SEQ ID NO: 288 operably linked to a human EF1 promoter and a polyadenylation sequence. The transposon further comprised an open reading frame with nucleotide sequence SEQ ID NO: 309 encoding a glutamine synthetase gene with amino acid sequence SEQ ID NO: 308, operably linked to a heterologous promoter and heterologous 3′UTR and polyadenylation signal sequence. A second transposon (with nucleotide sequence SEQ ID NO: 289) comprised an open reading frame encoding a polypeptide comprising a mature light chain with polypeptide sequence SEQ ID NO: 286 operably linked to a human CMV promoter and a polyadenylation sequence, and an open reading frame encoding a polypeptide comprising a mature heavy chain with polypeptide sequence SEQ ID NO: 288 operably linked to a human CMV promoter and a polyadenylation sequence. The transposon further comprised an open reading frame with nucleotide sequence SEQ ID NO: 309 encoding a glutamine synthetase gene with amino acid sequence SEQ ID NO: 308, operably linked to a heterologous promoter and heterologous 3′UTR and polyadenylation signal sequence. The three guide strand sequences in multi-hairpin amiRNA with nucleotide sequence SEQ ID NO: 209 are all complementary to different sequences within the natural 3′ UTR of the Cricetulus griseus glutamine synthetase gene. Thus, expression of the glutamine synthetase gene from the transposons comprising the antibody-encoding sequences should not be affected by the anti-glutamine synthetase multi-hairpin amiRNA gene.

Both transposons further comprised a left end comprising a 5′-TTAA-3′ target sequence immediately followed by an ITR with nucleotide sequence SEQ ID NO: 423 (which is an embodiment of SEQ ID NO: 421) and additional nucleotide sequence SEQ ID NO: 417 and a right end comprising nucleotide sequence SEQ ID NO: 417 immediately followed by an ITR with nucleotide sequence SEQ ID NO: 424 (which is an embodiment of SEQ ID NO: 422) immediately followed by a 5′-TTAA-3′ target sequence. The transposons were configured so that the glutamine synthetase gene and the genes for both antibody chains, as well as all necessary operably linked control elements were transposable by a corresponding transposase.

Transposons were co-transfected with mRNA encoding transposase with polypeptide sequence SEQ ID NO: 472 into four different CHO cell lines: one in which both genomic copies of the gene comprised inactivating deletions, and the other three were clonal cell lines #23, #38 and #129, in which glutamine synthetase was inhibited using a multi-hairpin amiRNA, as described in Sections 6.2.1 and 6.2.2. The corresponding transposase for these transposons is different than the transposase used to transpose the first transposon, described in Section 6.2.1, which comprised the amiRNA gene for inhibiting the natural glutamine synthetase gene in the CHO cell. This ensured that the first transposon was not excised or inactivated by the action of the second transposase. The pools of transfected cells were grown in the absence of glutamine added to the media until their viability reached 95%. They were then grown in a 14 day fed-batch using Sigma Advanced Fed Batch media. Protein concentration in the supernatant was measured using an Octet. Results are shown in Table 9. The amount of antibody produced by cells in which glutamine synthetase expression had initially been inhibited by engineering mutations into the genomic copies of the genes (Table 9 rows 4 and 8) were comparable with the amount of antibody produced by the 3 cell lines in which glutamine synthetase expression was initially inhibited by the amiRNA gene (compare rows 1-3 with row 4, and rows 5-7 with row 8). The attenuated glutamine synthetase gene in the second transposon is thus capable of selecting for the same high level of expression of other genes on the second transposon in cells whose glutamine synthetase expression has been inhibited by interfering RNA as in those whose glutamine synthetase was inhibited by direct genetic mutation of the glutamine synthetase gene.

We conclude that in mammalian cells in which glutamine synthetase expression has been reduced by integrating into the genome a first transposon comprising a multi-hairpin amiRNA gene comprising nucleotide sequence SEQ ID NO: 209, cells whose genomes comprise a second transposon can be selected by using a gene encoding glutamine synthetase as a selectable marker on the second transposon. The second transposon comprised additional genes expressible in the mammalian cell to produce an antibody. The productivity of this glutamine synthetase knock-down cell line is comparable with the productivity of a cell line in which the glutamine synthetase was inactivated by genomic mutations.

### 6.2.4 Stability of Antibody Expression from a CHO Cell where Glutamine Synthetase has been Knocked Down Using a Multi-Hairpin amiRNA

The pool of cells obtained by transfecting clone 129 from Section 6.2.2 with the antibody-expressing transposon with nucleotide sequence SEQ ID NO: 290 (as described in Section 6.2.3 and shown in Table 9 row 7) was passaged for 30 or 60 population doublings to assess the stability of expression in the presence or absence of G418, the selection initially used to introduce the glutamine-synthetase-inhibiting multi-hairpin amiRNA. A clonal cell line is regarded as “stable” if its productivity after 60 population doublings is still at least 70% of the original productivity. Pools of CHO cells whose genomes include antibody-encoding genes typically show some additional decline in productivity as they are passaged as a result of population dynamics: lower producing cells tend to grow more quickly as they have a lower metabolic burden, and they take over the pool.

After passaging cells were grown in a 14 day fed-batch using Sigma Advanced Fed Batch media. Protein concentration in the supernatant was measured using an Octet. Results are shown in Table 10. Column F shows the antibody titer produced at day 14, column G shows the titer as a percentage of the unpassaged pool (row 1). Table 10 shows that cell pools passaged for 30 or 60 population-doublings in the presence of G418 produced 89% and 85% respectively of the day 14 antibody titer produced by the unpassaged pool. In the absence of G418, stability was even better: even after 60 population-doublings in the absence of G418, the cell pool still produced close to 95% of the day 14 antibody titer produced by the unpassaged pool. All of these titers are substantially above what is generally considered the threshold for “clonal stability”.

We conclude that if a gene encoding an essential enzyme is inhibited using genomically-integrated multi-hairpin amiRNA genes, and if the genomic integration of a second polynucleotide comprising a complementing selectable marker provides an alternative way for the cell to perform the inhibited essential function, then the expression of other genes encoded on the second polynucleotide can be stably maintained.

### 6.3 Sialidase Knockdown with Micro RNAs

### 6.3.1 Neu2-Targeting microRNAs

As described in Section 5.6, multi-hairpin amiRNAs may be used to inhibit sialidases and thereby increase the sialic acid content of polypeptides produced by a cell. A gene encoding a multi-hairpin amiRNA, with nucleotide sequence SEQ ID NO: 568, comprised three hairpins; the first hairpin comprised guide strand sequence SEQ ID NO: 92, immediately followed by loop sequence SEQ ID NO: 241 and passenger strand sequence SEQ ID NO: 177, the second hairpin comprised guide strand sequence SEQ ID NO: 91, immediately followed by loop sequence SEQ ID NO: 241 and passenger strand sequence SEQ ID NO: 176, the third hairpin comprised guide strand sequence SEQ ID NO: 90, immediately followed by loop sequence SEQ ID NO: 241 and passenger strand sequence SEQ ID NO: 175. Each of these three guide strand sequences was a 22 base sequence that was an exact reverse complement of a different region within the Cricetulus griseus glutamine synthetase mRNA. Each passenger strand sequence was complementary to its corresponding guide strand sequence, except that the bases in the passenger strand sequences corresponding to the 5′ base of the guide strand and the twelfth base of the guide strand were changed to be non-complementary. The first and twelfth bases of guide strand with SEQ ID NO: 92 are A and G respectively, the corresponding bases in the corresponding passenger strand sequence SEQ ID NO: 177 are C and A respectively. The first and twelfth bases of guide strand with SEQ ID NO: 91 are T and T respectively, the corresponding bases in the corresponding passenger strand sequence SEQ ID NO: 176 are C and C respectively. The first and twelfth bases of guide strand with SEQ ID NO: 90 are T and G respectively, the corresponding bases in the corresponding passenger strand sequence SEQ ID NO: 175 are C and A respectively. Each hairpin in multi-hairpin amiRNA with nucleotide sequence SEQ ID NO: 568 further comprised additional stem-stabilizing sequences, with stem sequence SEQ ID NO: 255 immediately preceding the guide strand sequence, and stem sequence SEQ ID NO: 256 immediately following the passenger strand sequence. Multi-hairpin amiRNA sequences with nucleotide sequence SEQ ID NO: 568 further comprised an unstructured sequence with SEQ ID NO: 251 to the 5′ of the first hairpin, and an unstructured sequence with SEQ ID NO: 253 to the 3′ of the third hairpin. Multi-hairpin amiRNA sequence with nucleotide sequence SEQ ID NO: 568 further comprised an unstructured sequence with nucleotide sequence SEQ ID NO: 272 between the first and second hairpins, and an unstructured sequence with nucleotide sequence SEQ ID NO: 273 between the second and third hairpins. Each guide strand sequence is different, and each is complementary to the mRNA for Cricetulus griseus Neu2 (nucleotide sequence SEQ ID NO: 570).

The multi-hairpin amiRNA was cloned into a piggyBac-like transposon to the 3′ of a spacer polynucleotide with nucleotide sequence SEQ ID NO: 280, and operably linked to a PGK promoter with nucleotide sequence SEQ ID NO: 385. The nucleotide sequence of the multi-hairpin amiRNA gene is given as SEQ ID NO: 593. The piggyBac-like transposon further comprised a selectable marker conferring resistance to puromycin with amino acid sequence SEQ ID NO: 302. The piggyBac-like transposon further comprised a target sequence 5′-TTAA-3′ immediately followed by an ITR with nucleotide sequence SEQ ID NO: 427, immediately followed by further transposon end sequences with nucleotide sequence SEQ ID NO: 425. The piggyBac-like transposon further comprised nucleotide sequence SEQ ID NO: 426, immediately followed by a second ITR with nucleotide sequence SEQ ID NO: 428, immediately followed by the target sequence 5′-TTAA-3′. The transposon was configured so that the multi-hairpin amiRNA, the spacer polynucleotide and the gene encoding the selectable marker, as well as all necessary operably linked control elements, were transposable by a corresponding transposase. The full nucleotide sequence of the transposon comprising the multi-hairpin amiRNA gene and selectable marker is given as SEQ ID NO: 594.

The transposon was co-transfected with mRNA encoding transposase with polypeptide sequence SEQ ID NO: 502 into a CHO cell line expressing an Fc fusion to a growth factor receptor (the “sialidase substrate”). This protein is naturally sialylated and was used to determine the activity of sialidases during the cell culturing process. The pool of transfected cells were grown in the presence of 10 μg/ml puromycin until their viability reached 95%.

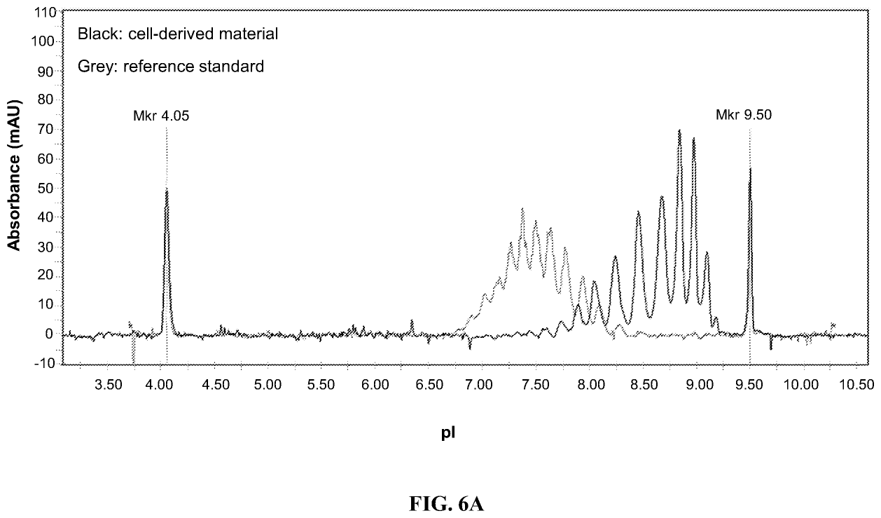

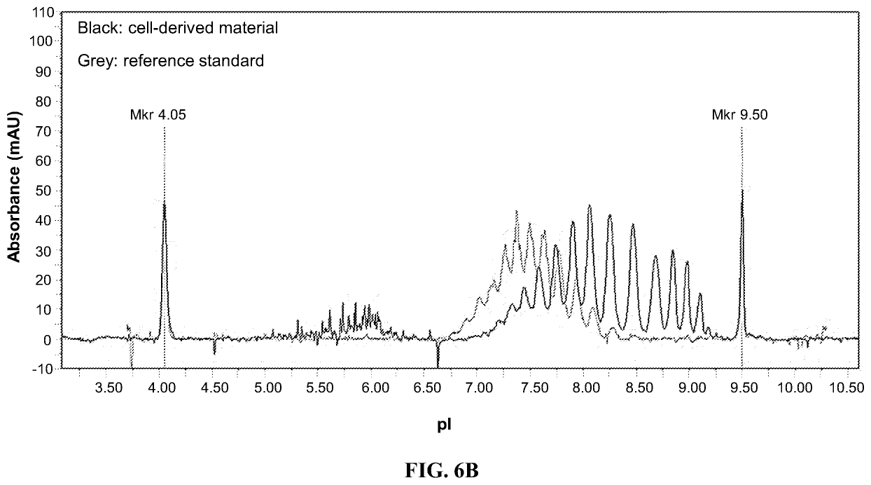

After the transfected cell pools had recovered to >95% viability, we tested their ability to produce sialylated protein. A pool of cells transfected with the transposon with nucleotide sequence SEQ ID NO: 594, and a pool of cells transfected with a control transposon without any amiRNA sequences were grown in a 14 day fed-batch using Sigma Advanced Fed Batch media. Protein was purified from the culture supernatant using protein A affinity chromatography, reduced with dithiothreitol, and analyzed by capillary isoelectric focusing (cIEF). The cIEF traces are shown in FIGS. 6A-B. Peaks correspond to different protein glycoforms. Peaks at the higher pI (on the x axis) correspond to less heavily sialylated protein species, peaks at the lower pI correspond to more sialylated protein species.

FIG. 6A shows that there is a substantial difference between the glycoforms seen in the highly sialylated reference standard (grey line), and the protein produced from the CHO line containing no amiRNA gene. In contrast, FIG. 6B shows that when protein was purified from the pool of cells transfected with the transposon comprising a multi-hairpin amiRNA designed to inhibit the expression of neu2 sialidases, there is a significant shift of the glycoform peaks to more heavily sialylated species, substantially increasing the overlap of the cIEF trace with the reference standard trace. We conclude that stable introduction of amiRNA multihairpin with nucleotide sequence SEQ ID NO: 568 into a CHO cell substantially reduces the removal of sialic acid from proteins produced by CHO cells. We observed essentially identical results when cells were transfected with multi-hairpin amiRNA genes comprising just two neu2-targetting hairpins: one guide strand with nucleotide sequence SEQ ID NO: 91, immediately followed by loop sequence SEQ ID NO: 241 and passenger strand sequence SEQ ID NO: 176, a second guide strand with nucleotide sequence SEQ ID NO: 90, immediately followed by loop sequence SEQ ID NO: 241 and passenger strand sequence SEQ ID NO: 175. We also expressed these hairpins along with between one and seven additional hairpins for inhibition of as many as four additional genes, and in each case we observed the same shift in glycan structures indicating inhibition of sialidase activity. We conclude that an advantageous amiRNA gene for inhibition of sialidases comprises a first guide strand with nucleotide sequence SEQ ID NO: 91 and a second guide strand with nucleotide sequence SEQ ID NO: 90.

### 6.4 Improving Heterologous Interferon Expression by Inhibiting Expression of the Interferon Receptor

### 6.4.1 Interferon Receptor-Targeting microRNAs

As described in Section 5.7, multi-hairpin amiRNAs may be used to inhibit one or both subunits of the interferon receptor and thereby reduce interferon-mediated retardation of cell growth in cells expressing interferon. We prepared five polynucleotides, four of which comprised multi-hairpin amiRNAs with guide strands complementary to one or both subunits of the interferon receptor, and the fifth comprising no amiRNA sequences as a control.

One multi-hairpin amiRNA, with nucleotide sequence SEQ ID NO: 598, comprised four hairpins; the first hairpin comprised a guide strand with nucleotide sequence SEQ ID NO: 95, immediately followed by a loop with nucleotide sequence SEQ ID NO: 241 and passenger strand with nucleotide sequence SEQ ID NO: 180, the second hairpin comprised a guide strand with nucleotide sequence SEQ ID NO: 97, immediately followed by a loop with nucleotide sequence SEQ ID NO: 241 and passenger strand with nucleotide sequence SEQ ID NO: 182, the third hairpin comprised a guide strand with nucleotide sequence SEQ ID NO: 96, immediately followed by a loop with nucleotide sequence SEQ ID NO: 241 and a passenger strand with nucleotide sequence SEQ ID NO: 181, the fourth hairpin comprised a guide strand with nucleotide sequence SEQ ID NO: 98, immediately followed by a loop with nucleotide sequence SEQ ID NO: 241 and a passenger strand with nucleotide sequence SEQ ID NO: 183. Each of these four guide strand sequences was a 22 base sequence that was an exact reverse complement of a different region within the Cricetulus griseus interferon receptor subunit 1 mRNA (with nucleotide sequence SEQ ID NO: 19. Each passenger strand sequence was complementary to its corresponding guide strand sequence, except that the bases in the passenger strand sequences corresponding to the 5′ base of the guide strand and the twelfth base of the guide strand were changed to be non-complementary.

One multi-hairpin amiRNA, with nucleotide sequence SEQ ID NO: 599, comprised four hairpins; the first hairpin comprised a guide strand with nucleotide sequence SEQ ID NO: 103, immediately followed by a loop with nucleotide sequence SEQ ID NO: 241 and passenger strand with nucleotide sequence SEQ ID NO: 188; the second hairpin comprised a guide strand with nucleotide sequence SEQ ID NO: 104, immediately followed by a loop with nucleotide sequence SEQ ID NO: 241 and passenger strand with nucleotide sequence SEQ ID NO: 189; the third hairpin comprised a guide strand with nucleotide sequence SEQ ID NO: 102, immediately followed by a loop with nucleotide sequence SEQ ID NO: 241 and a passenger strand with nucleotide sequence SEQ ID NO: 187; the fourth hairpin comprised a guide strand with nucleotide sequence SEQ ID NO: 105, immediately followed by a loop with nucleotide sequence SEQ ID NO: 241 and a passenger strand with nucleotide sequence SEQ ID NO: 190. Each of these four guide strand sequences was a 22 base sequence that was an exact reverse complement of a different region within the Cricetulus griseus interferon receptor subunit 2 mRNA (with nucleotide sequence SEQ ID NO: 20). Each passenger strand sequence was complementary to its corresponding guide strand sequence, except that the bases in the passenger strand sequences corresponding to the 5′ base of the guide strand and the twelfth base of the guide strand were changed to be non-complementary.

One multi-hairpin amiRNA, with nucleotide sequence SEQ ID NO: 600, comprised five hairpins; the first hairpin comprised a guide strand with nucleotide sequence SEQ ID NO: 103, immediately followed by a loop with nucleotide sequence SEQ ID NO: 241 and passenger strand with nucleotide sequence SEQ ID NO: 188; the second hairpin comprised a guide strand with nucleotide sequence SEQ ID NO: 95, immediately followed by a loop with nucleotide sequence SEQ ID NO: 241 and passenger strand with nucleotide sequence SEQ ID NO: 180; the third hairpin comprised a guide strand with nucleotide sequence SEQ ID NO: 104, immediately followed by a loop with nucleotide sequence SEQ ID NO: 241 and a passenger strand with nucleotide sequence SEQ ID NO: 189; the fourth hairpin comprised a guide strand with nucleotide sequence SEQ ID NO: 97, immediately followed by a loop with nucleotide sequence SEQ ID NO: 241 and a passenger strand with nucleotide sequence SEQ ID NO: 182; the fifth hairpin comprised a guide strand with nucleotide sequence SEQ ID NO: 102, immediately followed by a loop with nucleotide sequence SEQ ID NO: 241 and a passenger strand with nucleotide sequence SEQ ID NO: 187. Two of these five guide strand sequences were a 22 base sequence that was an exact reverse complement of a different region within the Cricetulus griseus interferon receptor subunit 1 mRNA (with nucleotide sequence SEQ ID NO: 19); three of these five guide strand sequences were a 22 base sequence that was an exact reverse complement of a different region within the Cricetulus griseus interferon receptor subunit 2 mRNA (with nucleotide sequence SEQ ID NO: 20). Each passenger strand sequence was complementary to its corresponding guide strand sequence, except that the bases in the passenger strand sequences corresponding to the 5′ base of the guide strand and the twelfth base of the guide strand were changed to be non-complementary.

One multi-hairpin amiRNA, with nucleotide sequence SEQ ID NO: 601, comprised four hairpins; the first hairpin comprised a guide strand with nucleotide sequence SEQ ID NO: 101, immediately followed by a loop with nucleotide sequence SEQ ID NO: 241 and passenger strand with nucleotide sequence SEQ ID NO: 186; the second hairpin comprised a guide strand with nucleotide sequence SEQ ID NO: 99, immediately followed by a loop with nucleotide sequence SEQ ID NO: 241 and passenger strand with nucleotide sequence SEQ ID NO: 184; the third hairpin comprised a guide strand with nucleotide sequence SEQ ID NO: 102, immediately followed by a loop with nucleotide sequence SEQ ID NO: 241 and a passenger strand with nucleotide sequence SEQ ID NO: 187; the fourth hairpin comprised a guide strand with nucleotide sequence SEQ ID NO: 106, immediately followed by a loop with nucleotide sequence SEQ ID NO: 241 and a passenger strand with nucleotide sequence SEQ ID NO: 191. Two of these four guide strand sequences were a 22 base sequence that was an exact reverse complement of a different region within the Cricetulus griseus interferon receptor subunit 1 mRNA (with nucleotide sequence SEQ ID NO: 19); two of these four guide strand sequences were a 22 base sequence that was an exact reverse complement of a different region within the Cricetulus griseus interferon receptor subunit 2 mRNA (with nucleotide sequence SEQ ID NO: 20). Each passenger strand sequence was complementary to its corresponding guide strand sequence, except that the bases in the passenger strand sequences corresponding to the 5′ base of the guide strand and the twelfth base of the guide strand were changed to be non-complementary.

Each of the five polynucleotides was a transposon which further comprised a left end comprising a 5′-TTAA-3′ target sequence immediately followed by an ITR with SEQ ID NO: 423 (which is an embodiment of SEQ ID NO: 421) and additional sequence with SEQ ID NO: 417 and a right end comprising SEQ ID NO: 419 immediately followed by an ITR with SEQ ID NO: 424 (which is an embodiment of SEQ ID NO: 422) immediately followed by a 5′-TTAA-3′ target sequence. Each transposon further comprised a gene encoding a glutamine synthetase selectable marker. The multi-hairpin amiRNA sequences were incorporated into the 3′ UTR of the glutamine synthetase ORF. The construct lacking amiRNA sequences comprised an intron sequence that provided a comparable level of glutamine synthetase attenuation. Each transposon further comprised the same promoter operably linked to an open reading frame encoding human interferon beta. The nucleotide sequences of transposons comprising multi-hairpin amiRNA sequences SEQ ID NO: 598-601 are given as SEQ ID NOs: 602-605 respectively. The nucleotide sequence of the transposons lacking multi-hairpin amiRNA sequences is given as SEQ ID NO: 606.

Transposons were co-transfected with mRNA encoding transposase with polypeptide sequence SEQ ID NO: 502 into a CHO host lacking a functional glutamine synthetase gene. The pool of transfected cells were grown in the absence of glutamine until their viability reached 95%. They were then grown in a 10 day fed-batch culture. Interferon beta levels in the culture supernatant were measured using an ELISA assay. The amount of interferon beta produced in two independent cultures are shown in Table 11.

Table 11 shows that the control transposon expressed 48 μg/ml of interferon beta. The transposon comprising a multi-hairpin amiRNA (with nucleotide sequence SEQ ID NO: 598) with guides complementary only to subunit 1 of the interferon receptor resulted in a small decrease in interferon beta expression (compare rows 1 and 2 in Table 11). However the transposon comprising a multi-hairpin amiRNA (with nucleotide sequence SEQ ID NO: 599) with guides complementary only to subunit 2 of the interferon receptor, and both transposons comprising a multi-hairpin amiRNA (with nucleotide sequences SEQ ID NO: 600 and 601) with guides complementary to both subunits of the interferon receptor resulted in as much as twice the expression levels of interferon beta as the control transposon. We conclude that reducing expression of the interferon receptor using multi-hairpin amiRNAs ameliorates the effects of interferon expression on CHO cells and allows them to express higher levels of human interferon beta.

### 6.5 Engineering of Dihydrofolate Reductase Knockdown with Micro RNAs

### 6.5.1 Dihydrofolate Reductase-Targeting microRNAs

As described in Section 5.9, multi-hairpin amiRNA with nucleotide sequence SEQ ID NO: 210 comprised 3 guide strand sequences complementary to 3 different sequences in the Chinese Hamster Cricetulus griseus dihydrofolate reductase mRNA. Multi-hairpin amiRNA, with nucleotide sequence SEQ ID NO: 210 comprised three hairpins; the first hairpin comprised guide strand sequence SEQ ID NO: 82, immediately followed by loop sequence SEQ ID NO: 241 and passenger strand sequence SEQ ID NO: 167, the second hairpin comprised guide strand sequence SEQ ID NO: 83, immediately followed by loop sequence SEQ ID NO: 241 and passenger strand sequence SEQ ID NO: 168, the third hairpin comprised guide strand sequence SEQ ID NO: 84, immediately followed by loop sequence SEQ ID NO: 241 and passenger strand sequence SEQ ID NO: 169. Each of these three guide strand sequences was a 22 base sequence that was an exact reverse complement of a different region within the Cricetulus griseus dihydrofolate reductase mRNA. Each passenger strand sequence was complementary to its corresponding guide strand sequence, except that the bases in the passenger strand sequences corresponding to the 5′ base of the guide strand and the twelfth base of the guide strand were changed to be non-complementary. Each hairpin in multi-hairpin amiRNA with nucleotide sequence SEQ ID NO: 210 further comprised additional stem-stabilizing sequences, with stem sequence SEQ ID NO: 255 immediately preceding the guide strand sequence, and stem sequence SEQ ID NO: 256 immediately following the passenger strand sequence. Multi-hairpin amiRNA sequences with nucleotide sequence SEQ ID NO: 210 further comprised an unstructured sequence with SEQ ID NO: 251 to the 5′ of the first hairpin, and an unstructured sequence with SEQ ID NO: 253 to the 3′ of the third hairpin. Multi-hairpin amiRNA sequence with nucleotide sequence SEQ ID NO: 210 further comprised an unstructured sequence with SEQ ID NO: 272 between the first and second hairpins, and an unstructured sequence with SEQ ID NO: 273 between the second and third hairpins. Each guide strand sequence is different, and each is complementary to the mRNA for Cricetulus griseus dihydrofolate reductase (SEQ ID NO: 11).

The multi-hairpin amiRNA was cloned into a piggyBac-like transposon to the 3′ of a spacer polynucleotide with nucleotide sequence SEQ ID NO: 280, and operably linked to a PGK promoter with nucleotide sequence SEQ ID NO: 386. The nucleotide sequence of the multi-hairpin amiRNA gene is given as SEQ ID NO: 635. The piggyBac-like transposon further comprised a selectable marker conferring resistance to G418/neomycin with amino acid sequence SEQ ID NO: 296. The piggyBac-like transposon further comprised a target sequence 5′-TTAA-3′ immediately followed by an ITR with nucleotide sequence SEQ ID NO: 448 (which is an embodiment of SEQ ID NO: 564), immediately followed by further transposon end sequences with nucleotide sequence SEQ ID NO: 445. The piggyBac-like transposon further comprised nucleotide sequence SEQ ID NO: 446, immediately followed by a second ITR with nucleotide sequence SEQ ID NO: 449 (which is an embodiment of SEQ ID NO: 447), immediately followed by the target sequence 5′-TTAA-3′. The transposon was configured so that the multi-hairpin amiRNA, the spacer polynucleotide and the gene encoding the selectable marker, as well as all necessary operably linked control elements, were transposable by a corresponding transposase. The full sequence of the transposon comprising the multi-hairpin amiRNA gene and selectable marker is given as SEQ ID NO: 597.

The transposon was co-transfected with mRNA encoding transposase with polypeptide sequence SEQ ID NO: 523 into a CHO cell line with intact dihydrofolate reductase genes. The pool of transfected cells were grown in the presence of 800 μg/ml G418 plus 5 mM glutamine plus HT until their viability reached 95%.

### 6.5.2 A Clonal Cell Line Comprising Genomically Integrated Multi-Hairpin amiRNA Directed Toward Dihydrofolate Reductase

A monoclonal line (C426) was derived from the pool transfected with the transposon comprising multi-hairpin amiRNA with nucleotide sequence SEQ ID NO: 210 and selected with 800 μg/ml G418 described in Section 6.5.1. Growth of this clonal cell line in the presence and absence of HT and in the absence of HT and presence of 50 nM MTX was compared with the growth of a control cell line from which C426 was derived.

Cells were transferred to Sigma Advanced Fed Batch media with 5 uM glutamine and to an initial a density of 0.3×10⁶ live cells/ml. Parallel cultures were grown (a) in the presence of HT, (b) in the absence of HT and (c) in the absence of HT and the presence of 50 nM MTX. At day 4, all cultures were diluted to adjust viable cell densities to 0.3×10⁶ cell/ml. At day 8, all cultures were again diluted to adjust viable cell densities to 0.3×10⁶ cell/ml. At day 13, all cultures except for the culture of C426 with 50 nM MTX were again diluted to adjust viable cell densities to 0.3×10⁶ cell/ml.

Table 12 shows that the clonal cell line C426 behaved similarly to the control cell line in the presence (compare rows 3 and 6) or absence (compare rows 4 and 7) of HT. Thus, DHFR was not inhibited to a level where the cells required HT supplementation of the media to grow. However, when 50 nM MTX was included in the growth media, it exerted a strong cytostatic effect on C426 but not on the control cells. After day 8, 50 nM MTX prevented any increases in viable cell density in C426 (Table 12 row 5). In contrast, the control cells increased their viable cell density to 2.5×10⁶ cell/ml by day 13, when they were diluted back to 0.3×10⁶ cell/ml and again reached 4.74×10⁶ cell/ml by day 18. We conclude that stable integration of multi-hairpin amiRNA with nucleotide sequence SEQ ID NO: 210 into the genome of a CHO cell sensitizes cells to the presence of MTX in the growth media.

### 6.5.3 Expression of an Antibody by Using MTX Selection in a CHO Cell where Dihydrofolate Reductase has been Knocked Down Using a Multi-Hairpin amiRNA

DHFR selection was used to integrate transposons for antibody expression into the monoclonal line C426 and a control cell line (DG44) in which both genomic copies of the dihydrofolate reductase gene comprised inactivating mutations.

A gene transfer transposon (with nucleotide sequence SEQ ID NO: 636) comprised an open reading frame encoding a polypeptide comprising a mature light chain with polypeptide sequence SEQ ID NO: 286 operably linked to a murine CMV promoter and a polyadenylation sequence, and an open reading frame encoding a polypeptide comprising a mature heavy chain with polypeptide sequence SEQ ID NO: 288 operably linked to a murine CMV promoter and a polyadenylation sequence. The transposon further comprised an open reading frame with nucleotide sequence SEQ ID NO: 637 encoding a dihydrofolate reductase gene with amino acid sequence SEQ ID NO: 293, operably linked to a heterologous promoter and heterologous 3′UTR and polyadenylation signal sequence. The three guide strand sequences in multi-hairpin amiRNA with nucleotide sequence SEQ ID NO: 210 are all complementary to different sequences within the natural 3′ UTR of the Cricetulus griseus dihydrofolate reductase gene. The introduced DHFR gene on transposon with SEQ ID NO: 636 comprises a heterologous 3′UTR which lacks complementary sequences to the guide strand sequences in multihairpin amiRNA with nucleotide sequence SEQ ID NO: 210. Thus expression of the dihydrofolate reductase gene from the transposon comprising the antibody-encoding sequences should not be affected by the anti-dihydrofolate reductase multi-hairpin amiRNA gene.

The gene transfer transposon further comprised a left end comprising a 5′-TTAA-3′ target sequence immediately followed by an ITR with nucleotide sequence SEQ ID NO: 423 (which is an embodiment of SEQ ID NO: 421) and additional nucleotide sequence SEQ ID NO: 417 and a right end comprising nucleotide sequence SEQ ID NO: 417 immediately followed by an ITR with nucleotide sequence SEQ ID NO: 424 (which is an embodiment of SEQ ID NO: 422) immediately followed by a 5′-TTAA-3′ target sequence. The transposons were configured so that the dihydrofolate reductase gene and the genes for both antibody chains, as well as all necessary operably linked control elements were transposable by a corresponding transposase.

The gene transfer transposon with nucleotide sequence SEQ ID NO: 636 was co-transfected with mRNA encoding transposase with polypeptide sequence SEQ ID NO: 472 into two different CHO cell lines: one in which both genomic copies of the gene comprised inactivating deletions, and the other c426, in which dihydrofolate reductase was inhibited using a multi-hairpin amiRNA, as described in Sections 6.5.1 and 6.5.2. The corresponding transposase for these transposons is different than the transposase used to transpose the first transposon, described in Section 6.5.1, which comprised the amiRNA gene for inhibiting the natural dihydrofolate reductase gene in the CHO cell. This ensured that the first transposon was not excised or inactivated by the action of the second transposase. The pools of transfected cells were grown in media lacking HT and supplemented with 50 nM MTX until their viability reached 95%. They were then grown in a 14 day fed-batch using Sigma Advanced Fed Batch media. Protein concentration in the supernatant was measured using an Octet. Results are shown in Table 13. The amount of antibody produced by DG44 cells in which cellular dihydrofolate reductase expression was inhibited by genomic mutations (Table 13 row 1) was comparable with the amount of antibody produced by the c426 cell line in which cellular dihydrofolate reductase expression was inhibited by the multihairpin amiRNA gene (row 2). The viability of DG44 cells fell below 70% by day 12, so the culture had to be stopped. In contrast the viability of the c426 cells remained high until day 14, allowing the culture to progress for longer. The dihydrofolate reductase gene in the second transposon is thus capable of selecting for the same high level of expression of other genes on the second transposon in cells whose dihydrofolate reductase expression has been inhibited by interfering RNA as in those whose dihydrofolate reductase was inhibited by direct genetic mutation of the dihydrofolate reductase gene.

We conclude that in mammalian cells in which dihydrofolate reductase expression has been reduced by integrating into the genome a first transposon comprising a multi-hairpin amiRNA gene comprising nucleotide sequence SEQ ID NO: 210, cells whose genomes comprise a second transposon can be selected by using a gene encoding dihydrofolate reductase as a selectable marker on the second transposon. The second transposon comprised additional genes expressible in the mammalian cell to produce an antibody. The productivity of this dihydrofolate reductase knock-down cell line is comparable with the productivity of a cell line in which the dihydrofolate reductase was inactivated by genomic mutations.

### Brief Description of Tables

Table 1. Constructs used to generate the data shown in FIGS. 3A-G. Transposons were constructed as described in Section 6.1.1.1. The multi-hairpin amiRNA whose SEQ ID NO is shown in column C was operably linked to the Pol II promoter shown in column B. The corresponding mass spectroscopy trace is shown in the panel of FIGS. 3A-G indicated in column D.

Table 2. Inhibition of antibody fucosylation with amiRNAs targeting GMD and GFT. Transposons were constructed as described in Section 6.1.1.4. The amiRNA SEQ ID NO is shown in column A. Following a 14-day fed batch antibody production run, the percentage of antibody that was afucosylated is shown in column B, the percentage that was fucosylated is shown in column C. BDL=below detection limit.

Table 3. Inhibition of antibody fucosylation in HEK cells with multi-hairpin amiRNAs directed toward different target genes. Transposons were constructed, transfected into HEK cells and selected as described in Section 6.1.1.5. Gene transfer polynucleotides comprised amiRNAs directed toward the genes listed in column A. The multi-hairpin amiRNA had the sequence given by the SEQ ID NO shown in column B; the number of hairpins present in the multi-hairpin amiRNA is shown in column C. Recovered pools were transiently transfected with genes encoding an antibody with mature light chain polypeptide sequence SEQ ID NO: 286 and mature heavy chain polypeptide sequence SEQ ID NO: 287. Following a 7-day culture, the culture supernatant contained the concentration of antibody shown in column F. The percentage of antibody that was afucosylated is shown in column D, the percentage that was fucosylated is shown in column E. BDL=below detection limit.

Table 4. Inhibition of antibody fucosylation in clonal HEK cell lines with multi-hairpin amiRNAs directed toward FUT8. Clonal cell lines were generated from the pools shown in Table 3 rows 3 and 4. The name of the cell line is shown in column A. Clonal lines were transiently transfected with genes encoding an antibody with mature light chain polypeptide sequence SEQ ID NO: 286 and mature heavy chain polypeptide sequence SEQ ID NO: 287. Following a 7-day culture, the culture supernatant contained the concentration of antibody shown in column D. The percentage of antibody that was afucosylated is shown in column B, the percentage that was fucosylated is shown in column C.

Table 5. Inhibition of antibody fucosylation with different numbers of amiRNA hairpins. Transposons were constructed as described in Section 6.1.2.1. The SEQ ID NO of the amiRNA gene including the glutamine synthetase ORF and the globin polyA sequence is given in column A. The Pol II promoter shown in column B was operably linked to the amiRNA whose SEQ ID NO is shown in column C. The amiRNA comprised the number of hairpins shown in column D. Following a 14-day fed batch antibody production run, the culture supernatant contained the concentration of antibody shown in column E. The percentage of antibody that was afucosylated is shown in column F, the percentage that was fucosylated is shown in column G. BDL=below detection limit.

Table 6. Inhibition of antibody fucosylation with multi-hairpin amiRNAs driven by different promoters. Transposons were constructed as described in Section 6.1.2.2. The sequence of the selectable marker glutamine synthetase gene, including multi-hairpin amiRNA sequences in the 3′ UTR, is shown in column A. The Pol II promoter shown in column B was operably linked to the inhibitory 5′ UTR shown in column C which was operably linked to a glutamine synthetase gene. In the 3′ UTR of the glutamine synthetase gene was placed the amiRNA whose SEQ ID NO is shown in column D.

Following a 14-day fed batch antibody production run, the culture supernatant contained the concentration of antibody shown in column E. The percentage of antibody that was afucosylated is shown in column F, the percentage that was fucosylated is shown in column G. BDL=below detection limit.

Table 7. Growth of cells with amiRNA targeted toward glutamine synthetase in the absence of glutamine. Cells were transfected with transposons comprising the multi-hairpin amiRNA with SEQ ID NO shown in row 1 and selected by addition of G418 or puromycin at the concentration shown in row 2, as described in Section 6.2.1. After cells had recovered to >95% viability, cells were transferred into glutamine-free media at 0.3×10⁶ viable cells per ml of media. Viable cell densities were measured at various times after the beginning of the experiment: the number of days after initiation of the experiment are shown in column A. At day 4, cells were diluted back to 0.3×10⁶ live cells/ml (row 5 is before dilution, row 6 is after dilution). Columns B-E show viable cell densities x 10⁶ live cells/ml.

Table 8. Growth of clonal cell lines with amiRNA targeted toward glutamine synthetase in the absence of glutamine. The pool transfected with a transposon comprising multi-hairpin amiRNA with SEQ ID NO: 209 was cloned, and three clonal lines (clone ID shown in row 1) were grown in the presence or absence of glutamine (glutamine concentration is shown in row 2). Growth was compared with the growth of a cell line comprising inactivating mutations in both genomic copies of the glutamine synthetase gene (columns E and I, indicated as GS KO in line 1). Cells were inoculated at 0.3×10⁶ viable cells per ml of media. Viable cell densities were measured at various times after the beginning of the experiment: the number of days after initiation of the experiment are shown in column A. Columns B-I show viable cell densities x 10⁶ live cells/ml.

Table 9. Expression of an antibody in a glutamine synthetase knockdown cell. The four cell lines described in Section 6.2.2 and shown in Table 8 were transfected with two different transposons comprising open reading frames encoding the heavy and light chains of an antibody, with SEQ ID NO shown in column B, as described in Section 6.2.3. Clone IDs are indicated in column 1: three clones were derived from a pool of cells with two intact genomic copies of the glutamine synthetase gene that had been transfected with multi-hairpin amiRNA with nucleotide sequence SEQ ID NO: 209, in the fourth line both genomic copies of the glutamine synthetase gene comprised inactivating mutations (indicated as GS KO in column 1). Transposon SEQ ID NOs are indicated in column 2. Cells were selected as described in Section 6.2.3. After recovery they were inoculated for a 14-day fed batch, with samples taken after 7, 10, 12 and 14 days for titer measurement by Octet. Antibody titers measured in the culture supernatant are shown in μg/ml in columns C (day 7), D (day 10), E (day 12) and F (day 14).

Table 10. Stability of expression of an antibody from a glutamine synthetase knockdown cell. The cell pool in which clonal cell line #129 was transfected with transposon with sequence nucleotide SEQ ID NO: 290, as described in Section 6.2.3 and shown in Table 9 row 7, were tested for stability by passaging the cells for 0, 30 and 60 population doublings, as shown in column B. Cells were passaged in the presence or absence of G418, whose concentration is shown in column A. After passaging they were inoculated for a 14-day fed batch, with samples taken after 7, 10, 12 and 14 days for titer measurement by Octet. Antibody titers measured in the culture supernatant are shown in μg/ml in columns C (day 7), D (day 10), E (day 12) and F (day 14). The productivity at day 14 is expressed as a % of the productivity of the cell pool that had not undergone passaging (row 1).

Table 11. Expression of human interferon beta from CHO cells. Transposons for the expression of human interferon beta were constructed, transfected and expressed as described in Section 6.4.1. The amiRNA SEQ ID NO is shown in column A. Following a 10-day fed batch of duplicate cultures for each sample, the concentration of interferon was measured by ELISA. Measured interferon concentrations in culture supernatants are show in columns B and C. The average concentration from duplicate cultures is shown in column D.

Table 12. Growth of a clonal cell line with amiRNA targeted toward dihydrofolate reductase. A clonal cell line whose genome comprises a transposon comprising multi-hairpin amiRNA with SEQ ID NO: 210 (C426) was grown in the presence or absence of HT, or in the absence of HT and the presence of 50 nM MTX. Growth was compared with a control CHO-Kl cell line from which C426 was derived. Cells were inoculated at 0.3×10⁶ viable cells per ml of media and cultured as described in Section 6.5.2. Viable cell densities were measured at various times after the beginning of the experiment: the cell line is shown in column A, the presence of HT is indicated in column B and the concentration of MTX is shown in column C. Columns D-L show viable cell densities x 10⁶ live cells/ml. Days since the beginning of the experiment are shown in row 1, results for c426 in rows 3-5, results for the control line in rows 6-8.

Table 13. Expression of an antibody in a dihydrofolate reductase knockdown cell. The two cell lines described in Section 6.5.3 were transfected with a transposon comprising open reading frames encoding the heavy and light chains of an antibody, with nucleotide sequence SEQ ID NO: 636. Cell lines used are indicated in column 1. Cells were selected as described in Section 6.5.3. After recovery they were inoculated for a 14 day fed batch, with samples taken after 7, 10, 12 and 14 days for titer measurement by Octet. Antibody titers measured in the culture supernatant are shown in μg/ml in columns B (day 7), C (day 10), D (day 12) and E (day 14).

TABLE 1

| | A | B | C | D |
|---|---|---|---|---|
| | Construct name | Promoter | amiRNA SEQ ID NO | FIG. 1 panel |
| 1 | none | N/A | none | A |
| 2 | 344641 | EF1 | 193 | B |
| 3 | 344646 | EF1 | 194 | C |
| 4 | 344651 | EF1 | 195 | D |
| 5 | 344645 | CMV | 193 | E |
| 6 | 344650 | CMV | 194 | F |
| 7 | 344655 | CMV | 195 | G |

TABLE 2

| | A | B | C |
|---|---|---|---|
| | SEQ ID NO: | G0 + G1% (area) | G0F + G1F (% area) |
| 1 | none | 25 | 75 |
| 2 | 200 | 100 | BDL |

TABLE 3

| | A | B | C | D | E | F |
|---|---|---|---|---|---|---|
| | Targeted | SEQ | No of | G0 + G1 % | G0F + G1F | Titer |
| | genes | ID NO: | hairpins | (area) | (% area) | (mg/L) |
| 1 | none | N/A | N/A | BDL | 100 | 233 |
| 2 | none | N/A | N/A | 7 | 93 | 237 |
| 3 | FUT8 | 202 | 3 | 90 | 10 | 353 |
| 4 | FUT8 | 202 | 3 | 89 | 11 | 316 |
| 5 | GMD, GFT | 204 | 4 | 100 | BDL | 126 |
| 6 | GMD, GFT | 204 | 4 | 100 | BDL | 120 |

TABLE 4

| | | B | C | D |
|---|---|---|---|---|
| | A | G0 + G1% | G0F + G1F | Titer |
| | Sample | (area) | (% area) | (mg/L) |
| 1 | HEK 293 | 6 | 94 | 217 |
| 2 | HEK 293 | 10 | 90 | 224 |
| 3 | clonal line 1 | 56 | 44 | 208 |
| 4 | clonal line 1 | 59 | 41 | 225 |
| 5 | clonal line 2 | 80 | 20 | 371 |
| 6 | clonal line 2 | 81 | 19 | 379 |
| 7 | clonal line 3 | 87 | 13 | 116 |
| 8 | clonal line 3 | 87 | 13 | 134 |
| 9 | clonal line 4 | 94 | 6 | 258 |
| 10 | clonal line 4 | 90 | 10 | 248 |

TABLE 5

| | A | B | C | D | E | F | G |
|---|---|---|---|---|---|---|---|
| | GS/amiRNA | Promoter | amiRNA | No of | Titer | G0 % | G0F |
| | SEQ ID NO | SEQ ID NO | SEQ ID NO | hairpins | (mg/L) | (area) | (% area) |
| 1 | 549 | 383 | 194 | 3 | 163 | 100 | BDL |
| 2 | 550 | 350 | 194 | 3 | 443 | 100 | BDL |
| 3 | 551 | 383 | 197 | 1 | 514 | 47.3 | 52.7 |
| 4 | 552 | 383 | 196 | 2 | 770 | 89.9 | 10.1 |
| 5 | 548 | 383 | 194 | 3 | 835 | 100 | BDL |

TABLE 6

| | A | B | C | D | E | F | G |
|---|---|---|---|---|---|---|---|
| | GS/amiRNA gene | Promoter | 5′ UTR | amiRNA | Titer | G0 % | G0F |
| | SEQ ID NO | SEQ ID NO | SEQ ID NO | SEQ ID NO | (mg/L) | (area) | (% area) |
| 1 | 562 | 393 | none | none | 2,130 | 18 | 82 |
| 2 | 553 | 383 | 402 | 194 | 1,837 | 100 | BDL |
| 3 | 554 | 383 | 403 | 194 | 1,933 | 100 | BDL |
| 4 | 555 | 393 | 402 | 194 | 821 | 100 | BDL |
| 5 | 556 | 399 | 403 | 194 | 1,178 | 100 | BDL |
| 6 | 563 | 393 | none | none | 4,200 | 19 | 81 |
| 7 | 557 | 383 | 403 | 194 | 3,100 | 100 | BDL |

TABLE 7

| | A | B | C | D | E |
|---|---|---|---|---|---|
| 1 | SEQ ID NO | 741 | 741 | none | none |
| 2 | Selection | 1000 ug/ml G418 | 600 ug/ml G418 | 8 ug/ml puromycin | 6 ug/ml puromycin |
| | Day | VCD | VCD | VCD | VCD |
| 3 | 0 | 0.30 | 0.30 | 0.30 | 0.30 |
| 4 | 1 | 0.31 | 0.45 | 0.38 | 0.41 |
| 5 | 4 | 0.30 | 0.47 | 0.99 | 0.85 |
| 6 | 4 | 0.30 | 0.30 | 0.30 | 0.30 |
| 7 | 6 | 0.26 | 0.21 | 1.11 | 1.00 |
| 8 | 8 | 0.02 | 0.01 | 2.00 | 2.54 |

TABLE 8

| | A | B | C | D | E | F | G | H | I |
|---|---|---|---|---|---|---|---|---|---|
| 1 | clone # | 23 | 38 | 129 | n/a | 23 | 38 | 129 | n/a |
| 2 | glutamine (mM) | 0 | 0 | 0 | 0 | 5 | 5 | 5 | 5 |
| 3 | 0 | 0.30 | 0.30 | 0.30 | 0.30 | 0.30 | 0.30 | 0.30 | 0.30 |
| 4 | 4 | 0.40 | 0.37 | 0.32 | 0.22 | 3.76 | 6.63 | 4.34 | 5.96 |
| 5 | 5 | 0.36 | 0.36 | 0.30 | 0.06 | 3.93 | 6.96 | 5.58 | 5.21 |
| 6 | 6 | 0.23 | 0.28 | 0.25 | 0.05 | 4.13 | 6.70 | 5.79 | 5.75 |
| 7 | 7 | 0.16 | 0.21 | 0.19 | not done | 3.61 | 6.14 | 5.79 | 5.17 |
| 8 | 10 | 0.09 | 0.06 | 0.03 | 0.06 | 0.68 | 0.74 | 3.17 | 1.98 |

TABLE 9

| | | B | C | D | E | F |
|---|---|---|---|---|---|---|
| | A | Transposon | day | day | day | day |
| | Host cells | SEQ ID NO | 7 | 10 | 12 | 14 |
| 1 | amiRNA clone#23 | 289 | 1,517 | 2,669 | 2,915 | 3,324 |
| 2 | amiRNA clone#38 | 289 | 1,638 | 3,083 | 3,480 | 4,193 |
| 3 | amiRNA clone#129 | 289 | 1,827 | 3,023 | 3,236 | 3,729 |
| 4 | GS KO | 289 | 715 | 1,586 | 2,174 | 2,637 |
| 5 | amiRNA clone#23 | 290 | 1,482 | 2,084 | 2,133 | 2,244 |
| 6 | amiRNA clone#38 | 290 | 1,363 | 2,146 | 2,151 | 2,273 |
| 7 | amiRNA clone#129 | 290 | 1,328 | 2,286 | 2,575 | 3,044 |
| 8 | GS KO | 290 | 1,059 | 1,618 | 1,802 | 2,019 |

TABLE 10

| | A | B | C | D | E | F | G |
|---|---|---|---|---|---|---|---|
| | G418 Concentration | Population doublings | Day 7 | Day 10 | Day 12 | Day 14 | % of control |
| 1 | 400 ug/ml | 0 | 2,031 | 2,425 | 3,286 | 3,355 | 100 |
| 2 | 400 ug/ml | 30 | 1,123 | 1,999 | 2,887 | 2,997 | 89.3 |
| 3 | 400 ug/ml | 60 | 1,132 | 1,909 | 2,743 | 2,869 | 85.5 |
| 4 | 0 | 30 | 1,605 | 2,350 | 3,241 | 3,418 | 101.9 |
| 5 | 0 | 60 | 1,348 | 2,144 | 3,174 | 3,179 | 94.8 |

TABLE 11

| | A | B | C | D |
|---|---|---|---|---|
| | amiRNA SEQ | Titer | Titer | Average Titer |
| | ID NO | (ug/ml) | (ug/ml) | (ug/ml) |
| 1 | none | 48 | 48.3 | 48.2 |
| 2 | 598 | 22.8 | 46.1 | 34.5 |
| 3 | 599 | 92.9 | 105.7 | 99.3 |
| 4 | 600 | 80.3 | 79.2 | 79.8 |
| 5 | 601 | 97.3 | 78.6 | 88.0 |

TABLE 12

| | A | B | C | D | E | F | G | H | I | J | K | L |
|---|---|---|---|---|---|---|---|---|---|---|---|---|
| 1 | Day | n/a | n/a | 4 | 6 | 8 | 8 | 11 | 13 | 13 | 15 | 18 |
| 2 | Cell line | HT | MTX (nM) | n/a | n/a | n/a | n/a | n/a | n/a | n/a | n/a | n/a |
| 3 | C426 | yes | 0 | 0.30 | 0.38 | 5.07 | 0.30 | 2.16 | 5.62 | 0.30 | 1.17 | 7.47 |
| 4 | C426 | no | 0 | 0.30 | 0.24 | 3.12 | 0.30 | 2.91 | 5.04 | 0.30 | 0.63 | 6.55 |
| 5 | C426 | no | 50 | 0.30 | 0.04 | 1.87 | 0.30 | 0.28 | 0.26 | 0.26 | 0.13 | 0.26 |
| 6 | control | yes | 0 | 0.30 | 0.64 | 5.94 | 0.30 | 4.94 | 9.01 | 0.30 | 2.02 | 7.96 |
| 7 | control | no | 0 | 0.30 | 0.67 | 5.87 | 0.30 | 4.69 | 8.74 | 0.30 | 1.59 | 5.80 |
| 8 | control | no | 50 | 0.30 | 0.08 | 1.19 | 0.30 | 1.35 | 2.46 | 0.30 | 0.69 | 4.74 |

TABLE 13

| | A | B | C | D | E |
|---|---|---|---|---|---|
| | Host cells | day 7 | day 10 | day 12 | day 14 |
| 1 | amiRNA c426 | 953 | 2,106 | 2,933 | 3,391 |
| 2 | DG44 | 2,371 | 3,237 | 3,362 | n/d |

### 7. REFERENCES

All references cited herein are incorporated herein by reference in their entirety and for all purposes to the same extent as if each individual publication or patent or patent application was specifically and individually indicated to be incorporated by reference in its entirety for all purposes. U.S. 62/846,847, filed May 13, 2019, U.S. 62/870,321, filed Jul. 3, 2019, U.S. 62/981,417 filed Feb. 25, 2020, U.S. 63/019,733 filed May 4, 2020, PCT/US2020/032381 filed May 11, 2020, and U.S. Ser. No. 16/872,051 filed May 11, 2020, described related subject matter and are incorporated by reference in their entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled. To the extent different content is associated with an accession number or other reference at different times, the content in effect as of the earlier of the application filing date or filing date of earliest priority application disclosing the accession number in question is meant. Unless otherwise apparent from the context any element, embodiment, step, feature or aspect of the invention can be performed in combination with any other.

What is claimed is:

1. A polynucleotide comprising
a) a segment encoding a multi-hairpin amiRNA sequence, wherein the segment comprises
i) a first guide strand sequence comprising a contiguous 19 nucleotide sequence that is perfectly complementary to a first target site in a natural mammalian cellular mRNA of SEQ ID NO: 11 and a first passenger strand sequence comprising a contiguous 19 nucleotide sequence that is at least 78% complementary to the first guide strand sequence, wherein the first guide strand and first passenger strand sequence are separated by between 5 and 35 nucleotides;
ii) a second guide strand sequence comprising a contiguous 19 nucleotide sequence that is perfectly complementary to a second target site different than the first target site in the same natural mammalian cellular mRNA as the first guide strand sequence and a second passenger strand sequence comprising a contiguous 19 nucleotide sequence that is at least 78% complementary to the second guide strand sequence, wherein the second guide strand and second passenger strand sequences are separated by between 5 and 35 nucleotides, and wherein the first and second guide strand sequences are different from each other; and
b) a eukaryotic promoter that is active in a mammalian cell and is transcribed by RNA polymerase II or RNA polymerase III operably linked to the segment encoding the amiRNA sequence, wherein the amiRNA sequence can be expressed and fold into multiple hairpins;
wherein the first and second guide strand sequences are selected from SEQ ID NOs: 82-84 and 607-616.

2. The polynucleotide of claim 1, wherein the first guide strand sequence is a 19-22 nucleotide sequence perfectly complementary to the natural mammalian cellular mRNA and the first passenger strand sequence has the same length as the first guide sequence.

3. The polynucleotide of claim 1, wherein the first guide strand sequence is a 19-22 nucleotide sequence perfectly complementary to the natural mammalian cellular mRNA and the first passenger strand sequence is shorter than the first guide sequence.

4. The polynucleotide of any preceding claim, wherein the first and second target sites do not overlap.

5. The polynucleotide of any preceding claim, wherein the segment encoding the multi-hairpin amiRNA sequence further comprises a third guide strand sequence comprising a contiguous 19 nucleotide sequence that is perfectly complementary to the same natural mammalian cellular mRNA as the first and second guide strand sequences and a third passenger strand sequence comprising a contiguous 19 nucleotide sequence that is at least 78% complementary to the third guide strand sequence, wherein the third guide strand and third passenger strand sequences are separated by between 5 and 35 nucleotides, and wherein the first, second and third guide strand sequences are different from each other.

6. The polynucleotide of any preceding claim, further comprising two transposon ends flanking the segment and the promoter, wherein the segment and the promoter are transposable by a corresponding transposase.

7. The polynucleotide of claim 6, wherein each transposon end comprises a sequence selected from SEQ ID NOs: 421 and 422, or from SEQ ID NOs: 427 and 428, or from SEQ ID NOs: 431 and 432, or from SEQ ID NOs: 433 and 434, or from SEQ ID NOs: 439 and 440, or from SEQ ID NOs: 443 and 444, or from SEQ ID NOs: 447 and 564, or from SEQ ID NOs: 452 and 453, or from SEQ ID NOs: 460 and 461, or from SEQ ID NOs: 528 and 529.

8. The polynucleotide of claim 1, wherein the polynucleotide comprises a sequence selected from SEQ ID NO: 210 and 627.

9. A mammalian cell comprising the polynucleotide of any preceding claim integrated into its genome.

10. The mammalian cell of claim 9, wherein the multi-hairpin amiRNA sequence is expressed and inhibits expression of the natural cellular mRNA, and whereby the growth of the cell in the presence of 50 nM methotrexate cell is inhibited relative to the growth of an otherwise identical cell whose genome does not comprise the multi-hairpin amiRNA.

11. A mammalian cell comprising
a) the polynucleotide of any one of claims 1-8 integrated into its genome, wherein the multi-hairpin amiRNA sequence is expressed and inhibits expression of the natural cellular mRNA and
b) a second polynucleotide comprising a gene encoding dihydrofolate reductase expressible in the mammalian cell, wherein expression of the gene compensates for the inhibition of the expression of the natural cellular mRNA, whereby the cell grows without the exogenous provision of hypoxanthine and thymidine and in the presence of at least 10 nM methotrexate.

12. The mammalian cell of claim 11, wherein the second polynucleotide further comprises a second gene expressible in the mammalian cell.

13. A method of selecting for integration of a nucleic acid encoding a target protein into the genome of a cell comprising;
a) culturing a population of mammalian cells according to claim 11 or 12 in the presence of hypoxanthine and thymidine required by the cell to grow due to inhibition of expression of the natural cellular mRNA by the multi-hairpin amiRNA sequence;
b) transfecting the population of cells with a second polynucleotide comprising a gene encoding a dihydrofolate reductase expressible in the mammalian cells and a second gene encoding the target protein, wherein expression of the dihydrofolate reductase compensates for the inhibition of the expression of the natural cellular mRNA thereby restoring capacity to grow without hypoxanthine and thymidine and in the presence of at least 10 nM methotrexate;
c) culturing the transfected cells with a reduced concentration or absence of the hypoxanthine and thymidine, and optionally the presence of between 10 nM and 2 uM methotrexate wherein transfected cells surviving culturing have integrated the second polynucleotide into their genomes and can thereby express the target protein.
Synthetic amiRNA UTR

14. A polynucleotide comprising
a) an open reading frame operably linked to a first promoter that is active in a eukaryotic cell,
b) a polyadenylation signal sequence that is active in a eukaryotic cell,
c) a sequence selected from SEQ ID NOs: 558-561, located between the open reading frame and the polyadenylation signal sequence,
wherein the open reading frame does not encode Cricetulus griseus alpha-(1,6)-fucosyl transferase or Cricetulus griseus glutamine synthetase.

15. A method of inhibiting expression of an open reading frame in a eukaryotic cell, comprising introducing into the eukaryotic cell (i) the polynucleotide of claim 14 and (ii) a polynucleotide encoding a multi-hairpin amiRNA comprising a sequence selected from SEQ ID NOs: 193, 194, 195 and 209 or the multi-hairpin amiRNA, wherein the multi-hairpin amiRNA inhibits expression of the open reading frame.

16. The method of claim 15, wherein the polynucleotide encoding the multi-hairpin amiRNA is operably linked to a second promoter that is active in the cell.

17. The method of claim 16, wherein the second promoter is inducible.

18. The method of claim 16, wherein the second promoter is constitutive.

19. The method of any one of claims 15-18, wherein the eukaryotic cell is a mammalian cell.

20. The method of any one of claims 15-18, wherein the eukaryotic cell is a human cell.

21. The method of any one of claims 15-18, wherein the eukaryotic cell is a rodent cell.

22. A cell comprising (i) the polynucleotide of claim 14 and (ii) a polynucleotide encoding a multi-hairpin amiRNA comprising a sequence selected from SEQ ID NOs: 193, 194, 195 and 209 or the multi-hairpin amiRNA.
General Multi-Hairpin amiRNA

23. A polynucleotide comprising
a) a segment encoding a multi-hairpin amiRNA sequence, wherein the segment comprises
i) a first guide strand sequence comprising a contiguous 19 nucleotide sequence that is perfectly complementary to a first target site of a natural mammalian cellular mRNA and a first passenger strand sequence comprising a contiguous 19 nucleotide sequence that is at least 78% complementary to the first guide strand sequence, wherein the first guide strand and first passenger strand sequence are separated by between 5 and 35 nucleotides;
ii) a second guide strand sequence comprising a contiguous 19 nucleotide sequence that is perfectly complementary to a second target site different than the first target site of the same natural mammalian cellular mRNA as the first guide strand sequence and a second passenger strand sequence comprising a contiguous 19 nucleotide sequence that is at least 78% complementary to the second guide strand sequence, wherein the second guide strand and second passenger strand sequence are separated by between 5 and 35 nucleotides, and wherein the first and second guide strand sequence are different from each other; and
b) a eukaryotic promoter that is active in a mammalian cell and is transcribed by RNA polymerase II or RNA polymerase III, operably linked to the segment encoding the amiRNA sequence, wherein the amiRNA sequence can be expressed and fold into multiple hairpins.

24. The polynucleotide of claim 23, wherein the multi-hairpin amiRNA sequence reduces expression of the natural cellular mRNA to a greater extent than a control polynucleotide expressing tandem copies of the amiRNA hairpin comprising the first guide strand sequence, or a control polynucleotide expressing tandem copies of the amiRNA hairpin comprising the second guide strand sequence of the polynucleotide of claim 23.

25. The polynucleotide of claim 23 or 24, wherein the first guide strand sequence is a 19-22 nucleotide sequence perfectly complementary to the natural mammalian cellular mRNA and the first passenger strand sequence has the same length as the first guide sequence.

26. The polynucleotide of claim 23 or 24, wherein the first guide strand sequence is a 19-22 nucleotide sequence perfectly complementary to the natural mammalian cellular mRNA and the first passenger strand sequence is shorter than the first guide sequence.

27. The polynucleotide of any one of claims 23-26, wherein the first and second target sites do not overlap.

28. The polynucleotide of any one of claims 23-27, wherein the segment encoding the multi-hairpin amiRNA sequence further comprises a third guide strand sequence comprising a contiguous 19 nucleotide sequence that is perfectly complementary to the same natural mammalian cellular mRNA as the first and second guide strand sequences and a third passenger strand sequence comprising a contiguous 19 nucleotide sequence that is at least 78% complementary to the third guide strand sequence, wherein the third guide strand and third passenger strand sequence are separated by between 5 and 35 nucleotides, and wherein the first, second and third guide strand sequences are different from each other.

29. The polynucleotide of any one of claims 23-28, further comprising two transposon ends flanking the multi-hairpin amiRNA segment and the promoter, wherein the segment and the promoter are transposable by a corresponding transposase.

30. The polynucleotide of claim 29, wherein each transposon end comprises a sequence selected from SEQ ID NOs: 421 and 422, or from SEQ ID NOs: 427 and 428, or from SEQ ID NOs: 431 and 432, or from SEQ ID NOs: 433 and 434, or from SEQ ID NOs: 439 and 440, or from SEQ ID NOs: 443 and 444, or from SEQ ID NOs: 564 and 447, or from SEQ ID NOs: 452 and 453, or from SEQ ID NOs: 456 and 457, or from SEQ ID NOs: 460 and 461, or from SEQ ID NOs: 528 and 529.

31. The polynucleotide of any one of claims 23-30, further comprising an open reading frame operably linked to the promoter, wherein the multi-hairpin amiRNA sequence is expressed from the promoter in a 3′ UTR following the open reading frame.

32. The polynucleotide of claim 31, wherein the open reading frame encodes a selectable marker.

33. The polynucleotide of claim 31, wherein the open reading frame encodes a fluorescent protein.

34. The polynucleotide of claim 32, wherein the selectable marker provides a growth advantage to the cell either by allowing the cell to synthesize a metabolically useful substance, or to survive in the presence of a harmful substance such as an antibiotic, enzyme inhibitor or cellular poison.

35. The polynucleotide of claim 32, wherein the selectable marker is selected from a dihydrofolate reductase, a glutamine synthetase, an aminoglycoside 3′-phosphotransferase, a puromycin acetyltransferase, a blasticidin acetyltransferase, a blasticidin deaminase, a hygromycin B phosphotransferase or a zeocin-binding protein.

36. The polynucleotide of any one of claims 23-35, wherein the promoter is an EF1a promoter, a promoter from the immediate early genes 1, 2 or 3 of cytomegalovirus, a promoter for eukaryotic elongation factor 2, a glyceraldehyde 3-phosphate dehydrogenase promoter, an actin promoter, a phosphoglycerokinase promoter, a ubiquitin promoter, a herpes simplex virus thymidine kinase promoter or a simian virus 40 promoter.

37. The polynucleotide of any one of claims 23-35, wherein the promoter is at least 95% identical to a nucleotide sequence selected from SEQ ID NOs: 310-399 and 404-409

38. The polynucleotide of any one of claims 23-37, wherein each passenger strand sequence is not complementary to its corresponding guide strand sequence at the position corresponding to the first base of the guide strand sequence.

39. The polynucleotide of any one of claims 23-38, wherein each passenger strand sequence is not complementary to its corresponding guide strand sequence at the position corresponding to the twelfth base of the guide strand sequence.

40. The polynucleotide of any one of claims 23-39, wherein each 5-35 nucleotide unstructured loop sequence between a guide strand sequence and its corresponding passenger strand sequence comprises a sequence selected from SEQ ID NOs: 241-250.

41. The polynucleotide of any one of claims 23-40, wherein each guide strand-passenger strand hairpin further comprises additional sequences immediately to the 5′ and 3′ of the hairpin, wherein the additional sequence are SEQ ID NO: 255 to the 5′ and SEQ ID NO: 256 to the 3′, or SEQ ID NO: 257 to the 5′ and SEQ ID NO: 258 to the 3′, or SEQ ID NO: 259 to the 5′ and SEQ ID NO: 260 to the 3′, or SEQ ID NO: 261 to the 5′ and SEQ ID NO: 262 to the 3′, or SEQ ID NO: 263 to the 5′ and SEQ ID NO: 264 to the 3′, or SEQ ID NO: 265 to the 5′ and SEQ ID NO: 266 to the 3′, or SEQ ID NO: 267 to the 5′ and SEQ ID NO: 268 to the 3′, or SEQ ID NO: 269 to the 5′ and SEQ ID NO: 270 to the 3′.

42. The polynucleotide of any one of claims 23-41, wherein the polynucleotide is integrated into the genome of a mammalian cell.

43. The polynucleotide of claim 23, which is effective to reduce expression of a target gene encoding the mRNA, or the function or the activity of the mRNA or a protein expressed therefrom, to less than 20% of the level in a control mammalian cell in which the polynucleotide is not expressed.

44. The polynucleotide of claim 42 or 43, wherein the mammalian cell is a hamster cell.

45. The polynucleotide of claim 42 or 43, wherein the mammalian cell is a human cell.

46. The mammalian cell of claim 42.

47. The mammalian cell of claim 46, wherein the expression of the target gene or the function or the activity of the product of the target gene is reduced to less than 20% of its normal level, compared with an equivalent mammalian cell whose genome does not comprise the polynucleotide.
Sialidase amiRNA

48. A polynucleotide comprising
a) a segment encoding a multi-hairpin amiRNA sequence, wherein the segment comprises
i) a first guide strand sequence comprising a contiguous 19 nucleotide sequence that is perfectly complementary to a first target site of a natural mammalian cellular mRNA and a first passenger strand sequence comprising a contiguous 19 nucleotide sequence that is at least 78% complementary to the first guide strand sequence, wherein the first guide strand and first passenger strand sequence are separated by between 5 and 35 nucleotides;
ii) a second guide strand sequence comprising a contiguous 19 nucleotide sequence that is perfectly complementary to a second target site different than the first target site of the same natural mammalian cellular mRNA as the first guide strand sequence and a second passenger strand sequence comprising a contiguous 19 nucleotide sequence that is at least 78% complementary to the second guide strand sequence, wherein the second guide strand and second passenger strand sequence are separated by between 5 and 35 nucleotides, and wherein the first and second guide strand sequence are different from each other; and
b) a eukaryotic promoter that is active in a mammalian cell and is transcribed by RNA polymerase II or RNA polymerase III, operably linked to the segment encoding the amiRNA sequence, wherein the amiRNA sequence can be expressed and fold into multiple hairpins;
wherein the natural mammalian cellular mRNA encodes an enzyme that reduces protein sialylation.

49. The polynucleotide of claim 48, wherein the natural mammalian cellular mRNA encodes a sialidase.

50. The polynucleotide of claim 49, wherein the natural mammalian cellular mRNA comprises a sequence that is at least 98% identical to a sequence selected from SEQ ID NOs: 13-18 or from SEQ ID NOs: 570-571.

51. The polynucleotide of claim 49, wherein the first and second guide strand sequences are selected from SEQ ID NOs: 85-89 or 565.

52. The polynucleotide of claim 49, wherein the first and second guide strand sequences are selected from SEQ ID NOs: 90-94.

53. The polynucleotide of claim 49, wherein the polynucleotide comprises a sequence selected from SEQ ID NOs: 212-225 or 567-569 comprising or encoding the multi-hairpin amiRNA sequence.
Sialidase Method

54. A method for increasing sialylation in a mammalian cell, comprising introducing into the mammalian cell
a) the polynucleotide of any one of claims 49-53 flanked by transposon ends; and
b) a corresponding transposase, wherein the transposase integrates the polynucleotide into the genome of the mammalian cell, whereby the mammalian cell produces a secreted protein with an increased level of sialylation relative to a control cell whose genome lacks the polynucleotide.

55. The method of claim 54, wherein the corresponding transposase is introduced as a polynucleotide encoding the transposase.

56. The method of claim 55, wherein the polynucleotide encoding the transposase is an mRNA.

57. The method of claim 55, wherein the polynucleotide encoding the transposase is DNA, and comprises an open reading frame encoding the transposase operably linked to a promoter active in the mammalian cell.

58. The method of claim 54, wherein the transposase is provided as transposase protein.

59. The method of any one of claims 54-58, wherein the genome of the mammalian cell further comprises a heterologous polynucleotide encoding the secreted protein, and the secreted protein is not naturally produced by the cell.

60. The method of any one of claims 54-59, further comprising
a) introducing into the cell the heterologous polynucleotide encoding the secreted protein, wherein the secreted protein is not naturally produced by the cell

61. The method of claim 60, wherein the polynucleotide of (a) is introduced into the cell before the polynucleotide of (c).

62. The method of claim 60, wherein the polynucleotide of (c) is introduced into the cell before the polynucleotide of (a).

63. The method of claim 60, wherein the polynucleotide of (a) is introduced into the cell at the same time as the polynucleotide of (c).

64. The method of claim 60, wherein the polynucleotide of (a) is carried on the same DNA molecule as the polynucleotide of (c).

65. The method of any one of claims 54-64, further comprising purifying the secreted protein.

66. The method of any one of claims 54-65, further comprising identifying the cell with the polynucleotide integrated into its genome.

67. The method of any one of claims 54-66, wherein the mammalian cell is a human cell

68. The method of any one of claims 54-66, wherein the mammalian cell is a CHO cell.

69. A mammalian cell produced by the method of any one of claims 54-68.

70. A mammalian cell comprising the polynucleotide of any one of claims 48-53, wherein the polynucleotide is expressed to produce the multi-hairpin amiRNA sequence, which inhibits expression of the enzyme that reduces protein sialylation.

71. The mammalian cell of claim 70, further comprising a heterologous polynucleotide encoding a secreted protein not naturally produced by the cell, wherein sialylation of the secreted protein is increased compared with expression in a control cell lacking the polynucleotide expressed to produce the amiRNA sequence.
LPL amiRNA

72. A polynucleotide comprising
a) a segment encoding a multi-hairpin amiRNA sequence, wherein the segment comprises
i) a first guide strand sequence comprising a contiguous 19 nucleotide sequence that is perfectly complementary to a first target site in a natural mammalian cellular mRNA of SEQ ID NO: 22 and a first passenger strand sequence comprising a contiguous 19 nucleotide sequence that is at least 78% complementary to the first guide strand sequence, wherein the first guide strand and first passenger strand sequence are separated by between 5 and 35 nucleotides;
ii) a second guide strand sequence comprising a contiguous 19 nucleotide sequence that is perfectly complementary to a second target site different than the first target site in the same natural mammalian cellular mRNA as the first guide strand sequence and a second passenger strand sequence comprising a contiguous 19 nucleotide sequence that is at least 78% complementary to the second guide strand sequence, wherein the second guide strand and second passenger strand sequences are separated by between 5 and 35 nucleotides, and wherein the first and second guide strand sequences are different from each other; and
b) a eukaryotic promoter that is active in a mammalian cell and is transcribed by RNA polymerase II or RNA polymerase III operably linked to the segment encoding the amiRNA sequence, wherein the amiRNA sequence can be expressed and fold into multiple hairpins;
wherein the natural mammalian cellular mRNA encodes a fatty acid hydrolase.

73. The polynucleotide of claim 72, wherein the natural mammalian cellular mRNA comprises a sequence that is at least 98% identical to SEQ ID NO: 572 or 590-592.

74. The polynucleotide of claim 73, wherein the first and second guide strand sequences are selected from SEQ ID Nos: 573-578.

75. The polynucleotide of claim 73, wherein the polynucleotide comprises a sequence selected from SEQ ID NOs: 585-589.
LPL Method

76. A method for reducing lipoprotein lipase in a mammalian cell, comprising introducing into a mammalian cell
a) the polynucleotide of any one of claims 72-75; and
b) a corresponding transposase, wherein the transposase integrates the polynucleotide into the genome of the cell, wherein expression of lipoprotein lipase is reduced.

77. The method of claim 76, wherein a level of the lipoprotein contaminating a secreted protein produced by the cell is reduced.

78. The method of claim 76 or 77, wherein the corresponding transposase is introduced as a polynucleotide encoding the transposase.

79. The method of claim 78, wherein the polynucleotide encoding the transposase is an mRNA.

80. The method of claim 78, wherein the polynucleotide encoding the transposase is DNA, and comprises an open reading frame encoding the transposase that is operably linked to a promoter that is active in the mammalian cell.

81. The method of claim 76, wherein the transposase is provided as a transposase protein.

82. The method of any one of claims 76-81, wherein the genome of the mammalian cell further comprises a gene encoding the secreted protein, and the secreted protein is not naturally produced by the cell.

83. The method of claim 77, further comprising:
c) introducing into the cell the gene encoding the secreted protein.

84. The method of claim 83, wherein the polynucleotide of (a) is introduced into the cell before the gene of (c).

85. The method of claim 83, wherein the gene of (c) is introduced into the cell before the polynucleotide of (a).

86. The method of claim 83, wherein the polynucleotide of (a) is introduced into the cell at the same time as the gene of (c).

87. The method of claim 86, wherein the polynucleotide of (a) is carried on the same DNA molecule as the gene of (c).

88. The method of any one of claims 77-87, further comprising purifying the secreted protein.

89. The method of claim 76, further comprising identifying a cell whose genome comprises the polynucleotide of claim 72.

90. The method of any one of claims 76-89, wherein the mammalian cell is a CHO cell.

91. A mammalian cell produced by the method of any one of claims 76-90.
IFN amiRNA

92. The polynucleotide of any one of claims 23-45, wherein the natural mammalian cellular mRNA encodes a subunit of an interferon receptor.

93. The polynucleotide of claim 92, wherein the natural mammalian cellular mRNA comprises a sequence that is at least 98% identical to a sequence selected from SEQ ID NOs: 19-22.

94. The polynucleotide of claim 92, wherein the first and second guide strand sequences are selected from SEQ ID NOs: 95-101.

95. The polynucleotide of claim 92, wherein the first and second guide strand sequences are selected from SEQ ID NOs: 102-107.

96. The polynucleotide of claim 92, wherein the polynucleotide comprises a sequence selected from SEQ ID NOs: 226-240.

97. The polynucleotide of any one of claims 92-96, further comprising an open reading frame encoding an interferon polypeptide, operably linked to a promoter active in a mammalian cell.
IFN Method

98. A method for reducing expression of an interferon receptor in a mammalian cell, comprising introducing into the mammalian cell
a) the polynucleotide of any one of claims 92-97 flanked by transposon ends; and
b) a corresponding transposase, wherein the transposase integrates the polynucleotide into the genome of the cell, and the polynucleotide expresses an amiRNA that reduces expression of the interferon receptor.

99. The method of claim 98, wherein the corresponding transposase is introduced as a polynucleotide encoding the transposase.

100. The method of claim 99, wherein the polynucleotide encoding the transposase is an mRNA.

101. The method of claim 99, wherein the polynucleotide encoding the transposase is DNA, and comprises an open reading frame encoding the transposase that is operably linked to a promoter that is active in the mammalian cell.

102. The method of claim 98, wherein the transposase is provided as transposase protein.

103. The method of any one of claims 98-102, wherein the genome of the mammalian cell further comprises a heterologous polynucleotide encoding an interferon polypeptide, expressible in the cell.

104. The method of claim 103, further comprising
a) introducing into the cell the heterologous polynucleotide encoding the interferon polypeptide.

105. The method of claim 104, wherein the polynucleotide of (a) is introduced into the cell before the polynucleotide of (c).

106. The method of claim 104, wherein the polynucleotide of (c) is introduced into the cell before the polynucleotide of (a).

107. The method of claim 104, wherein the polynucleotide of (a) is introduced into the cell at the same time as the polynucleotide of (c).

108. The method of claim 104, wherein the polynucleotide of (a) is carried on the same DNA molecule as the polynucleotide of (c).

109. The method of any one of claims 103-108, further comprising purifying the interferon.

110. The method of any one of claims 98-109, further comprising identifying the cell whose genome comprises the polynucleotide that expresses an amiRNA that reduces expression of an interferon receptor.

111. The method of any one of claims 98-110, wherein the mammalian cell is a human cell.

112. The method of any one of claims 98-110, wherein the mammalian cell is a CHO cell.

113. The mammalian cell produced by the method of any one of claims 98-112.

114. A mammalian cell comprising the polynucleotide of any one of claims 92-97, wherein the polynucleotide is expressed to produce the multi-hairpin amiRNA sequence, which inhibits expression of the subunit of the interferon receptor.

115. The mammalian cell of claim 114 further comprising a heterologous polynucleotide encoding an interferon, which is expressed with reduced toxicity to the cell compared with a control cell lacking the polynucleotide expressed to produce the amiRNA sequence.
Modification of gene to include target sites for amiRNA

116. A method of inhibiting expression of a gene in a mammalian cell, comprising modifying the mammalian cell so it expresses an mRNA encoded by the gene fused to a segment including first and second target sites different from each other; introducing in the mammalian cell a polynucleotide comprising
a) a segment encoding a multi-hairpin amiRNA sequence, wherein the segment comprises
i) a first guide strand sequence comprising a contiguous 19 nucleotide sequence that is perfectly complementary to the first target site and a first passenger strand sequence comprising a contiguous 19 nucleotide sequence that is at least 78% complementary to the first guide strand sequence, wherein the first guide strand and first passenger strand sequence are separated by between 5 and 35 nucleotides;
ii) a second guide strand sequence comprising a contiguous 19 nucleotide sequence that is perfectly complementary to the second target site and a second passenger strand sequence comprising a contiguous 19 nucleotide sequence that is at least 78% complementary to the second guide strand sequence, wherein the second guide strand and second passenger strand sequence are separated by between 5 and 35 nucleotides, and wherein the first and second guide strand sequence are different from each other; and
b) a eukaryotic promoter that is active in a mammalian cell and is transcribed by RNA polymerase II or RNA polymerase III, operably linked to the segment encoding the amiRNA sequence, wherein the amiRNA sequence can be expressed and fold into multiple hairpins, wherein the multi-hairpin amiRNA sequence binds to the first and second target sites via the first and second guide strand sequences inhibiting expression of the gene.

117. The method of claim 116, wherein the segment including the first and second target sites is fused within the 3′ UTR of the mRNA.
